SEQUENCE LISTING (66 sequences)

SEQ ID NO: 1 - DNA, 19 bp, Artificial Sequence
  Synthetic
aagagcaacaaatctgact

SEQ ID NO: 2 - DNA, 58 bp, Artificial Sequence
  Synthetic
aagagcaacagtgctgtgcctggagcaacaaatctgactaagagcaacaaatctgact

SEQ ID NO: 3 - DNA, 52 bp, Artificial Sequence
  Synthetic
aagagcaacagtgctggagcaacaaatctgactaagagcaacaaatctgact

SEQ ID NO: 4 - DNA, 53 bp, Artificial Sequence
  Synthetic
aagagcaacagtgcctggagcaacaaatctgactaagagcaacaaatctgact

SEQ ID NO: 5 - DNA, 38 bp, Artificial Sequence
  Synthetic
aagagcaacagtgctgactaagagcaacaaatctgact

SEQ ID NO: 6 - DNA, 60 bp, Artificial Sequence
  Synthetic
aagagcaacagtgctgtgggcctggagcaacaaatctgactaagagcaacaaatctgact

SEQ ID NO: 7 - DNA, 57 bp, Artificial Sequence
  Synthetic
aagagcaacagtgctggcctggagcaacaaatctgactaagagcaacaaatctgact

SEQ ID NO: 8 - DNA, 60 bp, Artificial Sequence
  Synthetic
aagagcaacagtgctgtgtgcctggagcaacaaatctgactaagagcaacaaatctgact

SEQ ID NO: 9 - DNA, 79 bp, Artificial Sequence
  Synthetic
cgtggccttagctgtgctcgcgctactctctctttctgcctggaggctatccagcgtgagtctctcctaccctcccgct

SEQ ID NO: 10 - DNA, 78 bp, Artificial Sequence
  Synthetic
cgtggccttagctgtgctcgcgctactctctctttcgcctggaggctatccagcgtgagtctctcctaccctcccgct

SEQ ID NO: 11 - DNA, 75 bp, Artificial Sequence
  Synthetic
cgtggccttagctgtgctcgcgctactctctctttctggaggctatccagcgtgagtctctcctaccctcccgct

SEQ ID NO: 12 - DNA, 84 bp, Artificial Sequence
  Synthetic
cgtggccttagctgtgctcgcgctactctctctttctggatagcctggaggctatccagcgtgagtctctcctaccctcccgct

SEQ ID NO: 13 - DNA, 55 bp, Artificial Sequence
  Synthetic
cgtggccttagctgtgctcgcgctatccagcgtgagtctctcctaccctcccgct

SEQ ID NO: 14 - DNA, 82 bp, Artificial Sequence
  Synthetic
cgtggccttagctgtgctcgcgctactctctctttctgtggcctggaggctatccagcgtgagtctctcctaccctcccgct

SEQ ID NO: 15 - RNA, 100 nt, Artificial Sequence
  Synthetic
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 16 - RNA, 96 nt, Artificial Sequence
  Synthetic
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugc

SEQ ID NO: 17 - RNA, 114 nt, Artificial Sequence
  Synthetic
  misc_feature (1)..(17): n is a, c, g, or u
  misc_feature (18)..(30): may be absent
  misc_feature (108)..(114): may be absent
nnnnnnnnnnnnnnnnnnnnnnnnnnnnnnguuuuagagcuagaaauagcaaguuaaaauaaggcuaguccguuaucaacuugaaaaaguggcaccgagucggugcuuuuuuuu

SEQ ID NO: 18 - RNA, 100 nt, Artificial Sequence
  Synthetic
agagcaacagugcuguggccguuuuagagcuagaaauagcaaguuaaaauaaggcuaguccguuaucaacuugaaaaaguggcaccgagucggugcuuuu

SEQ ID NO: 19 - RNA, 20 nt, Artificial Sequence
  Synthetic
agagcaacagugcuguggcc

SEQ ID NO: 20 - RNA, 100 nt, Artificial Sequence
  Synthetic
gcuacucucucuuucuggccguuuuagagcuagaaauagcaaguuaaaauaaggcuaguccguuaucaacuugaaaaaguggcaccgagucggugcuuuu

SEQ ID NO: 21 - RNA, 20 nt, Artificial Sequence
  Synthetic
gcuacucucucuuucuggcc

SEQ ID NO: 22 - RNA, 100 nt, Artificial Sequence; identical to SEQ ID NO: 18
  Synthetic
  misc_feature (1)..(4): modified with 2′-O-methyl phosphorothioate
  misc_feature (97)..(100): modified with 2′-O-methyl phosphorothioate

SEQ ID NO: 23 - RNA, 20 nt, Artificial Sequence
  Synthetic
  misc_feature (1)..(4): modified with 2′-O-methyl phosphorothioate
agagcaacagugcuguggcc

SEQ ID NO: 24 - RNA, 100 nt, Artificial Sequence; identical to SEQ ID NO: 20
  Synthetic
  misc_feature (1)..(4): modified with 2′-O-methyl phosphorothioate
  misc_feature (97)..(100): modified with 2′-O-methyl phosphorothioate

SEQ ID NO: 25 - RNA, 20 nt, Artificial Sequence
  Synthetic
  misc_feature (1)..(4): modified with 2′-O-methyl phosphorothioate
gcuacucucucuuucuggcc

SEQ ID NO: 26 - DNA, 20 bp, Artificial Sequence
  Synthetic
agagcaacagtgctgtggcc

SEQ ID NO: 27 - DNA, 20 bp, Artificial Sequence
  Synthetic
gctactctctctttctggcc

SEQ ID NO: 28 - DNA, 23 bp, Artificial Sequence
  Synthetic
agagcaacagtgctgtggcctgg

SEQ ID NO: 29 - DNA, 23 bp, Artificial Sequence
  Synthetic
gctactctctctttctggcctgg

SEQ ID NO: 30 - protein, 22 aa, Artificial Sequence
  Synthetic
MLLLVTSLLLCELPHPAFLLIP

SEQ ID NO: 31 - protein, 21 aa, Artificial Sequence
  Synthetic
MALPVTALLLPLALLLHAARP

SEQ ID NO: 32 - protein, 23 aa, Artificial Sequence
  Synthetic
IYIWAPLAGTCGVLLLSLVITLY

SEQ ID NO: 33 - DNA, 126 bp, Artificial Sequence
  Synthetic
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 34 - protein, 42 aa, Artificial Sequence
  Synthetic
KRGRKKLLYIFKQPFMRPVQTTQEEDGCSCRFPEEEEGGCEL

SEQ ID NO: 35 - DNA, 120 bp, Artificial Sequence
  Synthetic
tcaaagcggagtaggttgttgcattccgattacatgaatatgactcctcgccggcctgggccgacaagaaaacattaccaaccctatgcccccccacgagacttcgctgcgtacaggtcc

SEQ ID NO: 36 - protein, 40 aa, Artificial Sequence
  Synthetic
SKRSRLLHSDYMNMTPRRPGPTRKHYQPYAPPRDFAAYRS

SEQ ID NO: 37 - DNA, 336 bp, Artificial Sequence
  Synthetic
cgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 38 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - DNA, 1518 bp, Artificial Sequence
  Synthetic
atgcttcttttggttacgtctctgttgctttgcgaacttcctcatccagcgttcttgctgatccccgatattcagatgactcagaccaccagtagcttgtctgcctcactgggagaccgagtaacaatctcctgcagggcaagtcaagacattagcaaatacctcaattggtaccagcagaagcccgacggaacggtaaaactcctcatctatcatacgtcaaggttgcattccggagtaccgtcacgattttcaggttctgggagcggaactgactattccttgactatttcaaacctcgagcaggaggacattgcgacatatttttgtcaacaaggtaataccctcccttacactttcggaggaggaaccaaactcgaaattaccgggtccaccagtggctctgggaagcctggcagtggagaaggttccactaaaggcgaggtgaagctccaggagagcggccccggtctcgttgcccccagtcaaagcctctctgtaacgtgcacagtgagtggtgtatcattgcctgattatggcgtctcctggataaggcagcccccgcgaaagggtcttgaatggcttggggtaatatggggctcagagacaacgtattataactccgctctcaaaagtcgcttgacgataataaaagataactccaagagtcaagttttccttaaaatgaacagtttgcagactgacgataccgctatatattattgtgctaaacattattactacggcggtagttacgcgatggattattgggggcaggggacttctgtcacagtcagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 40 - protein, 506 aa, Artificial Sequence
  Synthetic
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - DNA, 800 bp, Artificial Sequence
  Synthetic
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttca

SEQ ID NO: 42 - DNA, 804 bp, Artificial Sequence
  Synthetic
tggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 43 - DNA, 1178 bp, Artificial Sequence
  Synthetic
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 44 - DNA, 66 bp, Artificial Sequence
  Synthetic
atgcttcttttggttacgtctctgttgctttgcgaacttcctcatccagcgttcttgctgatcccc

SEQ ID NO: 45 - protein, 22 aa, Artificial Sequence
  Synthetic
MLLLVTSLLLCELPHPAFLLIP

SEQ ID NO: 46 - DNA, 735 bp, Artificial Sequence
  Synthetic
gatattcagatgactcagaccaccagtagcttgtctgcctcactgggagaccgagtaacaatctcctgcagggcaagtcaagacattagcaaatacctcaattggtaccagcagaagcccgacggaacggtaaaactcctcatctatcatacgtcaaggttgcattccggagtaccgtcacgattttcaggttctgggagcggaactgactattccttgactatttcaaacctcgagcaggaggacattgcgacatatttttgtcaacaaggtaataccctcccttacactttcggaggaggaaccaaactcgaaattaccgggtccaccagtggctctgggaagcctggcagtggagaaggttccactaaaggcgaggtgaagctccaggagagcggccccggtctcgttgcccccagtcaaagcctctctgtaacgtgcacagtgagtggtgtatcattgcctgattatggcgtctcctggataaggcagcccccgcgaaagggtcttgaatggcttggggtaatatggggctcagagacaacgtattataactccgctctcaaaagtcgcttgacgataataaaagataactccaagagtcaagttttccttaaaatgaacagtttgcagactgacgataccgctatatattattgtgctaaacattattactacggcggtagttacgcgatggattattgggggcaggggacttctgtcacagtcagtagt

SEQ ID NO: 47 - protein, 245 aa, Artificial Sequence
  Synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 48 - DNA, 261 bp, Artificial Sequence
  Synthetic
gctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgc

SEQ ID NO: 49 - DNA, 252 bp, Artificial Sequence
  Synthetic
tttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgc

SEQ ID NO: 50 - protein, 84 aa, Artificial Sequence
  Synthetic
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 51 - protein, 120 aa, Artificial Sequence
  Synthetic
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 52 - protein, 107 aa, Artificial Sequence
  Synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 53 - protein, 18 aa, Artificial Sequence
  Synthetic
GSTSGSGKPGSGEGSTKG

SEQ ID NO: 54 - DNA, 4358 bp, Artificial Sequence
  Synthetic
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatgcttcttttggttacgtctctgttgctttgcgaacttcctcatccagcgttcttgctgatccccgatattcagatgactcagaccaccagtagcttgtctgcctcactgggagaccgagtaacaatctcctgcagggcaagtcaagacattagcaaatacctcaattggtaccagcagaagcccgacggaacggtaaaactcctcatctatcatacgtcaaggttgcattccggagtaccgtcacgattttcaggttctgggagcggaactgactattccttgactatttcaaacctcgagcaggaggacattgcgacatatttttgtcaacaaggtaataccctcccttacactttcggaggaggaaccaaactcgaaattaccgggtccaccagtggctctgggaagcctggcagtggagaaggttccactaaaggcgaggtgaagctccaggagagcggccccggtctcgttgcccccagtcaaagcctctctgtaacgtgcacagtgagtggtgtatcattgcctgattatggcgtctcctggataaggcagcccccgcgaaagggtcttgaatggcttggggtaatatggggctcagagacaacgtattataactccgctctcaaaagtcgcttgacgataataaaagataactccaagagtcaagttttccttaaaatgaacagtttgcagactgacgataccgctatatattattgtgctaaacattattactacggcggtagttacgcgatggattattgggggcaggggacttctgtcacagtcagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 55 - protein, 1368 aa, Artificial Sequence [sequence omitted]

SEQ ID NO: 56 - protein, 300 aa, Artificial Sequence
  Synthetic
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 57 - protein, 452 aa, Artificial Sequence
  Synthetic
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 124 aa, Artificial Sequence
  Synthetic
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSAS

SEQ ID NO: 59 - protein, 213 aa, Artificial Sequence
  Synthetic
EIVLTQSPATLSLSPGERATLSCRASQSVSSYLAWYQQKPQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 107 aa, Artificial Sequence [sequence omitted]

SEQ ID NO: 61 - protein, 5 aa, Artificial Sequence
  Synthetic
SFAMS

SEQ ID NO: 62 - protein, 17 aa, Artificial Sequence
  Synthetic
AISGSGGGTYYADSVKG

SEQ ID NO: 63 - protein, 13 aa, Artificial Sequence
  Synthetic
DKILWFGEPVFDY

SEQ ID NO: 64 - protein, 11 aa, Artificial Sequence
  Synthetic
RASQSVSSYLA

SEQ ID NO: 65 - protein, 7 aa, Artificial Sequence
  Synthetic
DASNRAT

SEQ ID NO: 66 - protein, 9 aa, Artificial Sequence
  Synthetic
QQRSNWPPT

(19) United States
(12) Patent Application Publication
(10) Pub. No.: US 2022/0023344 A1
(43) Pub. Date: Jan. 27, 2022

(54) ALLOGENEIC CELL THERAPY OF ACUTE LYMPHOBLASTIC LEUKEMIA USING GENETICALLY ENGINEERED T CELLS TARGETING CD19

(71) Applicant: CRISPR Therapeutics AG, Zug (CH)

(72) Inventors: Jonathan Alexander TERRETT, Cambridge, MA (US); Demetrios KALAITZIDIS, Cambridge, MA (US); Ewelina MORAWA, Cambridge, MA (US); Mark BENTON, Cambridge, MA (US)

(21) Appl. No.: 17/359,041

(22) Filed: Jun. 25, 2021

Related U.S. Application Data
(60) Provisional application No. 63/164,685, filed on Mar. 23, 2021.
(60) Provisional application No. 63/044,456, filed on Jun. 26, 2020.
(60) Provisional application No. 63/044,388, filed on Jun. 26, 2020.

(51) Int. Cl. A61K 35/17; A61K 39/395; A61K 38/17; A61K 9/00; A61K 31/7076; A61K 31/675; A61P 35/02

(52) U.S. Cl. CPC A61K 35/17; A61K 39/3955; A61K 38/177; A61K 38/1774; A61K 2039/5156; A61K 31/7076; A61K 31/675; A61P 35/02; A61K 9/0019

(57) ABSTRACT

Methods for treating a B cell malignancy comprising a population of genetically engineered immune cells (e.g., T cells), which express a chimeric antigen receptor (CAR) specific to CD19 and optionally an NK cell inhibitor (e.g., daratumumab). The genetically engineered immune cells may comprise a disrupted TRAC gene, a disrupted β2M gene, or both.

### CROSS REFERENCE TO RELATED APPLICATIONS

This application claims the benefit of the filing dates of U.S. Provisional Application No. 63/044,456, filed Jun. 26, 2020, U.S. Provisional Application No. 63/044,388, filed Jun. 26, 2020, and U.S. Provisional Application No. 63/164,685, filed Mar. 23, 2021. The entire contents of each of the prior applications are incorporated by reference herein.

### SEQUENCE LISTING PARAGRAPH

The application contains a Sequence Listing that has been filed electronically in the form of a text file, created Jun. 24, 2021, and named “095136-0335-027US1_SEQ.TXT” (59,854 bytes), the contents of which are incorporated by reference herein in their entirety.

### BACKGROUND OF THE INVENTION

Chimeric antigen receptor (CAR) T cell therapies are adoptive T cell therapeutics used to treat human malignancies. Although CAR T cell therapy has led to tremendous clinical success, including durable remission in relapsed/refractory non-Hodgkin lymphoma (NHL) and pediatric acute lymphoblastic leukemia (ALL), the approved products are autologous and require patient-specific cell collection and manufacturing. Because of this, some patients have experienced disease progression or death while awaiting treatment. Allogeneic CAR T cell therapy, comprising disrupted MHC Class-I complexes, presents an attractive off-the-shelf option to autologous CAR T cell therapy. The disrupted MHC Class I in the allogeneic T cells, however, renders the CAR T cells susceptible to elimination by the host immune system, for example, by natural killer (NK) cell-mediated immune response. Accordingly, there remains a need for improved CAR T cell therapy.

### SUMMARY OF THE INVENTION

The present disclosure is based, at least in part, on the unexpected finding that a natural killer (NK) cell inhibitor (e.g., an anti-CD38 antibody such as daratumumab) in combination with genetically engineered T cells expressing an anti-CD19 chimeric antigen receptor (CAR) (e.g., CTX110 cells) reduced tumor burden and extended survival rates in a xenograft mouse model of acute lymphoblastic leukemia. Further, it was found that, unexpectedly, a natural killer (NK) cell inhibitor (e.g., an anti-CD38 antibody such as daratumumab) successfully depleted NK cells both in vitro and in vivo, but did not affect T cell numbers, and did not activate CAR T cells, which express surface CD38. Moreover, in a human clinical trial, the combination of anti-CD19 CAR-T cells (CTX110 cells) and an NK cell inhibitor (daratumumab) achieved complete response in at least one patient for at least 3 months after the treatment. The NK cell inhibitor also successfully prolonged survival and enhanced expansion of the MHC Class-I deficient CAR-T cells, which would otherwise be vulnerable to clearance by NK cells.

In addition, the present disclosure is also based, at least in part, on the development of allogeneic cell therapy for acute lymphoblastic leukemia (ALL) using genetically engineered T cells (e.g., CTX110 cells, a.k.a., TC1 cells) expressing an anti-CD19 chimeric antigen receptor (CAR) and having disrupted TRAC gene and B2M gene. The allogeneic CAR-T cell therapy disclosed herein showed treatment efficacies in human patients having ALL, including complete responses in certain patients and long durability of responses. Further, the allogeneic CAR-T cell therapy disclosed herein exhibited desired pharmacokinetic features in the human patients, including prolonged CAR-T cell expansion and persistence after infusion.

Accordingly, one aspect of the present disclosure features a method for treating a B-cell malignancy in a human patient, the method comprising: (i) administering to a human patient having a first dose of B-cell malignancy a natural killer (NK) cell inhibitor, and (ii) administering to the human patient a first dose of a population of genetically engineered T cells after step (ii). The population of genetically engineered T cells comprising T cells that comprise (a) a nucleic acid coding for a chimeric antigen receptor (CAR) that binds CD19. Optionally, the method may further comprise, between step (i) and step (ii), (iii) subjecting the human patient to a lymphodepletion treatment.

In some embodiments, the genetically engineered T cells are deficient in expression of MHC Class-I. In some instances, the population of genetically engineered T cells comprise T cells that comprise (b) a disrupted beta 2-microglobulin (β2M) gene. Alternatively or in addition, the population of genetically engineered T cells comprising T cells that comprise (c) a disrupted T cell receptor alpha constant (TRAC) gene.

In some embodiments, the first dose of the population of genetically engineered T cells is administered to the human patient at a dose of about 1×10⁷ to about 1×10⁹ CAR⁺ T cells. In some examples, the first dose of the population of genetically engineered T cells is about 3×10⁷ CAR⁺ T cells. In some examples, the first dose of the population of genetically engineered T cells is about 1×10⁸ CAR⁺ T cells. In some examples, the first dose of the population of genetically engineered T cells is about 3×10⁸ CAR⁺ T cells. In some examples, the first dose of the population of genetically engineered T cells is about 4.5×10⁸ CAR⁺ T cells. In some examples, the first dose of the population of genetically engineered T cells is about 6×10⁸ CAR⁺ T cells. In some examples, the first dose of the population of genetically engineered T cells is about 9×10⁸ CAR⁺ T cells. The population of genetically engineered T cells administered to the human patient per dose contains no more than 7×10⁴ TCR⁺ T cells/kg.

In some embodiments, the NK cell inhibitor comprises an anti-CD38 antibody. In some examples, the anti-CD38 antibody is daratumumab. In some instances, the first dose of daratumumab is about 16 mg/kg via intravenous infusion. In some examples, the first dose of daratumumab is about 16 mg/kg via intravenous infusion, which is administered to the human patient at 8 mg/kg per day for two consecutive days. Alternatively, the first dose of daratumumab is about 1800 mg via subcutaneous injection. In some embodiments, the first dose of NK cell inhibitor is administered to the human patient at least one day prior to the lymphodepletion treatment. For example, the first dose of the population of genetically engineered T cells may be administered to the human patient within 10 days after the first dose of the NK cell inhibitor.

Any of the methods disclosed herein may further comprise (iv) administering to the human patient at least one subsequent dose of the NK cell inhibitor after step (ii). In some embodiments, step (iv) comprises administering to the human patient a second dose of the NK inhibitor 24 to 32 days after the first dose of the population of the genetically engineered T cells. Optionally, step (iv) may further comprise a third dose of the NK inhibitor 56 to 64 days after the first dose of the population of genetically engineered T cells. The human patient may show stable disease (SD) or better at least 4 weeks after the first dose of the population of genetically engineered T cells. In some examples, the NK inhibitor is daratumumab. The second dose and/or the third dose of daratumumab may be 16 mg/kg by intravenous infusion. Alternatively, the second dose and/or the third dose of daratumumab is 1800 mg by subcutaneous injection.

In some embodiments, the lymphodepletion treatment in step (iii) comprises co-administration to the human patient fludarabine at about 30 mg/m² and cyclophosphamide at about 500 mg/m² per day for three days. Prior to step (iii), the human patient may not show one or more of the following features: (a) significant worsening of clinical status, (b) requirement for supplemental oxygen to maintain a saturation level of greater than 91%, (c) uncontrolled cardiac arrhythmia, (d) hypotension requiring vasopressor support, (e) active infection, (f) grade ≥2 acute neurological toxicity, and (g) unresolved infusion reaction due to the NK cell inhibitor.

In some instances, step (iii) may be performed about 2-7 days prior to step (ii). In some examples, after step (iii) and prior to step (ii), the human patient may not show one or more of the following features: (a) active uncontrolled infection; (b) worsening of clinical status compared to the clinical status prior to step (iii); and (c) grade ≥2 acute neurological toxicity.

Any of the methods disclosed herein may further comprise (v) monitoring the human patient for development of acute toxicity after step (ii); and (vi) managing the acute toxicity if the acute toxicity occurs. In some embodiments, step (v) is performed for at least 28 days after administration of the population of genetically engineered T cells. Exemplary acute toxicity comprises tumor lysis syndrome (TLS), cytokine release syndrome (CRS), immune effector cellassociated neurotoxicity syndrome (ICANS), B cell aplasia, hemophagocytic lymphohistiocytosis (HLH), cytopenia, graft-versus-host disease (GvHD), hypertension, viral encephalitis, renal insufficiency, or a combination thereof.

In some embodiments, the B cell malignancy is non-Hodgkin lymphoma. Examples include diffuse large B cell lymphoma (DLBCL), high grade B cell lymphoma with MYC and BCL2 and/or BCL6 rearrangement, transformed follicular lymphoma (FL), and grade 3b FL. In some examples, DLBCL is DLBCL not otherwise specified (NOS). In some examples, the human patient has at least one measurable lesion that is fluorodeoxyglucose positron emission tomography (PET)-positive. In some examples, the B cell malignancy is refractory and/or relapsed. In some examples, the human patient has undergone one or more lines of prior anti-cancer therapies. For example, the human patient may have undergone two or more lines of prior anti-cancer therapies. Exemplary prior anti-cancer therapies comprise an anti-CD20 antibody, an anthracycline-containing regimen, or a combination thereof.

In some instances, the human patient has refractory or relapsed transformed FL and has undergone at least one line of chemotherapy for disease after transformation to DLBCL. In some instances, the B cell malignancy is refractory, and the human patient has progressive disease on last therapy, or has stable disease following at least two cycles of therapy with duration of stable disease of up to 6 months. In some examples, the human patient has failed prior autologous hematopoietic stem cell transplantation (HSCT) or ineligible for prior autologous HSCT. In some examples, the human patient is subject to an additional anti-cancer therapy after treatment with the population of genetically engineered T cells.

In some instances, the human patient has one or more of the following features: (a) has an Eastern Cooperative Oncology Group (ECOG) performance status 0 or 1; (b) adequate renal, liver, cardiac, and/or pulmonary function; (c) free of prior gene therapy or modified cell therapy; (d) free of prior treatment comprising an anti-CD19 antibody; (e) free of prior allogeneic HSCT; (f) free of detectable malignant cells from cerebrospinal fluid; (g) free of brain metastases; (h) free of prior central nervous system disorders; (i) free of unstable angina, arrhythmia, and/or myocardial infarction; (j) free of uncontrolled infection; (k) free of immunodeficiency disorders or autoimmune disorders that require immunosuppressive therapy; and (l) free of infection by human immunodeficiency virus, hepatitis B virus, or hepatitis C virus. Alternatively or in addition, the human patient is not diagnosed for Burkitt's lymphoma or leukemia.

In some instances, the method disclosed herein may further comprise administering to the human patient having NHL at least one subsequent dose of the population of genetically engineered T cells. In some examples, the first dose of the population of genetically engineered T cells is at least 3×10⁸ CAR⁺ T cells. The human patient may receive a second dose of the population of genetically engineered T cells about 4-8 weeks after the first dose of the population of genetically engineered T cells. Such a human patient may achieve stable disease (SD), particle response (PR), or complete response (CR) at least 4 weeks after the first dose. In some instances, the human patient receives a subsequent lymphodepletion treatment about 2-7 days prior to each of the subsequent dose of the population of the genetically engineered T cells. If the human patient experiences significant cytopenias after steps (i)-(iii), the human patient may not receive subsequent lymphodepletion treatment prior to each of the subsequent dose of the population of the genetically engineered T cells.

In other examples, the first dose of the population of genetically engineered T cells is at least 4.5×10⁸ CAR⁺ T cells or at least 6×10⁸ CAR⁺ T cells. The human patient may receive a second dose of the population of genetically engineered T cells about 7-9 days after the first dose of the population of genetically engineered T cells. The human patient may not receive a subsequent lymphodepletion treatment prior to the second dose of the population of genetically engineered T cells. The human patient may receive a third dose of the population of genetically engineered T cells about 4-8 weeks after the first dose of the population of genetically engineered T cells. Such a human patient may achieve stable disease (SD), particle response (PR), or complete response (CR) at least 4 weeks after the first dose. In some instances, the human patient receives a subsequent lymphodepletion treatment about 2-7 days prior to the third dose of the population of the genetically engineered T cells. If the human patient experiences significant cytopenias after steps (i)-(iii), the human patient may not receive subsequent lymphodepletion treatment prior to the third dose of the population of the genetically engineered T cells.

In any of the methods disclosed above, the human patient may not receive subsequent doses of the NK inhibitor. Alternatively, one or more subsequent doses of the NK inhibitor may be administered to the human patient. Alternatively or in addition, the subsequent dose(s) of the genetically engineered T cells may be about 3×10⁷, about 1×10⁸, about 3×10⁸, about 4.5×10⁸, about 6×10⁸, or about 9×10⁸ CAR+ T cells.

In other embodiments, the human patient has B-cell acute lymphoblastic leukemia (ALL). In some instances, the human patient has refractory and/or relapsed B cell ALL. For example, the human patient may: (a) has undergone two or more lines of prior anti-cancer therapies; (b) has bone marrow relapse after allogeneic hematopoietic stem cell transplantation (HSCT); (c) is Philadelphia chromosome-positive (Ph+), and is intolerant to or ineligible for tyrosine kinase inhibitor (TKI) therapy, or has progressed after at least 1 line of TKI therapy; (d) has bone marrow involvement with <50% blasts; and/or (e) is bone marrow minimal residue disease (MRD) positive with <5% blasts.

In some instances, the human patient has one or more of the following features: (a) has an Eastern Cooperative Oncology Group (ECOG) performance status 0 or 1; (b) adequate renal, liver, cardiac, and/or pulmonary function; (c) free of prior gene therapy or modified cell therapy; (d) free of prior treatment comprising an anti-CD19 antibody; (e) free of prior allogeneic HSCT; (f) free of detectable malignant cells from cerebrospinal fluid; (g) free of brain metastases; (h) free of prior central nervous system disorders; (g) free of unstable angina, arrhythmia, and/or myocardial infarction; (h) free of uncontrolled infection; (i) free of immunodeficiency disorders or autoimmune disorders that require immunosuppressive therapy; and (j) free of infection by human immunodeficiency virus, hepatitis B virus, or hepatitis C virus. Alternatively or in addition, the human patient does not have isolated extramedullary disease.

In some examples, the method disclosed above may further comprise administering to the human patient at least one subsequent dose of the population of genetically engineered T cells. In some examples, the first dose of the population of genetically engineered T cells is at least 3×10⁷ CAR+ T cells, at least 1×10⁸ CAR⁺ T cells or at least 3×10⁸ CAR⁺ T cells. Alternatively or in addition, the subsequent dose(s) is about 3×10⁷, about 1×10⁸, about 3×10⁸, about 4.5×10⁸, about 6×10⁸, or about 9×10⁸ CAR+ T cells.

In some instances, the human patient has a decrease in bone marrow blast count of at least 50% about 4 weeks after the first dose of the population of genetically engineered T cells. The human patient may receives a second dose of the population of genetically engineered T cells about 4 to 8 weeks after the first dose of the population of genetically engineered T cells. In some instances, the human patient is in a morphologic remission and is MRD-remains positive. In some instances, the human patient shows progressive disease (PD) and had prior response. The human patient may receive a subsequent lymphodepletion treatment about 2-7 days prior to each of the subsequent dose of the population of the genetically engineered T cells. If the human patient experiences significant cytopenias after steps (i)-(iii), the human patient may not receive subsequent lymphodepletion treatment prior to each of the subsequent dose of the population of the genetically engineered T cells.

In another aspect, the present disclosure provides a method for treating acute lymphoblastic leukemia (ALL) in a human patient, the method comprising: (i) subjecting a human patient having ALL to a lymphodepletion treatment; and (ii) administering to the human patient a first dose of a population of genetically engineered T cells after step (i). The population of genetically engineered T cells comprising T cells comprise (a) a nucleic acid coding for a chimeric antigen receptor (CAR) that binds CD19. In some embodiments, the dose of the population of genetically engineered T cells is administered to the human patient at a dose of about 1×10⁷ to about 1×10⁹ CAR+ T cells. The population of genetically engineered T cells may comprise T cells comprising (b) a disrupted T cell receptor alpha constant (TRAC) gene, and/or (c) a disrupted beta 2-microglobulin (β2M) gene. In some examples, the population of genetically engineered T cells comprise T cells comprising both (b) and (c).

In some examples, the first dose of the population of genetically engineered T cells is about 3×10⁷ CAR⁺ T cells. In some examples, the first dose of the population of genetically engineered T cells is about 1×10⁸ CAR⁺ T cells. In some examples, the first dose of the population of genetically engineered T cells is about 3×10⁸ CAR⁺ T cells. In some examples, the first dose of the population of genetically engineered T cells is about 4.5×10⁸ CAR⁺ T cells. In some examples, the first dose of the population of genetically engineered T cells is about 6×10¹ CAR⁺ T cells. In some examples, the first dose of the population of genetically engineered T cells is about 9×10⁸ CAR⁺ T cells. In some examples, the first dose of the population of the genetically engineered T cells is at least 1×10⁸ CAR+ T cells. In other examples, the first dose of the population of genetically engineered T cells is at least about 3×10⁸ CAR⁺ T cells. The population of genetically engineered T cells administered to the human patient per dose contains no more than 7×10⁴ TCR⁺ T cells/kg.

In some instances, the lymphodepletion treatment in step (i) comprises co-administration to the human patient fludarabine at about 30 mg/m² and cyclophosphamide at about 500 mg/m² per day for three days. Prior to step (i), the human patient may not show one or more of the following features: (a) significant worsening of clinical status, (b) requirement for supplemental oxygen to maintain a saturation level of greater than 91%, (c) uncontrolled cardiac arrhythmia, (d) hypotension requiring vasopressor support, (e) active infection, and (f) grade ≥2 acute neurological toxicity. In some examples, step (i) is performed about 2-7 days prior to step (ii). In some examples, after step (i) and prior to step (ii), the human patient may not show one or more of the following features: (a) active uncontrolled infection; (b) worsening of clinical status compared to the clinical status prior to step (i); and (c) grade ≥2 acute neurological toxicity.

Any of the methods disclosed above may further comprise (iii) monitoring the human patient for development of acute toxicity after step (ii); and (iv) managing the acute toxicity if occurs. In some examples, step (iii) may be performed for at least 28 days after the first dose of the population of genetically engineered T cells. Exemplary acute toxicity comprises tumor lysis syndrome (TLS), cytokine release syndrome (CRS), immune effector cell-associated neurotoxicity syndrome (ICANS), B cell aplasia, hemophagocytic lymphohistiocytosis (HLH), cytopenia, graft-versus-host disease (GvHD), hypertension, renal insufficiency, viral encephalitis, or a combination thereof.

In some examples, the the human patient has B-cell ALL. For example, the human patient has refractory and/or relapsed B cell ALL. In some instances, the human patient may: (a) has undergone two or more lines of prior anti-cancer therapies; (b) has bone marrow relapse after allogeneic hematopoietic stem cell transplantation (HSCT); (c) is Philadelphia chromosome-positive (Ph+), and is intolerant to or ineligible for tyrosine kinase inhibitor (TKI) therapy, or has progressed after at least 1 line of TKI therapy; (d) has bone marrow involvement with <50% blasts; and/or (e) is bone marrow minimal residue disease (MRD) positive with <5% blasts. In some examples, the prior anti-cancer therapies comprise a chemotherapy, an allogeneic stem cell transplantation, or a combination thereof; optionally wherein the chemotherapy comprises vincristine, corticosteroids, an anthracycline-containing regimen, or a combination thereof. In some examples, the human patient is subject to an additional anti-cancer therapy after treatment with the population of genetically engineered T cells.

The ALL patient may have one or more of the following features: (a) has an Eastern Cooperative Oncology Group (ECOG) performance status 0 or 1; (b) adequate renal, liver, cardiac, and/or pulmonary function; (c) free of prior gene therapy or modified cell therapy; (d) free of prior treatment comprising an anti-CD19 antibody; (e) free of prior allogeneic HSCT; (f) free of detectable malignant cells from cerebrospinal fluid; (g) free of brain metastases; (h) free of prior central nervous system disorders; (i) free of unstable angina, arrhythmia, and/or myocardial infarction; (j) free of uncontrolled infection; (k) free of immunodeficiency disorders or autoimmune disorders that require immunosuppressive therapy; (l) free of infection by human immunodeficiency virus, hepatitis B virus, or hepatitis C virus; (m) free of Burkitt's lymphoma or leukemia; and (n) free of isolated extramedullary disease.

In some embodiments, the method disclosed above may further comprise (iii) administering to the human patient at least one subsequent dose of the population of genetically engineered T cells. In some examples, the human patient has a decrease in bone marrow blast count of at least 50% about 4 weeks after the first dose of the population of genetically engineered T cells. In some examples, the human patient may receive a second dose of the population of genetically engineered T cells about 4 to 8 weeks after the first dose of the population of genetically engineered T cells. In some instances, the second dose of the population of genetically engineered T cells is administered to the human patient about 4 weeks (e.g., on Day 28) after the first dose, which optionally is concurrent with a dose of the NK cell inhibitor such as daratumumab. The human patient may be in a morphologic remission and is MRD-remains positive. Alternatively or in addition, the human patient shows progressive disease (PD) and had prior response. In some instances, the human patient receives a subsequent lymphodepletion treatment about 2-7 days prior to each of the subsequent dose of the population of the genetically engineered T cells. If the human patient experiences significant cytopenias after steps (i)-(iii), the human patient does not receive subsequent lymphodepletion treatment prior to each of the subsequent dose of the population of the genetically engineered T cells. In some examples, the subsequent dose(s) is about 3×10⁷, about 1×10⁸, about 3×10⁸, about 4.5×10⁸, about 6×10⁸, or about 9×10⁸ CAR+ T cells.

In any of the methods disclosed herein, the CAR that binds CD19 may comprise an anti-CD19 single chain variable fragment (scFv) that comprises the same heavy chain complementary determining regions (CDRs) as those in a heavy chain variable region set forth in SEQ ID NO: 51, and the same light chain CDRs as those in a light chain variable region set forth in SEQ ID NO: 52. In some embodiments, the CAR comprises an anti-CD19 single chain variable fragment (scFv) that comprises a heavy chain variable region set forth in SEQ ID NO: 51, and a light chain variable region set forth in SEQ ID NO: 52. In some examples, the anti-CD19 scFv comprises the amino acid sequence of SEQ ID NO: 47. In some specific examples, the CAR that binds CD19 comprises the amino acid sequence of SEQ ID NO: 40.

In some embodiments, the nucleic acid encoding the CAR is inserted in the disrupted TRAC gene. In some examples, the disrupted TRAC gene comprises a deletion of a fragment comprising the nucleotide sequence of SEQ ID NO: 26. The nucleic acid encoding the anti-CD19 CAR may be inserted at the site of the deletion in the disrupted TRAC gene. In one example, the disrupted TRAC gene comprises the nucleotide sequence of SEQ ID NO: 54. In some embodiments, the disrupted β2M gene in the population of genetically engineered T cells comprises at least one of the nucleotide sequence set forth in SEQ ID NOs: 9-14. In some embodiments, the population of the genetically engineered T cells is allogeneic to the human patient. In some embodiments, at least 90% of the T cells in the population of genetically engineered T cells do not express a detectable level of TCR surface protein. For example, at least 70% of the T cells in the population of genetically engineered T cells do not express a detectable level of TCR surface protein. In some examples, at least 50% of the T cells in the population of genetically engineered T cells do not express a detectable level of B2M surface protein. Alternatively or in addition, at least 30% of the T cells in the population of genetically engineered T cells express a detectable level of the CAR.

In some examples, at least 99.5% of the T cells in the population of genetically engineered T cells do not express a detectable level of TCR surface protein. Alternatively or in addition, at least 70% of the T cells in the population of genetically engineered T cells do not express a detectable level of B2M surface protein. For example, at least 85% of the T cells in the population of the genetically engineered T cells may not express a detectable level of B2M surface protein. Alternatively or in addition, at least 50% of the T cells in the population of genetically engineered T cells express a detectable level of the CAR. For example, at least 70% of the T cells in the population of genetically engineered T cells express a detectable level of the CAR.

In some embodiments, the population of genetically engineered T cells are administered to the human patient via intravenous infusion. In some examples, the population of genetically engineered T cells are suspended in a cryopreservation solution.

Also within the scope of the present disclosure are pharmaceutical compositions for use in treating a B-cell malignancy, the pharmaceutical composition comprising any of the population of genetically engineered T cells disclosed herein (e.g., the CTX110 cells), as well as use of the genetically engineered T cells for manufacturing a medicament for use in treating a B-cell malignancy as disclosed herein (e.g., NHL or ALL). The use of the genetically engineered T cells may be in combination with an NK cell inhibitor such as an anti-CD38 antibody, for example, daratumumab.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1B provide flow cytometry plots showing CD38 expression on CAR T cells. FIG. 1A provides flow cytometry plots showing fluorescent minus one (FMO) control stained cells were used to set the gate for measuring CD38+ cells. FIG. 1B shows CD38 expression on anti-CD19 CAR T cells as measured by flow cytometry.

FIGS. 2A-2D are flow cytometry plots showing CD38 expression on normal immune cells (PBMCs) collected from a healthy donor (Donor 3469), and cultured in media alone or media supplemented with 10% complement. FIG. 2A shows the percentage of CD38 expressing T cells from PBMCs cultured in media alone. FIG. 2B shows the percentage of CD38 expressing T cells from PBMCs cultured in media supplemented with 10% complement. FIG. 2C shows the percentage of CD38 expressing NK cells from PBMCs cultured in media alone. FIG. 2D shows the percentage of CD38 expressing NK cells from PBMCs cultured in media supplemented with 10% complement.

FIGS. 3A-3D are flow cytometry plots showing CD38 expression on normal immune cells (PBMCs) collected from a healthy donor (Donor 3383) and cultured in media alone or media supplemented with 10% complement. FIG. 3A shows the percentage of CD38 expressing T cells from PBMCs cultured in media alone. FIG. 3B shows the percentage of CD38 expressing T cells from PBMCs cultured in media supplemented with 10% complement. FIG. 3C shows the percentage of CD38 expressing NK cells from PBMCs cultured in media alone. FIG. 3D shows the percentage of CD38 expressing NK cells from PBMCs cultured in media supplemented with 10% complement.

FIGS. 4A-4D are flow cytometry plots showing CD38 expression on normal immune cells (PBMCs) collected from a healthy donor (Donor 3469) after in vitro culture for 72 hours in either media alone or media supplemented with 10% complement. FIG. 4A shows the percentage of CD38 expressing T cells from PBMCs cultured in media alone for 72 hours. FIG. 4B shows the percentage of CD38 expressing T cells from PBMCs cultured in media supplemented with 10% complement for 72 hours. FIG. 4C shows the percentage of CD38 expressing NK cells from PBMCs cultured in media alone for 72 hours. FIG. 4D shows the percentage of CD38 expressing NK cells from PBMCs cultured in media supplemented with 10% complement for 72 hours.

FIGS. 5A-5D are flow cytometry plots showing CD38 expression on normal immune cells (PBMCs) collected from a healthy donor (Donor 3383) after in vitro culture for 72 hours in either media alone or media supplemented with 10% complement. FIG. 5A shows the percentage of CD38 expressing T cells from PBMCs cultured in media alone for 72 hours. FIG. 5B shows the percentage of CD38 expressing T cells from PBMCs cultured in media supplemented with 10% complement for 72 hours. FIG. 5C shows the percentage of CD38 expressing NK cells from PBMCs cultured in media alone for 72 hours. FIG. 5D shows the percentage of CD38 expressing NK cells from PBMCs cultured in media supplemented with 10% complement for 72 hours.

FIGS. 6A-6B are graphs showing the frequency and number of NK cells from PBMCs collected from a healthy donor and cultured for 96 hours with daratumumab (Dara) or control isotype mAb (Hu IgG1k), with or without 10% complement. Daratumumab was used at doses of 0.01, 0.1, or 1 μg/mL. FIG. 6A shows the frequency of NK cells after these treatments. FIG. 6B shows the number of NK cells after these treatments.

FIGS. 7A-7B are graphs showing the frequency and number of T cells from PBMCs collected from a healthy donor and cultured for 96 hours with daratumumab (Dara) or control isotype mAb (Hu IgG1k), with or without 10% complement. Daratumumab was used at doses of 0.01, 0.1, or 1 μg/mL. FIG. 7A shows the frequency of T cells after these treatments. FIG. 7B shows the number of T cells after these treatments.

FIGS. 8A-8N are flow cytometry plots measuring the early activation marker CD69 expression in anti-CD19 CAR T cells after a 24 hour co-culture with daratumumab at 0.01, 0.1 or 1 μg/mL and with addition of 2 μg/mL goat anti-human antibody. Expression of CD69 markers after treatment with control isotype mAb (IgG1k) and with addition of 2 μg/mL goat anti-human antibody were also measured. FIG. 8A shows CD69 expression in anti-CD19 CAR T cells after a 24 hour co-culture with no treatment. FIG. 8B shows CD69 expression in anti-CD19 CAR T cells after a 24 hour co-culture with daratumumab at 0.01 μg/mL. FIG. 8C shows CD69 expression in anti-CD19 CAR T cells after a 24 hour co-culture with daratumumab at 0.1 μg/mL. FIG. 8D shows CD69 expression in anti-CD19 CAR T cells after a 24 hour co-culture with daratumumab at 1 μg/mL. FIG. 8E shows CD69 expression in anti-CD19 CAR T cells after a 24 hour co-culture with 2 μg/mL goat anti-human antibody. FIG. 8F shows CD69 expression in anti-CD19 CAR T cells after a 24 hour co-culture with daratumumab at 0.01 μg/mL and 2 μg/mL goat anti-human antibody. FIG. 8G shows CD69 expression in anti-CD19 CART cells after a 24 hour co-culture with daratumumab at 0.1 μg/mL and 2 μg/mL goat anti-human antibody. FIG. 8H shows CD69 expression in anti-CD19 CAR T cells after a 24 hour co-culture with daratumumab at 1 μg/mL and 2 μg/mL goat anti-human antibody. FIG. 8I shows CD69 expression in anti-CD19 CAR T cells after a 24 hour co-culture with IgG1k at 0.01 μg/mL. FIG. 8J shows CD69 expression in anti-CD19 CAR T cells after a 24 hour co-culture with IgG1k at 0.1 μg/mL. FIG. 8K shows CD69 expression in anti-CD19 CAR T cells after a 24 hour co-culture with IgG1k at 1 μg/mL. FIG. 8L shows CD69 expression in anti-CD19 CAR T cells after a 24 hour co-culture with IgG1k at 0.01 μg/mL and 2 μg/mL goat anti-human antibody. FIG. 8M shows CD69 expression in anti-CD19 CAR T cells after a 24 hour co-culture with IgG1k at 0.1 μg/mL and 2 μg/mL goat anti-human antibody. FIG. 8N shows CD69 expression in anti-CD19 CAR T cells after a 24 hour co-culture with IgG1k at 1 μg/mL and 2 μg/mL goat anti-human antibody.

FIG. 9 is a graph depicting NK cell numbers as measured by flow cytometry of blood collected from mice that were injected with 100 μg per mouse of daratumumab and/or 0.4×10⁶ NK cells per mouse. Error bars represent standard error of mean (SEM) where n=3.

FIG. 10 is a graph depicting weekly bioluminescence measurements of immune-deficient mice intravenously injected with 0.5×10⁶Nalm6 cells/mouse, the measurements collected after another intravenous injection of the mice with NK cells, PBS, daratumumab (DARA), IgG1, and/or anti-CD19 CAR T cells (4×10⁶cells/mouse). Error bars represent standard error of mean (SEM) where n=3.

FIG. 11 is a graph depicting the estimated daratumumab plasma concentration after a single dose or three consecutive doses. The dashed line indicates the approximate EC₉₀ for natural killer cell cytotoxicity.

FIG. 12 is a series of flow cytometry plots of human primary T-cells, TRAC−/B2M−CD19 CAR+ T cells (CYX 110), 8 days post-editing. The graphs show reduced surface expression of TRAC and B2M. TCR/MHC Class-I double knockout cells express high levels of the CAR transgene (bottom panel). Negative selection of CTX110 cells with purification beads leads to a reduction in TCR positive cells (right panel).

FIG. 13 is a graph depicting high editing rates achieved at the TRAC and B2M loci in TRAC−/B2M−CD19CAR+ T cells (CTX110). Surface expression of TCR and MHC Class-I, which is the functional output of gene editing, was measured and plotted as editing percentage on the y-axis. High efficiency (e.g., greater than 50%) site-specific integration and expression of the CAR from the TRAC locus were detected. These data demonstrate greater than 50% efficiency for the generation of TRAC−/B2M−/anti-CD19CAR+ T cells.

FIG. 14 is a Kaplan-Meier survival plot demonstrating increased survival of NOG Nalm6 mice treated with CTX110 cells on day 4, in comparison to control mice receiving no treatment on day 1.

FIG. 15 is a Kaplan-Meier survival plot demonstrating an increase survival of mice bearing a disseminated Nalm6 B-cell acute lymphoblastic leukemia (B-ALL) after treatment with different concentrations of CTX110, in comparison to control mice receiving no treatment.

FIG. 16 is a graph depicting a statistically significant inhibition in tumor cell expansion in the disseminated Nalm6 B-cell acute lymphoblastic leukemia (B-ALL) tumor model following treatment with CTX110 cells.

FIG. 17 is a Kaplan-Meier survival plot of healthy mice treated with CTX110 cells or various control cells (PBMCs or electroporated (EP) T cells) after radiation, or mice that only received radiation (“RT only”).

FIG. 18 is a graph showing percentage of body weight change of the mice treated as described in FIG. 13.

FIG. 19 is a Kaplan-Meier survival plot of healthy mice treated with a low dose (2×10⁷) or high dose (4×10⁷) of CTX110 cells, or unedited T cells after radiation, or mice that only received radiation (“Vehicle-RT”).

FIG. 20 is a graph showing percentage of body weight change of the mice treated in FIG. 15, in addition to mice that were not irradiated and not dosed with cells (“Vehicle—no RT”).

FIG. 21 is a bar graph showing percentage of CD27+CD45RO− cells within the unedited CD8+ T cell subset of peripheral blood cells from six different donors.

FIG. 22 provides flow cytometry results of TCRαβ and B2M expression on CTX110 cells before and after depletion of TCRαβ+ cells.

FIG. 23 is a graph the percentage loss of protein for TCR− and MHC Class-I- (B2M) after gene editing, and percentage of cells expressing an anti-CD19 CAR in edited CTX110 cells from individual lots of CTX110 production.

FIG. 24 provides graphs showing the percentage of PD1+(top left), LAG3+ (top right), TIM3+ (bottom left) or CD57+ (bottom right) in the T cell population from six different donors before and after editing.

FIG. 25 is a graph showing the percentage of cell lysis of CD19-positive cell lines (Nalm6; Raji; and K562-CD19) and CD19-negative cells (K562) when co-cultured at different ratios with CTX110 cells or unedited T cells.

FIG. 26 is a graph showing the number of viable CTX110 cells when cultured in the presence of T-cell media (serum+IL2+IL7; Complete Media), media containing serum but no IL2 or IL7 cytokines (5% Serum, No cytokines) or no serum or cytokines (No Serum, No Cytokines). Cells were counted on the indicated days post gene editing. Mean values from three lots shown ±SD.

FIG. 27 is a schematic depicting the clinical study design to evaluate CTX110 cells to human subjects having CD19+ adult B cell ALL. LD chemotherapy comprises co-administration of fludarabine 30 mg/m² and cyclophosphamide 500 mg/m² IV daily for 3 days. Subjects with B cell ALL may have a second dose of CTX110 on Day 28 with or without LD chemotherapy if they meet the protocol specified criteria (if the subject has a decrease in BM blast count at Day 28 of at least 50%) or if subjects who are in morphologic remission remain MRD positive. Subjects may be redosed upon disease progression if a subject had prior objective response. ALL: acute lymphoblastic leukemia; D: day; IV: intravenously; LD: lymphodepleting; M: month; MRD: minimal residual disease.

FIG. 28 is a schematic depicting the clinical study design to evaluate evaluate CTX110 cells administered after daratumumab infusion and lymphodepletion to human subjects having CD19+ malignancies, for example, NHL (e.g., DLBCL NOS, high grade B cell lymphoma with MYC and BCL2 and/or BCL6 rearrangements, grade 3b FL, or transformed FL) or B-cell ALL. Subjects receive an IV infusion (16 mg/kg) or SC injection (1800 mg) of daratumumab administered followed by LD chemotherapy (co-administration of fludarabine 30 mg/m² and cyclophosphamide 500 mg/m² IV daily for 3 days). Daratumumab is administered at least 1 day prior to starting LD chemotherapy and within 10 days of CTX110 infusion. CTX110 are administered 48 hours to 7 days after LD chemotherapy. For subjects (e.g., NHL patients) who achieve stable disease or better on Day 28, 2 additional doses of daratumumab (16 mg/kg IV or 1800 mg SC) may be administered at the Day 28 (±4 days) and Month 2 (±4 days) visits. Subjects with NHL who achieve SD or better may receive a second planned dose of CTX110 on Day 28 (4-8 weeks after the first dose) with daratumumab and LD chemotherapy if they meet the protocol-specified criteria. Subjects with B cell ALL may receive a second planned dose of CTX110 on Day 28 with daratumumab and LD chemotherapy if they meet the protocol specified criteria (if the subject has a decrease in BM blast count at Day 28 of at least 50%) or are in morphologic remission and remain MRD positive. For both NHL and ALL patients, additional CTX110 dose may be administered after PD if a subject has had prior objective response and if they meet the protocol-specified criteria (can redose CTX110 without daratumumab administration). D: day; Dara: daratumumab; DLBCL: diffuse large B cell lymphoma; DLT: dose-limiting toxicity; FL: follicular lymphoma; IV: intravenously; LD: lymphodepleting; M: month; NHL: non-Hodgkin lymphoma; NOS: not otherwise specified.

FIG. 29 is a schematic depicting the clinical study design to evaluate multiple doses of CTX110 cells with or without LD in human patients having NHL, such as DLBCL NOS, high grade B cell lymphoma with MYC and BCL2 and/or BCL6 rearrangements, grade 3b FL, or transformed FL. LD chemotherapy comprises co-administration of fludarabine 30 mg/m² and cyclophosphamide 500 mg/m² IV daily for 3 days. Subjects receive an IV infusion (16 mg/kg) or SC injection (1800 mg) of daratumumab administered followed by LD chemotherapy (co-administration of fludarabine 30 mg/m² and cyclophosphamide 500 mg/m² IV daily for 3 days). Daratumumab is administered at least 1 day prior to starting LD chemotherapy and within 10 days of CTX110 infusion. CTX110 are administered 48 hours to 7 days after LD chemotherapy. Subjects are administered a planned second dose of CTX110 on Day 8 (+2 days) without LD chemotherapy and another planned dose of CTX110 on Day 28 with daratumumab and LD chemotherapy if the protocol specified criteria are met. Subjects may be redosed upon disease progression if they have had prior objective response and meet the protocol specified criteria. D: day; DLBCL: diffuse large B cell lymphoma; DLT: dose-limiting toxicity; FL: follicular lymphoma; IV: intravenously; LD: lymphodepleting; M: month; NHL: non-Hodgkin lymphoma; NOS: not otherwise specified.

FIGS. 30A and 30B include diagrams showing effects of daratumumab in enhancing CAR-T cell expansion and reducing NK cells in vivo. FIG. 30A: CAR-T cell counts in patients. FIG. 30B: NK cell counts in patients.

### DETAILED DESCRIPTION OF THE INVENTION

Cluster of Differentiation 19 (CD19) is an antigenic determinant detectable on leukemia precursor cells. The human and murine amino acid and nucleic acid sequences can be found in a public database, such as GenBank, UniProt and Swiss-Prot. For example, the amino acid sequence of human CD19 can be found as UniProt/Swiss-Prot Accession No. P15391 and the nucleotide sequence encoding of the human CD19 can be found at Accession No. NM_001178098. CD19 is expressed on most B lineage cancers, including, e.g., acute lymphoblastic leukemia, chronic lymphocyte leukemia and non-Hodgkin's lymphoma. It is also an early marker of B cell progenitors. See, e.g., Nicholson et al., Mol. Immun. 34 (16-17): 1157-1165 (1997). As such, CD19 is a target for treating various CD19⁺ diseases such as CD19⁺ malignancies.

In some aspects, the present disclosure provides a combined therapy of anti-CD19 CAR⁺ T cells and NK cell inhibitor for treating CD19+ B cell malignancy, for example, non-Hodgkin lymphoma (NHL) and adult B cell ALL. Without wishing to be bound by theory, it is believed that CAR T cells with disrupted MHC Class I are not able to provide the required MHC Class I-NK KIR receptor binding that prevents NK-cells from eliminating MHC-Class I sufficient cells, i.e., self-cells. Thus, allogeneic CAR T cells with disrupted MHC Class I are susceptible to elimination by NK-mediated immune surveillance. It was discovered that the administration of an NK cell inhibitor, using an anti-CD38 monoclonal antibody as an example, resulted in a reduction of NK cell numbers. The depletion of NK cells, in turn, protects the allogeneic CAR T cell from host NK-mediated cell lysis. The combination of CAR T cell therapy and NK cell inhibitors thus presents an improvement over the existing CAR T cell therapy.

It was demonstrated that T cells isolated from PBMCs also express CD38 protein on the cell surface. Surprisingly, the addition of an anti-CD38 monoclonal antibody at doses that depleted NK cells did not affect T cell numbers, even after multi-day culture with an anti-CD38 monoclonal antibody. Nor does the addition of anti-CD38 monoclonal antibody at doses that depleted NK cell numbers induce CAR T cell activation. Accordingly, without wishing to be bound by theory, it is believed that anti-CD38 monoclonal antibody treatment is NK cell-specific, and induces reduction of NK cells without causing undesirable non-specific CAR T cell activation or elimination. The addition of an NK cell inhibitor, such as an anti-CD38 monoclonal antibody, represents an improvement to existing CAR T cell therapy.

It was further demonstrated that the effect of the anti-CD38 antibody on NK cells was not complement-dependent, as the addition of complement to co-culture of anti-CD38 antibody and PBMC did not affect the magnitude of NK cell depletion. More importantly, the addition of complement did not result in the depletion of T cells or affected CAR T cell activation status. Accordingly, without wishing to be bound by theory, it is believed that administration of an NK cell inhibitor, such as an anti-CD38 antibody, in combination with a CAR T cell therapy improves CAR T cell persistence and efficacy.

Further, it was reported herein that an NK cell inhibitor (daratumumab) successfully enhanced expansion of anti-CAR⁺ T cells (CTX110 cells) and prolonged survival of the anti-CAR⁺ T cells, specifically MHC-I deficient CAR⁺ T cells. At least one human patient receiving the combined therapy achieved complete response for at least three months after the treatment.

Accordingly, provided herein are methods for treating a B-cell malignancy in a human patient using a population of genetically engineered immune cells such as T cells, which express an anti-CD19 CAR (e.g., SEQ ID NO: 40, encoded by SEQ ID NO:39). Such genetically engineered T cells may further comprise a disrupted TRAC gene, a disrupted B2M, or a combination thereof. The nucleic acid encoding the anti-CD19 CAR and optionally comprising a promoter sequence and one or more regulatory elements may be inserted in the disrupted TRAC gene locus, e.g., replacing the segment of SEQ ID NO: 26 in the TRAC gene. The human patient is subject to a lymphodepletion treatment prior to administration of the population of genetically engineered T cells.

Also provided herein are methods for treating ALL (e.g., adult B cell ALL) in a human patient using the anti-CD19 CAR⁺ T cells as disclosed herein. The human patient may be subject to a lymphodepletion treatment prior to administration of the population of genetically engineered T cells.

### I. Anti-CD19 CAR T Cells

Disclosed herein are anti-CD19 CAR T cells (e.g., CTX110 cells) for use in treating B cell malignancies. In some embodiments, the anti-CD19 CAR T cells are human T cells expressing an anti-CD19 CAR. In some instances, such anti-CD19 CAR T cells may be deficient in expression of MHC Class-I subunits, for example, have a disrupted β2M gene. Such anti-CD19 CAR-T cells have a much lower expression level of MHC Class-I molecules relative to anti-CD19 Car-T cells that normal in MHC Class-I expression (e.g., less than 50%, less than 40%, less than 30%, less than 20%, less than 10%, less than 5% or lower in MHC Class-I expression relative to the counterpart CAR-T cells). In other instances, the anti-CD19 CAR T cells may have a disrupted TRAC gene, a disrupted B2M gene, or a combination thereof. In specific examples, the anti-CD19 CAR T cells express an anti-CD19 CAR and have endogenous TRAC and B2M genes disrupted.

(i) Anti-CD19 Chimeric Antigen Receptor (CAR)

The genetically engineered immune cells such as T cells disclosed here express a chimeric antigen receptor (CAR) that binds CD19 (an anti-CD19 CAR). A chimeric antigen receptor (CAR) refers to an artificial immune cell receptor that is engineered to recognize and bind to an antigen expressed by undesired cells, for example, disease cells such as cancer cells. A T cell that expresses a CAR polypeptide is referred to as a CAR T cell. CARs have the ability to redirect T-cell specificity and reactivity toward a selected target in a non-MHC-restricted manner. The non-MHC-restricted antigen recognition gives CAR-T cells the ability to recognize an antigen independent of antigen processing, thus bypassing a major mechanism of tumor escape. Moreover, when expressed on T-cells, CARs advantageously do not dimerize with endogenous T-cell receptor (TCR) alpha and beta chains.

There are various generations of CARs, each of which contains different components. First generation CARs join an antibody-derived scFv to the CD3zeta (ζ or z) intracellular signaling domain of the T-cell receptor through hinge and transmembrane domains. Second generation CARS incorporate an additional co-stimulatory domain, e.g., CD28, 4-1BB (41BB), or ICOS, to supply a costimulatory signal. Third-generation CARs contain two costimulatory domains (e.g., a combination of CD27, CD28, 4-1BB, ICOS, or OX40) fused with the TCR CD3ζ chain. Maude et al., Blood. 2015; 125(26):4017-4023; Kakarla and Gottschalk, Cancer J. 2014; 20(2):151-155). Any of the various generations of CAR constructs is within the scope of the present disclosure.

Generally, a CAR is a fusion polypeptide comprising an extracellular domain that recognizes a target antigen (e.g., a single chain fragment (scFv) of an antibody or other antibody fragment) and an intracellular domain comprising a signaling domain of the T-cell receptor (TCR) complex (e.g., CD3ζ) and, in most cases, a co-stimulatory domain. (Enblad et al., Human Gene Therapy. 2015; 26(8):498-505). A CAR construct may further comprise a hinge and transmembrane domain between the extracellular domain and the intracellular domain, as well as a signal peptide at the N-terminus for surface expression. Examples of signal peptides include MLLLVTSLLLCELPHPAFLLIP (SEQ ID NO: 30) and MALPVTALLLPLALLLHAARP (SEQ ID NO: 31). Other signal peptides may be used.

The anti-CD19 CAR may comprise an anti-CD19 single-chain variable fragment (scFv) specific for CD19, followed by hinge domain and transmembrane domain (e.g., a CD8 hinge and transmembrane domain) that is fused to an intracellular co-signaling domain (e.g., a CD28 co-stimulatory domain) and a CD3ζ signaling domain. Exemplary components for use in constructing the anti-CD19 CAR disclosed herein can be found in the Sequence Table provided below.

Antigen Binding Extracellular Domain

The antigen-binding extracellular domain is the region of a CAR polypeptide that is exposed to the extracellular fluid when the CAR is expressed on cell surface. In some instances, a signal peptide may be located at the N-terminus to facilitate cell surface expression. In some embodiments, the antigen binding domain can be a single-chain variable fragment (scFv, which may include an antibody heavy chain variable region (V_{H}) and an antibody light chain variable region (V_{L}) (in either orientation). In some instances, the V_{H} and V_{L} fragment may be linked via a peptide linker. The linker, in some embodiments, includes hydrophilic residues with stretches of glycine and serine for flexibility as well as stretches of glutamate and lysine for added solubility. The scFv fragment retains the antigen-binding specificity of the parent antibody, from which the scFv fragment is derived. In some embodiments, the scFv may comprise humanized V_{H} and/or V_{L} domains. In other embodiments, the V_{H} and/or V_{L} domains of the scFv are fully human.

The antigen-binding extracellular domain in the CAR polypeptide disclosed herein is specific to CD19 (e.g., human CD19). In some examples, the antigen-binding extracellular domain may comprise a scFv extracellular domain capable of binding to CD19. The anti-CD19 scFv may comprise a heavy chain variable domain (V_{H}) having the same heavy chain complementary determining regions (CDRs) as those in SEQ ID NO: 51 and a light chain variable domain (V_{L}) having the same light chain CDRs as those in SEQ ID NO: 52. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/). In some examples, the anti-CD19 scFv comprises the V_{H} of SEQ ID NO: 51 and/or the V_{L} of SEQ ID NO: 52. In specific examples, the anti-CD19 scFv may comprise the amino acid sequence of SEQ ID NO: 47.

Transmembrane Domain

The anti-CD19 CAR polypeptide disclosed herein may contain a transmembrane domain, which can be a hydrophobic alpha helix that spans the membrane. As used herein, a “transmembrane domain” refers to any protein structure that is thermodynamically stable in a cell membrane, preferably a eukaryotic cell membrane. The transmembrane domain can provide stability of the CAR containing such.

In some embodiments, the transmembrane domain of a CAR as provided herein can be a CD8 transmembrane domain. In other embodiments, the transmembrane domain can be a CD28 transmembrane domain. In yet other embodiments, the transmembrane domain is a chimera of a CD8 and CD28 transmembrane domain. Other transmembrane domains may be used as provided herein. In one specific example, the transmembrane domain in the anti-CD19 CAR is a CD8α transmembrane domain having the amino acid sequence of SEQ ID NO: 32.

Hinge Domain

In some embodiments, a hinge domain may be located between an extracellular domain (comprising the antigen binding domain) and a transmembrane domain of a CAR, or between a cytoplasmic domain and a transmembrane domain of the CAR. A hinge domain can be any oligopeptide or polypeptide that functions to link the transmembrane domain to the extracellular domain and/or the cytoplasmic domain in the polypeptide chain. A hinge domain may function to provide flexibility to the CAR, or domains thereof, or to prevent steric hindrance of the CAR, or domains thereof.

In some embodiments, a hinge domain may comprise up to 300 amino acids (e.g., 10 to 100 amino acids, or 5 to 20 amino acids). In some embodiments, one or more hinge domain(s) may be included in other regions of a CAR. In some embodiments, the hinge domain may be a CD8 hinge domain. Other hinge domains may be used.

Intracellular Signaling Domains

Any of the anti-CD19 CAR constructs disclosed herein contain one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three (3) immunoreceptor tyrosine-based activation motif (ITAM)s, which transmit an activation signal to the T cell after the T cell is engaged with a cognate antigen. In many cases, CD3ζ provides a primary T cell activation signal but not a fully competent activation signal, which requires a co-stimulatory signaling. In some examples, the anti-CD19 CAR construct disclosed herein comprise a CD3ζ cytoplasmic signaling domain, which may have the amino acid sequence of SEQ ID NO: 38.

In some embodiments, the anti-CD19 CAR polypeptides disclosed herein may further comprise one or more co-stimulatory signaling domains. For example, the co-stimulatory domains of CD28 and/or 4-1BB may be used to transmit a full proliferative/survival signal, together with the primary signaling mediated by CD3ζ. In some examples, the CAR disclosed herein comprises a CD28 co-stimulatory molecule, for example, a CD28 co-stimulatory signaling domain having the amino acid sequence of SEQ ID NO: 36. In other examples, the CAR disclosed herein comprises a 4-1BB co-stimulatory molecule, for example, a 4-1BB co-stimulatory signaling domain having the amino acid sequence of SEQ ID NO: 34.

In specific examples, an anti-CD19 CAR disclosed herein may include a CD3ζ signaling domain (e.g., SEQ ID NO: 38) and a CD28 co-stimulatory domain (e.g., SEQ ID NO: 36).

It should be understood that methods described herein encompasses more than one suitable CAR that can be used to produce genetically engineered T cells expressing the CAR, for example, those known in the art or disclosed herein. Examples can be found in, e.g., International Application Number PCT/IB2018/001619, filed May 11, 2018, which published as WO 2019/097305A2, and International Application Number PCT/IB2019/000500, filed May 10, 2019, the relevant disclosures of each of the prior applications are incorporated by reference herein for the purpose and subject matter referenced herein.

In specific examples, the anti-CD19 CAR disclosed herein may comprise the amino acid sequence of SEQ ID NO: 40, which may be encoded by the nucleotide sequence of SEQ ID NO: 39. See the sequence table provided below.

In the genetically engineered T cells disclosed herein, a nucleic acid comprising the coding sequence of the anti-CD19 CAR, and optionally regulatory sequences for expression of the anti-CD19 CAR (e.g., a promoter such as the EF1α promoter provided in the sequence Table) may be inserted into a genomic locus of interest. In some examples, the nucleic acid is inserted in the endogenous TRAC gene locus, thereby disrupting expression of the TRAC gene. In specific examples, the nucleic acid may replace a fragment in the TRAC gene, for example, a fragment comprising the nucleotide sequence of SEQ ID NO: 26.

(ii) Knock-Out of TRAC and B2M Genes

The anti-CD19 CAR-T cells disclosed herein may further have a disrupted TRAC gene, a disrupted B2M gene, or a combination thereof. The disruption of the TRAC locus results in loss of expression of the T cell receptor (TCR) and is intended to reduce the probability of Graft versus Host Disease (GvHD), while the disruption of the β2M locus results in lack of expression of the major histocompatibility complex type I (MHC I) proteins and is intended to improve persistence by reducing the probability of host rejection. The addition of the anti-CD19 CAR directs the modified T cells towards CD19-expressing tumor cells.

As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a β2M gene edit may be considered a β2M knockout cell if β2M protein cannot be detected at the cell surface using an antibody that specifically binds β2M protein.

In some embodiments, a disrupted gene may be described as comprising a mutated fragment relative to the wild-type counterpart. The mutated fragment may comprise a deletion, a nucleotide substitution, an addition, or a combination thereof. In other embodiments, a disrupted gene may be described as having a deletion of a fragment that is present in the wild-type counterpart. In some instances, the 5′ end of the deleted fragment may be located within the gene region targeted by a designed guide RNA such as those disclosed herein (known as on-target sequence) and the 3′ end of the deleted fragment may go beyond the targeted region. Alternatively, the 3′ end of the deleted fragment may be located within the targeted region and the 5′ end of the deleted fragment may go beyond the targeted region.

In some instances, the disrupted TRAC gene in the anti-CD19 CAR-T cells disclosed herein may comprise a deletion, for example, a deletion of a fragment in Exon 1 of the TRAC gene locus. In some examples, the disrupted TRAC gene comprises a deletion of a fragment comprising the nucleotide sequence of SEQ ID NO: 26, which is the target site of TRAC guide RNA TA-1. See sequence table below. In some examples, the fragment of SEQ ID NO: 26 may be replaced by a nucleic acid encoding the anti-CD19 CAR. Such a disrupted TRAC gene may comprise the nucleotide sequence of SEQ ID NO: 54.

The disrupted B2M gene in the anti-CD19 CAR-T cells disclosed herein may be generated using the CRISPR/Cas technology. In some examples, a B2M gRNA provided in the sequence table below can be used. The disrupted B2M gene may comprise a nucleotide sequence of any one of SEQ ID Nos: 9-14.

(iii) Exemplary Population of Anti-CD19 CAR-T Cells for Allogeneic Therapy

Also provided herein is population of genetically engineered immune cells (e.g., T cells such as human T cells) comprising the anti-CD19 CAR-T cells disclosed herein, which express any of the anti-CD19 CAR disclosed herein (e.g., the anti-CD19 CAR comprising the amino acid sequence of SEQ ID NO: 40), and a disrupted TRAC gene and/or a disrupted B2M gene as also disclosed herein. In some examples, the population of genetically engineered T cells are CTX110 cells, which are CD19-directed T cells having disrupted TRAC gene and B2M gene. The nucleic acid encoding the anti-CD19 CAR can be inserted in the disrupted TRAC gene at the site of SEQ ID NO: 26, which is replaced by the nucleic acid encoding the anti-CD19 CAR, thereby disrupting expression of the TRAC gene. The disrupted TRAC gene in the CTX110 cells may comprise the nucleotide sequence of SEQ ID NO: 54.

CTX110 cells can be produced via ex vivo genetic modification using the CRISPR/Cas9 (Clustered Regularly Interspaced Short Palindromic Repeats/CRISPR associated protein 9) technology to disrupt targeted genes (TRAC and B2M genes), and adeno-associated virus (AAV) transduction to deliver the anti-CD19 CAR construct. CRISPR-Cas9-mediated gene editing involves two guide RNAs (sgRNAs): TA-1 sgRNA (SEQ ID NO: 18), which targets the TRAC locus, and B2M-1 sgRNA (SEQ ID NO: 20), which targets the β2M locus. For any of the gRNA sequences provided herein, those that do not explicitly indicate modifications are meant to encompass both unmodified sequences and sequences having any suitable modifications.

The anti-CD19 CAR of CTX110 cells is composed of an anti-CD19 single-chain antibody fragment (scFv, which may comprise the amino acid sequence of SEQ ID NO: 47), followed by a CD8 hinge and transmembrane domain (e.g., comprising the amino acid sequence of SEQ ID NO: 32) that is fused to an intracellular co-signaling domain of CD28 (e.g., SEQ ID NO: 36) and a CD3ζ signaling domain (e.g., SEQ ID NO: 38). In specific examples, the anti-CD19 CAR in CTX110 cells comprises the amino acid sequence of SEQ ID NO: 40.

In some embodiments, at least 30% of a population of CTX110 cells express a detectable level of the anti-CD19 CAR. For example, at least 40%, at least 50%, at least 60%, at least 70%, at least 75%, at least 80%, at least 85%, at least 90%, or at least 95% of the CTX110 cells express a detectable level of the anti-CD19 CAR.

In some embodiments, at least 50% of a population of CTX110 cells may not express a detectable level of β2M surface protein. For example, at least 55%, at least 60%, at least 70%, at least 75%, at least 80%, at least 85%, at least 90%, or at least 95% of the CTX110 cells may not express a detectable level of β2M surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M surface protein.

Alternatively or in addition, at least 50% of a population of CTX110 cells may not express a detectable level of TCR surface protein. For example, at least 55%, at least 60%, at least 70%, at least 75%, at least 80%, at least 85%, at least 90%, or at least 95% of the CTX110 cells may not express a detectable level of TCR surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of TRAC surface protein. In specific examples, more than 90% (e.g., more than 99.5%) of the CTX110 cells do not express a detectable TCR surface protein.

In some embodiments, a substantial percentage of the population of CTX110 T cells may comprise more than one gene edit, which results in a certain percentage of cells not expressing more than one gene and/or protein.

For example, at least 50% of a population of CTX110 cells may not express a detectable level of two surface proteins, e.g., does not express a detectable level of β2M and TRAC proteins. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the CTX110 T cells do not express a detectable level of TRAC and B2M surface proteins. In another example, at least 50% of a population of the CTX110 cells do not express a detectable level of TRAC and B2M surface proteins.

In some embodiments, the population of CTX110 T cells may comprise more than one gene edit (e.g., in more than one gene), which may be an edit described herein. For example, the population of CTX110 T cells may comprise a disrupted TRAC gene via the CRISPR/Cas technology using the TA-1 TRAC gRNA. In some examples, the CTX110 cells may comprise a deletion in the TRAC gene relative to unmodified T cells. For example, the CTX110 T cells may comprise a deletion of the fragment AGAGCAACAGTGCTGTGGCC (SEQ ID NO: 26) in the TRAC gene. This fragment can be replaced by the nucleic acid encoding the anti-CD19 CAR (e.g., SEQ ID NO: 39). Alternatively or in addition, the population of CTX110 cells may comprise a disrupted β2M gene via CRISPR/Cas9 technology using the gRNA of B2M-1. Such CTX110 cells may comprise Indels in the β2M gene, which comprise one or more of the nucleotide sequences of SEQ ID NOs: 9-14. In specific examples, CTX110 cells comprise ≥30% CAR⁺ T cells, ≤50% B2M⁺ cells, and ≤30% TCRαβ⁺ cells. In additional specific examples, CTX110 cells comprise ≥30% CAR⁺ T cells, ≤30% B2M⁺ cells, and ≤0.5% TCRαβ⁺ cells.

See also WO 2019/097305A2, and WO2019215500, the relevant disclosures of each of which are incorporated by reference for the subject matter and purpose referenced herein.

(iv) Pharmaceutical Compositions

In some aspects, the present disclosure provides pharmaceutical compositions comprising any of the populations of genetically engineered anti-CD19 CAR T cells as disclosed herein, for example, CTX110 cells, and a pharmaceutically acceptable carrier. Such pharmaceutical compositions can be used in cancer treatment in human patients, which is also disclosed herein.

As used herein, the term “pharmaceutically acceptable” refers to those compounds, materials, compositions, and/or dosage forms which are, within the scope of sound medical judgment, suitable for use in contact with the tissues, organs, and/or bodily fluids of the subject without excessive toxicity, irritation, allergic response, or other problems or complications commensurate with a reasonable benefit/risk ratio. As used herein, the term “pharmaceutically acceptable carrier” refers to solvents, dispersion media, coatings, antibacterial agents, antifungal agents, isotonic and absorption delaying agents, or the like that are physiologically compatible. The compositions can include a pharmaceutically acceptable salt, e.g., an acid addition salt or a base addition salt. See, e.g., Berge et al., (1977) J Pharm Sci 66:1-19.

In some embodiments, the pharmaceutical composition further comprises a pharmaceutically acceptable salt. Non-limiting examples of pharmaceutically acceptable salts include acid addition salts (formed from a free amino group of a polypeptide with an inorganic acid (e.g., hydrochloric or phosphoric acids), or an organic acid such as acetic, tartaric, mandelic, or the like). In some embodiments, the salt formed with the free carboxyl groups is derived from an inorganic base (e.g., sodium, potassium, ammonium, calcium or ferric hydroxides), or an organic base such as isopropylamine, trimethylamine, 2-ethylamino ethanol, histidine, procaine, or the like).

In some embodiments, the pharmaceutical composition disclosed herein comprises a population of the genetically engineered anti-CD19 CAR-T cells (e.g., CTX110 cells) suspended in a cryopreservation solution (e.g., CryoStor® C55). The cryopreservation solution for use in the present disclosure may also comprise adenosine, dextrose, dextran-40, lactobionic acid, sucrose, mannitol, a buffer agent such as N-)2-hydroxethyl) piperazine-N′-(2-ethanesulfonic acid) (HEPES), one or more salts (e.g., calcium chloride, magnesium chloride, potassium chloride, potassium bicarbonate, potassium phosphate, etc.), one or more base (e.g., sodium hydroxide, potassium hydroxide, etc.), or a combination thereof. Components of a cryopreservation solution may be dissolved in sterile water (injection quality). Any of the cryopreservation solution may be substantially free of serum (undetectable by routine methods).

In some instances, a pharmaceutical composition comprising a population of genetically engineered anti-CD19 CAR-T cells such as the CTX110 cells suspended in a cryopreservation solution (e.g., substantially free of serum) may be placed in storage vials.

Any of the pharmaceutical compositions disclosed herein, comprising a population of genetically engineered anti-CD19 CAR T cells as also disclosed herein (e.g., CTX110 cells), which optionally may be suspended in a cryopreservation solution as disclosed herein may be stored in an environment that does not substantially affect viability and bioactivity of the T cells for future use, e.g., under conditions commonly applied for storage of cells and tissues. In some examples, the pharmaceutical composition may be stored in the vapor phase of liquid nitrogen at ≤−135 C. No significant changes were observed with respect to appearance, cell count, viability, % CAR⁺ T cells, % TCR⁺ T cells, and % B2M⁺ T cells after the cells have been stored under such conditions for a period of time.

### II. Preparation of Genetically Engineered Immune Cells

Any suitable gene editing methods known in the art can be used for making the genetically engineered immune cells (e.g., T cells such as CTX110 cells) disclosed herein, for example, nuclease-dependent targeted editing using zinc-finger nucleases (ZFNs), transcription activator-like effector nucleases (TALENs), or RNA-guided CRISPR-Cas9 nucleases (CRISPR/Cas9; Clustered Regular Interspaced Short Palindromic Repeats Associated 9). In specific examples, the genetically engineered immune cells such as CTX110 cells are produced by the CRISPR technology in combination with homologous recombination using an adeno-associated viral vector (AAV) as a donor template.

(i) CRISPR-Cas9-Mediated Gene Editing System

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crisprRNA (crRNA) and trans-activating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78)

crRNA drives sequence recognition and specificity of the CRISPR-Cas9 complex through Watson-Crick base pairing typically with a 20 nucleotide (nt) sequence in the target DNA. Changing the sequence of the 5′ 20nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM).

TracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells, and occurs at a relatively low frequency in most cell types. In many embodiments of the present disclosure, NHEJ is utilized as the repair operant.

(a) Cas9

In some embodiments, the Cas9 (CRISPR associated protein 9) endonuclease is used in a CRISPR method for making the genetically engineered T cells as disclosed herein. The Cas9 enzyme may be one from Streptococcus pyogenes, although other Cas9 homologs may also be used. It should be understood, that wild-type Cas9 may be used or modified versions of Cas9 may be used (e.g., evolved versions of Cas9, or Cas9 orthologues or variants), as provided herein. In some embodiments, Cas9 comprises a Streptococcus pyogenes-derived Cas9 nuclease protein that has been engineered to include C- and N-terminal SV40 large T antigen nuclear localization sequences (NLS). The resulting Cas9 nuclease (sNLS-spCas9-sNLS) is a 162 kDa protein that is produced by recombinant E. coli fermentation and purified by chromatography. The spCas9 amino acid sequence can be found as UniProt Accession No. Q99ZW2, which is provided herein as SEQ ID NO: 55.

(b) Guide RNAs (gRNAs)

CRISPR-Cas9-mediated gene editing as described herein includes the use of a guide RNA or a gRNA. As used herein, a “gRNA” refers to a genome-targeting nucleic acid that can direct the Cas9 to a specific target sequence within a TRAC gene or a β2M gene for gene editing at the specific target sequence. A guide RNA comprises at least a spacer sequence that hybridizes to a target nucleic acid sequence within a target gene for editing, and a CRISPR repeat sequence.

An exemplary gRNA targeting a TRAC gene is provided in SEQ ID NO: 18 or 22. See the sequence table below. See also WO 2019/097305A2, the relevant disclosures of which are incorporated by reference herein for the subject matter and purpose referenced herein. Other gRNA sequences may be designed using the TRAC gene sequence located on chromosome 14 (GRCh38: chromosome 14: 22,547,506-22,552,154; Ensembl; ENSG00000277734). In some embodiments, gRNAs targeting the TRAC genomic region and Cas9 create breaks in the TRAC genomic region resulting Indels in the TRAC gene disrupting expression of the mRNA or protein.

An exemplary gRNA targeting a β2M gene is provided in SEQ ID NO: 20 or 24. See the sequence table below. See also WO 2019/097305A2, the relevant disclosures of which are incorporated by reference herein for the purpose and subject matter referenced herein. Other gRNA sequences may be designed using the 62M gene sequence located on Chromosome 15 (GRCh38 coordinates: Chromosome 15: 44,711,477-44,718,877; Ensembl: ENSG00000166710). In some embodiments, gRNAs targeting the β2M genomic region and RNA-guided nuclease create breaks in the β2M genomic region resulting in Indels in the β2M gene disrupting expression of the mRNA or protein.

In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid (e.g., gRNA) is a double-molecule guide RNA. In some embodiments, the genome-targeting nucleic acid (e.g., gRNA) is a single-molecule guide RNA.

A double-molecule guide RNA comprises two strands of RNA molecules. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. A single-molecule guide RNA in a Type V system comprises, in the 5′ to 3′ direction, a minimum CRISPR repeat sequence and a spacer sequence.

The “target sequence” is in a target gene that is adjacent to a PAM sequence and is the sequence to be modified by Cas9. The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence.

For example, if the TRAC target sequence is 5′-AGAGCAACAGTGCTGTGGCC-3′ (SEQ ID NO: 26), then the gRNA spacer sequence is 5′-AGAGCAACAGUGCUGUGGCC-3′ (SEQ ID NO: 19). In another example, if the β2M target sequence is 5′-GCTACTCTCTCTITCTGGCC-3′ (SEQ ID NO: 27), then the gRNA spacer sequence is 5′-GCUACUCUCUCUUUCUGGCC-3′ (SEQ ID NO: 21). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM recognizable by a Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNNRG-3′, the target nucleic acid can be the sequence that corresponds to the Ns, wherein N can be any nucleotide, and the underlined NRG sequence is the S. pyogenes PAM. Examples are provided as SEQ ID NOs: 15-17.

The guide RNA disclosed herein may target any sequence of interest via the spacer sequence in the crRNA. In some embodiments, the degree of complementarity between the spacer sequence of the guide RNA and the target sequence in the target gene can be about 60%, 65%, 70%, 75%, 80%, 85%, 90%, 95%, 97%, 98%, 99%, or 100%. In some embodiments, the spacer sequence of the guide RNA and the target sequence in the target gene is 100% complementary. In other embodiments, the spacer sequence of the guide RNA and the target sequence in the target gene may contain up to 10 mismatches, e.g., up to 9, up to 8, up to 7, up to 6, up to 5, up to 4, up to 3, up to 2, or up to 1 mismatch.

Non-limiting examples of gRNAs that may be used as provided herein are provided in WO 2019/097305A2, and WO2019/215500, the relevant disclosures of each of which are herein incorporated by reference for the purposes and subject matter referenced herein. For any of the gRNA sequences provided herein, those that do not explicitly indicate modifications are meant to encompass both unmodified sequences and sequences having any suitable modifications.

The length of the spacer sequence in any of the gRNAs disclosed herein may depend on the CRISPR/Cas9 system and components used for editing any of the target genes also disclosed herein. For example, different Cas9 proteins from different bacterial species have varying optimal spacer sequence lengths. Accordingly, the spacer sequence may have 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the spacer sequence may have 18-24 nucleotides in length. In some embodiments, the targeting sequence may have 19-21 nucleotides in length. In some embodiments, the spacer sequence may comprise 20 nucleotides in length.

In some embodiments, the gRNA can be a sgRNA, which may comprise a 20 nucleotide spacer sequence at the 5′ end of the sgRNA sequence. In some embodiments, the sgRNA may comprise a less than 20 nucleotide spacer sequence at the 5′ end of the sgRNA sequence. In some embodiments, the sgRNA may comprise a more than 20 nucleotide spacer sequence at the 5′ end of the sgRNA sequence. In some embodiments, the sgRNA comprises a variable length spacer sequence with 17-30 nucleotides at the 5′ end of the sgRNA sequence.

In some embodiments, the sgRNA comprises no uracil at the 3′ end of the sgRNA sequence. In other embodiments, the sgRNA may comprise one or more uracil at the 3′ end of the sgRNA sequence. For example, the sgRNA can comprise 1-8 uracil residues, at the 3′ end of the sgRNA sequence, e.g., 1, 2, 3, 4, 5, 6, 7, or 8 uracil residues at the 3′ end of the sgRNA sequence.

Any of the gRNAs disclosed herein, including any of the sgRNAs, may be unmodified. Alternatively, it may contain one or more modified nucleotides and/or modified backbones. For example, a modified gRNA such as a sgRNA can comprise one or more 2′-O-methyl phosphorothioate nucleotides, which may be located at either the 5′ end, the 3′ end, or both.

In certain embodiments, more than one guide RNAs can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

It should be understood that more than one suitable Cas9 and more than one suitable gRNA can be used in methods described herein, for example, those known in the art or disclosed herein. In some embodiments, methods comprise a Cas9 enzyme and/or a gRNA known in the art. Examples can be found in, e.g., WO 2019/097305A2, and WO2019/215500, the relevant disclosures of each of which are herein incorporated by reference for the purposes and subject matter referenced herein.

(ii) AAV Vectors for Delivery of CAR Constructs to T Cells

A nucleic acid encoding an anti-CD19 CAR construct as disclosed herein can be delivered to a cell using an adeno-associated virus (AAV). AAVs are small viruses which integrate site-specifically into the host genome and can therefore deliver a transgene, such as CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, the AAV for use in delivering the CAR-coding nucleic acid is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

A nucleic acid encoding an anti-CD19 CAR can be designed to insert into a genomic site of interest in the host T cells. In some embodiments, the target genomic site can be in a safe harbor locus.

In some embodiments, a nucleic acid encoding the anti-CD19 CAR (e.g., via a donor template, which can be carried by a viral vector such as an adeno-associated viral (AAV) vector) can be designed such that it can insert into a location within a TRAC gene to disrupt the TRAC gene in the genetically engineered T cells and express the CAR polypeptide. Disruption of TRAC leads to loss of function of the endogenous TCR. For example, a disruption in the TRAC gene can be created with an endonuclease such as those described herein and one or more gRNAs targeting one or more TRAC genomic regions. Any of the gRNAs specific to a TRAC gene and the target regions can be used for this purpose, e.g., those disclosed herein.

In some examples, a genomic deletion in the TRAC gene and replacement by a CAR coding segment can be created by homology directed repair or HDR (e.g., using a donor template, which may be part of a viral vector such as an adeno-associated viral (AAV) vector). In some embodiments, a disruption in the TRAC gene can be created with an endonuclease as those disclosed herein and one or more gRNAs targeting one or more TRAC genomic regions, and inserting a CAR coding segment into the TRAC gene.

A donor template as disclosed herein can contain a coding sequence for a CAR. In some examples, the CAR-coding sequence may be flanked by two regions of homology to allow for efficient HDR at a genomic location of interest, for example, at a TRAC gene using CRISPR-Cas9 gene editing technology. In this case, both strands of the DNA at the target locus can be cut by a CRISPR Cas9 enzyme guided by gRNAs specific to the target locus. HDR then occurs to repair the double-strand break (DSB) and insert the donor DNA coding for the CAR. For this to occur correctly, the donor sequence is designed with flanking residues which are complementary to the sequence surrounding the DSB site in the target gene (hereinafter “homology arms”), such as the TRAC gene. These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced into a cell as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, can be inserted at a site nearby an endogenous promoter (e.g., downstream or upstream) so that its expression can be driven by the endogenous promoter. In other embodiments, the donor template may comprise an exogenous promoter and/or enhancer, for example, a constitutive promoter, an inducible promoter, or tissue-specific promoter to control the expression of the CAR gene. In some embodiments, the exogenous promoter is an EF1α promoter. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

To prepare the genetically engineered immune cells (e.g., T cells disclosed herein), immune cells such as T cells from a suitable source may be obtained, e.g., blood cells from a human donor, who may be a healthy donor or a patient need CAR-T cell therapy. The CTX110 cells can be made using blood cells from one or more healthy human donors. Manufacturing from healthy donor cells minimizes the risk of unintentionally transducing malignant lymphoma/leukemia cells and potentially may improve the functionality of the CAR T cells. The components of the CRISPR system (e.g., Cas9 protein and the gRNAs), optionally the AAV donor template, may be delivered into the host immune cells via conventional approaches. In some examples, the Cas9 and the gRNAs can form a ribonucleoprotein complex (RNP), which can be delivered to the host immune cells by electroporation. Optionally, the AAV donor template may be delivered to the immune cells concurrently with the RNP complex.

Alternatively, delivery of the RNPs and the AAV donor template can be performed sequentially. In some examples, the T cells may be activated prior to delivery of the gene editing components.

After delivery of the gene editing components and optionally the donor template, the cells may be recovered and expanded in vitro. Gene editing efficiency can be evaluated using routine methods for confirm knock-in of the anti-CD19 CAR and knock-out of the target genes (e.g., TRAC, B2M, or both). In some examples, TCRαβ⁺ T cells may be removed. Additional information for preparation of the genetically engineered immune cells disclosed herein such as the CTX110 cells can be found in U.S. Patent Application No. 62/934,991, the relevant disclosures of which are incorporated by reference for the purpose and subject matter referenced herein.

### III. NK Cell Inhibitors

NK cells play an important role in both innate and adaptive immunity, e.g., in mediating anti-tumor and anti-viral responses. NK cells have the ability to conduct “natural killing” of cellular targets without prior antigen sensitization. For example, NK cells have been observed to attack target cells having absent or altered expression of major histocompatibility complex (MHC) class I molecules. As such, CAR T cells (e.g., anti-CD19 CAR-T cells such as CTX110 cells) having a disrupted β2M gene, which encodes a component of MHC class I molecules, may be susceptible to NK cell mediated lysis, thereby reducing the persistence and subsequent efficacy of the CAR T cells.

Accordingly, in some embodiments, the present disclosure provides NK cell inhibitors for use in combination with anti-CD19 CAR T cells (e.g., CTX110 cells) for treating B cell malignancies. In some embodiments, the NK cell inhibitor is an anti-CD38 antibody, e.g., daratumumab.

The NK cell inhibitor to be used in the methods described herein can be a molecule that blocks, suppresses, or reduces the activity or number of NK cells, either directly or indirectly. The term “inhibitor” implies no specific mechanism of biological action whatsoever, and is deemed to expressly include and encompass all possible pharmacological, physiological, and biochemical interactions with NK cells whether direct or indirect. For the purpose of the present disclosure, it will be explicitly understood that the term “inhibitor” encompasses all the previously identified terms, titles, and functional states and characteristics whereby the NK cell itself, a biological activity of the NK cell (including but not limited to its ability to mediate cell killing), or the consequences of the biological activity, are substantially nullified, decreased, or neutralized in any meaningful degree, e.g., by at least 20%, 50%, 70%, 85%, 90%, 100%, 150%, 200%, 300%, or 500%, or by 10-fold, 20-fold, 50-fold, 100-fold, 1000-fold, or 10⁴-fold.

In some embodiments, an NK cell inhibitor reduces absolute NK cell numbers. In some embodiments, an NK cell inhibitor reduces NK cell frequency in peripheral blood mononuclear cells. In some embodiments, the NK cells are reduced by at least 10%, 20%, 30%, 40%, 50%, 60%, 70%, 80%, or 90%. In some embodiments, an NK cell inhibitor reduces the total number of NK cells in a subject compared to the total number of NK cells in the subject prior to receiving the NK cell inhibitor. In some embodiments, the NK cells are reduced to at least 20, 40, 60, 80, 100, 120, 140, 160, or 180 NK cells/μL of blood. In some embodiments, the NK cells are reduced to less than 200 NK cells/μL of blood.

In some embodiments, an NK cell inhibitor does not significantly reduce endogenous T cell numbers. In some embodiments, an NK cell inhibitor maintains endogenous T cell numbers at 85%, 90%, 95%, 100%, 105%, or 110% of T cell numbers relative to T cell numbers before NK cell inhibitor treatment. In some embodiments, an NK cell inhibitor maintains endogenous T cell numbers at about 1500 T cells/μL of blood. In some embodiments, an NK cell inhibitor maintains endogenous T cell number at about 1275, about 1350, about 1425, about 1500, about 1575, or about 1650 T cells/μL of blood. In some embodiments, an NK cell inhibitor does not significantly reduce the number of anti-CD19 CAR T cells. In some embodiments, an NK cell inhibitor increases the number of anti-CD19 CAR T cells compared to the number of such in the absence of the NK cell inhibitor. In some embodiments, an NK cell inhibitor does not significantly activate the anti-CD19 CAR T cells.

In some embodiments, an NK cell inhibitor reduces NK cell-mediated lysis of anti-CD19 CAR T cells. In some embodiments, an NK cell inhibitor reduces NK cell-mediated lysis of anti-CD19 CAR T cells by about 10%, 20%, 30%, 40%, 50%, 60%, 70%, 80%, 90% or 100% relative to NK cell-mediated lysis of anti-CD19 CAR T cells in the absence of the NK cell inhibitor. In some embodiments, an NK cell inhibitor reduces NK cell-mediated lysis of anti-CD19 CAR T cells in a subject.

In some embodiments, an NK cell inhibitor reduces an NK cell activity. In some embodiments, the disclosure provides methods for reducing NK cell activity in a subject by administering an NK cell inhibitor. In some embodiments, an NK cell inhibitor reduces antibody-dependent cell-mediated cytotoxicity (ADCC), antibody-dependent cellular phagocytosis (ADCP), complement dependent cytotoxicity (CDC), apoptosis, or combinations thereof.

In some embodiments, an NK cell inhibitor reduces NK cell-mediated antibody-dependent cell-mediated cytotoxicity (ADCC) of anti-CD19 CAR T cells. NK cells express Fc-receptors, e.g., FcγRIIIA and or FcγRIIC on their cell surfaces. The Fc-receptors bind the Fc portion of antibodies. Once bound, the Fc-receptors transmit activating signals through immune tyrosine-based activating motifs (ITAM), which results in downstream NK cell degranulation, cytokine secretion (e.g., IFN-γ), and cell lysis.

In some embodiments, an NK cell inhibitor reduces NK cell-mediated antibody-dependent cellular phagocytosis (ADCP) of engineered human CAR T cells. ADCP occurs when the Fc portion of an antibody engages the Fc-receptor, e.g., FcγRIIIA, FcγRIIA, or FcγRI on macrophages. The engagement of Fc receptors on macrophages triggers phagocytosis of target cells and results in macrophages engulfing and eliminating the target cells, e.g., engineered human CAR T cells.

In some embodiments, an NK cell inhibitor reduces NK cell-mediated complement dependent cytotoxicity (CDC) of anti-CD19 CAR T cells. Antibodies bound to a cell surface, e.g., NK cell surface, trigger complement activation through the classical pathway. Complement activation induces cell lysis, phagocytosis, chemotaxis, and immune cell activation. Complement component C1 recognizes Fc portion of antibodies and becomes activated upon antibody binding. C1 activation triggers a cascade of enzyme activation, cumulating into the cleavage and activation of complement component C3 into C3a and C3b. C3b is opsonized on cell surface and triggers downstream activation of C5b-C9 components to form membrane-attack complexes (MACs) on target cell membrane, resulting in membrane disruption and cell lysis.

In some embodiments, an NK cell inhibitor reduces NK cell-mediated apoptosis of anti-CD19 CAR T cells. Once NK cells recognize and engage target cells (e.g., anti-CD19 CAR T cells) through receptor binding, immunological synapses (ISs) are formed through cytoskeletal reorganization that polarizes microtubules formation, allowing transport and release of NK lytic enzymes into the target cells. Exemplary lytic enzymes include Granzyme B, perforin, FasL, TRAIL, and granulosyn. A serine protease, Granzyme B triggers apoptosis through caspase-dependent pathways by directly cleaving pro-apoptotic molecules such as caspase-8 and caspase-3. Granzyme B also induces apoptosis by cleaving the pro-apoptotic molecule, Bid, which causes cytochrome C release from mitochondria. Other than lytic degranulation, the Fas ligand (FasL) and TNR-related apoptosis-inducing ligand (TRAIL) molecules on NK cells also induce cell death. These receptors bind and activate death receptors TRAILR and Fas on target cells, and trigger a pro-apoptotic cascade involving caspases and IL1β-converting enzyme (ICE) proteases.

In addition to cytolytic functions, NK cells also exert their immunomodulatory function through the secretion of inflammatory and immunosuppressive cytokines. Upon contact with target cells, NK cells secrete Th1 cytokines, IFN-γ, TNF, GM-CSF, etc. These cytokines activate T cells, dendritic cells, macrophages, and neutrophils. NK cells additionally secrete chemokines, e.g., MIP-1α, MIP-10, RANTES, lymphotoxin, IL-8 (CXCL8), which attracts effector cells to the activation site. In some embodiments, an NK cell inhibitor reduces an immunomodulatory function of an NK cell. In some embodiments, an NK cell inhibitor reduces secretion of inflammatory cytokines, resulting in reduced activation induced cell death of anti-CD19 CAR T cells.

In vitro and in vivo experiments to determine NK cell activity are known in the art. Exemplary assays include cytolytic assays, ADCC assays, flow cytometry assays to determine cytokine secretion, apoptosis induction, degranulation, CDC or NK cell proliferation. See, e.g., Huang M et al., Hepatology (2013), 57:277-288; EP 2 658 871 B1; De Weers M et al., J. Immunol. (2011) 186:1840-8; EP 1 720 907 B1; U.S. Pat. Nos. 7,829,673; 9,944,711.

(i) Exemplary NK Cell Inhibitors

An NK cell inhibitor described herein includes, but is not limited to, a small molecule, a monoclonal antibody or an antigen binding fragment thereof, a polypeptide, a polynucleotide, or combinations thereof.

In some embodiments, an NK cell inhibitor is a small molecule. An exemplary small molecule NK inhibitor is ruxolitinib (Jakafi®). Ruxolitinib is a Janus kinase inhibitor used in the treatment of myelofibrosis. Ruxolitinib binds and inhibits protein tyrosine kinases JAK 1 and 2. Patients treated with ruxolitinib demonstrated increased infection rates. Ruxolitinib reduces NK cell proliferation, cytokine-induced receptor expression and NK cell function, for example, reduced killing, reduced degranulation, reduced IFN-γ production, and reduced cytokine signaling (Schonberg et al., Blood (2014), 124(21):3169). Ruxolitinib structure and methods of preparing ruxolitinib are found, e.g., in U.S. Pat. Nos. 7,598,257, 8,415,362, 8,722,693, 8,882,481, 8,829,013, and 9,079,912. Additional exemplary small molecule immunosuppressive drugs that inhibit NK cell functions are described in Pradier A et al., Front. Imunol. (2019), 10:556. In some embodiments, the NK cell inhibitor is ruxolitinib, cyclosporine A (CsA), tacrolimus (TAC), mycophenolic acid (MPA), mycophenolate mofetil (MMF), everolimus, or rapamycin.

In some embodiments, an NK cell inhibitor is a polypeptide. HLA-G is a non-classical class I antigen expressed in human placenta and thymic epithelial cells. Expression of the HLA-G antigen on the placenta protects the fetus from maternal immune rejection. Rouas-Freiss N. et al., Proc. Natl. Acad. Sci. (1997), 94:5249-5254. The HLA-G gene is alternatively spliced and transcribes HLA-G mRNAs encoding membrane-bound HLA-G (HLA-G1, HLA-G2, HLA-G3, and HLA-G4) and soluble HLA-G (HLA-G5, HLA-G6, and HLA-G7). HLA-G expression on cancer cells protects B cell lymphoma from NK mediated cell lysis. Similarly, transfection of HLA-G1 and HLA-G2 isoforms into K562 target cells abolished cytotoxicity mediated by NK-like YT2C2 T cell leukemia clone. Target cells transfected with extracellular HLA-G1, G2, G3, or G4 also inhibit cytotoxic activity of NK cells in a cell lysis assay. See e.g., EP1189627, Example 3. A recombinant fusion polypeptide comprising β2M-spacer-HLA-G5 formulated into microspheres and administered intraperitoneally into mice receiving allogeneic skin transplants was able to improve graft tolerance. See, e.g., EP 2 184 297 A1. Additional HLA-G recombinant proteins have been tested as potential treatments for tissue rejection. See, e.g., Favier B. et al., PLoS One (2011), 6(7):e21011; EP 2 264 067 A1. Accordingly, in some embodiments, the NK cell inhibitor is HLA-G1, HLA-G2, HLA-G3, GLA-G4, β2M-HLA-G5, HLA-G alpha 1 domain-Fc, or HLA-G alpha 1.

In some embodiments, an NK cell inhibitor is a polynucleotide. In some embodiments, the polynucleotide includes, but is not limited to a small interfering RNA (siRNA), a short hairpin RNA (shRNA), or antisense oligonucleotide (ASO). In some embodiments, the polynucleotide is formulated into lipid nanoparticles (LNP) for delivery into cells. In some embodiments, the polynucleotide is conjugated for delivery to specific cell types. For example, siRNA conjugated to trivalent N-acetylgalactosamine receptor (GalNAc), for targeting liver cells. In some embodiments, the siRNA is conjugated to CpG nucleotides, which bind receptors on dendritic cells or macrophages. In some embodiments, the polynucleotide is delivered in a vector. In some embodiments, the vector is a plasmid vectors or DNA minicircles, In some embodiments, the vector is a recombinant virus vector. In some embodiments, the recombinant virus is a recombinant poxvirus, a recombination herpesvirus, a recombinant adenovirus, a recombinant lentiviral, or a recombinant vesicular stomatitis virus (VSV), and combinations thereof. In a non-limiting example, the NK cell inhibitor is a shRNA targeting the NKG2D receptor. Huang M. et al., Hepatology (2013), 57:277-288. NK mediated cytolysis is reduced when a plasmid containing shRNA targeting three murine NKG2D was injected into mice. In another embodiment, a potassium channel tetramerization domain containing 9 (KCTD9) protein is elevated in NK cells of patients with viral hepatitis. Zhang X. et al., BMC Immunol. (2018), 19:20. Injection of plasmid encoding shRNA targeting KCTD9 into a mouse hepatitis model resulted in increased survival of the mice. In some embodiments, the NK cell inhibitor is NKG2D shRNA, or KCTD9 shRNA.

In some embodiments, an NK cell inhibitor is a monoclonal antibody. Non-limiting examples of antibodies that reduce NK cell activity are disclosed in AU2005321017B2 (anti-NKG2A antibody), US20030095965A1 (bivalent antibodies to CD94/NKG2 receptors), U.S. Pat. No. 9,211,328 (antibodies to NKG2D), and U.S. Pat. No. 7,829,673 (antibodies to CD38). Accordingly, in some embodiments the NK cell inhibitor is an anti-NKG2A antibody, a bivalent antibody to CD94/NKG2 receptors, an anti-NKG2D antibody, or an anti-CD38 antibody.

(ii) Anti-CD38 Antibodies

In some embodiments, an NK cell inhibitor for use in the combined therapy with anti-CD19 CAR-T cells such as CTX110 cells as disclosed herein is an anti-CD38 antibody. CD38, also known as cyclic ADP ribose hydrolase, is a 46-kDa type II transmembrane glycoprotein that synthesizes and hydrolyzes cyclic adenosine 5′-diphosphate-ribose, an intracellular calcium ion mobilizing messenger.

CD38 is overexpressed in hematologic malignancies, and on various immune cell populations including regulatory and activated T cells, B cells, myeloid-derived suppressor cells (MDSCs), and natural killer (NK) cells. Administration of an anti-CD38 antibody may suppress specific T cell, B cell, and/or NK cell subpopulations, which may mitigate the potential host immune response to anti-CD19 CAR T cells, thereby allowing increased expansion and persistence of anti-CD19 CAR T cells.

An amino acid sequence of an exemplary human CD38 protein is provided in SEQ ID NO: 56 (NCBI Reference Sequence: NP001766.2). An mRNA sequence encoding an exemplary human CD38 protein is provided in SEQ ID NO: 57 (NCBI Reference Sequence: NM_001775.4) (Homo sapiens CD38 molecule (CD38), transcript variant 1).

The present disclosure provides antibodies or antigen-binding fragments thereof that specifically bind CD38 (e.g., human CD38) for use in the methods described herein. An antibody (interchangeably used in plural form) as used herein is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) monoclonal antibodies, but also antigen-binding fragments (such as Fab, Fab′, F(ab′)2, Fv, single chain variable fragment (scFv)), mutants thereof, fusion proteins comprising an antibody portion, humanized antibodies, chimeric antibodies, diabodies, linear antibodies, single chain antibodies, single domain antibodies (e.g., camel or llama VHH antibodies), multi-specific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. These regions/residues that are responsible for antigen-binding can be identified from amino acid sequences of the V_{H}/V_{L} sequences of a reference antibody (e.g., an anti-CD38 antibody as described herein) by methods known in the art. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. As used herein, a CDR may refer to the CDR defined by any method known in the art. Two antibodies having the same CDR means that the two antibodies have the same amino acid sequence of that CDR as determined by the same method. See, e.g., Kabat, E. A. et al., (1991) Sequences of Proteins of Immunological Interest, Fifth Edition, U.S. Department of Health and Human Services, NIH Publication No. 91-3242, Chothia et al., (1989) Nature 342:877; Chothia, C. et al., (1987) J. Mol. Biol. 196:901-917, Al-Lazikani et al., (1997) J. Molec. Biol. 273:927-948; and Almagro, J. Mol. Recognit. 17:132-143 (2004). See also hgmp.mrc.ac.uk and bioinf.org.uk/abs.

An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antibody that specifically (or preferentially) binds to a CD38 epitope is an antibody that binds this CD38 epitope with greater affinity, avidity, more readily, and/or with greater duration than it binds to other CD38 epitopes or non-CD38 epitopes. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

An anti-CD38 antibody is an antibody capable of binding to CD38, which may inhibit CD38 biological activity and/or downstream pathway(s) mediated by CD38. In some examples, an anti-CD38 antibody used in the methods described herein suppresses CD38 biological activity by at least 20%, at least 40%, at least 50%, at least 75%, at least 90%, at least 100%, or by at least 2-fold, at least 5-fold, at least 10-fold, at least 20-fold, at least 50-fold, at least 100-fold, or at least 1000-fold.

The binding affinity of an anti-CD38 antibody to CD38 (such as human CD38) can be less than any of about 100 nM, about 50 nM, about 10 nM, about 1 nM, about 500 pM, about 100 pM, or about 50 pM to any of about 2 pM. Binding affinity can be expressed K_{D} or dissociation constant, and an increased binding affinity corresponds to a decreased K_{D}. One way of determining binding affinity of antibodies to CD38 is by measuring binding affinity of monofunctional Fab fragments of the antibody. To obtain Fab fragments, an antibody (for example, IgG) can be cleaved with papain or expressed recombinantly. The affinity of an anti-CD38 Fab fragment of an antibody can be determined by surface plasmon resonance (BIAcore3000™ surface plasmon resonance (SPR) system, BIAcore, INC, Piscaway N.J.). Kinetic association rates (kₒₙ) and dissociation rates (k_{off}) (generally measured at 25° C.) are obtained; and equilibrium dissociation constant (K_{D}) values are calculated as k_{off}/kₒₙ.

In some embodiments, an anti-CD38 antibody binds human CD38, and does not significantly bind a CD38 from another mammalian species. In some embodiments, the anti-CD38 antibody binds human CD38 as well as one or more CD38 from another mammalian species. In still other embodiments, the antibody binds CD38 and does not significantly cross-react with other proteins. The epitope(s) bound by the antibody can be continuous or discontinuous.

In some embodiments, an anti-CD38 antibody as described herein has a suitable binding affinity for the target antigen (e.g., CD38) or antigenic epitopes thereof. As used herein, “binding affinity” refers to the apparent association constant or K_{A}. The K_{A} is the reciprocal of the dissociation constant (K_{D}). The anti-CD38 antibody described herein may have a binding affinity (K_{D}) of at least 10₋₅, 10₋₆, 10⁻⁷, 10⁻⁸, 10⁻⁹, 10⁻¹⁰ M, or lower for the target antigen or antigenic epitope. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 105 fold. In some embodiments, any of the anti-CD38 antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

The antibodies to be used as provided herein can be murine, rat, human, or any other origin (including chimeric or humanized antibodies). In some examples, the anti-CD38 antibody is a human antibody or a humanized antibody. In some examples, the antibody comprises a modified constant region, such as a constant region that is immunologically inert, e.g., does not trigger complement mediated lysis, or does not stimulate antibody-dependent cell mediated cytotoxicity (ADCC).

Any of the antibodies described herein can be either monoclonal or polyclonal. A “monoclonal antibody” refers to a homogenous antibody population and a “polyclonal antibody” refers to a heterogenous antibody population. These two terms do not limit the source of an antibody or the manner in which it is made.

In some embodiments, an anti-CD38 antibody used in the methods described herein is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. A humanized antibody optimally also will comprise at least a portion of an immunoglobulin constant region or domain (Fc), typically that of a human immunoglobulin. Other forms of humanized antibodies have one or more CDRs (one, two, three, four, five, and/or six) which are altered with respect to the original antibody, which are also termed one or more CDRs “derived from” one or more CDRs from the original antibody. Humanized antibodies may also involve affinity maturation.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions can be used to substitute for the corresponding residues in the human acceptor genes.

In some embodiments, an anti-CD38 antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

Anti-CD38 antibodies have been tested in various pre-clinical and clinical studies, e.g., for NK/T cell lymphoma or T-cell acute lymphoblastic leukemia. Exemplary anti-CD38 antibodies tested for anti-tumor properties include SAR650984 (also referred to as isatuximab, chimeric mAb), which is in phase I clinical trials in patients with CD38+ B-cell malignancies (Deckert J. et al., Clin. Cancer. Res. (2014): 20(17):4574-83), MOR202 (also referred to as MOR03087, fully human mAb), and TAK-079 (fully human mAb).

In some embodiments, an anti-CD38 antibody for use in the present disclosure includes SAR650984 (isatuximab), MOR202, Ab79, Ab10, HM-025, HM-028, HM-034; as well as antibodies disclosed in U.S. Pat. Nos. 9,944,711, 7,829,673, WO2006/099875, WO 2008/047242, WO2012/092612, and EP1720907B1, the relevant disclosures of each of the prior patents and patent applications are herein incorporated by reference for the purposes and subject matter referenced herein.

In some embodiments, the anti-CD38 antibody disclosed herein may be a functional variant of any of the reference antibodies disclosed herein (e.g., daratumumab). Such a functional variant may comprise the same heavy chain and light chain complementary determining regions as the reference antibody. In some examples, the functional variant may comprise the same heavy chain variable region and the same light chain variable region as the reference antibody.

In some embodiments, the anti-CD38 antibody for use in the present disclosure is daratumumab. Daratumumab (also referred to as Darzalex®, HuMax-CD38, or IgG1-005) is a fully human IgGκ monoclonal antibody that targets CD38 and has been approved for treating multiple myeloma. It is used as a monotherapy or as a combination therapy for treating newly diagnosed or previously treated multiple myeloma patients. Daratumumab is described in U.S. Pat. No. 7,829,673 and WO2006/099875, the relevant disclosures of each of which are herein incorporated by reference for the purposes and subject matter referenced herein.

Also, within the scope of the present disclosure are functional variants of any of the exemplary antibodies as disclosed herein, e.g., daratumumab. A functional variant may contain one or more amino acid residue variations in the V_{H} and/or V_{L}, or in one or more of the HC CDRs and/or one or more of the V_{L} CDRs as relative to the exemplary antibody, while retaining substantially similar binding and biological activities (e.g., substantially similar binding affinity, binding specificity, inhibitory activity, anti-tumor activity, or a combination thereof) as the reference antibody.

In some instances, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art. See, e.g., Molecular Cloning: A Laboratory Manual, J. Sambrook et al., eds., Second Edition, Cold Spring Harbor Laboratory Press, Cold Spring Harbor, N.Y., 1989, or Current Protocols in Molecular Biology, Ausubel et al., eds., John Wiley & Sons, Inc., New York. Conservative substitutions of amino acids include substitutions made amongst amino acids within the following groups: (a) A→G, S; (b) R→K, H; (c) N→Q, H; (d) D→E, N; (e) C→S, A; (f) Q→N; (g) E→D, Q; (h) G→A; (i) H→N, Q; (j) I→L, V; (k) L→I, V; (l) K→R, H; (m) M→L, I, Y; (n) F→Y, M, L; (o) P→A; (p) S→T; (q) T→S; (r) W→Y, F; (s) Y→W, F; and (t) V→I, L.

Any of the anti-CD38 antibodies, including human antibodies or humanized antibodies, can be prepared by conventional approaches, for example, hybridoma technology, antibody library screening, or recombinant technology. See, for example, Harlow and Lane, (1998) Antibodies: A Laboratory Manual, Cold Spring Harbor Laboratory, New York, WO 87/04462, Morrison et al., (1984) Proc. Nat. Acad. Sci. 81:6851, and Queen et al., Proc. Natl. Acad. Sci. USA, 86:10029-10033 (1989).

(iii) Daratumumab

In some embodiments, the anti-CD38 antibody for use in the combined therapy with anti-CD19 CAR-T cells such as CTX110 cells can be daratumumab or a functional variant thereof (e.g., an antibody which binds to the same epitope as daratumumab). Daratumumab binds an epitope on CD38 that comprises two β-strands located at amino acids 233-246 and 267-280 of CD38. Experiments with CD38 mutant polypeptides show that the S274 amino acid residue is important for daratumumab binding. (van de Donk NWCJ et al., Immunol. Rev. (2016) 270:95-112). Daratumumab's binding orientation to CD38 allows for Fc-receptor mediated downstream immune processes.

Mechanisms of action attributed to Daratumumab as a lymphoma and multiple myeloma therapy includes Fc-dependent effector mechanisms such as complement-dependent cytotoxicity (CDC), natural killer (NK)-cell mediated antibody-dependent cellular cytotoxicity (ADCC) (De Weers M. et al., J. Immunol. (2011) 186:1840-8), antibody-mediated cellular phagocytosis (ADCP) (Overdijk M B. et al., MAbs (2015), 7(2):311-21), and apoptosis after cross-linking (van de Donk NWCJ and Usmani S Z, Front. Immunol. (2018), 9:2134).

The full heavy chain amino acid sequence of daratumumab is set forth in SEQ ID NO: 57 and the full light chain amino acid sequence of daratumumab is set forth in SEQ ID NO: 58. The amino acid sequence of the heavy chain variable region of daratumumab is set forth in SEQ ID NO: 59 and the amino acid sequence of the light chain variable region of daratumumab is set forth in SEQ ID NO: 60. Daratumumab includes the heavy chain complementary determining regions (HCDRs) 1, 2, and 3 (SEQ ID NOs: 61, 62, and 63, respectively), and the light chain CDRs (LCDRs) 1, 2, and 3 (SEQ ID NOs: 64, 65, and 66, respectively). In some embodiments, these sequences can be used to produce a monoclonal antibody that binds CD38. For example, methods for making daratumumab are described in U.S. Pat. No. 7,829,673 (incorporated herein by reference for the purpose and subject matter referenced herein).

In some embodiments, the anti-CD38 antibody comprises: (a) an immunoglobulin heavy chain variable region and (b) an immunoglobulin light variable region, wherein the heavy chain variable region and the light chain variable region defines a binding site (paratope) for CD38. In some embodiments, the heavy chain variable region comprises an HCDR1 comprising the amino acid sequence set forth in SEQ ID NO: 63, an HCDR2 comprising the amino acid sequence set forth in SEQ ID NO: 64; and an HCDR3 comprising the amino acid sequence in SEQ ID NO: 65, following the Kabat numbering scheme. The HCDR1, HCDR2, and HCDR3 sequences are separated by the immunoglobulin framework (FR) sequences.

In some embodiments, the anti-CD38 antibody comprises: (a) an immunoglobulin light chain variable region and (b) an immunoglobulin heavy chain variable region, wherein the light chain variable region and the heavy chain variable region defines a binding site (paratope) for CD38. In some embodiments, the light chain variable region comprises an LCDR1 comprising the amino acid sequence set forth in SEQ ID NO: 66, an LCDR2 comprising the amino acid sequence set forth in SEQ ID NO: 67; and an LCDR3 comprising the amino acid sequence in SEQ ID NO: 68. The LCDR1, LCDR2, and LCDR3 sequences are separated by the immunoglobulin framework (FR) sequences.

In some embodiments, the anti-CD38 antibody comprises an immunoglobulin heavy chain variable region (V_{H}) comprising the amino acid sequence set forth in SEQ ID NO: 60, and an immunoglobulin light chain variable region (V_{L}). In some embodiments, the anti-CD38 antibody comprises an immunoglobulin light chain variable region (V_{L}) comprising the amino acid sequence set forth in SEQ ID NO: 62, and an immunoglobulin heavy chain variable region (V_{H}). In some embodiments, the anti-CD38 antibody comprises an immunoglobulin heavy chain variable region (V_{H}) comprising the amino acid sequence set forth in SEQ ID NO: 60, and an immunoglobulin light chain variable region (V_{L}) comprising the amino acid sequence set forth in SEQ ID NO: 62, and an immunoglobulin heavy chain variable region (V_{H}).

In some embodiments, the anti-CD38 antibody comprises a V_{H} comprising an amino acid sequence that is at least 70%, 75%, 70%, 85%, 90%, 95%, 96%, 97%, 98%, and 99% identical to the amino acid sequence set forth in SEQ ID NO: 60, and comprises an V_{L} comprising an amino acid sequence that is at least 70%, 75%, 70%, 85%, 90%, 95%, 96%, 97%, 98%, and 99% identical to the amino acid sequence set forth in SEQ ID NO: 62.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul et al., J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the anti-CD38 antibody is a functional variant of daratumumab. In some examples, a functional variant comprises substantially the same V_{H} and V_{L} CDRs as daratumumab. For example, it may comprise only up to 8 (e.g., 8, 7, 6, 5, 4, 3, 2, or 1) amino acid residue variations in the total CDR regions of the antibody and binds the same epitope of CD38 with substantially similar affinity (e.g., having a K_{D} value in the same order) as daratumumab. In some instances, the functional variants may have the same heavy chain CDR3 as daratumumab, and optionally the same light chain CDR3 as daratumumab. Alternatively, or in addition, the functional variants may have the same heavy chain CDR2 as daratumumab. Such an anti-CD38 antibody may comprise a V_{H} fragment having CDR amino acid residue variations in only the heavy chain CDR1 as compared with the V_{H} of daratumumab. In some examples, the anti-CD38 antibody may further comprise a V_{L} fragment having the same V_{L} CDR3, and optionally same V_{L} CDR1 or V_{L} CDR2 as daratumumab. Alternatively or in addition, the amino acid residue variations can be conservative amino acid residue substitutions (see above disclosures).

In some embodiments, the anti-CD38 antibody may comprise heavy chain CDRs that are at least 80% (e.g., 85%, 90%, 95%, or 98%) sequence identity, individually or collectively, as compared with the V_{H} CDRs of daratumumab. Alternatively, or in addition, the anti-CD38 antibody may comprise light chain CDRs that are at least 80% (e.g., 85%, 90%, 95%, or 98%) sequence identity, individually or collectively, as compared with the V_{L} CDRs as daratumumab. As used herein, “individually” means that one CDR of an antibody shares the indicated sequence identity relative to the corresponding CDR of daratumumab. “Collectively” means that three V_{H} or V_{L} CDRs of an antibody in combination share the indicated sequence identity relative the corresponding three V_{H} or V_{L} CDRs of daratumumab.

In some embodiments, the anti-CD38 antibody binds to the same epitope bound by daratumumab on human CD38. In some embodiments, the anti-CD38 antibody competes with daratumumab for binding to human CD38.

Competition assays for determining whether an antibody binds to the same epitope as daratumumab, or competes with daratumumab for binding to CD38, are known in the art. Exemplary competition assays include immunoassays (e.g., ELISA assay, RIA assays), surface plasmon resonance, (e.g., BIAcore analysis), bio-layer interferometry, and flow cytometry.

A competition assay typically involves an immobilized antigen (e.g., CD38), a test antibody (e.g., CD38-binding antibody) and a reference antibody (e.g., daratumumab). Either one of the reference or test antibody is labeled, and the other unlabeled. In some embodiments, competitive binding is determined by the amount of a reference antibody bound to the immobilized antigen in increasing concentrations of the test antibody. Antibodies that compete with a reference antibody include antibodies that bind the same or overlapping epitopes as the reference antibody. In some embodiments, the test antibodies bind to adjacent, non-overlapping epitopes such that the proximity of the antibodies causes a steric hindrance sufficient to affect the binding of the reference antibody to the antigen.

A competition assay can be conducted in both directions to ensure that the presence of the label or steric hindrance does not interfere or inhibit binding to the epitope. For example, in the first direction, the reference antibody is labeled and the test antibody is unlabeled. In the second direction, the test antibody is labeled, and the reference antibody is unlabeled. In another embodiment, in the first direction, the reference antibody is bound to the immobilized antigen, and increasing concentrations of the test antibody are added to measure competitive binding. In the second direction, the test antibody is bound to the immobilized antigen, and increasing concentrations of the reference antibody are added to measure competitive binding.

In some embodiments, two antibodies can be determined to bind to the same epitope if essentially all amino acid mutations in the antigen that reduce or eliminate the binding of one antibody reduce or eliminate binding of the other. Two antibodies can be determined to bind to overlapping epitopes if only a subset of the mutations that reduce or eliminate the binding of one antibody reduces or eliminates the binding of the other.

In some embodiments, the heavy chain of any of the anti-CD38 antibodies as described herein (e.g., daratumumab) may further comprise a heavy chain constant region (CH) or a portion thereof (e.g., CH1, CH2, CH3, or a combination thereof). The heavy chain constant region can of any suitable origin, e.g., human, mouse, rat, or rabbit. Alternatively or in addition, the light chain of the anti-CD38 antibody may further comprise a light chain constant region (CL), which can be any CL known in the art. In some examples, the CL is a kappa light chain. In other examples, the CL is a lambda light chain. Antibody heavy and light chain constant regions are well known in the art, e.g., those provided in the IMGT database (www.imgt.org) or at www.vbase2.org/vbstat.php., the relevant disclosures of which are herein incorporated by reference for the purposes and subject matter referenced herein.

Daratumumab or a functional variant thereof can be prepared by a conventional method, for example, by recombinant technology.

It should be understood that the described antibodies are only exemplary and that any anti-CD38 antibodies can be used in the compositions and methods disclosed herein. Methods for producing antibodies are known to those of skill in the art.

IV. Allogeneic CAR T Cell Therapy for Treatment of B Cell Malignancies, Optionally in Combination with an NK Cell Inhibitor

In some aspects, provided herein are methods for treating a human patient having a B cell malignancy using a population of any of the genetically engineered anti-CD19 CAR T cells such as the CTX110 T cells as disclosed herein, optionally in combination with an NK cell inhibitor such as daratumumab.

The allogeneic anti-CD19 CAR T cell therapy in combination with an NK cell inhibitor may comprise three stages of treatment: (i) an NK cell inhibitor treatment, which comprises giving one or more doses of the NK cell inhibitor to a suitable human patient, (ii) a conditioning regimen (lymphodepleting treatment), which comprises giving one or more doses of one or more lymphodepleting agents to the human patient, and (iii) a treatment regimen (allogeneic anti-CD19 CAR T cell therapy), which comprises administration of the population of allogeneic anti-CD19 CAR T cells such as the CTX110 T cells as disclosed herein to the human patient. In some embodiments, the treatment further comprises redosing the human patient with at least one subsequent dose of the NK cell inhibitor. Alternatively or in addition, the treatment may further comprise redosing the human patient with at least one subsequent dose of the anti-CD19 CAR-T cells (e.g., up to two subsequent doses or up to three doses in total). In some instances, the redosing may be accompanied with a lymphodepleting treatment prior to the redosing. In other instances, the redosing may not be accompanied with a prior lymphodepleting treatment.

In some embodiments, the combined therapy may include the NK cell inhibitor treatment and the anti-CD19 CAR-T treatment without a lymphodepletion treatment prior to the CAR-T treatment.

Also provided herein is a treatment of ALL (e.g., adult B cell ALL) by one or more doses of any of the populations of anti-CD19 CAR-T cells disclosed herein. The human patient is subject to a lymphodepletion treatment prior to the first dose of the population of genetically engineered T cells. In some instances, the subsequent dose(s) may be accompanied with a lymphodepleting treatment prior to the redosing. In other instances, the subsequent dose(s) may not be accompanied with a prior lymphodepleting treatment.

(a) Patient Population

A human patient may be any human subject for whom diagnosis, treatment, or therapy is desired. A human patient may be of any age. In some embodiments, the human patient is an adult (e.g., a person who is at least 18 years old). In some embodiments, the human patient is an adult. In some embodiments, the human patient is ≥18 to ≤60 years old. In some examples, the human patient may be older than 60. In some embodiments, the human patient is ≥60 to ≤70 years old. In some embodiments, the human patient is ≥18 to ≤70 years old. In some examples, the human patient may have a body weight of 50 kg or higher. In some embodiments, the human patient can be a child.

A human patient to be treated by the methods described herein can be a human patient having, suspected of having, or a risk for having a B cell malignancy, e.g., CD19⁺ B cell malignancy. A subject suspected of having a B cell malignancy might show one or more symptoms of B cell malignancy, e.g., unexplained weight loss, fatigue, night sweats, shortness of breath, or swollen glands. A subject at risk for a B cell malignancy can be a subject having one or more of the risk factors for B cell malignancy, e.g., a weakened immune system, age, male, or infection (e.g., Epstein-Barr virus infection). A human patient who needs the anti-CD19 CAR T cell (e.g., CTX110 T cell) and the NK cell inhibitor combination treatment may be identified by routine medical examination, e.g., physical examination, laboratory tests, biopsy (e.g., bone marrow biopsy and/or lymph node biopsy), magnetic resonance imaging (MRI) scans, or ultrasound exams.

Non-Hodgkin Lymphoma (NHL)

In some embodiments, the CD19+ B cell malignancy is a non-Hodgkin lymphoma (NHLs), which are a heterogeneous group of malignancies originating from B lymphocytes, T lymphocytes, or natural killer (NK) cells. The World Health Organization defines more than 60 different subcategories of NHL based on cell type in which the cancer originates, histology, mutational profiling, and protein markers on the cellular surface, and NHL is the 10th most common malignancy worldwide (Chihara et al., 2015; Trask et al., 2012). NHL accounts for 4.3% of all new cancer cases reported and is the 8th leading cause of cancer deaths in the United States. The major subtypes of NHL include diffuse large B cell lymphoma (DLBCL), chronic lymphocytic leukemia (CLL), and follicular lymphoma (FL; (Teras et al., 2016; Trask et al., 2012). CD19 expression is ubiquitous on B cell malignancies and maintained among indolent and aggressive subtypes of NHL (Scheuermann and Racila, 1995), which has contributed to the increase of development of CD19-directed therapies in these indications.

In some examples, B cell malignancies that may be treated using the methods described herein include, but are not limited to, diffuse large B cell lymphoma (DLBCL), high grade B cell lymphoma with MYC and BCL2 and/or BCL6 rearrangement, transformed follicular lymphoma (FL), grade 3b FL, or Richter's transformation of chronic lymphocytic leukemia (CLL). In some examples, the B cell malignancy is DLBCL, e.g., high grade DLBCL or DLBCL not otherwise specified (NOS). In some examples, the B cell malignance is acute lymphocytic leukemia (ALL). In some examples, the B cell malignancy is transformed FL or grade 3b FL. In some examples, the human patient has at least one measurable lesion that is fluorodeoxyglucose positron emission tomography (PET)-positive. In some examples, the human patient may have a refractory NHL disease with bulky presentation (high-risk subjects).

DLBCL is the most common type of NHL, accounting for 30-40% of diagnosed cases (Sehn and Gascoyne, 2015). Approximately 30-50% achieve cure with first-line chemoimmunotherapy consisting of rituximab, cyclophosphamide, doxorubicin, vincristine, and prednisone (R-CHOP; (Coiffier et al., 2010; Maurer et al., 2016). However, approximately 20% are refractory to R-CHOP and 30% relapse following complete response (CR; (Maurer et al., 2016).

FL is a heterogeneous disease, usually indolent, and accounts for about 20% of reported NHL. The course is characterized by initial response to therapies followed by relapse and, at times, transformation to a more aggressive form of lymphoma. It is generally considered incurable at more advanced stages, although the 10-year survival rate is 71% for subjects with early-stage disease and 0 to 1 risk factors based on Follicular Lymphoma International Prognostic Index score (Solal-Céligny et al., 2004). FL is divided into grades 1-3 based on histologic assessment and proportion of centrocytes to centroblasts, and grade 3 is subdivided into 3a and 3b. FL grade 3b is now considered a biologically distinct entity, with frequent absence of t(14;18) and CD10 expression, and increased p53 and MUM1/IRF4 expression (Horn et al., 2011). A large retrospective analysis of more than 500 FL cases further confirmed that the clinical course of FL grade 3b is similar to FL grade 1-2, whereas FL grade 3b has a clinical course more similar to that of DLBCL (Kahl and Yang, 2016; Wahlin et al., 2012). Because of this, FL grade 3b is typically managed similarly to DLBCL (Kahl and Yang, 2016).

In some embodiments, the human patient to be treated has DLBCL and exhibits pararectal mass, retroperitoneal mass, diffuse lymph nodes (LN), lytic lesions, tonsillar lesion, or a combination thereof. Alternatively or in addition, the human patient may have bone marrow diffusion. In other examples, the human patient is free of bone marrow diffusion. In some embodiments, the human patient to be treated has transformed FL. Such a human patient may exhibit diffuse LN. In some instances, the human patient may have bone marrow diffusion. In other instances, the human patient may be free of bone marrow diffusion.

A human patient to be treated by methods described herein may be a human patient that has relapsed following a treatment and/or that has been become resistant to a treatment and/or that has been non-responsive to a treatment. As used herein, “relapsed” or “relapses” refers to a B cell malignancy such as those disclosed herein (e.g., NHL or ALL disclosed herein) that returns following a period of complete response. Progressive disease refers to an instance when a disease worsens after the last evaluation (e.g., stable disease or partial response). In some embodiments, progression occurs during the treatment. In some embodiments, relapse occurs after the treatment. A lack of response may be determined by routine medical practice. For example, the human patient to be treated by methods described herein may be a human patient that has had one or more lines of prior anti-cancer therapies. In some instances, the human patient may have undergone two or more lines of prior anti-cancer therapies, e.g., a chemotherapy, an immunotherapy, a surgery, or a combination thereof. In some examples, the prior anti-cancer therapies may comprise an anti-CD20 antibody therapy, an anthracycline-containing therapy, or a combination thereof.

In some instances, the human patient has a refractory B cell malignancy. As used herein, “refractory” refers to a B cell malignancy such as those disclosed herein (e.g., NHL or ALL disclosed herein) that does not respond to or becomes resistant to a treatment. A human patient having a refractory B cell malignancy may have progressive disease on last therapy, or has stable disease following at least two cycles of therapy with duration of stable disease of up to 6 months (e.g., up to 5 months, up to 4 months, or up to 3 months or up to 2 months or up to 1 month). In some instances, the human patient may have undergone a prior autologous hematopoietic stem cell transplantation (HSCT) and showed no response to such (failed) or have progressed or relapsed after achieving some response. In other instances, the human patient may not be eligible for prior autologous HSCT.

A human patient may be screened to determine whether the patient is eligible to undergo an NK cell inhibitor treatment and/or a conditioning regimen (lymphodepleting treatment) and/or an allogeneic anti-CD19 CAR-T cell therapy as disclosed herein.

For example, a human patient who is eligible to undergo an NK cell inhibitor treatment and/or a conditioning regimen and/or an allogeneic anti-CD19 CAR-T cell therapy may show one or more of the following features: (a) has an Eastern Cooperative Oncology Group (ECOG) performance status 0 or 1; (b) adequate renal, liver, cardiac, and/or pulmonary function; (c) free of prior gene therapy or modified cell therapy; (d) free of prior treatment comprising an anti-CD19 antibody; (e) free of prior allogeneic HSCT; (f) free of detectable malignant cells from cerebrospinal fluid; (g) free of brain metastases; (h) free of prior central nervous system disorders; (i) free of unstable angina, arrhythmia, and/or myocardial infarction; (j) free of uncontrolled infection; (k) free of immunodeficiency disorders or autoimmune disorders that require immunosuppressive therapy; (1) free of infection by human immunodeficiency virus, hepatitis B virus, or hepatitis C virus; and (m) free of a known contraindication to NK cell inhibitor treatment.

Alternatively or in addition, a human patient who is eligible for NK cell inhibitor treatment does not show one or more of the following features: (a) significant worsening of clinical status, (b) requirement for supplemental oxygen to maintain a saturation level of greater than 90%, (c) uncontrolled cardiac arrhythmia, (d) hypotension requiring vasopressor support, (e) active infection, (f) grade ≥2 acute neurological toxicity, and (g) unresolved reaction to NK cell inhibitor treatment.

A human patient may be screened and excluded from the NK cell inhibitor regimen and/or the conditioning regimen and/or treatment regimen based on such screening results. For example, a human patient may be excluded from an NK cell inhibitor treatment (e.g., daratumumab), if the patient has a known contraindication to the NK cell inhibitor (e.g., daratumumab). In another example, a human patient may be excluded from NK cell inhibitor treatment and/or a conditioning regimen and/or the allogeneic anti-CD19 CAR-T cell therapy, if the patient meets one or more of the following exclusion criteria: (a) prior treatment with any gene therapy or genetically modified cell therapy (e.g., CAR T cells), (b) prior treatment with a CD19-directed antibody, bispecific T cell engage, or antibody-drug conjugate, unless there is confirmed CD19 expression (e.g., by immunohistochemistry or flow cytometry) after progression or relapse following most recent CD19-directed treatment, (c) prior allogeneic HSCT treatment, (d) known contraindication to NK cell inhibitor treatment and/or lymphodepletion treatment and/or any excipient of anti-CD19 CAR+ T cells, (e) detectable malignant cells from cerebrospinal fluid (CSF) or magnetic resonance imaging (MRI) indicating brain metastases during screening, or a history of central nervous system (CNS) involvement by malignancy (CSF or imaging), (f) history of a seizure disorder, cerebrovascular ischemia/hemorrhage, dementia, cerebellar disease, or any autoimmune disease with CNS involvement, (g) unstable angina, clinically significant arrhythmia, or myocardial infarction within 6 months of a treatment described herein (e.g., NK cell inhibitor treatment, lymphodepletion treatment, anti-CD19 CAR+ T cell treatment), (h) presence of bacterial, viral, or fungal infection that is uncontrolled or requires IV anti-infectives, (i) presence of infection by human immunodeficiency virus, hepatitis B virus, or hepatitis C virus, (j) previous or concurrent malignancy, except basal cell or squamous cell skin carcinoma, adequately resected and in situ carcinoma of cervix, or a previous malignancy that was completely resected and has been in remission for ≥5 years, (k) radiation therapy within 14 days of enrollment, (l) use of systemic antitumor therapy or investigational agent within 14 days or 5 half-lives, whichever is longer, of enrollment. Exceptions are made for 1) prior inhibitory/stimulatory immune checkpoint molecule therapy, which is prohibited within 3 half-lives of enrollment, and 2) rituximab use within 30 days prior to screening is prohibited, (m) primary immunodeficiency disorder or active autoimmune disease requiring steroids and/or other immunosuppressive therapy, (n) diagnosis of significant psychiatric disorder or other medical condition that, in the opinion of the healthcare providers, could impede the subject's ability to undergo treatment, and (o) women who are pregnant or breastfeeding.

In some examples, an NHL patient (e.g., any subtype disclosed herein) for treatment by any of the methods disclosed herein may meet the inclusion and exclusion criteria disclosed in Example 11 below.

Acute Lymphoblastic Leukemia (ALL)

In some embodiments, A human patient to be treated by the methods described herein can be a human patient having, suspected of having, or a risk for having acute lymphoblastic leukemia (ALL). ALL is a hematologic malignancy characterized by highly proliferative immature lymphoid cells in the bone marrow and peripheral blood. In adults, ALL accounts for approximately 20% of all leukemias, the second most common, with an incidence of more than 6,500 cases per year in the United States alone (Terwilliger and Abdul-Hay, 2017). ALL carries a poor prognosis in adults, with a 5-year overall survival of 24% in patients ages 40 to 59 years, and 18% in patients ages 60 to 69 years (Terwilliger and Abdul-Hay, 2017; Wang et al., 2015).

A subject suspected of having ALL might show one or more symptoms of ALL, e.g., fever, fatigue, pale skin, frequent infections, bone pain, shortness of breath, unexplained weight loss or swollen glands. A subject at risk for ALL can be a subject having one or more of the risk factors for ALL, e.g., previous cancer treatment, exposure to radiation, or a genetic predisposition. A human patient who needs the anti-CD19 CAR T cell (e.g., CTX110 T cell) treatment may be identified by routine medical examination, e.g., physical examination, laboratory tests, biopsy (e.g., bone marrow biopsy and/or lymph node biopsy), magnetic resonance imaging (MRI) scans, or ultrasound exams.

Methods described herein may be used to treat B-cell ALL. In some examples, the B-cell ALL is B-cell ALL with recurrent genetic abnormalities or B-cell ALL not otherwise specified (NOS). Examples of B-cell ALL with recurrent genetic abnormalities include, but are not limited to, B-cell ALL with hypodiploidy, B-cell ALL with hyperdiploidy, B-cell ALL with t(9;22)(q34;q11.2)[BCR-ABL1] (aka, Philadelphia chromosome-positive (Ph+) ALL), B-cell ALL with t(v;11q23)[MLL rearranged], B-cell ALL with t(12;21)(p13;q22)[ETV6-RUNX1], B-cell ALL with t(1;19)(q23;p13.3)[TCF3-PBX1], B-cell ALL with t(5;14)(q31;q32)[1L3-IGH], B-cell ALL with intrachromosomal amplification of chromosome 21 (iAMP21), and B-cell ALL with translocations involving tyrosine kinases or cytokine receptors (‘BCR-ABL1-like ALL’). In some examples, the ALL is histologically confirmed (e.g., histologically confirmed B-cell ALL). In some embodiments, the human patient has bone marrow involvement with ≥5% blasts.

For example, the human patient to be treated by methods described herein may be a human patient that has had one or more lines of prior anti-cancer therapies. In some instances, the human patient may have undergone two or more lines of prior anti-cancer therapies, e.g., a chemotherapy, an immunotherapy, a surgery, or a combination thereof. In some examples, the prior anti-cancer therapies may comprise a multi-agent chemotherapy (e.g., vincristine, corticosteroids, an anthracycline, or a combination thereof), an allogeneic stem cell transplantation therapy, or a combination thereof. In some instances, the patient may have bone marrow relapse after one or more lines of prior anti-cancer therapies (e.g., any bone marrow relapse after allogeneic HSCT). In some instances, the patient (e.g., the patient is Philadelphia chromosome-positive (Ph+)) may have progressed after at least one line of tyrosine kinase inhibitor (TKI) therapy, a treatment involving blinatumomab, or the patient may be intolerant to or ineligible for TKI therapy. In some instances, the patient has bone marrow involvement with <50% blasts.

In some instances, the human patient has a refractory ALL. A human patient having a refractory ALL may have progressive disease on last therapy. In some instances, a human patient having a refractory ALL may have undergone two or more lines of prior anti-cancer treatment.

A human patient may be screened to determine whether the patient is eligible to undergo a conditioning regimen (lymphodepleting treatment) and/or an allogeneic anti-CD19 CAR-T cell therapy as disclosed herein.

For example, a human patient who is eligible to undergo a conditioning regimen and/or an allogeneic anti-CD19 CAR-T cell therapy may show one or more of the following features: (a) has an Eastern Cooperative Oncology Group (ECOG) performance status 0 or 1; (b) adequate renal, liver, cardiac, and/or pulmonary function; (c) free of prior gene therapy or modified cell therapy; (d) free of prior treatment comprising an anti-CD19 antibody; (e) free of prior allogeneic HSCT; (f) free of detectable malignant cells from cerebrospinal fluid; (g) free of brain metastases; (h) free of prior central nervous system disorders; (i) free of unstable angina, arrhythmia, and/or myocardial infarction; (j) free of uncontrolled infection; (k) free of immunodeficiency disorders or autoimmune disorders that require immunosuppressive therapy; and kl) free of infection by human immunodeficiency virus, hepatitis B virus, or hepatitis C virus.

Alternatively or in addition, a human patient who is eligible for lymphodepletion treatment does not show one or more of the following features: (a) significant worsening of clinical status, (b) requirement for supplemental oxygen to maintain a saturation level of greater than 90%, (c) uncontrolled cardiac arrhythmia, (d) hypotension requiring vasopressor support, (e) active infection, and (f) grade ≥2 acute neurological toxicity.

In yet another example, a human patient who is eligible for a lymphodepletion treatment regimen does not show one or more of the following features: (a) active uncontrolled infection, (b) worsening of clinical status compared to the clinical status prior to lymphodepletion treatment, and (c) grade ≥2 acute neurological toxicity.

A human patient may be screened and excluded from the conditioning regimen and/or treatment regimen based on such screening results. For example, a human patient may be excluded from a conditioning regimen and/or an allogeneic anti-CD19 CAR-T cell therapy, if the patient meets one or more of the following exclusion criteria: (a) prior treatment with any gene therapy or genetically modified cell therapy (e.g., CAR T cells), (b) prior treatment with a CD19-directed antibody, bispecific T cell engage, or antibody-drug conjugate, unless there is confirmed CD19 expression (e.g., by immunohistochemistry or flow cytometry) after progression or relapse following most recent CD19-directed treatment, (c) prior allogeneic HSCT treatment, lymphodepletion treatment, and/or any excipient of anti-CD19 CAR+ T cells, (e) detectable malignant cells from cerebrospinal fluid (CSF) or magnetic resonance imaging (MRI) indicating brain metastases during screening, or a history of central nervous system (CNS) involvement by malignancy (CSF or imaging), (f) history of a seizure disorder, cerebrovascular ischemia/hemonrhage, dementia, cerebellar disease, or any autoimmune disease with CNS involvement, (g) unstable angina, clinically significant arrhythmia, or myocardial infarction within 6 months of a treatment described herein (e.g., lymphodepletion treatment, anti-CD19 CAR+ T cell treatment), (h) presence of bacterial, viral, or fungal infection that is uncontrolled or requires IV anti-infectives, (i) presence of infection by human immunodeficiency virus, hepatitis B virus, or hepatitis C virus, (j) previous or concurrent malignancy, except basal cell or squamous cell skin carcinoma, adequately resected and in situ carcinoma of cervix, or a previous malignancy that was completely resected and has been in remission for ≥5 years, (k) radiation therapy within 14 days of enrollment, (1) use of systemic antitumor therapy or investigational agent within 14 days or 5 half-lives, whichever is longer, of enrollment. Exceptions are made for 1) prior inhibitory/stimulatory immune checkpoint molecule therapy, which is prohibited within 3 half-lives of enrollment, and 2) rituximab use within 30 days (e.g., within 14 days) prior to screening is prohibited, (m) primary immunodeficiency disorder or active autoimmune disease requiring steroids and/or other immunosuppressive therapy, (n) diagnosis of significant psychiatric disorder or other medical condition that, in the opinion of the healthcare providers, could impede the subject's ability to undergo treatment, (o) women who are pregnant or breastfeeding, (p) diagnosis of Burkitt's lymphoma/leukemia, and (q) isolated extramedullary disease.

In some examples, a human patient having received a prior allogeneic HSCT treatment may be eligible for a conditioning regimen and/or an allogeneic anti-CD19 CAR-T cell therapy, if the following criteria are meet: (a) it has been more than 6 months from the human patient's prior allogeneic HSCT treatment at the time of screening, (b) there is no evidence of acute or chronic GvHD, (c) the human patient has recovered from any HSCT-related toxicities, (d) the human patient has been off immunosuppressive therapies for at least 3 months prior to screening, (e) and the human patient has not received donor lymphocyte infusion for at least 2 months prior to screening.

In some examples, a B cell ALL patient for treatment by any of the methods disclosed herein may meet the inclusion and exclusion criteria provided in Example 11 below.

(b) NK Cell Inhibitor Treatment

In some embodiments, a human patient suitable for the treatment methods disclosed herein may receive an NK cell inhibitor to reduce or deplete the immune suppressor cells and/or the immune effector cells (e.g., NK cells) of the subject. In some instances, the human patient may receive an anti-CD38 antibody (e.g., daratumumab) to reduce or deplete the CD38-positive immune suppressor cells and/or the CD38-positive immune effector cells.

CTX110 is an allogeneic CAR T cell with disruption of the B2M locus resulting in elimination of MHC class I expression on the cell surface, NK cells can potentially detect and clear these “non-self” MHC class I negative cells (Paul and Lal, 2017). The suppression of specific NK cell subpopulations with daratumumab in addition to LD chemotherapy may reduce the potential host immune response to an allogeneic CAR T cell product, and therefore allow increased expansion and persistence of CTX110.

Preliminary nonclinical data reported herein support the addition of daratumumab to CTX110 as an exemplary treatment for the target diseases disclosed herein. Increasing concentrations of daratumumab (up to 1 μg/mL) did not kill CTX110 cells after 72 hours in vitro in the presence of 10% human complement. In addition, a pharmacological model was established, which integrates established immune effector cell interactions and clinical data from daratumumab. The model uses PK and pharmacodynamic data to make informed predictions regarding the plasma concentration of daratumumab required to achieve immunomodulation. A dose of 16 mg/kg daratumumab infused prior to fludarabine and cyclophosphamide is predicted to further suppress NK cell activity beyond chemotherapy alone, and redosing at approximately a 28-day frequency will maintain a mean trough concentration >EC₉₀ for NK cell cytotoxicity (FIG. 12). This model establishes that the addition of daratumumab to the lymphodepletion regimen and repeat dosing is a rational strategy to overcome potential alloimmune rejection by NK cells.

A “NK cell inhibitor” can be any molecule capable of reducing, depleting, or eliminating endogenous immune suppressor cells and/or endogenous immune effector cells when administered to a subject. A “anti-CD38 antibody” can be any antibody or fragment thereof capable of reducing, depleting, or eliminating endogenous CD38-positive immune suppressor cells and/or endogenous CD38-positive immune effector cells when administered to a subject.

In some embodiments, the NK cell inhibitor is administered in an amount effective in reducing the number of endogenous immune suppressor cells and/or endogenous immune effector cells by at least 10%, 20%, 30%, 40%, 50%, 60%, 70%, 80%, 90%, 95%, 96%, 96%, 97%, 98%, or at least 99% as compared to the number of endogenous immune suppressor cells and/or endogenous immune effector cells prior to administration of the NK cell inhibitor. In some embodiments, the NK cell inhibitor is administered in an amount effective in reducing the number of endogenous immune suppressor cells and/or endogenous immune effector cells such that the number of endogenous immune suppressor cells and/or endogenous immune effector cells in the subject is below the limits of detection. In some embodiments, the subject is administered at least one (e.g., 2, 3, 4, 5 or more) doses of the NK cell inhibitor.

In some embodiments, the anti-CD38 antibody (e.g., daratumumab) is administered in an amount effective in reducing the number of endogenous CD38-positive immune suppressor cells and/or endogenous CD38-positive immune effector cells by at least 10%, 20%, 30%, 40%, 50%, 60%, 70%, 80%, 90%, 95%, 96%, 96%, 97%, 98%, or at least 99% as compared to the number of endogenous CD38-positive immune suppressor cells and/or endogenous CD38-positive immune effector cells prior to administration of the anti-CD38 antibody. In some embodiments, the anti-CD38 antibody is administered in an amount effective in reducing the number of endogenous CD38-positive immune suppressor cells and/or endogenous CD38-positive immune effector cells such that the number of endogenous CD38-positive immune suppressor cells and/or endogenous CD38-positive immune effector cells in the subject is below the limits of detection. In some embodiments, the subject is administered at least one (e.g., 2, 3, 4, 5 or more) doses of the anti-CD38 antibody.

In some embodiments, the human patient may receive additional doses of daratumumab. Alternatively, the human patient may receive no additional doses of daratumumab. In other embodiments, a human patient is given one dose of darabumumab with no additional doses.

In some examples, a human patient is given daratumumab treatment comprising three doses as provided in Example 11 below. Such a human patient may exhibit stable disease at least 4 weeks after receiving an infusion of the anti-CD19 CAR T cells as disclosed herein. In some instances, the second dose of daratumumab may be administered to the patient at about 4 weeks after the first dose (Day 1) of the anti-CD19 CAR T cells such as CTX110 (e.g., on Day 28±5 days). In some instances, the third dose of daratumumab may be administered to the patient at about 2M after the first dose of the first dose of the anti-CD19 CAR T cells such as CTX110 (e.g., on Day 60±5).

The human patient may receive any suitable amount of any suitable NK cell inhibitor. In one example, the human patient receives daratumumab at about 8-32 mg/kg (e.g., about 16 mg/kg) via intravenous infusion. To facilitate administration, the dose of the NK cell inhibitor may be split over 2 consecutive days. For example, the human patient receives daratumumab at about 4-16 mg/kg (e.g., about 8 mg/kg) for two consecutive days. Alternatively, the human patient may receive daramumab at about 1500 mg to about 2500 mg (e.g., about 1800 mg) via subcutaneous (SC) injection.

The human patient may then be administered any of the conditioning regimens and any of the anti-CD19 CAR+ T cells within a suitable period after the NK cell inhibitor is administered as disclosed herein. For example, the human patient may receive a dose of the NK cell inhibitor at least 1 day prior to starting a conditioning regimen, and within about 10 days of receiving the anti-CD19 CAR+ T cells (e.g., CTX110 cells). In some examples, the human patient may receive a dose of the NK cell inhibitor at least 1 day, at least 2 days, at least 3 days, at least 4 days, at least 5 days, or at least 6 days prior to starting a conditioning regimen, and within 10 days, within 9 days, within 8 days, within 7 days, or within 6 days of receiving the anti-CD19 CAR+ T cells.

In some embodiments, the method described herein involves administering at least one subsequent dose of the NK cell inhibitor to a human patient having stable disease or better after administration of the anti-CD19 CAR⁺ T cells. For example, a human patient may be evaluated for disease progression and/or tumor response (e.g., by positron emission tomography (PET)/computed tomography (CT)) after administration of the anti-CD19 CAR+ T cells (e.g., 24-32 days after administration), and the human patient may then be administered a subsequent dose of the NK cell inhibitor if the human patient achieved stable disease or better. In some examples, a subsequent dose of the NK cell inhibitor may be administered 24-32 days (e.g., 24 days, 25 days, 26 days, 27 days, 28 days, 29 days, 30 days, 31 days, or 32 days) after the human patient is administered the anti-CD19 CAR+ T cells. The human patient may then be administered another subsequent dose of the NK cell inhibitor 56-64 days (e.g., 56 days, 57 days, 58 days, 59 days, 60 days, 61 days, 62 days, 63 days, or 64 days) after the human patient is administered the anti-CD19 CAR+ T cells.

In some instances, the subsequent dose(s) of the NK cell inhibitor (e.g., daratumumab) may be the same as the first dose. Alternatively, the subsequent dose(s) of the NK cell inhibitor (e.g., daratumumab) may be lower than the first dose.

In some embodiments, the method described herein involves premedication of the human patient prior to administering an NK cell inhibitor to reduce the human patient's reaction to the NK cell inhibitor. Premedication may include, but is not limited to, corticosteroids (e.g., methylprednisolone), antipyretics (e.g., acetaminophen), antihistamines, or combinations thereof. The human subject may be premedicated about 1-3 hours prior to administration of an NK cell inhibitor. For example, to reduce the risk of infusion reactions to daratumumab, the human patient may be premedicated with methylprednisolone (e.g., 100 mg administered intravenously), acetaminophen (e.g., 650-1,000 mg administered orally), and diphenhydramine hydrochloride (e.g., 25-50 mg administered intravenously or orally).

During and/or following each dosing of NK cell inhibitor, a human patient may be monitored for potential adverse events associated with administration of the NK cell inhibitor such as daratumumab, for example, anaphylactic reaction, life-threatening (e.g., grade 4) reaction, or a combination thereof. See also Example 11 below. For human patients having low-grade reactions (e.g., grade 1, grade 2, or grade 3 reaction), NK cell inhibitor treatment may be resumed after appropriate care and symptom resolution. If the human patient has an unresolved reaction to the NK cell inhibitor treatment, then treatment of the human patient with the conditioning regimen may be delayed.

(c) Conditioning Regimen (Lymphodepleting Therapy)

Any human patients suitable for the treatment methods disclosed herein may receive a lymphodepleting therapy to reduce or deplete the endogenous lymphocyte of the subject.

Lymphodepletion refers to the destruction of endogenous lymphocytes and/or T cells, which is commonly used prior to immunotransplantation and immunotherapy. Lymphodepletion can be achieved by irradiation and/or chemotherapy. A “lymphodepleting agent” can be any molecule capable of reducing, depleting, or eliminating endogenous lymphocytes and/or T cells when administered to a subject. In some embodiments, the lymphodepleting agents are administered in an amount effective in reducing the number of lymphocytes by at least 10%, 20%, 30%, 40%, 50%, 60%, 70%, 80%, 90%, 95%, 96%, 96%, 97%, 98%, or at least 99% as compared to the number of lymphocytes prior to administration of the agents. In some embodiments, the lymphodepleting agents are administered in an amount effective in reducing the number of lymphocytes such that the number of lymphocytes in the subject is below the limits of detection. In some embodiments, the subject is administered at least one (e.g., 2, 3, 4, 5 or more) lymphodepleting agents.

In some embodiments, the lymphodepleting agents are cytotoxic agents that specifically kill lymphocytes. Examples of lymphodepleting agents include, without limitation, fludarabine, cyclophosphamide, bendamustin, 5-fluorouracil, gemcitabine, methotrexate, dacarbazine, melphalan, doxorubicin, vinblastine, cisplatin, oxaliplatin, paclitaxel, docetaxel, irinotecan, etopside phosphate, mitoxantrone, cladribine, denileukin diftitox, or DAB-IL2. In some instances, the lymphodepleting agent may be accompanied with low-dose irradiation. The lymphodepletion effect of the conditioning regimen can be monitored via routine practice.

In some embodiments, the method described herein involves a conditioning regimen that comprises one or more lymphodepleting agents, for example, fludarabine and cyclophosphamide. A human patient to be treated by the method described herein may receive multiple doses of the one or more lymphodepleting agents for a suitable period (e.g., 1-5 days) in the conditioning stage. The patient may receive one or more of the lymphodepleting agents once per day during the lymphodepleting period. In one example, the human patient receives fludarabine at about 20-50 mg/m² (e.g., 30 mg/m²) per day for 2-4 days (e.g., 3 days) and cyclophosphamide at about 500-750 mg/m² (e.g., 500 or 750 mg/m²) per day for 2-4 days (e.g., 3 days). In specific examples, the human patient may receive fludarabine at about 30 mg/m² and cyclophosphamide at about 500 mg/m² per day for three days. In other specific examples, the human patient may receive fludarabine at about 30 mg/m² and cyclophosphamide at about 750 mg/m² per day for three days.

The human patient may then be administered any of the anti-CD19 CAR T cells such as CTX110 cells within a suitable period after the lymphodepleting therapy as disclosed herein. For example, a human patient may be subject to one or more lymphodepleting agent about 2-7 days (e.g., for example, 2, 3, 4, 5, 6, 7 days) before administration of the anti-CD19 CAR+ T cells (e.g., CTX110 cells). In some instances, a human patient is administered the anti-CD19 CAR+ T cells (e.g., CTX110 cells) within about 4-5 days after the lymphodepleting therapy.

Since the allogeneic anti-CD19 CAR-T cells such as CTX110 cells can be prepared in advance and may be stored at the treatment site, the lymphodepleting therapy as disclosed herein may be applied to a human patient having a B cell malignancy within a short time window (e.g., within 2 weeks) after the human patient is identified as suitable for the allogeneic anti-CD19 CAR-T cell therapy disclosed herein. For example, the first dose of the lymphodepleting therapy (e.g., fludarabine at about 30 mg/m² and cyclophosphamide at about 500 mg/m² or 750 mg/m²) may be administered to the human patient within two weeks (e.g., within 10 days, within 9 days, within 8 days, within 7 days, within 6 days, within 5 days, within 4 days, within 3 days, within two days, or less) after the human patient is identified as suitable for the allogeneic anti-CD19 CAR-T cell therapy. In some examples, the lymphodepleting therapy may be performed to the human patient within 24-72 hours (e.g., within 24 hours) after the human patient is identified as suitable for the treatment. The patient can then be administered the CAR-T cells within 2-7 days (e.g., for example, 2, 3, 4, 5, 6, or 7 days) after the lymphodepleting treatment. This allows for timely treatment of the human patient with the allogeneic anti-CD19 CAR-T cells disclosed herein such as CTX110 cells after disease diagnosis and/or patient identification without delay (e.g., delay due to preparation of the therapeutic cells). In certain instances, a patient may receive the treatment during inpatient hospital care. In certain instances, a patient may receive the treatment in outpatient care.

Prior to any of the lymphodepletion steps, a human patient may be screened for one or more features to determine whether the patient is eligible for lymphodepletion treatment. For example, prior to lymphodepletion, a human patient eligible for lymphodepletion treatment does not show one or more of the following features: (a) significant worsening of clinical status, (b) requirement for supplemental oxygen to maintain a saturation level of greater than 90%, (c) uncontrolled cardiac arrhythmia, (d) hypotension requiring vasopressor support, (e) active infection, (f) grade ≥2 acute neurological toxicity, and unresolved reaction to NK cell inhibitor treatment (e.g., unresolved infusion reaction to daratumumab treatment).

Following lymphodepletion, a human patient may be screened for one or more features to determine whether the patient is eligible for treatment with anti-CD19 CAR T cells such as the CTX110 cells. For example, prior to anti-CD19 CAR T cell treatment and after lymphodepletion treatment, a human patient eligible for anti-CD19 CAR T cells treatment does not show one or more of the following features: (a) active uncontrolled infection, (b) worsening of clinical status compared to the clinical status prior to lymphodepletion treatment, and (c) grade ≥2 acute neurological toxicity.

In some examples, a human patient subject to the lymphodepletion treatment may meet the eligibility criteria provided in Example 11.

In some instances, the lymphodepletion treatment may not be performed to human patients who receive both an NK cell inhibitor (e.g., daratumumab) and a population of the anti-CD19 CAR T cells such as CTX110 cells.

(d) Administration of Anti-CD19 CAR T Cells

Administering anti-CD19 CAR T cells may include placement (e.g., transplantation) of a genetically engineered T cell population as disclosed herein (e.g., the CTX110 cells) into a human patient as also disclosed herein by a method or route that results in at least partial localization of the genetically engineered T cell population at a desired site, such as a tumor site, such that a desired effect(s) can be produced. The genetically engineered T cell population can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to several weeks or months, to as long as several years, or even the life time of the subject, i.e., long-term engraftment. In certain instances, a patient may receive the genetically engineered T cell population (e.g., CTX110 cells) during inpatient hospital care. In certain instances, a patient may receive genetically engineered T cell population (e.g., CTX110 cells) in outpatient care.

For example, in some aspects described herein, an effective amount of the genetically engineered T cell population can be administered via a systemic route of administration, such as an intraperitoneal or intravenous route.

In some embodiments, the genetically engineered T cell population is administered systemically, which refers to the administration of a population of cells other than directly into a target site, tissue, or organ, such that it enters, instead, the subject's circulatory system and, thus, is subject to metabolism and other like processes. Suitable modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous.

An effective amount refers to the amount of a genetically engineered T cell population needed to prevent or alleviate at least one or more signs or symptoms of a medical condition (e.g., a B cell malignancy), and relates to a sufficient amount of a genetically engineered T cell population to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

An effective amount of a genetically engineered T cell population may comprise about 1×10⁷ anti-CD19 CAR+ cells to about 1×10⁹ anti-CD19 CAR+ cells, e.g., about 1×10⁷ cells to about 1×10⁹ cells that express a CAR that binds CD19 (CAR⁺ cells), for example, CAR⁺ CTX110 cells. In some embodiments, the effective amount of the anti-CD19 CAR+ T cells may range from about 3×10⁷ to about 1×10⁸ CAR+ T cells, about 3×10⁷ to about 3×10⁸ CAR+ T cells, about 3×10⁷ to about 4.5×10⁸ CAR+ T cells, or about 3×10⁷ to about 6×10⁸ CAR+ T cells. In other embodiments, the effective amount of the anti-CD19 CAR+ T cells may range from about 1×10⁸ to about 3×10⁸ CAR+ T cells, about 1×10⁸ to about 4.5×10⁸ CAR+ T cells, or about 1×10⁸ to about 6×10⁸ CAR+ T cells. In yet other embodiments, the effective amount of the anti-CD19 CAR+ T cells may range from about 3×10⁸ to about 4.5×10⁸ CAR+ T cells or about 3×10⁸ to about 6×10⁸ CAR+ T cells. In some embodiments, the effective amount of the anti-CD19 CAR+ T cells may range from about 4.5×10⁸ to about 6×10⁸ CAR+ T cells. In some embodiments, the effective amount of the anti-CD19 CAR⁺ T cells may range from about 6.0×10⁸ to about 7.5×10⁸ anti-CD19 CAR⁺ T cells. In some embodiments, the effective amount of the anti-CD19 CAR⁺ T cells may range from about 6.0×10⁸ to about 1×10⁹ (e.g., 9.0×10⁸) CAR⁺ T CTX110 cells. In some embodiments, the effective amount of the anti-CD19 CAR⁺ T cells may range from about 7.5×10⁸ to about 9.0×10⁸ CAR⁺ T cells.

In some embodiments, an effective amount of a genetically engineered T cell population may comprise a dose of the genetically engineered T cell population, e.g., a dose comprising about 1×10⁷ CTX110 cells to about 1×10⁹ CTX110 cells. In some embodiments, the effective amount of the CAR⁺ CTX110 cells may range from about 3×10⁷ to about 1×10⁸ CAR+ CTX110 cells, about 3×10⁷ to about 3×10⁸ CAR+ CTX110 cells, about 3×10⁷ to about 4.5×10⁸ CAR+ CTX110 cells, or about 3×10⁷ to about 6×10⁸ CAR+ CTX110 cells. In other embodiments, the effective amount of the anti-CD19 CAR+ CTX110 cells may range from about 1×10⁸ to about 3×10⁸ CAR+ CTX110 cells, about 1×10⁸ to about 4.5×10⁸ CAR+ CTX110 cells, or about 1×10⁸ to about 6×10⁸ CAR+ CTX110 cells. In yet other embodiments, the effective amount of the anti-CD19 CAR+ CTX110 cells may range from about 3×10⁸ to about 4.5×10⁸ CAR+ CTX110 cells or about 3×10⁸ to about 6×10⁸ CAR+ CTX110 cells. In some embodiments, the effective amount of the anti-CD19 CAR+ CTX110 cells may range from about 4.5×10⁸ to about 6×10⁸ CAR+ CTX110 cells. In some embodiments, the effective amount of the anti-CD19 CAR+ CTX110 cells may range from about 6.0×10⁸ to about 7.5×10⁸ CAR+ CTX110 cells. In some embodiments, the effective amount of the anti-CD19 CAR+ CTX110 cells may range from about 6.0×10⁸ to about 1×10⁹ (e.g., 9.0×10⁸) CAR⁺ CTX110 cells. In some embodiments, the effective amount of the anti-CD19 CAR+ CTX110 cells may range from about 7.5×10⁸ to about 9.0×10⁸ CAR+ CTX110 cells.

In some embodiments, an effective amount of a genetically engineered T cell population may comprise at least 1×10⁷ CAR⁺ CTX110 cells. In some embodiments, an effective amount of a genetically engineered T cell population may comprise at least 3×10⁷ CAR+ CTX110 cells. In some embodiments, an effective amount of a genetically engineered T cell population may comprise at least 1×10⁸ CAR+ CTX110 cells. In some embodiments, an effective amount of a genetically engineered T cell population may comprise at least 3×10⁸ CAR⁺ CTX110 cells. In some embodiments, an effective amount of a genetically engineered T cell population may comprise at least 4.5×10⁸ CAR+ CTX110 cells. In some embodiments, an effective amount of a genetically engineered T cell population may comprise at least 6×10⁸ CAR⁺ CTX110 cells. In some embodiments, an effective amount of a genetically engineered T cell population may comprise at least 7.5×10⁸ CAR+ CTX110 cells. In some embodiments, an effective amount of a genetically engineered T cell population may comprise at least 1×10⁹ CAR⁺ CTX110 cells.

In some examples, an effective amount of a genetically engineered T cell population may be about 1×10⁷ CAR⁺ CTX110 cells. In some examples, an effective amount of a genetically engineered T cell population may be about 3×10⁷ CAR+ CTX110 cells. In some examples, an effective amount of a genetically engineered T cell population may be about 1×10⁸ CAR⁺ CTX110 cells. In some examples, an effective amount of a genetically engineered T cell population may be about 3×10⁸ CAR⁺ CTX110 cells. In some examples, an effective amount of a genetically engineered T cell population may be about 4.5×10⁸ CAR+ CTX110 cells. In some examples, an effective amount of a genetically engineered T cell population may be about 6×10⁸ CAR⁺ CTX110 cells. In some examples, an effective amount of a genetically engineered T cell population may be about 1×10⁹ CAR+ CTX110 cells.

The efficacy of anti-CD19 CAR T cell therapy described herein can be determined by the skilled clinician. An anti-CD19 CAR T cell therapy (e.g., involving CTX110 cells) is considered “effective”, if any one or all of the signs or symptoms of, as but one example, levels of CD19 are altered in a beneficial manner (e.g., decreased by at least 10%), or other clinically accepted symptoms or markers of a B cell malignancy are improved or ameliorated. Efficacy can also be measured by failure of a subject to worsen as assessed by hospitalization or need for medical interventions (e.g., progression of the B cell malignancy is halted or at least slowed). Methods of measuring these indicators are known to those of skill in the art and/or described herein. Treatment includes any treatment of a B cell malignancy in a human patient and includes: (1) inhibiting the disease, e.g., arresting, or slowing the progression of symptoms; or (2) relieving the disease, e.g., causing regression of symptoms; and (3) preventing or reducing the likelihood of the development of symptoms.

In some embodiments, the human patient may receive one or more subsequent doses of the anti-CD19 CAR T cells disclosed herein (e.g., the CTX110 cells), for example, up to two subsequent doses. A human patient eligible for redosing of the anti-CD19 CAR T cells may meet certain criteria as those disclosed herein. See Example 11 below. Such a human patient may be redosed with the anti-CD19 CAR-T cells upon progressive disease (PD) and had prior objective responses.

In some instances, a first subsequent dose of the anti-CD19 CAR-T cells may be given to the human patient about 4 to 8 weeks (e.g., about 4-6 weeks) after the first dose of the anti-CD19 CAR-T cells. Such a human patient may exhibit stable disease (SD), partial response (PR) or complete response (CR) at least about 4 weeks after the first dose. The human patient may receive a lymphodepletion treatment prior to each of the subsequent dose of the anti-CD19 CAR-T cells. Alternatively, the lymphodeletion treatment may not be performed, for example, if the human patient shows significant cytopenias.

In other instances, a human patient may receive a first subsequent dose of the anti-CD19 CAR T cells without lymphodeletion treatment about 7-12 days after the first dose (e.g., 7, 8, 9, 10, 11, or 12 days after the first dose). A second subsequent dose may be given to the patient about 4 to 8 weeks (e.g., about 4-6 weeks) after the first dose of the anti-CD19 CAR-T cells, if the patient meets certain criteria, for example, exhibiting stable disease (SD), partial response (PR) or complete response (CR) at least about 4 weeks after the first dose. The human patient may receive a lymphodepletion treatment prior to the second subsequent dose of the anti-CD19 CAR-T cells. Alternatively, the lymphodeletion treatment may not be performed, for example, if the human patient shows significant cytopenias.

Following each dosing of anti-CD19 CAR T cells, a human patient may be monitored for acute toxicities such as tumor lysis syndrome (TLS), cytokine release syndrome (CRS), immune effector cell-associated neurotoxicity syndrome (ICANS), B cell aplasia, hemophagocytic lymphohistiocytosis (HLH), cytopenia, graft-versus-host disease (GvHD), hypertension, renal insufficiency, or a combination thereof.

When a human patient exhibits one or more symptoms of acute toxicity, the human patient may be subjected to toxicity management. Treatments for patients exhibiting one or more symptoms of acute toxicity are known in the art. For example, a human patient exhibiting a symptom of CRS (e.g., cardiac, respiratory, and/or neurological abnormalities) may be administered an anti-cytokine therapy. In addition, a human patient that does not exhibit a symptom of CRS may be administered an anti-cytokine therapy to promote proliferation of anti-CD19 CAR T cells.

Alternatively, or in addition to, when a human patient exhibits one or more symptoms of acute toxicity, treatment of the human patient may be terminated. Patient treatment may also be terminated if the patient exhibits one or more signs of an adverse event (AE), e.g., the patient has an abnormal laboratory finding and/or the patient shows signs of disease progression.

The allogeneic anti-CD19 CAR T cell therapy (e.g., involving the CTX110 cells) described herein may also be used in combination therapies. For example, anti-CD19 CAR T cells treatment methods described herein may be co-used with other therapeutic agents, for treating a B cell malignancy, or for enhancing efficacy of the genetically engineered T cell population and/or reducing side effects of the genetically engineered T cell population.

(e) Exemplary Treatment Regimens

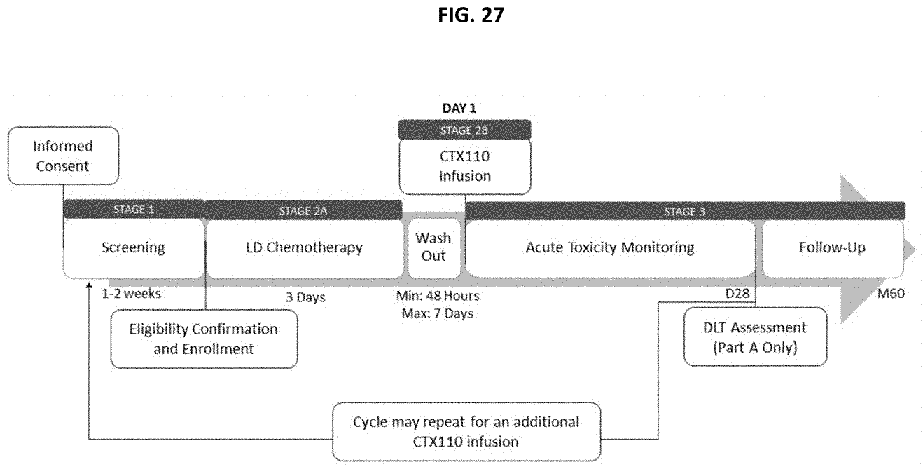

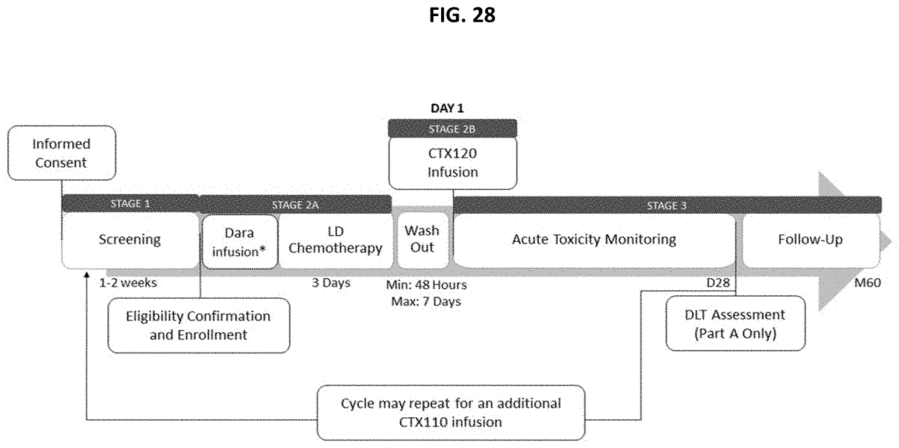

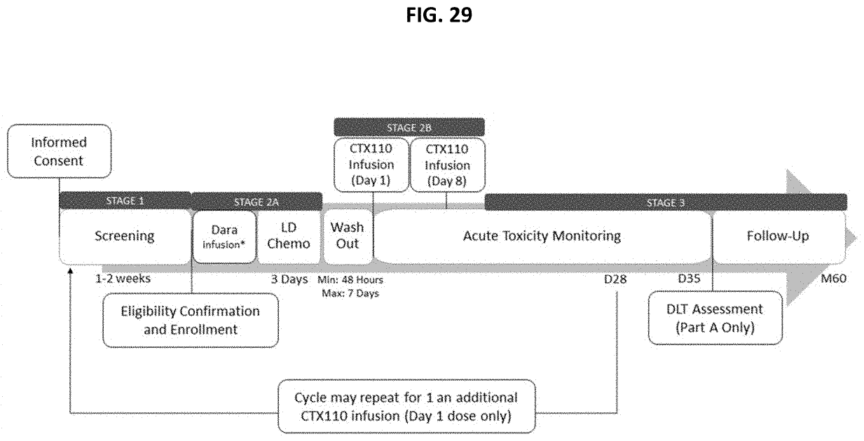

A human patient having a CD19+ B cell malignancy can be treated by any of the treatment methods disclosed herein, using the anti-CD19 CAR-T cells (e.g., CTX110), optionally in combination with an NK cell inhibitor such as daratumumab. Exemplary treatment regimens are provided in FIGS. 27-29. Provided below are some examples.

In some embodiments, a human patient having a NHL may be identified for the treatment disclosed herein. Such a human patient may have a NHL subtype such as diffuse large B cell lymphoma (DLBCL) not otherwise specified (NOS), high grade B cell lymphoma with MYC and BCL2 and/or BCL6 rearrangements, transformed follicular lymphoma (FL), or grade 3b FL. The human patient may meet the inclusion and exclusion criteria provided in Example 11 below in association with NHL patients. The human patient is treated by one dose of daratumumab at 16 mg/kg administered by IV infusion or 1800 mg by SC injection at least 1 day prior to starting lymphodepletion (LD) chemotherapy and within 10 days of CTX110 infusion (Day 1). To facilitate administration, the first 16 mg/kg IV dose may be split (to 8 mg/kg) over 2 consecutive days. If the human patient achieves SD or better on Day 28, 2 additional doses of daratumumab (16 mg/kg IV or 1800 mg SC) can be administered to the patient at Day 28 (±4 days) and Month 2 (±4 days). The LD chemotherapy includes co-administration of fludarabine 30 mg/m² and cyclophosphamide 500 mg/m² IV daily for 3 days. Both agents are started on the same day and administered for 3 consecutive days, and completed at least 48 hours (but no more than 7 days) prior to the CTX110 infusion. CTX110 may start at a dose of at least 3×10⁸ CAR+ T cells. In some instances, a second dose of CTX110, in association with an LD chemotherapy, can be administered to the patient who achieves SD, PR, or CR at Day 28 scan (e.g., based on Lugano criteria). In some examples, the additional dose may be administered without LD chemotherapy if the subject is experiencing significant cytopenias.

In some embodiments, a human patient having a NHL may be identified for the treatment disclosed herein. Such a human patient may have a NHL subtype such as diffuse large B cell lymphoma (DLBCL) not otherwise specified (NOS), high grade B cell lymphoma with MYC and BCL2 and/or BCL6 rearrangements, transformed follicular lymphoma (FL), or grade 3b FL. The human patient may meet the inclusion and exclusion criteria provided in Example 11 below in association with NHL patients. The human patient is treated by one dose of daratumumab at 16 mg/kg administered by IV infusion or 1800 mg by SC injection at least 1 day prior to starting lymphodepletion (LD) chemotherapy and within 10 days of CTX110 infusion (Day 1). To facilitate administration, the first 16 mg/kg IV dose may be split (to 8 mg/kg) over 2 consecutive days. If the human patient achieves SD or better on Day 28, 2 additional doses of daratumumab (16 mg/kg IV or 1800 mg SC) can be administered to the patient at Day 28 (±4 days) and Month 2 (±4 days). The LD chemotherapy includes co-administration of fludarabine 30 mg/m² and cyclophosphamide 500 mg/m² IV daily for 3 days. Both agents are started on the same day and administered for 3 consecutive days, and completed at least 48 hours (but no more than 7 days) prior to the CTX110 infusion. CTX110 may start at a dose of at least 4.5×10⁸ CAR+ T cells or at least 6×10⁸ CAR+ T cells. A second dose of CTX110 may be administered to the patient without LD chemotherapy on Day 8 (+1-5 days, e.g., +2 days) after the first CTX110 infusion. A third dose of CTX110 may be administered with LD chemotherapy 4 to 8 weeks after the first CTX110 infusion to the patient who achieves SD, PR or CR at Day 28 scan (based on Lugano criteria). The LD therapy may be omitted if the patient experiences significant cytopenias.

In some embodiments, a human patient having B cell acute lymphoblastic leukemia (ALL) (e.g., relapsed or refractory) can be identified for the treatment disclosed herein. The patient can be first treated by a LD chemotherapy, which may include co-administration of fludarabine 30 mg/m² and cyclophosphamide 500-750 mg/m² IV daily (e.g., 500 mg/m² daily or 750 mg/m² daily) for 3 days. Both agents are started on the same day and administered for 3 consecutive days and completed at least 48 hours (but no more than 7 days) prior to CTX110 infusion. CTX110 may start at a dose of at least 1.0×10⁸ CAR+ T cells or at least 3.0×10⁸ CAR+ T cells. At least one subsequent dose of the CTX110 cells may be administered to the patient if the patient has a decrease in BM blast count at Day 28 of at least 50%. A second dose of CTX110 may be administered to the patient 4 to 8 weeks after the first CTX110 infusion, if the patient is in a morphologic remission and/or minimal residual disease (MRD)-remains positive. The additional dose may be administered without LD chemotherapy if the subject is experiencing significant cytopenias.

In some embodiments, a human patient having B cell acute lymphoblastic leukemia (ALL) (e.g., relapsed or refractory) can be identified for the treatment disclosed herein. The patient may be first treated with one dose of daratumumab 16 mg/kg administered by IV infusion or 1800 mg administered by SC injection at least 1 day prior to starting LD chemotherapy and within 10 days of CTX110 infusion. To facilitate administration, the first 16 mg/kg IV dose may be split (to 8 mg/kg) over 2 consecutive days. If the patient achieves SD or better on Day 28, 2 additional doses of daratumumab (16 mg/kg IV or 1800 mg SC) may be administered at the Day 28 (±4 days) and Month 2 (±4 days) visits. The LD chemotherapy includes co-administration of fludarabine 30 mg/m²+cyclophosphamide 500-750 mg/m² (e.g., 500 mg/m² or 750 mg/m²) IV daily for 3 days. Both agents are started on the same day and administered for 3 consecutive days and completed at least 48 hours (but no more than 7 days) prior to CTX110 infusion. CTX110 may start at a dose of at least 1.0×10⁸ CAR+ T cells or at least 3.0×10⁸ CAR+ T cells, e.g., for patients having BM involvement with ≥5% blast. At least one subsequent dose of the CTX110 cells may be administered to the patient with darabumumab and LD chemotherapy if the patient has a decrease in BM blast count at Day 28 of at least 50% and <50% blasts in BM. For example, an additional dose of CTX110 with LD chemotherapy and daratumumab may be administered 4 to 8 weeks (e.g., 4 weeks or on Day 28) after the first CTX110 infusion for the patient. The additional dose may be administered without LD chemotherapy if the subject is experiencing significant cytopenias.

Option to redose of the anti-CD19 CAR-T cells such as CTX110 is available to a human patients for treatment by any of the methods disclosed herein after PD is the human patient had prior response. The redose may be performed after PD at least 2 months after the initial CTX110 infusion for NHL patients and greater than 4 weeks after the initial CTX infusion for adult ALL patients.

V. Kit for Allogeneic CAR-T Cell Therapy Optionally in Combination with an NK Cel Inhibitor for Treatment of B Cell Malignancies

The present disclosure also provides kits for use of a population of anti-CD19 CAR T cells such as CTX110 cells as described herein, optionally in combination with an NK cell inhibitor such as daratumumab in methods for treating a B cell malignancy (e.g., CD19+ B cell malignancy). Such kits may include one or more containers comprising a first pharmaceutical composition that comprises one or more lymphodepleting agents, a second pharmaceutical composition that comprises any nucleic acid or population of genetically engineered T cells (e.g., those described herein), optionally a third pharmaceutical composition that comprises one or more NK cell inhibitors (e.g., those described herein), and a pharmaceutically acceptable carrier. Kits comprising the genetically engineered CAR-T cells as disclosed herein, such at the CTX110 cells, may be stored and inventoried at the site of care, allowing for rapid treatment of human patients following diagnosis.

In some embodiments, the kit can comprise instructions for use in any of the methods described herein. The included instructions can comprise a description of administration of the first and/or second and/or third pharmaceutical compositions to a subject to achieve the intended activity in a human patient. The kit may further comprise a description of selecting a human patient suitable for treatment based on identifying whether the human patient is in need of the treatment. In some embodiments, the instructions comprise a description of administering the first, second, and third pharmaceutical compositions to a human patient who is in need of the treatment.

The instructions relating to the use of a population of anti-CD19 CAR T cells such as CTX110 T cells described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the population of genetically engineered T cells is used for treating, delaying the onset, and/or alleviating a T cell or B cell malignancy in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a population of the anti-CD19 CAR-T cells such as the CTX110 T cells as disclosed herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. 1985); Transcription and Translation (B. D. Hames & S. J. Higgins, eds. 1984); Animal Cell Culture (R. I. Freshney, ed. 1986); Immobilized Cells and Enzymes (IRL Press, 1986); and A practical Guide To Molecular Cloning (B. Perbal, John Wiley & Sons Inc., 1984).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Examples

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### Example 1: Preparation of CD19 Targeting Allogeneic CAR-T Cells

Allogeneic T cells expressing a chimeric antigen receptor (CAR) specific for CD19 were prepared from healthy donor peripheral blood mononuclear cells as described in US Publication No. US 2018-0325955, incorporated herein by reference. Briefly, primary human T cells were first electroporated with Cas9 or Cas9:sgRNA ribonucleoprotein (RNP) complexes targeting TRAC (AGAGCAACAGTGCTGTGGCC (SEQ ID NO: 26)) and B2M (GCTACTCTCTCTITCTGGCC (SEQ ID NO: 27)). The DNA double stranded break at the TRAC locus was repaired by homology directed repair with an AAV6-delivered DNA template containing right and left homology arms to the TRAC locus flanking a chimeric antigen receptor (CAR) cassette. The CAR comprised a single-chain variable fragment (scFv) derived from a murine antibody specific for CD19, a CD8 hinge region and transmembrane domain and a signaling domain comprising CD3z and CD28 signaling domains. The amino acid sequence of the CAR, and nucleotide sequence encoding the same, is set forth in SEQ ID NOs: 40 and 39, respectively. The gRNAs used in this Example comprise the following spacer sequences: TRAC gRNA spacer (AGAGCAACAGUGCUGUGGCC; SEQ ID NO: 19); and B2M gRNA spacer (GCUACUCUCUCUUUCUGGCC; SEQ ID NO: 21). A population of cells comprising TRAC⁻/β2M⁻/anti-CD19 CAR⁺ T cells are referred to herein as “CTX110 cells”.

With CRISPR/Cas9 editing technology, high frequency knockout of the constant region of the TCRα gene (TRAC) with ˜98% reduction of TCR surface expression in human primary T-cells from healthy donors, which aims to significantly impair graft-versus-host disease (GVHD), was achieved. High frequency knockout of the 0-2-microglobulin (B2M) gene could also be obtained, which aims to increase persistence in patients, potentially leading to increased potency overall. TRAC/B2M double knockout frequencies have been obtained in ˜80% of T cells without any subsequent antibody-based purification or enrichment. Human T cells expressing a CD19-specific CAR from within a disrupted TRAC locus, produced by homology-directed repair using an AAV6-delivered donor template, along with knockout of the B2M gene have been consistently produced at a high efficiency. This site-specific integration of the CAR protects against the potential outgrowth of CD3+CAR+ cells, further reducing the risk of GVHD, while also reducing the risk of insertional mutagenesis associated with retroviral or lentiviral delivery mechanisms. These engineered allogeneic CAR-T cells show CD19-dependent T-cell cytokine secretion and potent CD19-specific cancer cell lysis.

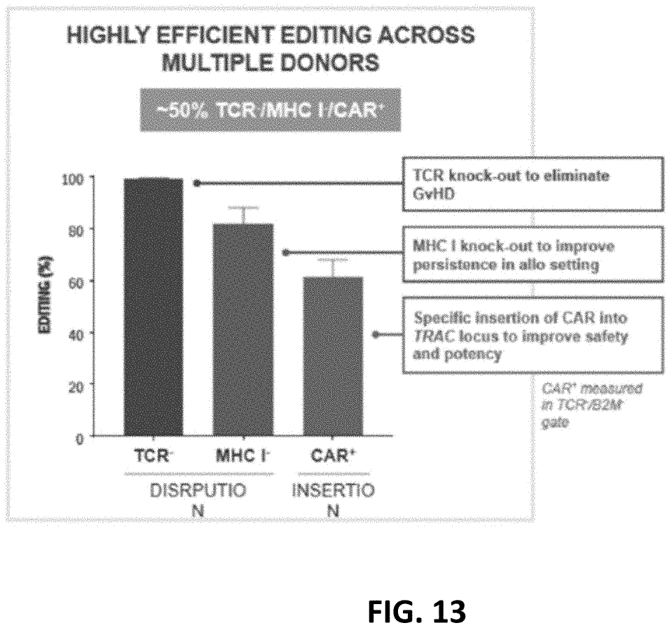

Upon purification, the population of CTX110 cells may comprise >99% TCR⁻ T cells. In some instances, the population of CTX110 cells may comprise >80% TCR⁻ and B2M⁻ T cells. The production of allogeneic anti-CD19 CAR-T product exhibited efficiency editing. For example, greater than 50% of the CTX110 cells can be TRAC⁻/B2M⁻/anti-CD19 CAR⁺. See also FIGS. 12 and 13.

### Example 2: CD38 Expression on Anti-CD19 CAR+ T Cells

CD38 cell expression on CAR T cells was measured by flow cytometry. Specifically, approximately fifteen days after the electroporation step described above, anti-CD19 CAR T cells prepared as described in Example 1 were stained with a panel of antibodies, and CD38 expression was measured.

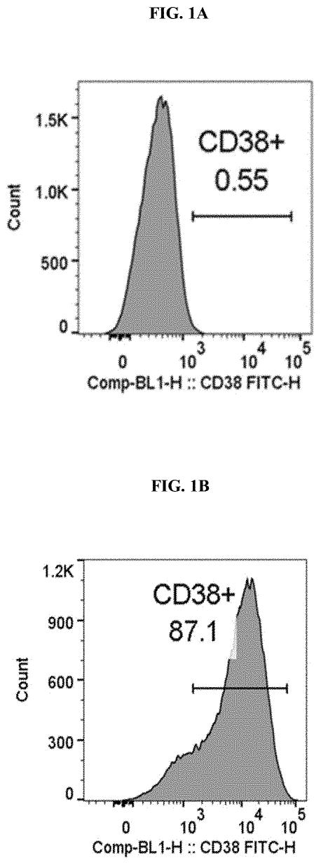

Live CAR T cells were gated by their forward scatter (FSC) and side scatter (SSC) profiles, and with a live/dead dye (cat #, L34965, ThermoFisher Scientific). The cells were then stained with a panel of antibodies: CD38 FITC (Clone HIT2, BioLegend), CD3 PE (UCHT1, Biolegend), CD4 APC/Cy7 (RPA-T4, Biolegend) and CD8 Pacific Blue (SK-1, Biolegend). CD3 T cells were then gated to measure CD38 expression. To establish gating cut-off for the CD38+ population, a fluorescent minus one (FMO) control staining was utilized (FIG. 1A). The data shows that a majority of anti-CD19 (FIG. 1B; 87.1%) CAR T cells expressed CD38.

### Example 3: CD38 Expression on NK Cells and T Cells from Normal PBMCs

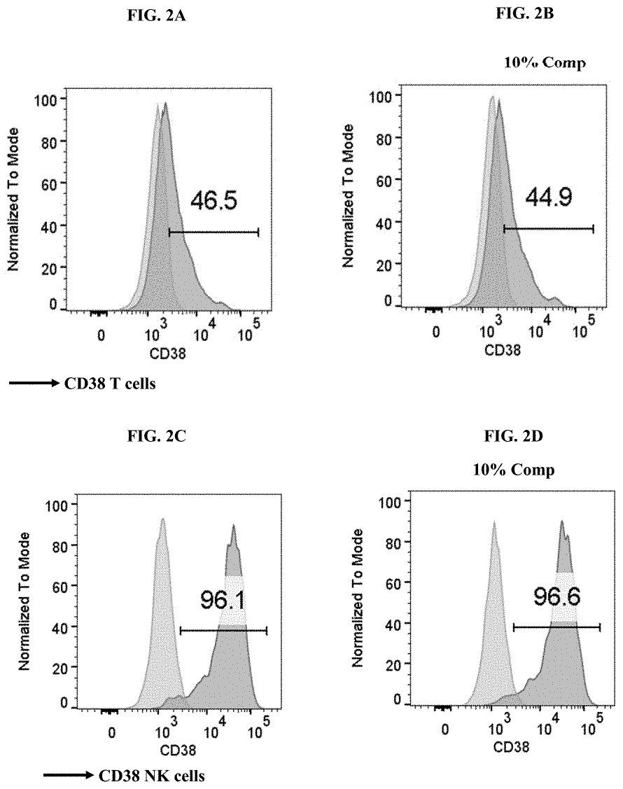

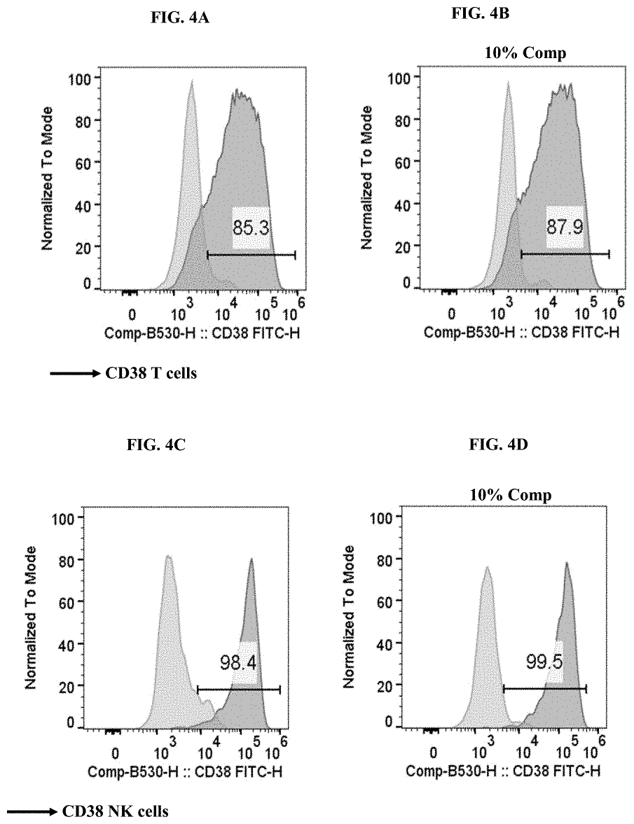

Peripheral blood mononuclear cells (PBMCs) were collected from two healthy donors (Donor 3469 and Donor 3383) to assess CD38 expression in normal immune cells. PBMCs were cultured in media (X-vivo medium (cat #04-744, Lonza) supplemented with 5% human AB serum (cat #, HP1022HI, Valley Biomedical), IL-2 and IL7), with or without 10% complement (pooled complement serum, Innovative Research, Inc.). As complement mediated lysis is triggered by anti-CD38 antibodies, the cells were cultured in media with or without complement to evaluate the effect of complement on the CD38+ cells. Flow cytometry was used to assess CD38 expression on NK cells (CD3−, CD56+) and T cells (CD3+) at Day 0 (FIGS. 2A-2D; Donor 3469 and 3A-3D; Donor 3383) and 72 hours (FIGS. 4A-4D; Donor 3469 and 5A-5D; Donor 3383) after in vitro culture. The antibody panel used for flow cytometry was CD3 PE (UCHT1, BioLegend), CD38 FITC (Clone HIT2, BioLegend), CD56 APC (HCD56, BioLegend), and CD69 PECY5 (FN50, BioLegend).

At Day 0 (1 hour after culture), the majority of NK cells and approximately half of T cells expressed CD38 on their cell surface. CD38 expression on T cells cultured in media alone or media supplemented with 10% complement was measured at 46.5% (FIG. 2A) and 44.9% (FIG. 2B), respectively. CD38 expression on NK cells cultured in media alone or media supplemented with 10% complement was measured at 96.1% (FIG. 2C) and 96.6% (FIG. 2D), respectively.

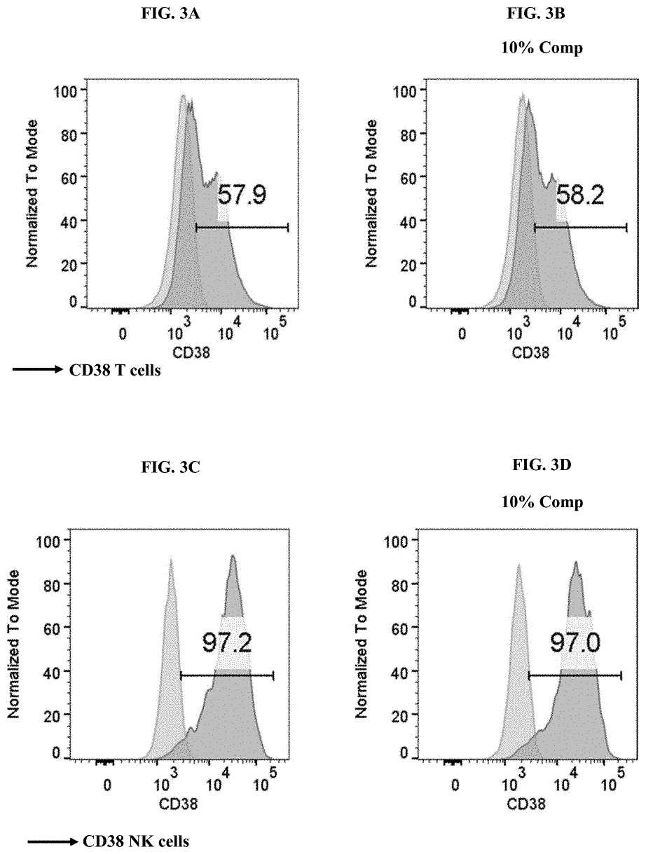

Similar results were obtained from using PBMCs collected from Donor 3383, e.g., CD38 expression on T cells was 57.9% (FIG. 3A; media alone) and 58.2% (FIG. 3B; media+ complement), and CD38 expression on NK cells was 97.2% (FIG. 3C; media alone) and 97.0% (FIG. 3D; media+10% complement).

After 72 hours of culture, the majority of NK cells and T cells expressed CD38 on their cell surface. When PBMCs from Donor 3469 were cultured in media alone or media supplemented with 10% complement, CD38 expression was detected in 98.4% (FIG. 4C; media alone) and 99.5% (FIG. 4D; media+10% complement) of NK cells. CD38 expression on T cells cultured in media alone or media supplemented with 10% complement was measured at 85.3% (FIG. 4A) and 87.9% (FIG. 4B), respectively.

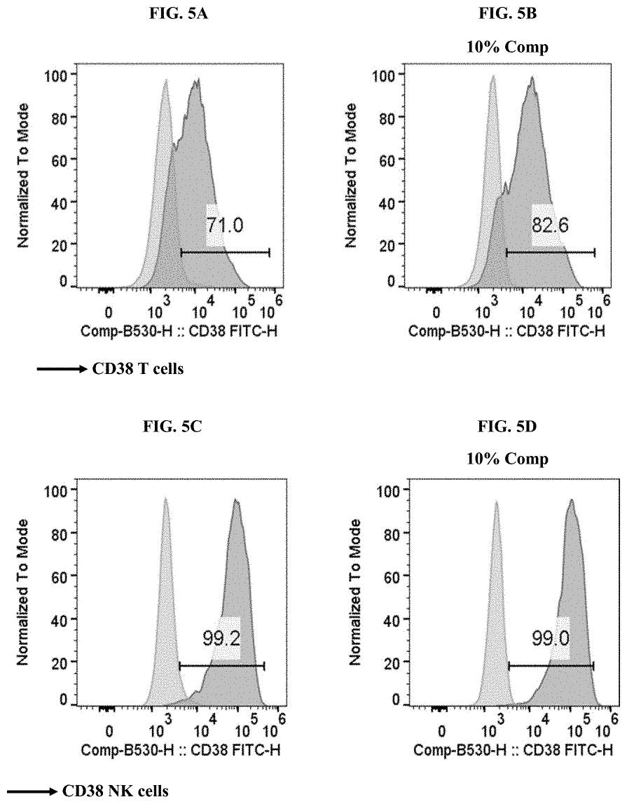

Similar results were obtained from using PBMCs collected from Donor 3383, e.g., CD38 expression on T cells was 71% (FIG. 5A; media alone) and 82.6% (FIG. 5B; media+ complement), and CD38 expression on NK cells was 99.2% (FIG. 5C; media alone) and 99% (FIG. 5D; media+10% complement).

Although complement-mediated lysis is reportedly elicited by anti-CD38 antibodies (e.g., daratumumab), the results described herein show that the presence of complement does not significantly affect cell numbers of CD38+NK cells or T cells.

### Example 4: Effect of Daratumumab on NK Cells and T Cells from PBMCs

This example analyzed the effect of anti-CD38 antibody (daratumumab; a.k.a., TAB-236) on cultures of NK cells and T cells from PBMCs of a healthy donor. PBMCs were cultured for 96 hours in media containing 0.01, 0.1, or 1 μg/mL of daratumumab. The effect of 10% complement on the cell cultures was also tested. Untreated cells and cells treated with 0.01, 0.1 or 1 μg/mL isotype control mAb (human IgG1k)(cat #403501, BioLegend) were used as controls. After 96 hours of culture, NK cell and T cell frequency and numbers were measured.

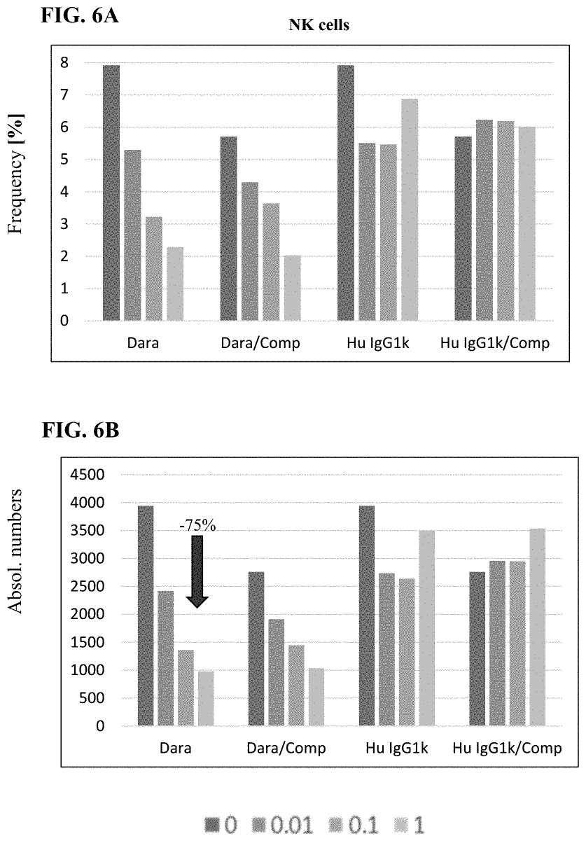

The addition of daratumumab to in vitro cultures resulted in a dose-dependent decrease of NK cell frequency (FIG. 6A) and numbers (FIG. 6B). At the highest dose tested (1 μg/mL), daratumumab reduced NK cell numbers by approximately 75% after 96 hours (FIG. 6B). This effect was specific to daratumumab, as treatment with an isotype control mAb did not significantly affect NK cell numbers (FIG. 6B). The reduction in NK cells is not complement dependent under the tested culture conditions, as the addition of 10% complement to the cell culture did not alter the effect of daratumumab on NK cell numbers (FIG. 6B). Similar results were obtained using PBMCs from a different donor, e.g., daratumumab reduced NK cell numbers ˜57% after only 72 hours (data not shown).

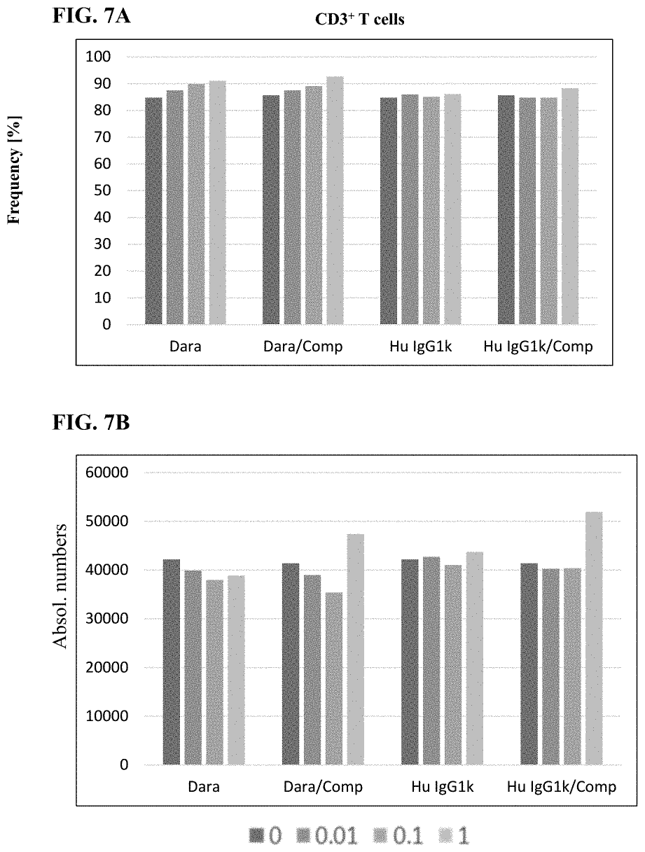

Daratumumab did not affect T cell frequency (FIG. 7A) or T cell numbers (FIG. 7B). Although CD38 expression was detected on T cells as described above, T cell frequency and numbers were surprisingly unaffected by the addition of daratumumab to the culture media (FIG. 7B).

Taken together, these results demonstrated that the addition of daratumumab to the cell culture depleted the number of NK cells, but not the number of T cells.

### Example 5: Daratumumab Treatment does not Activate CAR T Cells

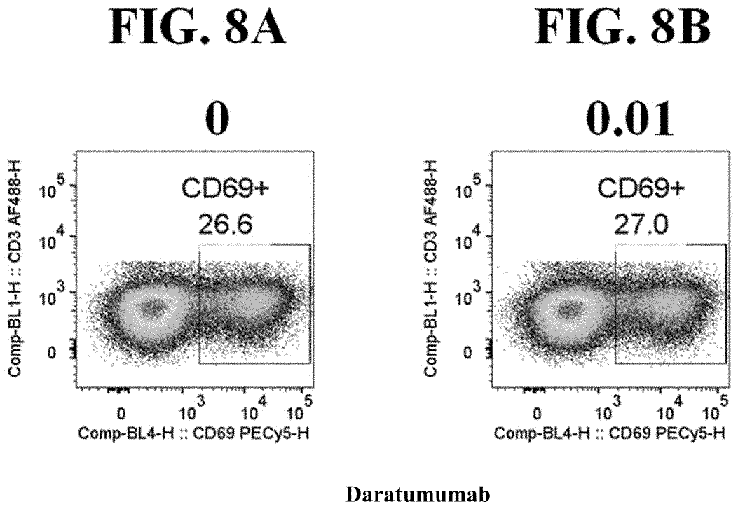

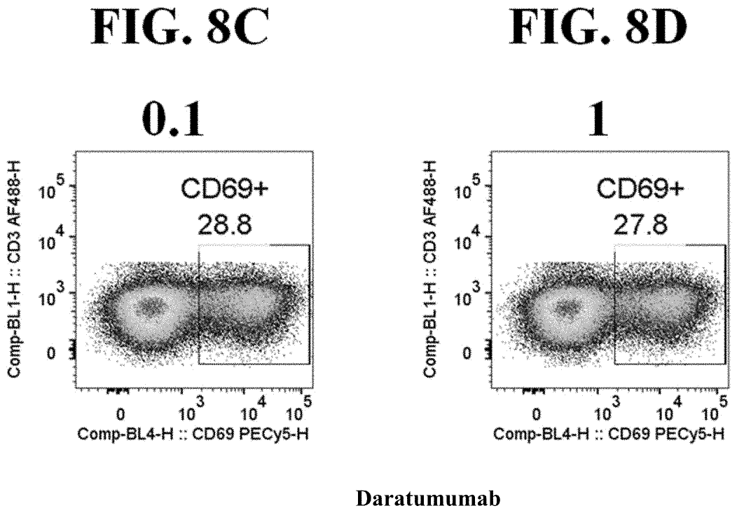

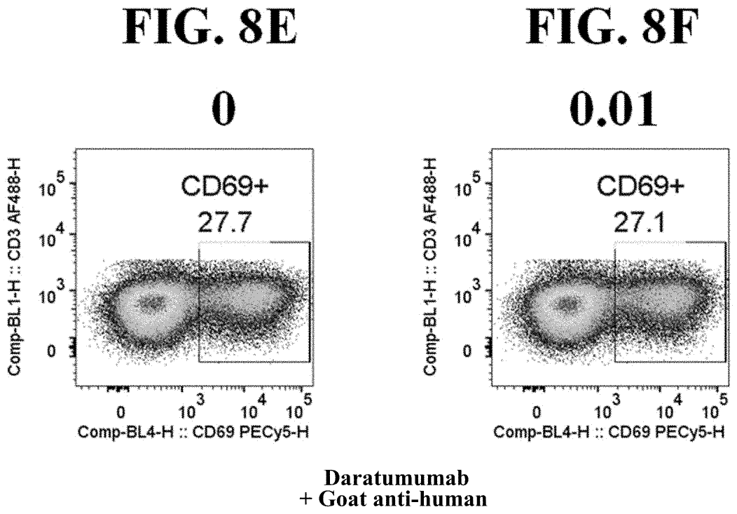

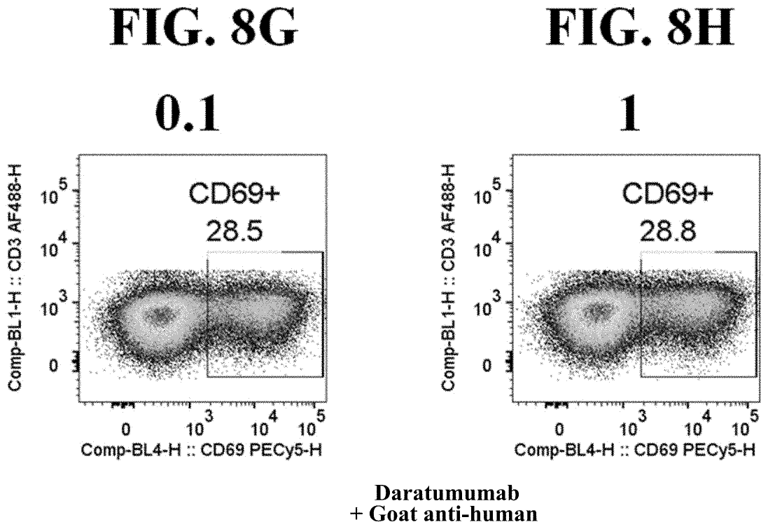

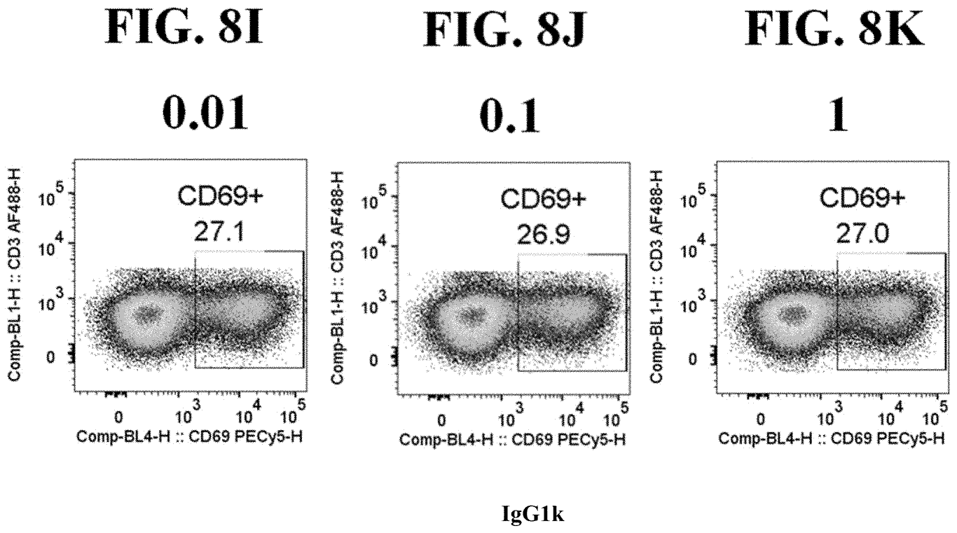

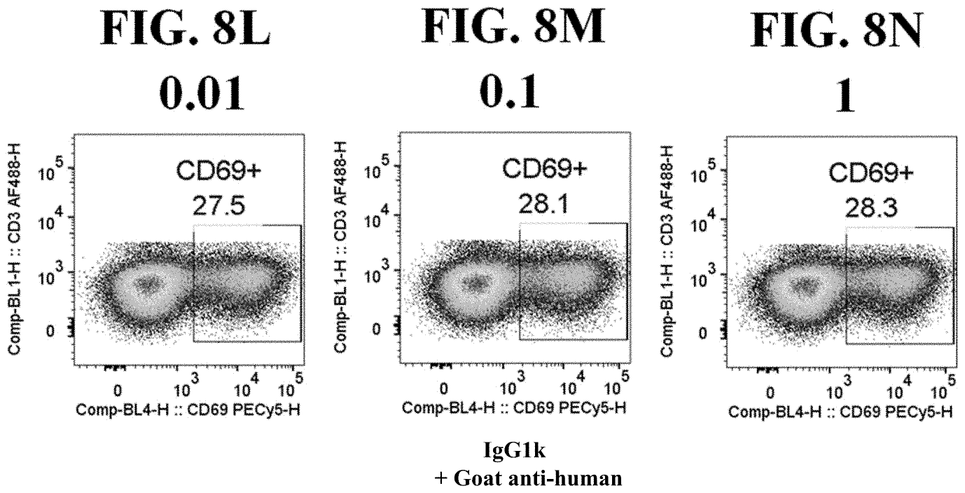

To determine whether daratumumab activates CAR T cells and causes subsequent proliferation or activation-induced cell death, anti-CD19 CAR T cells were cultured with daratumumab alone, or daratumumab with 2 μg/mL goat anti-human isotype control antibody for 24 hours. Daratumumab was used at a concentration of 0.01, 0.1, or 1 μg/mL. Cells were untreated or treated with IgG1k isotype control mAb as controls. Expression of the early activation marker, CD69, was assessed at the end of the 24 hour incubation period (FIGS. 8A-8N). As shown in the representative flow cytometry panels, CD69 expression was unchanged in all tested anti-CD19 CAR+ T cell populations, regardless of treatment.

These results demonstrate that despite the expression of CD38 cell surface marker (87.1%) on anti-CD19 CAR T cells (FIG. 1B), daratumumab treatment did not induce activation of the anti-CD19 CAR T cells.

### Example 6: In Vivo Effect of Daratumumab and Anti-CD19 CAR T Cells in Murine Xenograft Model of Acute Lymphoblastic Leukemia

A disseminated mouse model was utilized to further assess the in vivo efficacy of anti-CD19 CAR+ T cells in the presence of NK cells both with and without daratumumab.

Twenty-four 5-8 week old female CIEA NOG (NOD.Cg-Prkdc^{scid}I12rg^{tm1Sug}/JicTac) mice were individually housed in ventilated microisolator cages, maintained under pathogen-free conditions, 5-7 days prior to the start of the study. At the start of the study, the mice were divided into 12 treatment groups. The mice were inoculated intravenously (tail vein) to model disseminated disease. On Day 1 all mice received an intravenous injection of NALM6 tumor cells (0.5×10⁶ cells per mouse). NALM6 tumor cells used in this experiment were a human acute lymphoblastic leukemia (ALL) tumor cell line expressing GFP and luciferase. On Day 2, Groups 2-12 received an intravenous injection of NK cells, PBS, daratumumab (DARA) and/or IgG1. PBS and IgG1 were included as negative controls. Groups 1-3 and 7-8 also received an intravenous injection of anti-CD19 CAR T cells (4×10⁶ cells per mouse) on Day 4 of the study. The anti-CD19 CAR+ T cells injected were prepared as described in Example 1. Groups 3, 6, 9 and 12 were negative control groups treated with IgG1 instead of daratumumab. There were no unexpected effects of the IgG1 groups (data not shown). Details of the experimental groups are provided below in Table 1.

TABLE 1
Murine ALL Xenograft Treatment Groups.

| | | | | | | CAR T Cells | |
|---|---|---|---|---|---|---|---|
| | NALM6 Cells | NK Cells | | DARA | IgG1 | (CTX110) | |
| | 0.5 × 10⁶ | 4 × 10⁶ | | 100 | 100 | 4 × 10⁶ | |
| Group | cells/mouse | cells/mouse | PBS | μg/mouse | μg/mouse | cells/mouse | n |
| 1 | X | | | | | X | 5 |
| 2 | X | | | X | | X | 5 |
| 3 | X | | | | X | X | 5 |
| 4 | X | X | | | | | 5 |
| 5 | X | X | | X | | | 5 |
| 6 | X | X | | | X | | 5 |
| 7 | X | X | | | | X | 5 |
| 8 | X | X | | X | | X | 5 |
| 9 | X | X | | | X | X | 5 |
| 10 | X | | X | | | | 5 |
| 11 | X | | X | X | | | 5 |
| 12 | X | | X | | X | | 5 |

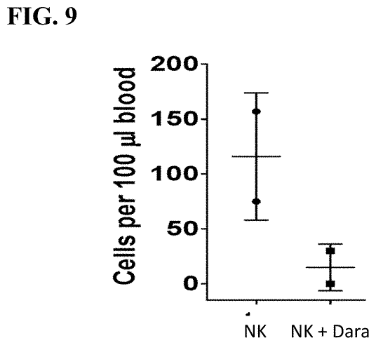

During the course of the study mice were monitored daily and body weight was measured two times weekly. Two weeks post injection, blood was collected from the mice and the number of cells was measured by flow cytometry to determine the effect of daratumumab on NK cells in circulation. FIG. 9 shows that DARA effectively decreased the number of NK cells in the in vivo mouse models.

Disease burden was measured by bioluminescent imaging for NALM6 tumor cells marked with lentiviral vectors expressing luciferase. In brief, mice were anesthetized and luciferin administered by intraperitoneal injection. The NALM6 leukemic cells marked with luciferase metabolized luciferin and emitted light detected and quantitated using methods employed by Translations Drug Development, LLC (Scottsdale, Ariz.) and described herein. Using this method, bioluminescence (BLI; total ROI, photon/s) was measured twice weekly beginning on Day 2 of the study allowing for leukemic burden to be measured and engraftment detected.

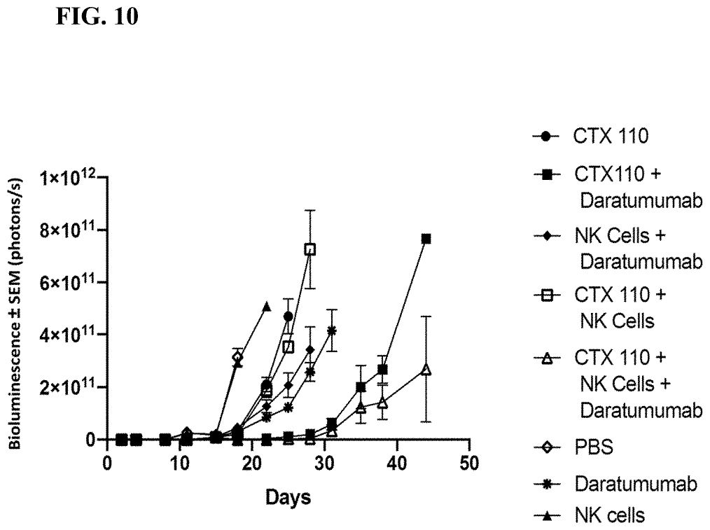

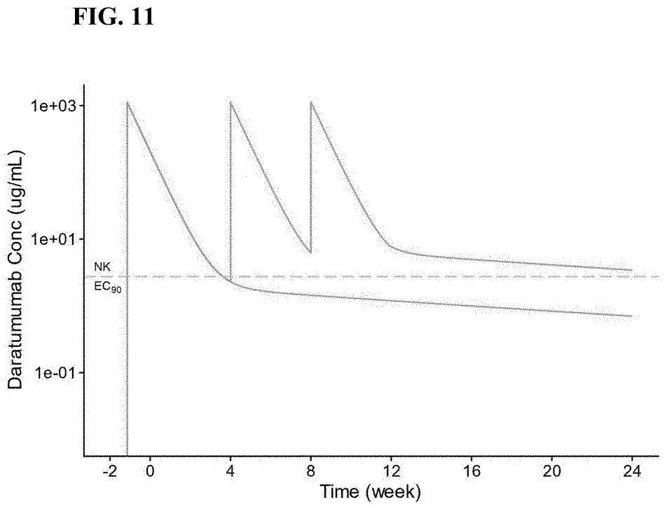

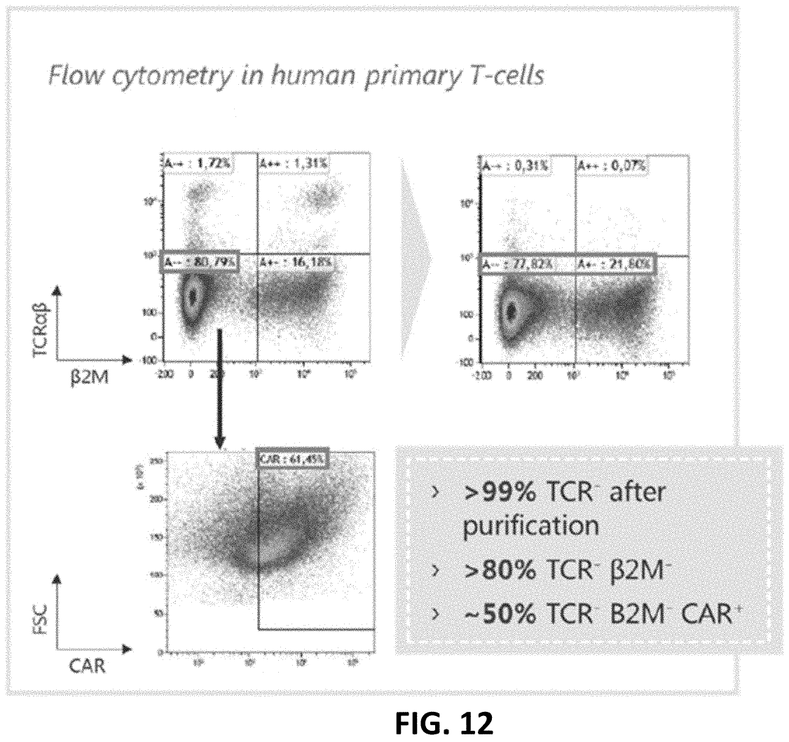

The control groups 10, 11 and 12, which did not receive NK cells, DARA or anti-CD19 CAR+ T cells, showed a rapid increase in bioluminescence at 15 days and did not survive beyond 20 days (FIG. 10). Treatment with anti-CD19 CAR+ T cells (Group 1; FIG. 10) delayed tumor progression increased survival compared to controls. The presence of NK cells did not appear to negatively affect the efficacy of anti-CD19 CAR+ T cells alone. However, the addition of daratumumab dramatically increased the efficiency of anti-CD19 CAR T cells in the presence of NK cells (Group 8; FIG. 10). Unexpectedly, daratumumab alone (Group 11) and daratumumab in the presence of NK cells (Group 5) also slowed tumor growth compared to controls and had a synergistic effect when combined with anti-CD19 CAR+ T cells (Group 2) (FIG. 10).

A significant endpoint (time to peri-morbidity) and the effect of T-cell engraftment were also assessed. The percentage of animal mortality and time to death were recorded for every group in the study. Mice were euthanized prior to reaching a moribund state. Mice were defined as moribund and sacrificed if one or more of the following criteria were met:

Loss of body weight of 20% or greater sustained for a period of greater than 1 week; Tumors that inhibit normal physiological function such as eating, drinking, mobility and ability to urinate and or defecate; prolonged, excessive diarrhea leading to excessive weight loss (>20%); or persistent wheezing and respiratory distress.

Animals were also considered moribund if there was prolonged or excessive pain or distress as defined by clinical observations such as: prostration, hunched posture, paralysis/paresis, distended abdomen, ulcerations, abscesses, seizures and/or hemorrhages. The effect of daratumumab on animal survival is provided below in Table 2 where statistical significance was determined using a Mann-Whitney test and p values were calculated compared to PBS control, (e.g., Group 10 vs. Group 1, Group 2, etc.).

TABLE 2
Effect of Daratumumab on Murine ALL
Xenograft Treatment Group Survival.

| | | Max | Median | |
|---|---|---|---|---|
| | | survival | Survival | Significance |
| Group | Treatment | (days) | (days) | (p) |
| 1 | CD19 CAR T | 28 | 25 | 0.0079 |
| 2 | CD19 CAR T + DARA | 44 | 42 | 0.0079 |
| 5 | NK cells + DARA | 31 | 30 | 0.0079 |
| 7 | CD19 CAR T + NK | 28 | 28 | 0.0079 |
| 8 | CD19 CAR T + NK + DARA | 44 | 42 | 0.0079 |
| 10 | PBS | 22 | 22 | 0.0079 |

Taken together, these results demonstrated that an NK cell inhibitor in combination with anti-CD19 CAR+ T cells reduced tumor burden and extended survival rates in a xenograft mouse model of acute lymphoblastic leukemia.

### Example 7: Assessment of CD19 Targeting CAR-T Cells Efficacy in Intravenous Disseminated Models in NOG Mice

To further assess the efficacy of TRAC⁻/B2M⁻/anti-CD19 CAR+ cells (CTX110), disseminated mouse models were utilized.

### (i) Intravenous Disseminated Nalm-6 Human Acute Lymphoblastic Leukemia Tumor Xenograft Model

The Intravenous Disseminated Model (Disseminated Model) using the Nalm-6 Human Acute Lymphoblastic Leukemia tumor cell line in NOG mice was used in to further demonstrate the efficacy of CTX110. Efficacy of CTX110 was evaluated in the Disseminated Model using methods employed by Translations Drug Development, LLC (Scottsdale, Ariz.) and described herein. In brief, 24, 5-8 week old female CIEA NOG (NOD.Cg-Prkdc^{scid}I12rg^{tm1Sug}/JicTac) mice were individually housed in ventilated microisolator cages, maintained under pathogen-free conditions, 5-7 days prior to the start of the study. At the start of the study, the mice were divided into 5 treatment groups as shown in Table 14. On Day 1 mice in Groups 2-4 received an intravenous injection of 0.5×10⁶Nalm6 cells/mouse. The mice were inoculated intravenously to model disseminated disease. On Day 4 (3 days post injection with the Nalm6 cells), treatment Groups 2-4 received a single 200 μl intravenous dose of CTX110 cells per Table 3.

TABLE 3
Treatment groups.

| Group | Nalm6 Cells (i.v.) | CTX110 Treatment (i.v.) | N |
|---|---|---|---|
| 1 | 0.5 × 10⁶ cells/mouse | None | 6 |
| 2 | 0.5 × 10⁶ cells/mouse | 1 × 10⁶ CAR⁺ cells/mouse | 6 |
| 3 | 0.5 × 10⁶ cells/mouse | 2 × 10⁶ CAR⁺ cells/mouse | 6 |
| 4 | 0.5 × 10⁶ cells/mouse | 4 × 10⁶ CAR⁺ cells/mouse | 6 |

During the course of the study, the mice were monitored daily and body weight was measured two times weekly as described above.

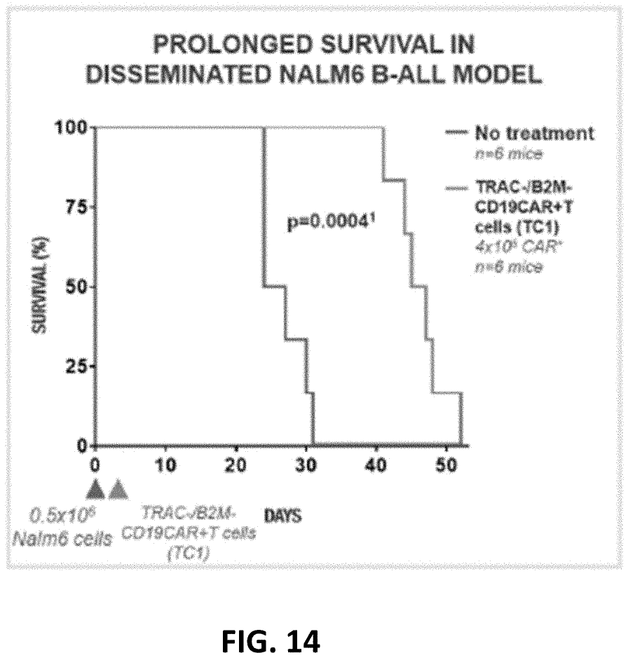

Similar to the Raji intravenous disseminated model (above), the Nalm6 Model also showed a statistically significant survival advantage in mice treated with TRAC⁻/B2M⁻/anti-CD19 CAR+ cells (CTX110) as shown in FIG. 14, p=0.0004. The effect of CTX110 treatment on survival in the Nalm6 disseminated model was also dose dependent (Table 4).

TABLE 4
Animal survival.

| | Nalm6 | CTX110 | Max | Median | |
|---|---|---|---|---|---|
| | Cells | Treatment | survival | Survival | |
| Group | (i.v.) | (i.v.) | (days) | (days) | Significance |
| 1 | Yes | No | 31 | 25.5 | |
| 2 | Yes | 1 × 10⁶ CAR⁺ | 32 | 31 | p = 0.03 |
| | | cells/mouse | | | |
| 3 | Yes | 2 × 10⁶ CAR⁺ | 38 | 36 | p = 0.0004 |
| | | cells/mouse | | | |
| 4 | Yes | 4 × 10⁶ CAR⁺ | 52 | 46 | p = 0.0004 |
| | | cells/mouse | | | |

### Example 8: Further Assessment of CD19 Targeting CAR-T Cells Efficacy in Intravenous Disseminated Models in NOG Mice

The purpose of this study was to evaluate the anti-tumor activity of anti-CD19 CAR+ T cells at multiple dose levels against the Nalm6-Fluc-GFP acute lymphoblastic leukemia tumor cell line in NOG mice. The mice were inoculated intravenously to model disseminated disease. Significant endpoint was time to peri-morbidity. Bioluminescent imaging was performed to monitor progression of disseminated disease.

In brief, 6 week old female, CIEA NOG (NOD.Cg-Prkdc^{scid}I12rg^{tm1Sug}/JicTac) mice were housed in ventilated microisolator cages, maintained under pathogen-free conditions, 5-7 days prior to the start of the study. On Day 1 mice received an intravenous inoculation of 5×10⁴ Nalm6-Fluc-GFP (Nalm6-Fluc-Neo/eGFP—Puro; Imanis Life Sciences (Rochester, Minn.)) cells/mouse. Three (3) days post inoculation with Nalm6-Fluc-GFP cells, the mice were divided into treatment groups and dosed with T cell populations comprising TRAC−/B2M−/anti-CD19 CAR+ T cells, as indicated in Table 5. Region of Interest values (ROI) values were captured and reported. Body weight was measured twice daily and bioluminescence was measured twice weekly starting on Day 4 (3 Days Post inoculation of Nalm6-Fluc-GFP cells) through Day 67, once weekly starting Day 74 to study end. To measure bioluminescence mice were injected intraperitoneally with 200 μl of D-Luciferin 150 mg/kg. Kinetics images were taken at the beginning of the study and as needed throughout to determine optimal post D-Luciferin dose and exposure time to image the mice. Mice were imaged by capturing luminescence signal (open emission) using an AMI 1000 imaging unit with software version 1.2.0 (Spectral Instruments Imaging Inc.; Tucson, Ariz.).

TABLE 5
Treatment groups.

| | | # of T Cells | Anti-CD19 | |
|---|---|---|---|---|
| Group | Anti-CD19 CAR T Cell | injected (iv) | CAR+ T cells | N |
| 1 | N/A | N/A | N/A | 5 |
| 2 | TRAC-/β2M-/anti-CD19 | 3 × 10⁶ | ~1.8 × 10⁶ | 5 |
| | | cells/mouse | | |
| 3 | TRAC-/β2M-/anti- CD19 | 6 × 10⁶ | ~3.6 × 10⁶ | 5 |
| | | cells/mouse | | |
| 4 | TRAC-/β2M-/anti- CD19 | 12 × 10⁶ | 7.2 × 10⁶ | 4 |
| | | cells/mouse | | |

Individual mice were euthanized at peri-morbidity (clinical signs suggesting high tumor burden (e.g., lack of motility, hunch back, hypoactivity) or 20% or greater body weight loss sustained for a period of greater than 1-week). Mice were euthanized prior to reaching a moribund state. The study was ended on Day 99 when the final mouse was euthanized as a long-term survivor.

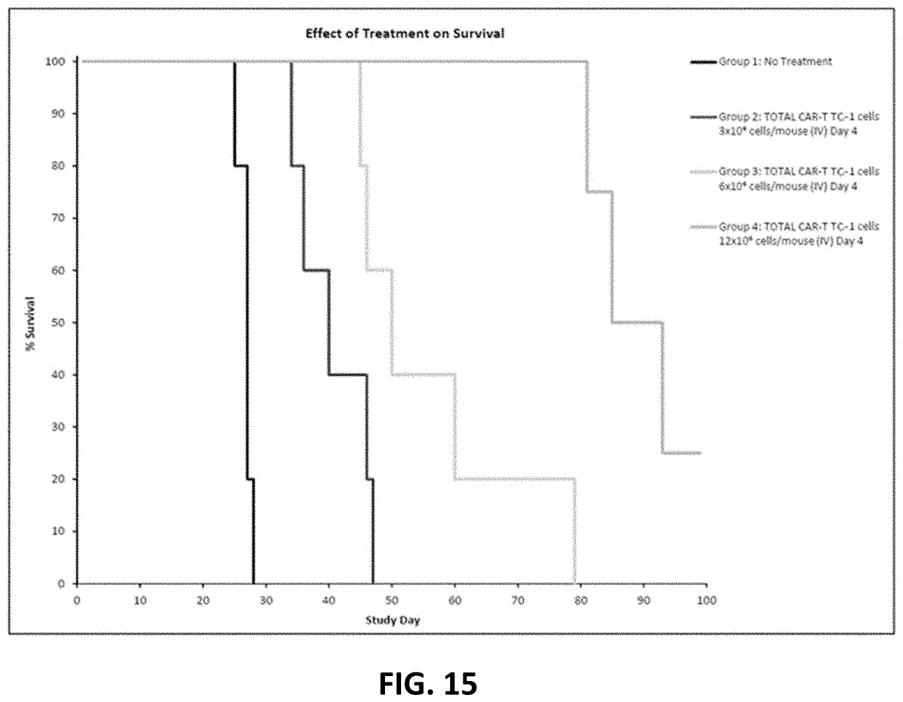

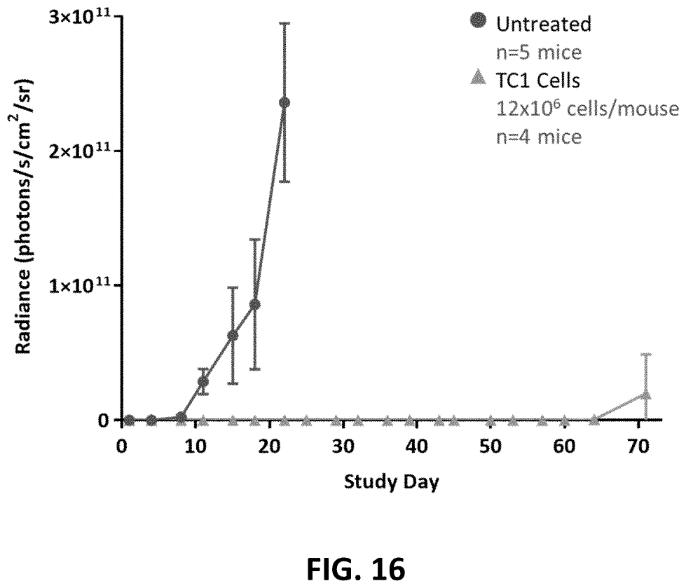

FIG. 15 shows prolonged survival of mice that received different doses of CTX110 cells relative to untreated mice. FIG. 16 shows low to undetectable levels of bioluminescence in mice that received the highest dose of CTX110 cells (12×10⁶ cells/mouse) and which resulted in the longest survival as shown in FIG. 15. At day 74 bioluminescence was detected in all 4 mice, indicative of tumor cell expansion in the treatment group.

Overall, these results show a single injection of CTX110 cells can prolong survival of mice that were administered a lethal dose of Nalm6 B-ALL cells. This prolonged survival is dose dependent with a graded survival response observed between low, middle and high doses of CTX110 cells.

### Example 9: Analysis of Graft Versus Host Disease in Mice Administered Allogeneic CD19 Targeting CAR T Cells

A study in mice was conducted to evaluate the potential for both unedited human T cells and CTX110 cells to cause graft versus host disease (GvHD). After total body irradiation with 200 cGy, NOG female mice were administered a single intravenous slow bolus injection of unedited human T cells or CTX110 cells. Animals were followed for up to 119 days after radiation only (Group 1) or radiation plus a single dose administration of PBMCs (Group 2), electroporated T cells (Group 3) or CTX110 cells (Group 4). Cells were administered approximately 6 hours post radiation on Day 1. Table 6 summarizes the groups and study design.

The endpoints of the study were survival, kinetics of appearance of GvHD symptoms, and body weight measurements.

TABLE 6
Treatment groups.

| | | | Total | Number of |
|---|---|---|---|---|
| Group | | Dose Level | Irradiation | Animals |
| Number | Test Article | (cells/mouse) | Dose | (Female) |
| 1 | Radiation Only | 0 | 200 cGy | 12 |
| 2 | Radiation + PBMCs | 6 × 10P⁶ | | 6 |
| 3 | Radiation + EP T cells | 3 × 10P⁷ | | 6 |
| 4 | Radiation + CTX110 | 3 × 10P⁷ | | 6 |
| | cells | | | |

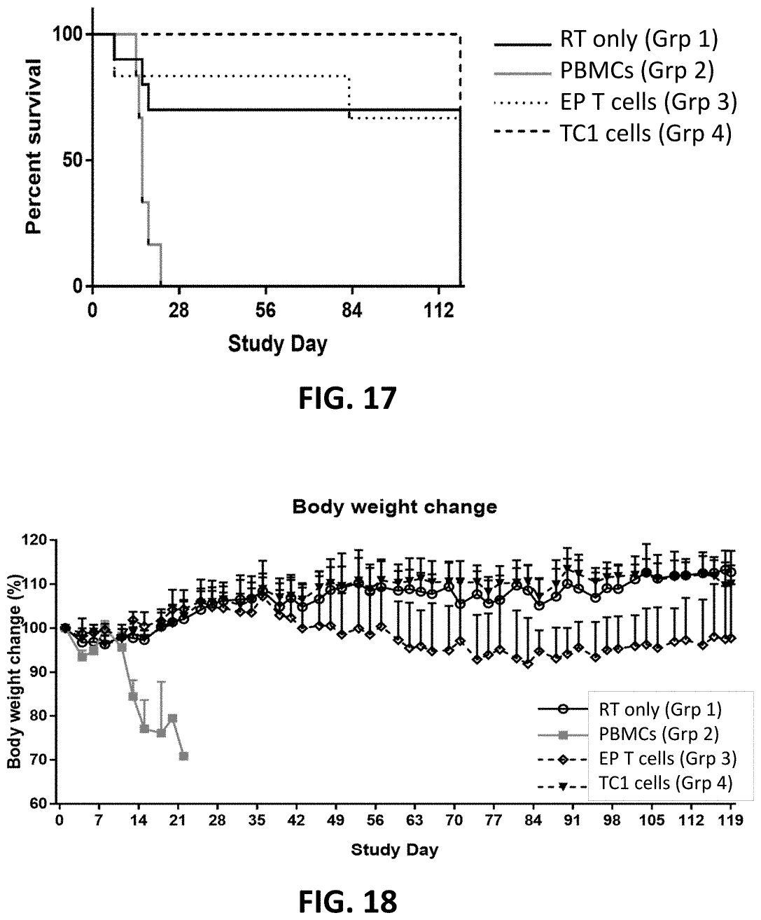

Mortality was observed in Group 1 (3 of 12 animals), Group 2 (6 of 6 animals) and Group 3 (2 of 6 animals) during the first 30 days post-treatment (FIG. 17). All animals in Group 4 (CTX110 cells) survived until scheduled necropsy (FIG. 17). Moribund animals in Groups 1, 2 and 3 experienced weight loss and/or clinical observations consistent with the development of GvHD (slight to severe cold to touch, slight to moderate emaciation, slight to marked hunched posture, severe weight loss, mild to severe alopecia, severe hypoactivity, moderate labored respiration, and marked tachypnea). Animals in Groups 1 and 4, and non-moribund animals in Group 3, experienced mild weight loss following radiation which improved over the course of the study (FIG. 18). No notable clinical observations were recorded.

This study demonstrated that unedited human PBMCs induce fatal GvHD in irradiated NOG mice in all animals (Group 2), with onset 2 to 3 weeks after administration of cells. In contrast, no mice that received CTX110 cells (Group 4) developed GvHD during the study (119 days), despite the higher number of cells that were administered to these animals (3×10⁷ CTX110 cells per mouse compared to 6×10⁶ PBMCs per mouse). The irradiation procedure induced transient weight loss in all groups and recovered in all groups that did not receive unedited PBMCs.

A second study was conducted to further evaluate the potential for both unedited human T cells and CTX110 cells to cause GvHD. Specifically, NOD/SCID/IL2Rγnull (NSG) female mice were administered a single intravenous slow bolus injection of unedited human T cells or CTX110 cells after a total body irradiation (total irradiation dose of 200 cGy, 160 cGy/min; targeted LDR_{0/140}R). The endpoints of this study were survival, kinetics of appearance of symptoms of GvHD and body weight measurements. Histopathology was also performed on all collected tissues. Exposure was assessed in mouse blood and tissues by flow cytometry and immunohistochemistry (IHC), where appropriate.

The cells were administered as a single dose via intravenous slow bolus as described in Table 7.

TABLE 7
Study Design.

| | | | | Total | | |
|---|---|---|---|---|---|---|
| Group | | Dose | Concentration | Irradiation | Number of Animals | |
| Number | Test Article | (Cells/Mouse) | (Cells/mL) | Dose | M | F |
| 1 | Vehicle - no RT^{a} | 0 | 0 | 0 cGy | 5 | 5 |
| 2 | Vehicle - RT^{a} | 0 | 0 | 200 cGy | 15 | 15 |
| 3 | Unedited T cells | 1 × 10⁷ | 4 × 10⁷ | | 15 | 15 |
| 4 | CTX110 - low dose | 2 × 10⁷ | 8 × 10⁷ | | 15 | 15 |
| 5 | CTX110 - high dose | 4 × 10⁷ | 16 × 10⁷ | | 15 | 15 |

^{a}Group 1 animals were not irradiated and were not dosed with cells (animals were administered with vehicle, PBS 1X). Group 2 animals were irradiated but were not dosed with cells (animals were administered with vehicle, phosphate-buffered saline [PBS]).

Animals were randomized into treatment groups by body weight using a validated preclinical software system (Provantis). Due to the large size of this study, dosing and necropsy activities were staggered over nine days. To minimize bias, animals from the control and CTX110 groups (Groups 4 and 5) were dosed and necropsied on the same day. Necropsy occurred on Study Day 85 for all groups.

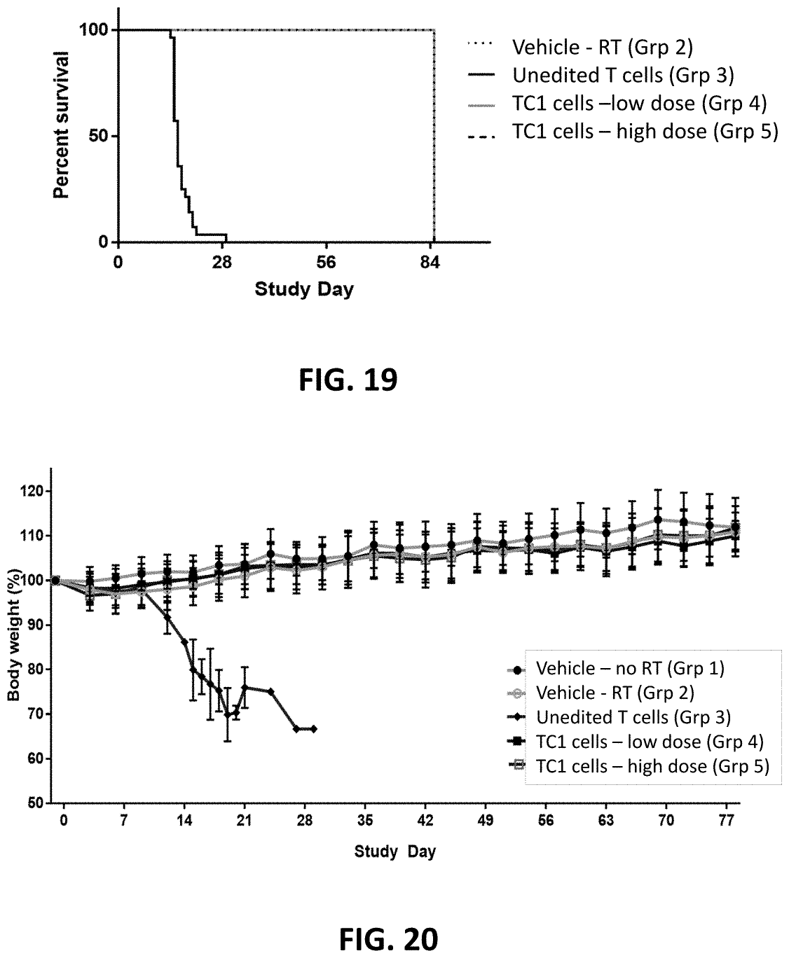

Mortality was observed for all animals that received unedited human T cells (Group 3), with onset at Day 14 (FIG. 19). All mice that received unedited human T cells (Group 3), were either found dead or sent to unscheduled euthanasia by Day 29. Clinical signs in these animals were consistent with the development of GvHD and included dull fur, slight to severe decreased activity, hunched back posture, slight to moderate thinness, and increased respiratory rate. Marked changes in hematology parameters were observed at euthanasia in mice that received unedited human T cells (Group 3), including decreases in red blood cells, hemoglobin, platelets, white blood cells and reticulocyte counts. Minimal to moderate inflammation was observed in the liver, lung, kidney, spleen, and thymus of Group 3 animals. Necrosis often accompanied inflammation in these tissues. These findings were consistent with the development of GvHD. Additionally, mild to severe hypocellularity in the femoral and sternal bone marrow was also present in the majority of Group 3 animals, which was likely attributable to the effects of total body irradiation. This was likely only observed in this group due to the early necropsy dates (2-4 weeks post-radiation), compared to 12 weeks for all other groups. Consistent with the presence of GvHD, immunohistochemical analysis of Group 3 animals revealed the presence of human CD45P⁺P cells in all tissues examined (kidney, liver, spleen, lung, skin, and the digestive tract). All animals in the other Groups survived until the scheduled necropsy.

Further, no significant weight loss was observed in Groups 1, 2, 4, or 5 (FIG. 20). No notable clinical observations that were consistent with GvHD, characterized by observations of at least two symptoms considered likely to denote GvHD, were recorded in these groups. Several animals from Groups 4 and 5 exhibited symptoms such as dull fur, slight to moderate decreased activity, and/or slight thinness throughout the study. Although these symptoms are often associated with GvHD, they did not appear to be CTX110-related as they were infrequently observed, transient and of short duration, and were also seen in some irradiated control animals (Group 2).

Overall the results from these two studies confirmed CTX110 cells do not induce graft versus host disease.

### Example 10: Preparation and Characterization of Developmental Lots of Allogeneic CD19 Targeting CAR T Cells

CTX110 cells for the purposes of the clinical study were prepared from healthy donor peripheral blood mononuclear cells obtained via a standard leukopheresis procedure. The mononuclear cells were enriched for T cells and activated with anti-CD3/CD28 antibody-coated beads, then electroporated with CRISPR-Cas9 ribonucleoprotein complexes and transduced with a CAR gene-containing recombinant adeno-associated virus (AAV) vector. The modified T cells were expanded in cell culture, purified, formulated into a suspension, and cryopreserved.

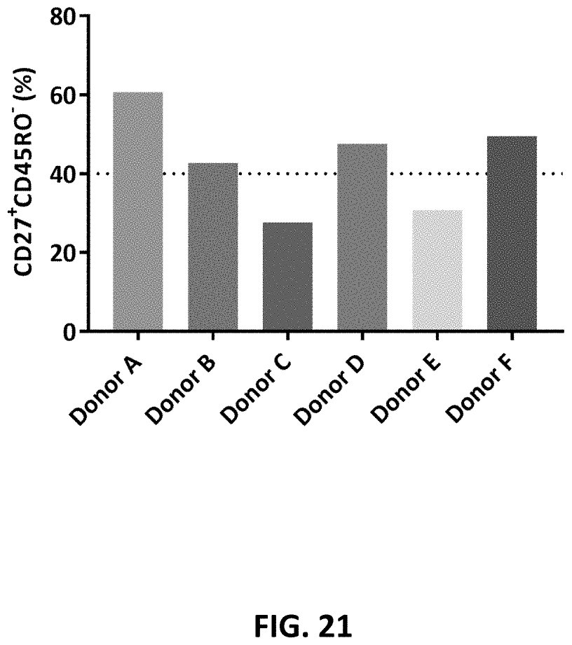

Prior to modifying the cells, T cells from six different healthy donors were evaluated for expression of various cell surface markers. CD27+CD45RO− T cells within the CD8+ subset were previously shown to correlate with complete responses in chronic lymphocytic leukemia (CLL) when treated with anti-CD19 CAR T cell therapy (Fraietta et al., Nat Med, Vol. 24(5): 563-571, 2018). Accordingly, the percent of CD27+CD45O− T cells within the CD8+ subset of six different donors was evaluated by flow cytometry. In brief, 1×10⁶ cells were incubated with Fab-Biotin or IgG-Biotin antibodies as a negative control. Cells were washed with staining buffer and incubated with mouse anti-IgG to capture excess primary antibodies. Cells were washed again and incubated with the full panel of secondary antibodies (CD8, Biolegend: Catalog #300924, CD45RO, Biolegend: Catalog #304230, CD27, Biolegend: Catalog #560612) and viability dye. Cells were washed a final time with staining buffer and run on the flow cytometer to capture various stained populations. FIG. 21 shows the levels of CD27+CD45RO− T cells within their CD8+ subsets. Allogeneic CAR-T manufacturing allows for the selection of donor input material with favorable characteristics, such as high CD27+CD45RO− cells in the CD8+ fraction of a donor of interest.

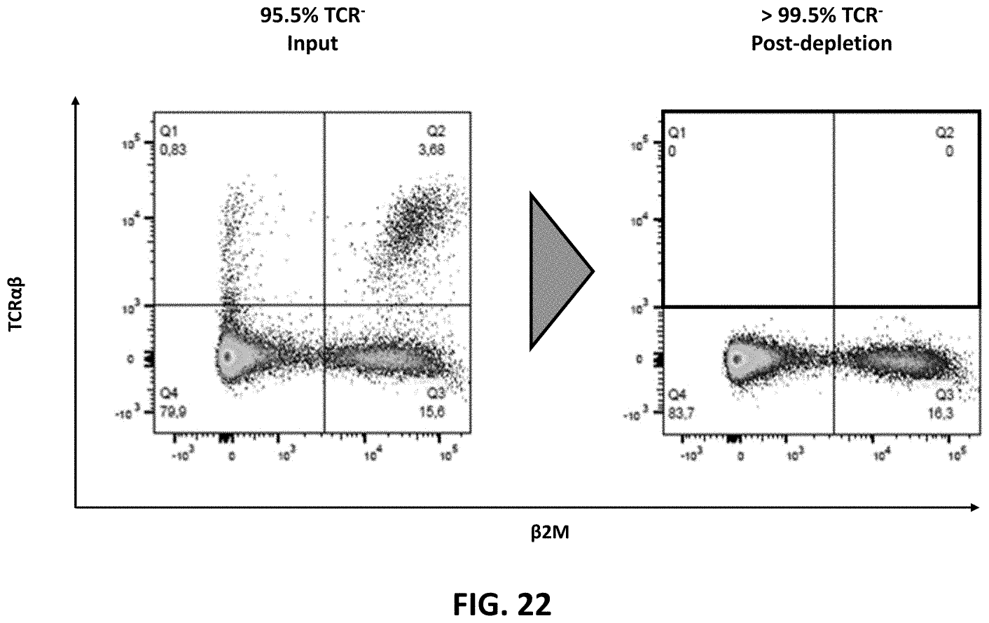

More specifically, leukopaks from 18 to 40 year-old male donors were used to isolate CD4+ and CD8+ T cells. After isolation, enrichment and activation of CD4+ and CD8+ T cells, cells were electroporated with ribonucleoprotein complexes comprising Cas9 nuclease protein, TRAC sgRNA (SEQ ID NO: 26) or B2M sgRNA (SEQ ID NO: 27). The TRAC and B2M ribonucleoprotein complexes were combined prior to electroporation. After electroporation, freshly thawed rAAV comprising a donor template (SEQ ID NO: 54) encoding the anti-CD19 CAR (SEQ ID NO: 40) was added to the cells, and cells were incubated. Cells were then expanded in culture and supplemented with rhIL-2 and rhIL-7 every three to four days. Cells set up for monitoring were tested for T cell identity and gene editing with a TCR panel (CD5, CD4, CD8, TCRαβ, B2M and CD45). Upon confirmation of T cell identity, TCRαβ depletion was performed by incubating the cells with a biotin-conjugated anti-TCRαβ antibody and anti-biotin beads. The depleted cells were recovered and formulated for administration. The resulting population of cells had less than 0.5% TCRαβ+ cells. FIG. 22 shows the analysis of TCRαβ+ cells before and after purification.

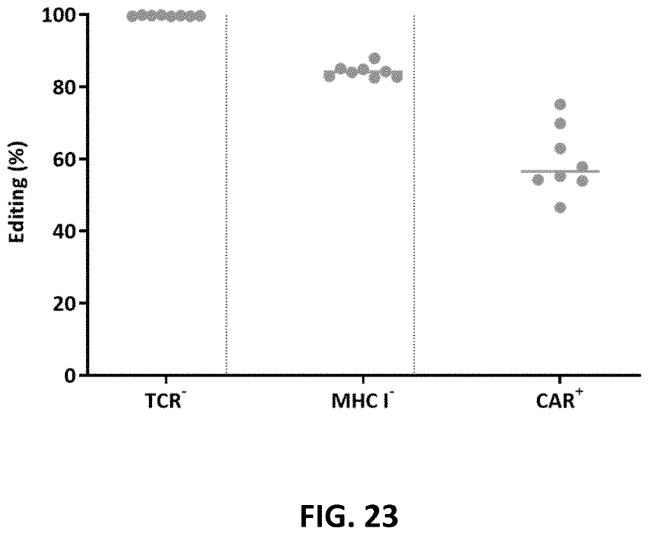

Eight development lots of CTX110 cells were tested for T cell identity. Average results from eight tested lots showed 84.58% knock-out of B2M (i.e., 15.42% B2M+ cells) and 99.98% of cells were TCR− (i.e., 0.2% TCR+), and ˜50% knock-in of anti-CD19 CAR (FIG. 23).

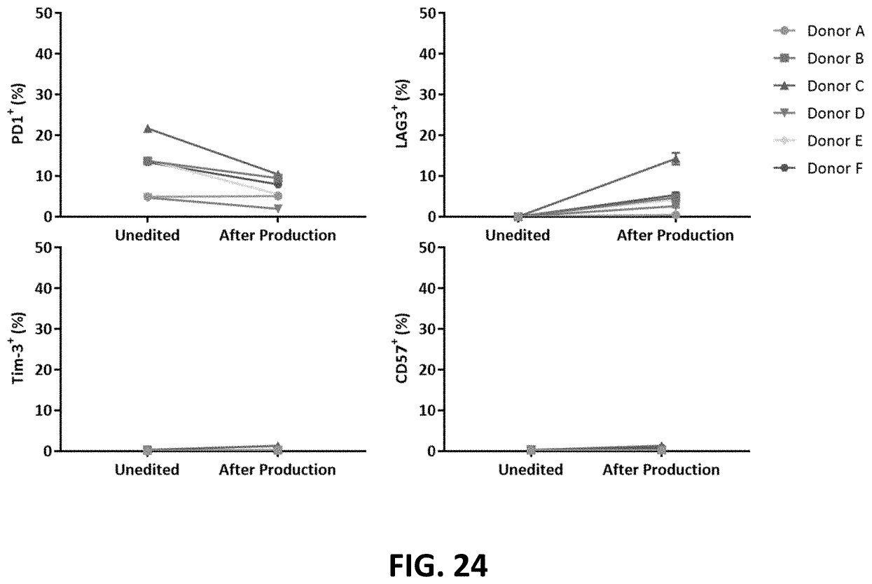

In addition, exhaustion and senescent markers were evaluated in donors before and after T cell editing. Specifically, the percentage of PD1+, LAG3+, TIM3+ and CD57+ cells were determined from total T cell populations. Expression of the markers was assessed by flow cytometry, as described above, using the following secondary antibodies: Mouse Anti-PD1 PeCy7, Biolegend, Catalog #329918; Mouse Anti-TIM3BV421, Biolegend, Catalog #345008; Mouse Anti-CD57 PerCp Cy5.5, Biolegend, Catalog #359622; and Mouse Anti-LAG3 PE, Biolegend, Catalog #369306. FIG. 24 shows that exhaustion or senescent markers never increased over 15% of the total T cell population after genome editing.

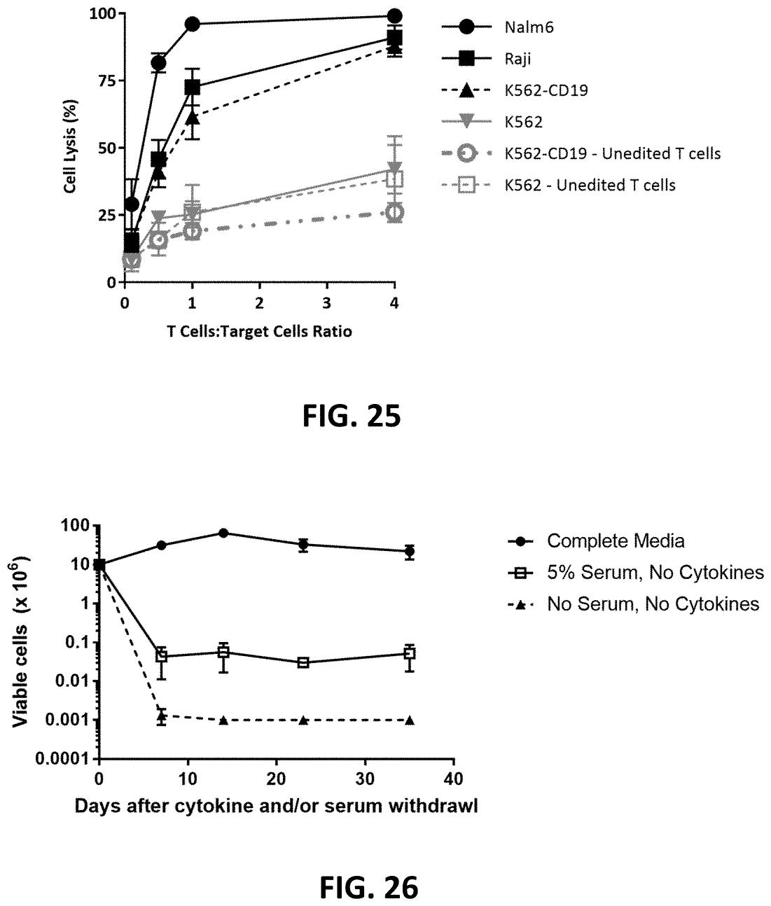

In addition, selective killing by three different lots of CTX110 cells was evaluated in vitro. Specifically, CTX110 cells were incubated with CD19-positive cell lines (K562−CD19; Raji; and Nalm6), or a CD19-negative cell line (K562). Killing was measured using a flow cytometry-based cytotoxicity assay after ˜24 hours. Specifically, target cells were labeled with 5 μM efluor670 (Thermo Fisher Scientific, Waltham, Mass.), washed and incubated overnight (50,000 target cells/well; 96-well U-bottom plate [Corning, Tewksbury, Mass.]) in co-cultures with CTX110 or control T cells at varying ratios (from 0.1:1 up to 4:1 T cells to target cells). The next day, wells were washed and media was replaced with 200 μL of fresh media containing a 1:500 dilution of 5 mg/mL 4′,6-diamidino-2-phenylindole (DAPI) (Thermo Fisher Scientific, Waltham, Mass.) to enumerate dead/dying cells. Finally, 25 μL of CountBright beads (Thermo Fisher Scientific) was added to each well, and cells were then analyzed by flow cytometry using a Novocyte flow cytometer (ACEA Biosciences, San Diego, Calif.). Flowjo software (v10, Flowjo, Ashland, Oreg.) was used to analyze flow cytometry data files (fcs files). TCRαβ+ T cells (unedited cells) were used as controls. CTX110 cells efficiently killed CD19-positive cells at higher rates than unedited T cells, and CD19-negative cells showed low levels of cell lysis in the presence of CTX110 cells that were no more than when co-cultured with unedited T cells (FIG. 25).

CTX110 cells produced from three unique donors were also used to assess growth in the absence of cytokine and/or serum. Specifically, CTX110 cells were grown in full T cell media for 14 days. On Day 0, cells from culture were grown either in complete T-cell media (containing X-VIVO 15 (Lonza, Basel, Switzerland), 5% human AB serum (Valley Biomedical, Winchester, Va.), IL-2 (Miltenyi, Bergisch Gladbach, Germany) and IL-7 (Cellgenix, Frieburg, Germany)) (Complete Media), media containing serum but no IL-2 or IL-7 cytokines (5% serum, no cytokines), or no serum or cytokines (No serum, No Cytokines). Cells were enumerated as above for up to 35 days after removal of cytokines and/or serum. No outgrowth of CTX110 cells was observed in the absence of cytokine and/or serum (FIG. 26).

For administration, CTX110 cells are resuspended in cryopreservative solution (CryoStor CS-5) and supplied in a 6 mL infusion vial. The total dose is contained in one or more vials. The infusion of each vial occurs within 20 minutes of thawing.

### Example 11: A Phase 1 Dose Escalation and Cohort Expansion Study of the Safety and Efficacy of and CD19 Allogeneic CRISPR-Cas9-Engineered T Cells (CTX110) in Subjects with Relapsed or Refractory B Cell Malignancies

CTX110 is a CD19-directed chimeric antigen receptor (CAR) T cell immunotherapy comprised of allogeneic T cells that are genetically modified ex vivo using CRISPR-Cas9 (clustered regularly interspaced short palindromic repeats/CRISPR-associated protein 9) gene editing components (single guide RNA and Cas9 nuclease). The modifications include targeted disruption of the T cell receptor (TCR) alpha constant (TRAC) and beta-2 microglobulin (B2M) loci, and the insertion of an anti-CD19 CAR transgene into the TRAC locus via an adeno-associated virus expression cassette. The anti-CD19 CAR (SEQ ID NO: 40) is composed of an anti-CD19 single-chain variable fragment comprising the SEQ ID NO: 47, the CD8 transmembrane domain of SEQ ID NO: 32, a CD28 co-stimulatory domain of SEQ ID NO: 36, and a CD3ζ (signaling domain of SEQ ID NO: 38.

CTX110 cells are prepared from healthy donor peripheral blood mononuclear cells obtained via a standard leukapheresis procedure. The mononuclear cells are enriched for T cells and activated with anti-CD3/CD28 antibody-coated beads, then electroporated with CRISPR-Cas9 ribonucleoprotein complexes, and transduced with a CAR gene-containing recombinant adeno-associated virus (AAV) vector. The modified T cells are expanded in cell culture, purified, formulated into a suspension, and cryopreserved. CTX110 can be stored onsite and thawed immediately prior to administration.

In this study, eligible human patients receive one or more doses of an intravenous (IV) infusion of CTX110, optionally in combination with daratumumab. In some instances, lymphodepleting (LD) chemotherapy is performed prior to the administration of CTX110.

### 1. Study Population

Dose escalation and cohort expansion include adult subjects with B cell malignancies. Subjects are assigned to independent dose escalation groups based on disease histology. Enrolled adult subjects include those with select subtypes of non-Hodgkin lymphoma (NHL), including diffuse large B cell lymphoma (DLBCL) not otherwise specified (NOS), high grade B cell lymphoma with MYC and BCL2 and/or BCL6 rearrangements, transformed follicular lymphoma (FL), grade 3b FL or Richter's transformation of CLL. Further, enrolled subjects include adults with relapsed or refractory B cell acute lymphoblastic leukemia (ALL).

### 2. Study Purpose and Rationale

The purpose of the Phase 1 dose escalation study is to evaluate the safety and efficacy of anti-CD19 allogeneic CRISPR-Cas9 engineered T cells (CTX110 cells) in subjects with relapsed or refractory B cell malignancies.

Outcomes for patients with relapsed/refractory B cell malignancies are historically poor. However, the use of autologous CAR T cell therapy in this setting has produced complete and durable responses where previous treatment options were palliative (June et al., (2018) Science, 359, 1361-1365; Maus and June, (2016) Clin Cancer Res, 22, 1875-1884; Neelapu et al., (2017) N Engi J Med, 377, 2531-2544; Schuster et al., (2019) N Engi J Med, 380, 45-56; Schuster et al., (2017) N Engi J Med, 377, 2545-2554). Autologous CAR T cell therapies require patient-specific cell collection and manufacturing. Unfortunately, some patients are not candidates to undergo leukapheresis, or they experience disease progression or death while awaiting treatment. An allogeneic off-the-shelf CAR T cell product such as CTX110 could provide the benefit of immediate availability, reduce manufacturing variability, and prevent individual subject manufacturing failures.

Further, patients treated with multiple rounds of chemotherapy may have T cells with exhausted or senescent phenotypes. The low response rates in patients with chronic lymphocytic leukemia (CLL) treated with autologous CAR T cell therapy have been partially attributed to the exhausted T cell phenotype (Fraietta et al., (2018) Nat Med, 24, 563-571; Riches et al., (2013) Blood, 121, 1612-1621). By starting with chemotherapy-naïve T cells from a healthy donor, allogeneic approaches could increase the consistency and potency of CAR T therapy as compared to autologous products.

The main barrier to the use of allogeneic CAR T cells has been the risk of graft versus host disease (GvHD). CRISPR Cas9 gene-editing technology allows for reliable multiplex cellular editing. The CTX110 manufacturing process couples the introduction of the CAR construct to the disruption of the TRAC locus through homologous recombination. The delivery and precise insertion of the CAR at the TRAC genomic locus using an AAV-delivered DNA donor template and HDR contrasts with the random insertion of genetic material using lentiviral and retroviral transduction methods. CAR gene insertion at the TRAC locus results in elimination of TCR in nearly all cells expressing the CAR, which minimizes risk of GvHD. Furthermore, manufacturing from healthy donor cells removes the risk of unintentionally transducing malignant B cells (Ruella et al., (2018) Nat Med, 24, 1499-1503). This first-in-human trial in subjects with relapsed/refractory B cell malignancies aims to evaluate the safety as well as efficacy of CTX110 with this CRISPR-Cas9-modified allogeneic CAR T cell approach.

CTX110, a CD19-directed genetically modified allogeneic T-cell immunotherapy, is manufactured from the cells of healthy donors; therefore, the resultant manufactured cells are intended to provide each subject with a consistent, final product of reliable quality. Furthermore, the manufacturing of CTX110, through precise delivery and insertion of the CAR at the TRAC site using AAV and homology-directed repair (HDR), does not present the risks associated with random insertion of lentiviral and retroviral vectors.

Daratumumab, an NK cell inhibitor, is co-used with CTX110 to protect the allogeneic CAR T cells from host NK-mediated cell lysis. The combination of CTX110 and NK cell inhibitor are expected to achieve superior therapeutic effects in treating the target B cell malignancy.

### 3. Study Objectives

Primary objective, Part A (Dose escalation): To assess the safety of escalating doses of CTX110 in combination with daratumumab and various lymphodepletion agents in subjects with relapsed or refractory B cell malignancies to determine the recommended Part B dose.

Primary objective, Part B (Cohort expansion): To assess the efficacy of CTX110 in subjects with relapsed or refractory B cell malignancies, as measured by objective response rate (ORR).

Secondary objectives (dose escalation and cohort expansion): To further characterize the efficacy, safety, and pharmacokinetics of CTX110.

To evaluate the changes over time in patient-reported outcomes (PROs) associated with CTX110.

Exploratory objectives (dose escalation and cohort expansion): To identify genomic, metabolic, and/or proteomic biomarkers associated with CTX110 that may indicate or predict clinical response, resistance, safety, or pharmacodynamic activity.

### 4. Study Eligibility

### 4.1 Inclusion Criteria

To be considered eligible to participate in this study, a subject must meet the inclusion criteria listed below (unless indicated as optional):

1. ≥18 years of age (for NHL patients) or ≥18 to ≤70 years of age (for ALL patients), optionally has a body weight >50 kg.

2. Able to understand and comply with protocol-required study procedures and voluntarily sign a written informed consent document.

3. Diagnosed with 1 of the following B cell malignancies:
NHL patients: Histologically confirmed B cell NHLs: DLBCL NOS, high grade B cell lymphoma with MYC and BCL2 and/or BCL6 rearrangements, transformed FL, or grade 3b FL.
Confirmation of tumor histology from local pathology lab (archival tissue from last relapse/progression [within 3 months of enrollment] or biopsy during screening).
At least 1 measurable lesion that is fluorodeoxyglucose positron emission tomography (PET)-positive, as defined by Lugano criteria (score of 4 or 5 on Lugano criteria 5-point scale) (see Tables 8 and 9). Previously irradiated lesions can be considered measurable only if progression is documented following completion of radiation therapy.
ALL patients: Histologically confirmed B cell ALL
   Bone marrow involvement with ≥5% blasts
   Bone marrow MRD positive (defined as >1×10, cells detected by flow cytometry or polymerase chain reaction [PCR]) or next generation sequencing [NGS]) including ClonoSeq in subjects with bone marrow ≤5% blasts.

4. Refractory or relapsed disease, as evidenced by the following cohort-specific criteria:
NHL: Two or more lines of prior therapy, including an anti-CD20 monoclonal antibody and an anthracycline-containing regimen, and have failed prior autologous hematopoietic stem cell transplantation (HSCT) or ineligible for or refused prior autologous HSCT. Subjects who have received autologous HSCT must have recovered from HSCT-related toxicities.
   For refractory disease, subjects must have progressive disease on last therapy, or have stable disease (MacMillan et al., 20210) following at least 2 cycles of therapy with duration of stable disease of up to 6 months.
   For subjects with transformed FL, subjects must have received at least 1 line of chemotherapy for disease after transformation to DLBCL.

Adult B cell ALL:
2 or more lines of prior therapy, or
Any bone marrow relapse after allogeneic HSCT; or
Philadelphia chromosome-positive (Ph+) if subjects are intolerant to or
ineligible for tyrosine kinase inhibitor (TKI) therapy or have progressed after at least 1 line of TKI therapy

5. Eastern Cooperative Oncology Group (ECOG) performance status 0 or 1 (see Table 26 below)

6. Meets criteria to undergo LD chemotherapy and CAR T cell infusion, and daratumumab infusion (if applicable).

7. Adequate organ function:
Renal: Estimated glomerular filtration rate >50 mL/min/1.73 m².
Liver: Aspartate transaminase or alanine transaminase <3×upper limit of normal (ULN); total bilirubin <1.5×ULN (for subjects with Gilbert's syndrome, total bilirubin <2 mg/dL).
Cardiac: Hemodynamically stable and left ventricle ejection fraction ≥45% by echocardiogram.
Pulmonary: Oxygen saturation level on room air >91% per pulse oximetry.

8. Female subjects of childbearing potential (postmenarcheal with an intact uterus and at least 1 ovary, who are less than 1 year postmenopausal) must agree to use acceptable method(s) of contraception from enrollment through at least 12 months after CTX110 infusion.

9. Male subjects must agree to use effective contraception from enrollment through at least 12 months after CTX110 infusion.

10. Refractory NHL disease with bulky presentation (high-risk subjects) may be included in an NHL cohort expansion (Part B). Refractory NHL disease with bulky presentation is defined as:
a single lesion with largest diameter ≥7.5 cm and/or sum of the product diameter (SPD) ≥5000 mm² (pre prior to LD chemotherapy) as assessed by local and/or central analysis; and/or
no history of response to any chemotherapy regimen (PR or better) and/or large B cell lymphoma diagnosis within 6 months of enrollment

The Lugano Classification provides a standardized way to assess imaging in lymphoma subjects. It is comprised of radiologic assessments of tumor burden on diagnostic CT, and metabolic assessments on F¹⁸ FDG-PET for FDG-avid histologies (see Tables 8 and 9).

TABLE 8
Lugano Classification Assessment Components.

| Diagnostic CT/MRI | F¹⁸ FDG-PET | |
|---|---|---|
| Target Lymph Nodes and Extra Nodal | 5 Point Scale (Deauville) PET Score | |
| Lesions | (Lymph Nodes and Extra Lymphatic Sites) * | |
| Up to 6 of the largest target nodes, nodal masses, | The 5-point scale scores the site of the most | |
| or other lymphomatous lesions that are | intense FDG uptake for the time point, as follows: | |
| measurable in two diameters (longest diameter | Score | Criteria |
| [LDi] and shortest diameter) should be identified | 1 | No uptake |
| from different body regions representative of the | 2 | Uptake ≤ mediastinum |
| subject's overall disease burden and include | 3 | Uptake > mediastinum but ≤ liver |
| mediastinal and retroperitoneal disease, if | 4 | Uptake moderately higher than liver |
| involved. | | (moderately indicates uptake greater |
| Nodal disease: Must have an LDi >1.5 cm | | than normal liver) |
| Extranodal disease: Must have an LDi >1.0 cm | 5 | Uptake markedly higher than liver |
| Non-Measured Lesions | | (markedly indicates much higher |
| All other lesions (including nodal, extranodal, | | than normal liver) |
| and assessable disease) should be followed as | | and/or |
| nonmeasured disease (e.g., cutaneous, GI, bone, | | New lesions |
| spleen, liver, kidneys, pleural or pericardial | X | New areas of uptake unlikely to be |
| effusions, ascites). | | related to lymphoma |

| | |
|---|---|
| Organ Enlargement (Spleen) | Bone Marrow: FDG uptake assessed as |
| The spleen is considered enlarged | No FDG uptake consistent with lymphoma |
| (splenomegaly) when >13 cm in the cranial to | Focal FDG uptake consistent with |
| caudal dimension. | lymphoma |
| New Lesions | Diffuse FDG uptake consistent with |
| Nodal disease: Must have an LDi >1.5 cm | lymphoma |
| Extranodal disease: Any size | |

CT: computed tomography; F¹⁸ FDG: fluorodeoxyglucose F18; LDi: longest diameter; MRI: magnetic resonance imaging; PET: positron emission tomography.
* See (Barrington et al., (2014) J Clin Oncol, 32, 3048-3058).

TABLE 9
Lugano Criteria for Response Assessment.
At each follow-up time point, a PET-based response and a CT-based response is made
per the definitions below.

| | | |
|---|---|---|
| Response and Site | PET-based Response | CT-based Response |

COMPLETE

| | Complete Metabolic Response* | Complete Radiologic Response |
|---|---|---|
| | ALL of the following | ALL of the following |
| Lymph nodes, | Score of 1, 2, or 3* | Lymph nodes: All <1.5 cm in |
| extranodal lesions | | longest diameter. |
| | | Extralymphatic disease absent. |
| Nonmeasured lesion | N/A | Absent |
| Organ enlargement | N/A | Spleen: normal size |
| New lesions | No new metabolically active lesions | None |
| | (new lesions drive score 5) | |
| Bone marrow | No FDG-avid disease in marrow | Normal by morphology; if |
| | | indeterminate, IHC negative. |

PARTIAL

| | | Partial Remission |
|---|---|---|
| | Partial Metabolic Response | ALL of the following |
| Lymph nodes, | Score of 4, or 5 with reduced uptake | ≥50% decrease in SPD of all |
| extranodal lesions | from baseline and residual masses of | target lesions from baseline |
| | any size | |
| Nonmeasured lesion | N/A | Absent, normal, or regressed, |
| | | but no increase |
| Organ enlargement | N/A | Spleen: ≥50% decrease from |
| | | baseline in enlarged portion |
| New lesions | None | None |
| Bone marrow | Residual uptake higher than uptake in | N/A |
| | normal marrow but reduced compared | |
| | with baseline (e.g., persistent focal | |
| | changes in the marrow with nodal | |
| | response) | |

NO RESPONSE/STABLE DISEASE

| | No Metabolic Response | Stable Disease |
|---|---|---|
| Lymph nodes, | Score of 4, or 5 with no significant | <50% decrease in SPD of all |
| extranodal lesions | change in FDG uptake from baseline | target lesion from baseline |
| | | No progression |
| Nonmeasured lesion | N/A | No increase consistent with |
| | | progression |
| Organ enlargement | N/A | Spleen: No increase |
| | | consistent with progression |
| New lesions | None | None |
| Bone marrow | No change from baseline | N/A |

PROGRESSION

| | | Progressive Disease |
|---|---|---|
| | Progressive Metabolic Response | ANY of the following |
| Lymph nodes, | Lymph nodes/nodal masses: Score | PPD Progression |
| extranodal lesions | of 4 or 5 with increased uptake | An individual node/extranodal |
| | compared to baseline. | lesion must be abnormal (nodal |
| | Extranodal lesions: New FDG avid | disease with LDi > 1.5 cm, |
| | foci consistent with lymphoma. | extranodal disease with and |
| | | LDi > 1.0 cm) with: |
| | | Increase of ≥50% from the |
| | | product of the perpendicular |
| | | diameters (PPD) from nadir AND |
| | | Increase in LDi or SDi from |
| | | nadir |
| | | ≥0.5 cm for lesions ≤2 cm |
| | | ≥1.0 cm for lesions >2 cm |
| Nonmeasured lesion | None | Unequivocal progression |
| Organ enlargement | None | Progression of pre-existing |
| | | splenomegaly: Splenic length |
| | | must increase by 50% of the |
| | | extent of its prior increase beyond |
| | | baseline (e.g., a 15-cm spleen |
| | | must increase to 16 cm). |
| | | New splenomegaly: Spleen |
| | | must increase by at least 2 cm |
| | | from baseline |
| | | Or |
| | | Recurrent splenomegaly |
| New lesions | New FDG-avid foci consistent with | Regrowth of previously |
| | lymphoma rather than another etiology | resolved lesions |
| | | New node > 1.5 cm in any axis |
| | | New extranodal site > 1.0 cm in |
| | | any axis |
| | | New extranodal site < 1.0 cm in |
| | | any axis or |
| | | unequivocal/attributable to |
| | | lymphoma |
| | | New assessable disease |
| | | unequivocal/attributable to |
| | | lymphoma of any size |
| Bone marrow | New/recurrent FDG-avid foci | New or recurrent involvement |

FDG: fluorodeoxyglucose; IHC: immunohistochemistry; LDi: longest diameter; N/A: not applicable; PPD: perpendicular diameters; SDi: shortest diameter; SPD: sum of the products of diameters.
*Deauville score of 3 represent a complete metabolic response (Barrington et al., (2014) J Clin Oncol, 32, 3048-3058).
Note:
It is recognized that in Waldeyer's ring or extranodal sites with high physiologic uptake or with activation within spleen or marrow (e.g., with chemotherapy or myeloid colony-stimulating factors), uptake may be greater than normal mediastinum and/or liver. In this circumstance, complete metabolic response may be inferred if uptake at sites of initial involvement is no greater than surrounding normal tissue even if the tissue has high physiologic uptake.

### 4.2 Exclusion Criteria

To be eligible for entry into the study, the subject must not meet any of the exclusion criteria listed below:

1. Eligible for and agrees to autologous HSCT.

2. Treatment with the following therapies as described below:
Prior treatment with any gene therapy or genetically modified cell therapy, including CAR T cells.
Prior treatment with a CD19-directed antibody, bispecific T cell engager, or antibody-drug conjugate, unless there is confirmed CD19 expression (by immunohistochemistry or flow cytometry) after progression or relapse following most recent CD19-directed treatment. The requirement to confirm CD19 expression is optional in subjects with bone marrow MRD-positive (defined as >1×10⁻⁴ cells detected by flow cytometry or polymerase chain reaction [PCR]) with ≤5% blasts.

3. Prior allogeneic HSCT. For subjects with B cell ALL, prior allogeneic HSCT is permissible if it has been more than 6 months from HSCT at the time of enrollment; there is no evidence of acute or chronic GvHD; and the subject has recovered from HSCT-related toxicities, has been off immunosuppressive therapies for at least 3 months prior to enrollment, and has not received donor lymphocyte infusion for at least 2 months prior to enrollment.

4. Diagnosis of Burkitt's lymphoma/leukemia

5. Known contraindication to daratumumab (Cohort B only), cyclophosphamide, fludarabine, or any of the excipients of CTX110 product.

6. Detectable malignant cells from cerebrospinal fluid (CSF) or magnetic resonance imaging (MRI) indicating brain metastases during screening, or a history of central nervous system (CNS) involvement by malignancy (CSF or imaging).
Subjects with B cell ALL with a prior history of CNS involvement with no evidence of current CNS disease during screening may be included, based on safety data.

7. History of a seizure disorder, cerebrovascular ischemia/hemorrhage, dementia, cerebellar disease, or any autoimmune disease with CNS involvement.

8. Unstable angina, clinically significant arrhythmia, or myocardial infarction within 6 months prior to screening.

9. Uncontrolled, acute life-threatening bacterial, viral, or fungal infection.

10. Positive for presence of human immunodeficiency virus (HIV) type 1 or 2, or active hepatitis B virus (HBV) or hepatitis C virus (HCV) infection. Subjects with prior history of HBV or HBC infection who have documented undetectable viral load (by quantitative polymerase chain reaction [PCR] or nucleic acid testing) are permitted. Infectious disease testing (HIV-1, HIV-2, HCV antibody and PCR, HBV surface antigen, HBV surface antibody, HBV core antibody) performed within 30 days of signing the informed consent form may be considered for subject eligibility.

11. Previous or concurrent malignancy, except basal cell or squamous cell skin carcinoma, adequately resected and in situ carcinoma of cervix, or a previous malignancy that was completely resected and has been in remission for ≥5 years.

12. Radiation therapy within 14 days of enrollment.

13. For NHL patients: use of systemic antitumor therapy or investigational agent within 14 days or 5 half-lives, whichever is longer, of enrollment. Exceptions are made for 1) prior inhibitory/stimulatory immune checkpoint molecule therapy, which is prohibited within 3 half-lives of enrollment, and 2) rituximab use within 30 days prior to screening is prohibited (however PET/CT needs to occur at least 2 weeks after last rituximab dose).
For adult B cell ALL patients: use of systemic antitumor therapy within 7 days of enrollment. Exceptions: 1) immunotherapy agents (i.e., rituximab, inotuzumab) must be stopped within 14 days of enrollment; 2) long-acting chemotherapy agents (e.g., pegylated asperigenase, methotrexate >25 mg/m²) must be stopped within 14 days of enrollment; and 3) investigational agent must be stopped after 5 half-lives have passed before enrolling. Subjects must have recovered to grade 1 Common Terminology Criteria for Adverse Events (CTCAE; National Cancer Institute, version 5.0) from acute toxicity (except hematological) of all previous therapy prior to enrollment. Steroids are permitted until 2 days before starting LD chemotherapy for maintenance or to allow for control of peripheral blood blasts.

14. Primary immunodeficiency disorder or active autoimmune disease requiring steroids and/or other immunosuppressive therapy.

15. Diagnosis of significant psychiatric disorder or other medical condition that could impede the subject's ability to participate in the study.

16. Women who are pregnant or breastfeeding.

17. Life expectancy of less than 6 weeks.

18. For ALL patients, exclusion of isolated extramedullary disease (defined as any patient with ≤5% blasts in the bone marrow and confirmation of the presence of clonal blasts in any tissue other than the medullary compartments)

### 5. Study Design

### 5.1 Investigational Plan

This is an open-label, multicenter, Phase 1 study evaluating the safety and efficacy of CTX110, in combination with daratumumab, in subjects with relapsed or refractory B cell malignancies. The study is divided into 2 parts: dose escalation (Part A) followed by cohort expansion (Part B).

Part A investigates escalating doses of CTX110 in adult subjects with 1 of the following NHL subtypes: DLBCL NOS, high grade B cell lymphoma with MYC and BCL2 and/or BCL6 rearrangements, grade 3b FL, or transformed FL, or in adult subjects having B cell ALL. In some instances, Daratumumab is to be administered to the subjects to induce an immune environment amenable to allogeneic CAR T cells. The use of a monoclonal antibody (median [±SD] half-life of 18±9 days) is intended to deepen and prolong the immunosuppressive effects achieved with LD chemotherapy alone.

Subjects in Cohort C (Subcohorts C1 and C2) receive 1 dose of daratumumab (Darzalex®, Janssen; an anti-CD38 monoclonal antibody) by IV infusion or SC injection prior to LD chemotherapy to achieve depletion of CD38-positive immune suppressor cells and CD38-positive immune effector cells (e.g., NK cells). For subjects in Subcohort C1 who achieve SD or better on Day 28, 2 additional doses of daratumumab (16 mg/kg by IV infusion or 1800 mg by SC injection) are administered at Day 28 (±4 days) and Month 2 (±4 days) visits. This treatment regimen explores the effect of adding daratumumab on CAR T cell expansion following CTX110 infusion and to maintain a mean trough concentration >90% effective concentration (>EC₉₀) for natural killer cell cytotoxicity (see below descriptions). Subjects in Subcohort C2 received a second planned dose of CTX110 on Day 28 with daratumumab and LD chemotherapy as specified in Table 10.

Cohort E aims at evaluating the safety and effect of planned redosing of CTX110 without and with LD chemotherapy in subjects with NHL. Subjects in Cohort E receives an initial CTX110 infusion with daratumumab and LD chemotherapy, with planned redosing of CTX110 without LD chemotherapy on Day 8 (+2 days) for subjects who meet safety parameters after the initial CTX110 infusion. A third planned redosing with CTX110 on Day 28 is presented in Table 10.

In summary, for all cohorts that enroll subjects with NHL (except C1), an additional dose of CTX110 with LD chemotherapy can be administered on Day 28 after the first CTX110 infusion to subjects who achieve SD or better at Day 28 scan (Table 10). For all cohorts, the Day 28 dose of CTX110 may be administered without LD chemotherapy if subject is experiencing significant cytopenias, as described herein. In Cohort E, the planned Day 8 dose of CTX110 may be administered without LD chemotherapy and daratumumab. For all cohorts, optional redosing after PD may be administered with LD chemotherapy.

Cohorts D and G include adult subjects with B cell ALL within the criteria of 2 subcohorts:
D1 and G1 include subjects with bone marrow involvement with ≥5% blasts.
D2 and G2 include subjects with bone marrow MRD-positive (defined as >1×10⁴ cells detected by flow cytometry or polymerase chain reaction [PCR]) with ≤5% blasts.

Cohort D evaluates escalating doses of CTX110 with LD chemotherapy, while Cohort G evaluates escalating doses of CTX110 with daratumumab and LD chemotherapy.

Cohorts D and G may include additional subjects to explore alternative LD dose regimen and cyclophosphamide may be administered at a dose of up to 750 mg/m² IV daily for 3 days if the CTX110 dose level is at or below the highest dose level that has been cleared.

The cohorts for dose escalation (Part A) are summarized in Table 10 below.

TABLE 10
Lymphodepletion Regimen and CTX110 Dosing (Part A Dose Cohorts)

| Cohort | Disease Subset | Treatment |
|---|---|---|
| C | Same as Cohort A | Stage 2A |
| (Subcohorts | | Lymphodepletion Regimen: |
| C1 and C2) | | Daratumumab + LD Chemotherapy |
| | | Daratumumab administration: One dose of |
| | | daratumumab 16 mg/kg by IV infusion or 1800 |
| | | mg by SC injection administered at least 1 day |
| | | prior to starting LD chemotherapy and within 10 |
| | | days of CTX110 infusion. To facilitate |
| | | administration, the first 16 mg/kg IV dose may be |
| | | split (to 8 mg/kg) over 2 consecutive days. |
| | | For subjects who achieve SD or better on Day 28, |
| | | 2 additionaldoses of daratumumab (16 mg/kg by |
| | | IV infusion or 1800 mg by SC injection) will be |
| | | administered at the Day 28 (±4 days)and Month 2 |
| | | (±4 days) visits.¹ |
| | | LD chemotherapy: Co-administration of |
| | | fludarabine 30 mg/m² + cyclophosphamide 500 |
| | | mg/m² IV daily for 3 days; both agents should be |
| | | started on the same day and administered for 3 |
| | | consecutive days, and completed at least 48 hours |
| | | (but no more than 7 days) prior to CTX110 |
| | | infusion. |
| | | Stage 2B |
| | | Cohort C1 |
| | | CTX110 dose on Day 1 starting at DL3 |
| | | No planned second dose of CTX110 on Day 28 |
| | | Cohort C2 |
| | | CTX110 dose on Day lstarting at the DL |
| | | cleared in Cohort C1 |
| | | A planned second dose of CTX110 on Day 28 (4- |
| | | 8 weeks after the first CTX110 dose) with |
| | | daratumumab(16 mg/kg by IV infusion or 1800 |
| | | mg by SC injection) and LD chemotherapy to |
| | | subjects who achieve SD or better at Day 28 scan |
| | | (based on Lugano criteria). The |
| | | Day 28 dose may be administered without LD |
| | | chemotherapy if the subject is experiencing |
| | | significantcytopenias. |
| | | Subcohorts C1 or C2 |
| | | Option to redose CTX110 with LD chemotherapy |
| | | after PD if subject had prior response. |
| D | Adult B cell ALL | Stage 2A |
| | | LD chemotherapy: Co-administration of |
| | | fludarabine 30 mg/m² + cyclophosphamide 500 |
| | | mg/m² IV daily for 3 days; both agents should be |
| | | started on the same day and administered for 3 |
| | | consecutive days and completed at least 48 hours |
| | | (but no more than 7 days) prior to CTX110 |
| | | infusion.² |
| | | Stage 2B |
| | | CTX110 dose on Day 1 starting at DL2 or DL3 |
| | | Option to redose CTX110 with LD chemotherapy |
| | | if subject has a decrease in BM blast count at Day |
| | | 28 of at least 50% and <50% blasts. Once at least |
| | | 3 subjects havebeen redosed, the second dose of |
| | | CTX110 may be administered on Day 28 with LD |
| | | chemotherapy for subsequent subjects who are in |
| | | morphologic remission and remain minimal |
| | | residual disease (MRD) positive. The Day 28 dose |
| | | may be administered without LD chemotherapy if |
| | | the subject is experiencing significant cytopenias. |
| | | Option to redose CTX110 with LD chemotherapy |
| | | after PD if subject had prior response. |
| E | Same as Cohort A | Stage 2A |
| | | Lymphodepletion Regimen: |
| | | Daratumumab + LD Chemotherapy |
| | | Daratumumab administration: One dose of |
| | | daratumumab 16 mg/kg administered by IV |
| | | infusion or 1800 mg administered by SC injection |
| | | at least 1 day prior to starting LDchemotherapy and |
| | | within 10 days of CTX110 infusion. To facilitate |
| | | administration, the first 16 mg/kg IV dose may be |
| | | split (to 8 mg/kg) over 2 consecutive days. |
| | | LD chemotherapy: Co-administration of |
| | | fludarabine 30 mg/m² + cyclophosphamide 500 |
| | | mg/m² IV daily for 3 days; both agents should be |
| | | started on the same day and administered for 3 |
| | | consecutive days and completed at least 48 hours |
| | | (but no more than 7 days) prior to CTX110 |
| | | infusion |
| | | Stage 2B |
| | | CTX110 dose on Day 1 starting at DL3.5 or DL4 |
| | | A second planned dose of CTX110 on Day 8 (+2 |
| | | days) without daratumumab and LD chemotherapy |
| | | for subjectsmeeting safety parameters (refer to |
| | | respective protocol section) |
| | | A third planned dose of CTX110 on Day 28 for |
| | | subjects who achieve SD or better at Day 28 scan |
| | | (based on Lugano criteria). The Day 28 CTX110 |
| | | dose administered |
| | | with daratumumab and LD chemotherapy. The |
| | | Day 28 dose may be administered without LD |
| | | chemotherapy if the subject is experiencing |
| | | significant cytopenias. |
| | | Option to redose CTX110 with LD chemotherapy |
| | | after PD if subject had prior response |
| G | Same as Cohort D | Stage 2A |
| (Subcohorts | Adult B cell ALL | Lymphodepletion Regimen: |
| G1 and G2) | | Daratumumab + LD Chemotherapy |
| | | Daratumumab administration: One dose of |
| | | daratumumab 16 mg/kg administered by IV |
| | | infusion or 1800 mg by SC injection at least 1 day |
| | | prior to starting LD chemotherapy andwithin 10 |
| | | days of CTX110 infusion.¹ To facilitate |
| | | administration, the first 16 mg/kg IV dose may be |
| | | split (to 8 mg/kg) over 2 consecutive days. |
| | | For subjects who achieve SD or better on Day 28, |
| | | 2 additionaldoses of daratumumab (16 mg/kg by |
| | | IV infusion or 1800 mg by SC injection) will be |
| | | administered at the Day 28 (±4 days)and Month 2 |
| | | (±4 days) visits.¹ |
| | | LD chemotherapy: Co-administration of |
| | | fludarabine 30 mg/m² + cyclophosphamide 500 |
| | | mg/m² IV daily for 3 days; completed at least 48 |
| | | hours (but no more than 7 days) prior to CTX110 |
| | | infusion.² |
| | | Stage 2B |
| | | Cohort G1 (BM involvement with ≥5% blasts) |
| | | CTX110 dose on Day 1 starting at DL2 or DL3 |
| | | Option for CTX110 redose on Day 28 with |
| | | daratumumaband LD chemotherapy if subject has |
| | | a decrease in BM blast count at Day 28 of at least |
| | | 50% and <50% blasts in BM. . . |
| | | Cohort G2 (BM MRD-positive) |
| | | CTX110 dose on Day 1 starting at the DL cleared in |
| | | G1 |
| | | Cohorts G1 and G2 |
| | | Once at least 3 subjects have been redosed, a |
| | | dose of CTX110 may be administered on Day |
| | | 28 with daratumumab and LD chemotherapy |
| | | for subjects who are in a morphologic remission |
| | | if MRD remains positive. The Day 28 dosemay |
| | | be administered without LD chemotherapy if |
| | | the subject is experiencing significant |
| | | cytopenias. |
| | | Option to redose CTX110 with LD chemotherapy |
| | | afterPD if subject had prior response. |

ALL: acute lymphoblastic leukemia;
CR: complete response;
DL: Dose Level;
DLBCL: diffuse large B cell lymphoma;
FL: follicular lymphoma;
IV: intravenously;
LD: lymphodepleting;
NOS: not otherwise specified;
MRD: minimal residual disease;
PD: progressive disease;
PR: partial response;
SD: stable disease.

Subjects should meet the criteria specified in the protocol prior to both the initiation of LD chemotherapy and infusion of CTX110 (all cohorts). Criteria for LD chemotherapy should be confirmed prior to infusion of daratumumab as applicable.

In Cohorts C and G, for subjects that do not meet the criteria for the Day 28 CTX110 planned second dose, the option to administer maintenance daratumumab after initial CXT110 infusion will be reevaluated after at least 9 subjects have been treated in Cohort C; daratumumab infusion must be discussed with the medical monitor for subjects with platelets below 25,000 cells/μL.

In Cohorts D and G (adult B cell ALL), additional subjects may be enrolled to explore alternative LD dose regimen and may be administered cyclophosphamide at a dose of up to 750 mg/m² IV daily for 3 days if the CTX110 dose level is at or below the highest dose level that has been cleared.

Where approved, daratumumab may be administered as a subcutaneous injection (1800 mg/30,000 units of hyaluronidase-fihj) per local prescribing information rather than as an IV infusion.

### 5.1.1 Study Design

Cohorts C and E comprise subjects with NHL, including DLBCL NOS, high grade B cell lymphoma with MYC and BCL2 and/or BCL6 rearrangements, transformed FL, and grade 3b FL. Cohorts D and G comprise subjects with adult B cell ALL.

The study is divided into 2 parts: dose escalation (Part A) followed by cohort expansion (Part B). Both parts of the study consist of 3 main stages: screening, treatment, and follow-up. Schematic depictions of the study schema are shown in FIGS. 27-29.

The 3 main stages of the study are as follows:

Stage 1—Screening to determine eligibility for treatment (1-2 weeks).

Stage 2—Treatment (Stage 2A+Stage 2B). See Table 10 above.

Subjects' clinical eligibility should be reconfirmed according to the criteria provided herein prior to both the initiation of LD chemotherapy and infusion of CTX110 and prior to infusion of daratumumab.

Stage 3—Follow up (5 years after the last CTX110 infusion).

For both dose escalation and cohort expansion, subjects must remain within proximity of the investigative site (i.e., 1-hour transit time) for 28 days after CTX110 infusion. During this acute toxicity monitoring period, subjects are routinely assessed for adverse events (AEs), including cytokine release syndrome (CRS), neurotoxicity, and GvHD. Toxicity management guidelines are provided in the study protocol. During dose escalation, all subjects are hospitalized for the first 7 days following CTX110 infusion, or longer if required by local regulation or site practice.

After the acute toxicity monitoring period, subjects are subsequently followed for up to 5 years after CTX110 infusion with physical exams, regular laboratory and imaging assessments, and AE evaluations. After completion of this study, subjects are required to participate in a separate long-term follow-up study for an additional 10 years to assess long-term safety and survival.

### 5.2 CTX110 Dose Escalation

CTX110 cells are administered IV using a flat dosing schema based on the number of CAR+ T cells. The starting dose is 1×10⁸ CAR+ T cells.

Dose escalation is to be performed using a standard 3+3 design. The following doses of CTX110, based on CAR+ T cells, may be evaluated in the study described herein (Table 11).

TABLE 11
Dose Escalation of CTX110.

| Dose Level | Total CAR+ T Cell Dose |
|---|---|
| −1 | 1 × 10⁷ |
| 1 | 3 × 10⁷ |
| 2 | 1 × 10⁸ |
| 3 | 3 × 10⁸ |
| 3.5 | 4.5 × 10⁸ |
| 4 | 6 × 10⁸ |

CAR: chimeric antigen receptor

The doses of CTX110 presented in Table 11, based on the total number of CAR+ T cells. Data from DL3 can be evaluated to determine whether dose escalation will continue with DL4. If subjects in DL4 experiences a DLT, the study may expand to treat subjects at DL4 or de escalate to a lower dose level (DL3.5) consisting of 4.5×10⁸ CAR+ T cells. Enrollment in subsequent cohorts (C, D, E, F, and G) may begin followed by dose escalation at higher dose levels only after assessment and confirmation of safety (dose at DL3 for Cohort C; at DL2 or DL3 for Cohorts D and G; and at DL3.5 or DL4 for Cohort E). There will be a dose limit of 7×10⁴ TCR+ cells/kg for all dose levels, which can be relied on to determine the minimum weight for dosing.

The DLT evaluation period begins with first CTX110 infusion and last for 28 days. For Cohort E, the DLT evaluation period lasts for 28 days after the second infusion, for a total of approximately 7 weeks (21 days from initial infusion+28 days from second infusion).

### 5.2.1 Dose-limiting Toxicity (DLT) Definitions

The DLT evaluation period begins with CTX110 infusion and last for 28 days. The first 3 subjects are to be treated in a staggered manner, such that the 2^{nd} and 3^{rd} subjects only receive CTX110 after the previous subject has completed the DLT evaluation period. In subsequent dose levels or expansion of the same dose level, cohorts of up to 3 subjects may be enrolled and dosed concurrently.

Subjects must receive CTX110 to be evaluated for DLT. If a subject discontinues the study any time prior to CTX110 infusion, the subject is not to be evaluated for DLT and a replacement subject is to be enrolled at the same dose level as the discontinued subject. If a DLT-evaluable subject has signs or symptoms of a potential DLT, the DLT evaluation period is to be extended according to the protocol-defined window to allow for improvement or resolution before a DLT is declared.

Toxicities are graded and documented according to National Cancer Institute Common Terminology Criteria for Adverse Events (CTCAE) version 5, except for CRS (Lee criteria), neurotoxicity (ICANS, immune effector cell-associated neurotoxicity syndrome criteria and CTCAE v5.0), and GvHD (Mount Sinai Acute GVHD International Consortium [MAGIC] criteria).

A DLT can be defined as any of the following events occurring during the DLT evaluation period that persists beyond the specified duration (relative to the time of onset):
Grade ≥2 GvHD that is steroid-refractory (e.g., progressive disease after 3 days of steroid treatment [e.g., 1 mg/kg/day], stable disease after 7 days, or partial response after 14 days of treatment).
Death during the DLT period (except due to disease progression).
Grade 4 neurotoxicity of any duration that is possibly related or related to CTX110
Any CTX110-related grade 3 or 4 toxicity that is clinically significant and does not improve within 72 hours

The following are NOT considered as DLTs:
Grade 3 or 4 CRS that improves to grade ≤2 within 72 hours.
Grade 3 or 4 neurotoxicity (e.g., encephalopathy, confusion) that improves to grade ≤2 within 14 days.
Grade 3 or 4 fever.
Bleeding in the setting of thrombocytopenia (platelet count ≤50×10⁹/L); documented bacterial infections or fever in the setting of neutropenia (absolute neutrophil count <1000/mm³).
Hypogammaglobulinemia (e.g., Grade 3 or Grade 4).
Grade 3 or 4 pulmonary toxicity that resolves to grade ≤2 within 7 days. For subjects intubated due to fluid overload from supportive care, this may be extended to 14 days.
Grade 3 or 4 liver function studies that improve to grade ≤2 within 14 days.
Grade 3 or 4 renal insufficiency that improves to grade ≤2 within 21 days.
Grade 3 or 4 thrombocytopenia or neutropenia is to be assessed retrospectively. After at least 6 subjects are infused, if 250% of subjects have prolonged cytopenias (i.e., lasting more than 28 days postinfusion) dose escalation will be suspended. For Cohorts D and G: Grade 3 cytopenias that were present at the start of LD chemotherapy may not be considered a DLT pending identification of another etiology.

AEs that have no plausible causal relationship with CTX110 are not to be considered DLTs.

### 5.3 CTX110 Redosing (Part A+Part B)

Subjects dosed with CTX110 have achieved objective responses without multi-log CAR T cell expansion in peripheral blood, suggesting a different biology and cell behavior than autologous CAR T cells. As allogeneic CAR T cells may be susceptible to more rapid clearance than autologous CAR T cells upon lymphocyte recovery, it therefore may be necessary to administer more than a single dose to clear any remaining cancerous cells. In order to achieve greater responses and prolonged durability, redosing is proposed in subjects that do not experience significant toxicity following the first infusion.

Redosing is also proposed based on the safety profile demonstrated with CTX110 to date, which includes 16 subjects treated at 5 different dose levels (DL1, DL2, DL3, DL3.5, and DL4). CTX110 has caused toxicities at severities and frequencies at or below those, which were observed with autologous CD19-directed CAR T cell therapies in NHL. There have been no infusion reactions or GvHD.

### 5.3.1. Redosing With CTX110

This study allows for up to three doses of CTX110.

Redosing may occur in the following 2 scenarios:
1. Planned redosing with or without LD chemotherapy based on timing or disease response criteria; this includes redosing scenarios in Cohorts C, D, E and G (see Table 12 below).
2. Redosing of CTX110 with LD chemotherapy after PD if the subject has had initial response after the first CTX110 infusion (all cohorts) or is MRD-positive (Cohorts D and G).

See Table 12.

TABLE 12
Redosing Schedule

| Cohort | Planned CTX110 Redosing |
|---|---|
| C2 | A planned second dose of CTX110 (4-8 weeks after the |
| | first dose) administered with LD chemotherapy to |
| | subjects who achieve SD or better at Day 28 scan |
| | (based on Lugano criteria). Option to redose without |
| | LD chemotherapy if subject is experiencing significant |
| | cytopenias. |
| D | Option to redose CTX110 with LD chemotherapy if |
| | subject has a decrease in BM blast count at Day 28 of |
| | at least 50% and <50% blasts. Once at least 3 subjects |
| | have been redosed, for subsequent subjects who are in |
| | morphologic remission and remain MRD positive, a Day |
| | 28 dose may be administered with LD chemotherapy. |
| E | Planned second dose of CTX110 on Day 8 (+2 days |
| | administered without LDchemotherapy. |
| | Planned third dose of CTX110 on Day 28 for subjects |
| | who achieve SD or better atDay 28 scan (based on |
| | Lugano criteria). Day 28 dose may be administered |
| | with daratumumab alone and without LD chemotherapy |
| | if the subject is experiencing significant cytopenias. |
| G | Option for second dose of TX110 on Day 28 with LD |
| | chemotherapy and daratumumab if subject has a |
| | decrease in BM blast count at Day 28 of at least |
| | 50%with <50% blasts in BM (Cohort G1). Once at least |
| | 3 subjects have been redosed, for subsequent subjects |
| | who are in morphologicremission and remain MRD |
| | positive, a Day 28 dose may be administered with |
| | LD chemotherapy and daratumumab (Cohorts G1 and G2). |

Planned Redosing in Cohorts C and E in Subjects with NHL

For planned second dose in Cohort C and planned third dose in Cohort E, subjects must meet the following criteria:
No prior DLT during dose escalation (if applicable)
No prior grade ≥3 CRS without resolution to grade ≤2 within 72 hours following CTX110 infusion
No prior GvHD following the initial CTX110 infusion
No prior grade ≥2 ICANS following CTX110 infusion that did not resolve

Additional redosing criteria are as follows at the time of LD chemotherapy and prior to second CTX110 infusion for subjects in Cohort E.
ECOG performance status 0 or 1
No requirement for supplemental oxygen to maintain a saturation level >91%
No new uncontrolled cardiac arrhythmia
No hypotension requiring vasopressor support or fluid bolus
No active uncontrolled infection (positive blood cultures for bacteria, fungus, or virus not responding to treatment)
Renal: Estimated glomerular filtration rate >50 mL/min/1.73 m²
Liver: AST or ALT <3×ULN; total bilirubin <1.5×ULN (for subjects with Gilbert's syndrome, total bilirubin <2 mg/dL)
No worsening of clinical status compared to prior CTX110 infusion that places the subject at increased risk of toxicity.
No new neurological symptoms suggesting CNS disease involvement. In the case of a normal finding on LP or MRI and following resolution of neurologic symptoms, redosing could be considered.
Women who are pregnant or breastfeeding are not eligible for redosing.

Subjects who are redosed should be followed per the Schedule of Assessments set forth in Tables 21-23, consistent with the initial dosing with the following considerations:
Echocardiogram (unless new cardiac signs or symptoms), brain MRI and lumbar puncture (unless new neurological symptoms concerning for progression) are not required.
Tissue biopsy should be obtained whenever possible to demonstrate CD19 expression. However, if not possible prior to the second planned dose, a biopsy of tumor should be performed if no response to second planned dose of CTX110 is observed.
PET/CT must be performed within 4 weeks of the second planned dose.
Bone marrow biopsy and aspirate must be repeated within 4 weeks of the second planned dose in subjects with initial bone marrow involvement.
   Redosing in Cohort C for Subjects with NHL

Subjects in Cohort C who achieved SD, PR, or CR at Day 28 (based on Lugano criteria) may receive a second planned CTX110 infusion 4 to 8 weeks after the first CTX110 infusion.

In subjects with cytopenias (ANC <1000/mm³ and/or platelets <25,000×10⁹/L), it may choose to redose without LD chemotherapy.

Redosing in Cohort E for Subjects with NHL

Subjects in Cohort E receive a second planned CTX110 infusion without LD chemotherapy on Day 8 (+2 days) after the first CTX110 infusion. Subjects who achieve SD or better at the Day 28 scan receive a third planned dose of CTX110 on Day 28.

Cohort E may begin with CTX110 infusion at DL3.5 after it has been deemed safe. A higher dose level (DL4) may be used based on assessment of toxicity profile. The maximum dose level may be DL4.

Subjects must meet eligibility criteria to receive the second dose on Day 8. The criteria are the same as those described for the third dose provided herein except that no ICANS of any grade following the first infusion is permitted. Also, no CRS of any grade following the first infusion is permitted except grade 1 CRS lasting <48 hours and the subject must be free of any symptoms for 48 hours prior to the second infusion.

Criteria for the planned Day 28 dose for Cohort E are the same as those described herein except that ICANS of any grade severity is not permitted.

Redosing for ALL Cohorts

For Cohorts D or G (adult B cell ALL), the above criteria apply except that the subject must have previously achieved a CR/CRi or at least 50% decrease in bone marrow blast count following initial CTX110 dose. In addition, subjects who have met these criteria but remain MRD-positive may be redosed at least 4 weeks after the initial dose. Subjects who are MRD-negative and become MRD-positive without increase in bone marrow blast count may be considered for redosing.

Redosing after Progressive Disease for all Cohorts

For all cohorts, a subject may be redosed with CTX110 after PD if the subject had prior clinical response after the first infusion. To be considered for redosing, subjects must have achieved evidence of clinical benefit, as demonstrated by a decrease in tumor size and/or FDG-avidity on a PET/CT scan after CTX110 infusion for subjects with NHL, and either concurrently or subsequently progressed or relapsed within 12 months of the initial or last CTX110 infusion. Redosing may occur only if disease extent is less than with initial CTX110 infusion and will proceed after consultation with the medical monitor.

The earliest time at which a subject could be redosed after PD is ≥2 months after the initial CTX110 infusion for NHL cohorts and >4 weeks after the initial CTX110 infusion for adult ALL cohort. Redosing in subjects with grade 3 or 4 neutropenia or thrombocytopenia who are >2 months post last CTX110 infusion may not be permitted unless the cytopenias can be clearly attributed to progressive disease or other reversible cause.

To be redosed with CTX110, subjects must meet the following criteria:
Confirmation tumor (NHL) or bone marrow (ALL) remains CD19⁺ at relapse (by flowcytometry or immunohistochemistry).
No prior DLT during dose-escalation (if applicable)
No prior grade ≥3 CRS without resolution to grade ≤2 within 72 hours following CTX110 infusion
No prior GvHD following CTX110 infusion
No prior grade ≥2 ICANS following CTX110 infusion
Meet initial study inclusion criteria (#1, #2, #5-10) and exclusion criteria (#2 [exceptprior treatment with CTX110]-15), as described herein
Meet criteria for LD chemotherapy and CTX110 infusion, as described herein

Subjects who are redosed after PD should be followed per the Schedule of Assessments [Screening to M24]) provided in Table 21, consistent with the initial dosing.

All Stage 1 screening assessments must be repeated, including the following additional considerations:
Brain MRI and lumbar puncture to be repeated if at high risk for CNS recurrence based on International Prognostic Index [IPI] criteria (Schmitz et al., 2016) or signs of CNS involvement are present at the time of redosing. Exception: adult B cell ALL subjects redosed within 2 months of CTX110 infusion without any new neurological symptoms after discussion with the medical monitor.
Echocardiogram: may be omitted if redosing occurs within 3 months of the CTX110 dose and if no new cardiac symptoms have occurred.
For NHL cohorts: The PET/CT scan demonstrating disease relapse/progression will serve as the new baseline for tumor response evaluation. Redosing must occur within 28 days of that scan. Bone marrow aspirate and biopsy must be repeated if it was not performed at the time of relapse/progression.
For adult B cell ALL cohorts: Bone marrow aspirate and biopsy within 14 days of LD chemotherapy.

Subjects who undergo redosing after PD will receive a lymphodepletion regimen and CTX110 dose that is identical to that previously received. Exception is made for subjects in Cohort C who may receive lymphodepletion.

In subjects who undergo redosing prior to disease progression, disease response assessments continue using the baseline PET/CT and bone marrow biopsy performed during screening. For subjects who are redosed after PD, disease response will be assessed relative to the most recent PET/CT scan and bone marrow prior to redosing.

### 6. Treatment

### 6.1 Daratumumab Infusion

Subjects receive 1 dose of daratumumab (16 mg/kg) by IV infusion or 1800 mg by SC injection administered at least 1 day prior to starting LD chemotherapy and within 10 days of CTX110 infusion. For subjects who achieve stable disease or better on Day 28, two additional doses of daratumumab (16 mg/kg) are to be administered at the Day 28 (±4 days) and Month 2 (±4 days) visits. Daratumumab administration (e.g., pre- and post-infusion medications, preparation, infusion rates, post-infusion monitoring) is performed according to the approved prescribing information. To facilitate administration, the first 16 mg/kg dose may be split (to 8 mg/kg) over 2 consecutive days. Confirmation of tumor response is based on the Day 28 visit positron emission tomography (PET)/computed tomography (CT) scan and the scan must be read before repeat dosing with daratumumab. If a subject experiences severe adverse events related to daratumumab, redosing is not permitted.

In addition, before repeat dosing with daratumumab, consultation with a medical practitioner is required for subjects with an occurrence of CRS or laboratory results that may suggest any degree of hemophagocytic lymphohistiocytosis or macrophage activation syndrome (HLH/MAS) or with clinical or laboratory signs of systemic inflammation before another daratumumab dose is given.

### 6.1.2 Daratumumab Infusion Reactions

To reduce the risk of administration reactions with daratumumab, 1 to 3 hours prior to administration subjects are premedicated with corticosteroids (e.g., IV methylprednisolone 100 mg, or equivalent), antipyretics (e.g., oral acetaminophen [paracetamol] 650 to 1,000 mg, or equivalent), and antihistamines (e.g., oral or IV diphenhydramine hydrochloride [or another H1-antihistamine] 25 to 50 mg, or equivalent).

Subjects are monitored frequently during the entire administration of daratumumab. For administration reactions of any grade/severity, infusion can be interrupted immediately, and symptoms managed. Therapy should be permanently discontinued if an anaphylactic reaction or life-threatening (grade 4) reaction occurs, and appropriate emergency care should be instituted. For subjects with grade 1, 2, or 3 reactions, after symptom resolution, the infusion rate can be reduced when restarting the infusion as described in the packaging insert (Daratumumab USPI, 2015).

To reduce the risk of delayed infusion reactions, oral corticosteroids (20 mg methylprednisolone or equivalent dose of an intermediate-acting or long-acting corticosteroid in accordance with local standards) can be administered to subjects following infusion as per prescribing information.

For the second or third dose of daratumumab, intermediate-acting corticosteroids (i.e., prednisone, methylprednisone) should be preferentially used to reduce the risk of interference with CTX110.

If a subject has an unresolved event of infusion reaction after daratumumab treatment, LD chemotherapy should be delayed and discussed with the medical monitor prior to proceeding.

### 6.1.3 Additional Daratumumab Administration Considerations

Daratumumab has been associated with herpes zoster (2%) and hepatitis B (1%) reactivation in patients with multiple myeloma (MM). To prevent herpes zoster reactivation, initiate antiviral prophylaxis within 1 week after infusion and continue for 3 months following treatment. For subjects with latent hepatitis B, consider hepatitis B prophylaxis prior to initiation of daratumumab and for 3 months following treatment (King et al., (2018) Asia Pac J Oncol Nurs, 5, 270-284).

Supportive care should be provided according to the approved prescribing information. Daratumumab binds to CD38 on red blood cells (RBCs) and results in a positive Indirect Antiglobulin Test (Indirect Coombs test). Blood type and screening should be performed per the approved prescribing information to prevent interference with blood compatibility testing.

### 6.2 Lymphodepleting Chemotherapy

All subjects receive LD chemotherapy prior to infusion of CTX110. LD chemotherapy consists of:
Fludarabine 30 mg/m² IV daily for 3 doses, AND
Cyclophosphamide 500 mg/m² IV daily for 3 doses.

Both agents can be started on the same day and administered for 3 consecutive days. Subjects should start LD chemotherapy (or daratumumab administration, for Cohorts C and G) within 7 days of study enrollment. Adult subjects with moderate impairment of renal function (creatinine clearance 30-70 mL/min/1.73 m²) should receive a reduced dose of fludarabine in accordance with applicable prescribing information.

The current full prescribing information for fludarabine and cyclophosphamide may be referenced for guidance regarding the storage, preparation, administration, supportive care instructions, and toxicity management associated with LD chemotherapy.

LD chemotherapy can be delayed if any of the following signs or symptoms are present:
Significant worsening of clinical status that, according to medial evaluation, increases the potential risk of AEs associated with LD chemotherapy.
Requirement for supplemental oxygen to maintain a saturation level >91%.
New uncontrolled cardiac arrhythmia.
Hypotension requiring vasopressor support.
Active infection: Positive blood cultures for bacteria, fungus, or virus not responding to treatment.
Grade ≥2 acute neurological toxicity.
Unresolved infusion reaction due to daratumumab treatment (Cohorts C and G only).

During Part A (dose escalation), if LD chemotherapy is delayed more than 30 days or the subject starts anticancer therapy, the subject will be replaced. For subjects whose toxicity(ies) are driven by underlying disease and require anticancer therapy, they must subsequently meet disease inclusion criteria, treatment washout, and end organ function criteria before restarting LD chemotherapy. Additionally, any subject who received anticancer therapy after enrollment (besides LD chemotherapy for Cohorts C, D, E, and G or daratumumab for Cohorts C and G) must have disease evaluation (including PET/CT scan) performed prior to starting LD chemotherapy (Cohorts D and E) or daratumumab (Cohorts C and G).

### 6.3 Administration of CTX110

Prior to the start of CTX110 infusion, the site pharmacy must ensure that 2 doses of tocilizumab and emergency equipment are available for each specific subject treated. Subjects should be premedicated per the site standard of practice with acetaminophen PO (i.e., paracetamol or its equivalent per site formulary) and diphenhydramine hydrochloride IV or PO (or another H1 antihistamine per site formulary) approximately 30 to 60 minutes prior to CTX110 infusion. Prophylactic systemic corticosteroids should not be administered, as they may interfere with the activity of CTX110.

CTX110 infusion is to be delayed if any of the following signs or symptoms are present:
New active uncontrolled infection.
Worsening of clinical status compared to prior to start of LD chemotherapy that places the subject at increased risk of toxicity.
Grade ≥2 acute neurological toxicity.

Each CTX110 infusion for Cohorts C, D, and G and the initial CTX110 infusion for Cohort E is to be administered at least 48 hours (but no more than 7 days) after the completion of LD chemotherapy. If CTX110 infusion is delayed by more than 10 days, LD chemotherapy must be repeated. Contact the CRISPR medical monitor if a subject's CTX110 infusion is delayed. See descriptions herein for re-dosing.

For Cohort E, the Day 8 CTX110 infusion may not be administered if any of the following signs or symptoms are present:
CRS following the first CTX110 infusion, except grade 1 CRS (per ASTCT criteria) lasting <48 hours (the subject must be free of any symptoms for 48 hours prior to the second infusion)
Any new neurological symptoms, including ICANS of any grade following the first infusion
Any cause that would put the subject at risk.

### 6.3.1 CTX110 Post-Infusion Monitoring

Following CTX110 infusion, subject's vitals should be monitored every 30 minutes for 2 hours after infusion or until resolution of any potential clinical symptoms.

Subjects in Part A are to be hospitalized for a minimum of 7 days after CTX110 infusion, or longer if required by local regulation or investigative site. In Parts A and B, subjects must remain in proximity of the investigative site (i.e., 1-hour transit time) for at least 28 days after CTX110 infusion. Management of acute CTX110-related toxicities should occur ONLY at the study site.

Subjects are monitored for signs of CRS, tumor lysis syndrome (TLS), neurotoxicity, GvHD, and other AEs according to the schedule of assessments (Tables 21-23). Guidelines for the management of CAR T cell-related toxicities are described in Section 7. Subjects should remain hospitalized until CTX110-related non-hematologic toxicities (e.g., fever, hypotension, hypoxia, ongoing neurological toxicity) return to grade 1. Subjects may remain hospitalized for longer periods if considered necessary by medical administrators.

### 6.4 Prior and Concomitant Medications

### 6.4.1 Allowed Medications

Necessary supportive measures for optimal medical care are given throughout the study, including IV antibiotics to treat infections, growth factors, blood components, etc., except for prohibited medications listed herein.

All concurrent therapies, including prescription and nonprescription medication, must be recorded from the date of signed informed consent through 3 months after CTX110 infusion. Beginning 3 months post-CTX110 infusion, only the following selected concomitant medications are collected: IV immunoglobulins, vaccinations, anticancer treatments (e.g., chemotherapy, radiation, immunotherapy), immunosuppressants (including steroids), and any investigational agents.

### 6.4.2 Prohibited Medications

The following medications are prohibited during certain periods of the study as specified below:
Corticosteroid therapy at a pharmacologic dose (≥5 mg/day of prednisone or equivalent doses of other corticosteroids) and other immunosuppressive drugs should be avoided after CTX110 administration unless medically indicated to treat new toxicity or as part of management of cytokine release syndrome (CRS) or neurotoxicity associated with CTX110, as described herein. Use of oral corticosteroids before and after daratumumab administration is permitted to prevent infusion reactions, as described herein.
Granulocyte-macrophage colony-stimulating factor (GM-CSF) following CTX110 infusion due to the potential to worsen symptoms of CRS.
Care should be taken with administration of granulocyte colony-stimulating factor (G-CSF) following CTX110 and should be avoided if possible until at least 21 days following CTX110 infusion.
Intrathecal CNS prophylaxis must be stopped at least 1 week prior to CTX110 infusion.
Any anticancer therapy (e.g., chemotherapy, immunotherapy, targeted therapy, radiation or other investigational agents) other than daratumumab (Cohort B) or LD chemotherapy (all cohorts) prior to disease progression.

### 7. Toxicity Management

### 7.1 General Guidance

Subjects must be closely monitored for at least 28 days after CTX110 infusion. Significant toxicities have been reported with autologous CAR T cell therapies and proactively monitor and treat all adverse events in accordance with protocol guidance are required.

The following general recommendations are provided based on prior experience with CD19-directed autologous CAR T cell therapies:
Fever is the most common early manifestation of cytokine release syndrome (CRS); however, subjects may also experience weakness, hypotension, or confusion as first presentation.
Diagnosis of CRS should be based on clinical symptoms and NOT laboratory values.
In subjects who do not respond to CRS-specific management, always consider sepsis and resistant infections. Subjects should be continually evaluated for resistant or emergent bacterial infections, as well as fungal or viral infections.
CRS, hemophagocytic lymphohistiocytosis (HLH), and tumor lysis syndrome (TLS) may occur at the same time following CAR T cell infusion. Subjects should be consistently monitored for signs and symptoms of all the conditions and managed appropriately. Hemophagocytic lymphohistiocytosis (HLH) observed signs and symptoms are a manifestation of CRS and will therefore not be graded separately.
Neurotoxicity may occur at the time of CRS, during CRS resolution, or following resolution of CRS. Grading and management of neurotoxicity are performed separately from CRS.
Tocilizumab must be administered within 2 hours from the time of order.

The safety profile of CTX110 is continually assessed throughout the study.

### 7.2 Toxicity-Specific Guidance

### 7.2.1 Infusion Reactions

Infusion reactions have been reported in autologous CD19-directed CAR T cell trials, including transient fever, chills, and/or nausea. Acetaminophen (paracetamol) and diphenhydramine hydrochloride (or another H1-antihistamine) may be repeated every 6 hours after CTX110 infusion, as needed, if an infusion reaction occurs. Nonsteroidal anti-inflammatory medications may be prescribed, as needed, if the subject continues to have fever not relieved by acetaminophen. Systemic steroids should not be administered except in cases of life-threatening emergency, as this intervention may have a deleterious effect on CAR T cells.

### 7.2.2 Febrile Reaction and Infection Prophylaxis

Infection prophylaxis should occur according to the institutional standard of care for patients with B cell malignancies in an immunocompromised setting. In the event of febrile reaction, an evaluation for infection should be initiated and the subject managed appropriately with antibiotics, fluids, and other supportive care as medically indicated and determined by the treating physician. Viral and fungal infections should be considered throughout a subject's medical management if fever persists. If a subject develops sepsis or systemic bacteremia following CTX110 infusion, appropriate cultures and medical management should be initiated. Additionally, consideration of CRS should be given in any instances of fever following CTX110 infusion within 30 days post-infusion.

For Cohorts C. E and G, prophylaxis for herpes zoster and hepatitis B reactivation in the setting of daratumumab treatment is strongly recommended, as per prescribing information.

### 7.2.3 Tumor Lysis Syndrome (TLS)

Subjects receiving CAR T cell therapy are at increased risk of TLS. Subjects should be closely monitored for TLS via laboratory assessments and symptoms from the start of LD chemotherapy until 28 days following CTX110 infusion. All subjects should receive prophylactic allopurinol (or a non-allopurinol alternative, such as febuxostat) and increased oral/IV hydration during screening and before initiation of LD chemotherapy. Prophylaxis can be stopped after 28 days following CTX110 infusion or once the risk of TLS passes. Sites should monitor and treat TLS as per their institutional standard of care, or according to published guidelines (Cairo and Bishop, (2004) Br J Haematol, 127, 3-11). TLS management, including administration of rasburicase, should be instituted promptly when clinically indicated.

### 7.2.4 Cytokine Release Syndrome (CRS)

CRS is a major toxicity reported with autologous CD19-directed CAR T cell therapy. CRS is due to hyperactivation of the immune system in response to CAR engagement of the target antigen, resulting in multi-cytokine elevation from rapid T cell stimulation and proliferation (Frey et al., (2014) Blood, 124, 2296; Maude et al., (2014) Cancer J, 20, 119-122). When cytokines are released, a variety of clinical signs and symptoms associated with CRS may occur, including cardiac, gastrointestinal (GI), neurological, respiratory (dyspnea, hypoxia), skin, cardiovascular (hypotension, tachycardia), and constitutional (fever, rigors, sweating, anorexia, headaches, malaise, fatigue, arthralgia, nausea, and vomiting) symptoms, and laboratory (coagulation, renal, and hepatic) abnormalities.

The goal of CRS management is to prevent life-threatening sequelae while preserving the potential for the antitumor effects of CTX110. Symptoms usually occur 1 to 14 days after autologous CAR T cell therapy, but the timing of symptom onset has not been fully defined for allogeneic CAR T cells.

CRS should be identified and treated based on clinical presentation and not laboratory cytokine measurements. If CRS is suspected, grading and management should be performed according to the recommendations in Tables 13-15, which are adapted from published guidelines (Lee et al., (2014) Blood, 124, 188-195). Since the development of the original Lee CRS grading criteria, physicians using CAR T cell therapies have gained further understanding of the presentation and time course of CRS. The recent American Society for Blood and Marrow Transplantation (ASBMT) consensus criteria (Lee et al., (2018) Biol Blood Marrow Transplant) recommend that grading should be based on the presence of fever with hypotension and/or hypoxia, and that other end organ toxicities should be managed separately with supportive care. Accordingly, in this protocol neurotoxicity is graded and managed using a different scale (see section entitled “Immune Effector Cell-Associated Neurotoxicity Syndrome (ICANS)”), and end organ toxicity in the context of CRS management refers only to hepatic and renal systems (as in the Penn Grading criteria; (Porter et al., (2018) J Hematol Oncol, 11, 35).

TABLE 13
Grading of CRS According to ASTCT Consensus
Criteria, Part A and Part B

| CRS | | | | |
|---|---|---|---|---|
| Parameter | Grade 1 | Grade 2 | Grade 3 | Grade 4 |
| Fever ¹ | Temperature | Temperature | Temperature | Temperature |
| | ≥38° C. | ≥38° C. | ≥38° C. | ≥38° C. |
| With | None | Not requiring | Requiring a | Requiring |
| hypo- | | vasopressors | vasopressor | multiple |
| tension | | | with or without | vasopressors |
| | | | vasopressin | (excluding |
| | | | | vasopressin) |
| And/or | None | Requiring low- | Requiring high- | Requiring |
| hypoxia ² | | flow nasal | flow nasal | positive |
| | | cannula ³ or | cannula ³, | pressure |
| | | blow-by | facemask, | (e.g., CPAP, |
| | | | nonrebreather | BiPAP, |
| | | | mask, or | intubation, |
| | | | Venturi mask | and |
| | | | | mechanical |
| | | | | ventilation) |

ASTCT: American Society for Transplantation and Cellular Therapy;
BiPAP: bilevel positive airway pressure;
C: Celsius;
CPAP: continuous positive airway pressure;
CRS: cytokine release syndrome
Note:
Organ toxicities associated with CRS may be graded according to CTCAE v5.0 but they do not influence CRS grading.
¹ Fever is defined as temperature ≥38° C. not attibutable to any other cause. In subjects who have CRS then receive antipyretics or anticytokine therapy such as tocilizumab or steroids, fever is no longer required to grade subsequent CRS severity. In this case, CRS grading is driven by hypotension and/or hypoxia.
² CRS grade is determined by the more severe event: hypotension or hypoxia not attributable to any other cause. For example, a patient with temperature of 39.5° C., hypotension requiring 1 vasopressor, and hypoxia requiring low-flow nasal cannula is classified as grade 3 CRS.
³ Low-flow nasal cannula is defined as oxygen delivered at ≤6 L/minute. Low flow also includes blow-by oxygen delivery, sometimes used in pediatrics. High-flow nasal cannula is definded as oxygen delivered at >6 L/minute.

TABLE 14
Cytokine Release Syndrome Grading and Management Guidance.

| CRS | | |
|---|---|---|
| Severity ¹ | Tocilizumab | Corticosteroids |
| Grade 1 | Tocilizumab ² may be | N/A |
| | considered in consultation | |
| | with the medical monitor. | |
| Grade 2 | Administer tocilizumab | If no improvement within |
| | 8 mg/kg IV over 1 hour | 24 hours after starting |
| | (not to exceed 800 mg) ² | tocilizumab, administer |
| | Repeat tocilizumab every | methylprednisolone 1 mg/kg |
| | 8 hours as needed if not | IV twice daily. |
| | responsive to IV fluids or | Continue corticosteroid use |
| | increasing supplemental | until the event is grade ≤1, |
| | oxygen. | then taper over 3 days. |
| | Limit to ≤3 doses in a | |
| | 24-hour period; maximum | |
| | total of 4 doses. | |
| Grade 3 | Per grade 2 | Per grade 2 |
| Grade 4 | Per grade 2 | Per grade 2 |
| | If no response to multiple | |
| | doses of tocilizumab and | |
| | steroids, consider using other | |
| | anticytokine therapies (e.g., | |
| | siltuximab, anakinra). | |

CRS: cytokine release syndrome; IV: intravenously; N/A: not applicable.
¹ See Lee et al., 2019.
² Refer to tocilizumab prescribing information.

TABLE 15
High-dose Vasopressors.

| Pressor | Dose* | |
|---|---|---|
| Norepinephrine monotherapy | ≥20 | μg/min |
| Dopamine monotherapy | ≥10 | μg/kg/min |
| Phenylephrine monotherapy | ≥200 | μg/min |
| Epinephrine monotherapy | ≥10 | μg/min |

| | |
|---|---|
| If on vasopressin | Vasopressin + norepinephrine |
| | equivalent of ≥10 μg/min** |
| If on combination vasopressors | Norepinephrine equivalent |
| (not vasopressin) | of ≥20 μg/min** |

*All doses are required for ≥3 hours.
**VASST Trial vasopressor equivalent equation: norepinephrine equivalent dose = [norepinephrine (μg/min)] + [dopamine (μg/min)/2] + [epinephrine (μg/min)] + [phenylephrine (μg/min)/10].

Throughout the duration of CRS, subjects should be provided with supportive care consisting of antipyretics, IV fluids, and oxygen. Subjects who experience grade ≥2 CRS (e.g., hypotension, not responsive to fluids, or hypoxia requiring supplemental oxygenation) should be monitored with continuous cardiac telemetry and pulse oximetry. For subjects experiencing grade 3 CRS, consider performing an echocardiogram to assess cardiac function. For grade 3 or 4 CRS, consider intensive care supportive therapy. Intubation for airway protection due to neurotoxicity (e.g., seizure) and not due to hypoxia should not be captured as grade 4 CRS. Similarly, prolonged intubation due to neurotoxicity without other signs of CRS (e.g., hypoxia) is not considered grade 4 CRS. An underlying infection in cases of severe CRS shall be considered, as the presentation (fever, hypotension, hypoxia) is similar. Resolution of CRS is defined as resolution of fever (temperature ≥38° C.), hypoxia, and hypotension (Lee et al., (2018) Biol Blood Marrow Transplant).

### 7.2.5 Neurotoxicity

Lumbar puncture is required for any grade ≥3 neurotoxicity and is strongly recommended for grade 1 and grade 2 events, if clinically feasible. Lumbar puncture must be performed within 48 hours of symptom onset, unless not clinically feasible.

Viral encephalitis (e.g., HHV-6 encephalitis; see below) must be considered in the differential diagnosis for subjects who experience neurocognitive symptoms after receiving CTX110. Whenever lumbar puncture is performed, in addition to the standard panel performed at site (which should include at least cell count, Gram stain, and Neisseria meningitidis), the following viral panel must be performed: CSF PCR analysis for HSV-1 and -2, enterovirus, varicella zoster virus (VZV), cytomegalovirus (CMV), and HHV-6. Results from the infectious disease panel must be available within 5 business days of the lumbar puncture in order to appropriately manage the subject. If a site is unable to perform the panel tests, it must be discussed with the medical monitor.

Dexamethasone 10 mg IV×4/day will be initiated for any Grade 2 ICANS in subjects with adult B cell ALL (Cohorts D1 and D2 and Cohort G).

Neurotoxicity has been observed with autologous CD19-directed CAR T cell therapies. It may occur at the time of CRS, during the resolution of CRS, or following resolution of CRS, and its pathophysiology is unclear. The ASTCT consensus further defined neurotoxicity associated with CRS as ICANS, a disorder characterized by a pathologic process involving the CNS following any immune therapy that results in activation or engagement of endogenous or infused T cells and/or other immune effector cells (Lee et al., 2019).

Signs and symptoms can be progressive and may include aphasia, altered level of consciousness, impairment of cognitive skills, motor weakness, seizures, and cerebral edema. ICANS grading (Table 16) was developed based on CAR T cell-therapy-associated TOXicity (CARTOX) working group criteria used previously in autologous CAR T cell trials (Neelapu et al., 2018). ICANS incorporates assessment of level of consciousness, presence/absence of seizures, motor findings, presence/absence of cerebral edema, and overall assessment of neurologic domains by using a modified assessment tool called the ICE (immune effector cell-associated encephalopathy) assessment tool (Table 17).

Evaluation of any new onset neurotoxicity should include a neurological examination (including ICE assessment tool, Table 18), brain MRI, and examination of the CSF (via lumbar puncture) as clinically indicated. If a brain MRI is not possible, all subjects should receive a non-contrast CT to rule out intracerebral hemorrhage. Electroencephalogram should also be considered as clinically indicated. Endotracheal intubation may be needed for airway protection in severe cases.

Non-sedating, anti-seizure prophylaxis (e.g., levetiracetam) should be considered in all subjects for at least 21 days following CTX110 infusion or upon resolution of neurological symptoms (unless the antiseizure medication is considered to be contributing to the detrimental symptoms). Subjects who experience ICANS grade ≥2 should be monitored with continuous cardiac telemetry and pulse oximetry. For severe or life-threatening neurologic toxicities, intensive care supportive therapy should be provided. Neurology consultation should always be considered. Monitor platelets and for signs of coagulopathy, and transfuse blood products appropriately to diminish risk of intracerebral hemorrhage. Table 16 provides neurotoxicity grading, Table 18 provides management guidance, and Table 17 provides neurocognitive assessment performed using the ICE assessment (see below). In addition to treatment guidelines provided in Table 18, nonsteroidal agents (e.g., anakinra, etc.) may be considered for ICANS management after discussion with the CRISPR medical monitor (Neill et al., 2020).

For subjects who receive active steroid management for more than 3 days, antifungal and antiviral prophylaxis is recommended to mitigate a risk of severe infection with prolonged steroid use. Consideration for antimicrobial prophylaxis should also be given.

TABLE 16
ICANS Grading.

| Neurotoxicity | | | | |
|---|---|---|---|---|
| Domain | Grade 1 | Grade 2 | Grade 3 | Grade 4 |
| ICE score ¹ | 7-9 | 3-6 | 0-2 | 0 (subject is |
| | | | | unarousable and |
| | | | | unable to undergo |
| | | | | ICE assessment) |
| Depressed level of | Awakens | Awakens to | Awakens only to | Subject is |
| consciousness ² | spontaneously | voice | tactile stimulus | unarousable or |
| | | | | requires vigorous or |
| | | | | repetitive tactile |
| | | | | stimuli to arise; |
| | | | | stupor or coma |
| Seizure | N/A | N/A | Any clinical seizure, | Life-threatening |
| | | | focal or generalized, | prolonged seizure (>5 |
| | | | that resolves | min) or repetitive |
| | | | rapidly, or | clinical or |
| | | | nonconvulsive | electrical seizures |
| | | | seizures on EEG | without return to |
| | | | that resolve with | baseline in between |
| | | | intervention | |
| Motor findings ³ | N/A | N/A | N/A | Deep focal motor |
| | | | | weakness such as |
| | | | | hemiparesis or |
| | | | | paraparesis |
| Elevated ICP/ | N/A | N/A | Focal/local edema | Diffuse cerebral |
| cerebral edema | | | on neuroimaging ⁴ | edema on |
| | | | | neuroimaging, |
| | | | | decerebrate or |
| | | | | decorticate |
| | | | | posturing, cranial |
| | | | | nerve VI palsy, |
| | | | | papilladema, or |
| | | | | Cushing's triad |

CTCAE: Common Terminology Criteria for Adverse Events; EEG: electroencephalogram; ICANS: immune effector cell-associated neurotoxicity syndrome; ICE: immune effector cell-associated encephalopathy (assessment tool); ICP: intracranial pressure; N/A: not applicable.
ICANS grade is determined by the most severe event (ICE score, level of consciousness, seizure, motor findings, raised ICP/cerebral edema) not attributable to any other cause.
¹ A subject with an ICE score of 0 may be classified as grade 3 ICANS if awake with global aphasia, but a subject with an ICE score of 0 may be classified as grade 4 ICANS if unarousable.
² Depressed level of consciousness should be attributable to no other cause (e.g., sedating medication).
³ Tremors and myoclonus associated with immune effector therapies should be graded according to CTCAE v5.0 but do not influence ICANS grading

TABLE 17
ICE Assessment.

| | | Maximum |
|---|---|---|
| Domain | Assessment | Score |
| Orientation | Orientation to year, month, city, | 4 points |
| | hospital | |
| Naming | Name 3 objects (e.g., point to | 3 points |
| | clock, pen, button) | |
| Following | Ability to follow commands (e.g., | 1 point |
| command | “Show me 2 fingers” or | |
| | “Close your eyes and stick out | |
| | your tongue”) | |
| Writing | Ability to write a standard sentence | 1 point |
| | (includes a noun and verb) | |
| Attention | Ability to count backward from 100 by 10 | 1 point |

ICE score will be reported as the total number of points (0-10) across all assessments. See disclosures below

The ICE assessment is performed at screening, before administration of CTX110 on Day 1, and on Days 2, 3, 5, 8, and 28. If a subject experiences CNS symptoms, the ICE assessment should continue to be performed approximately every 2 days until resolution of symptoms. To minimize variability, whenever possible the assessment should be performed by the same research staff member who is familiar with or trained in administration of the ICE assessment.

TABLE 18
ICANS Management Guidance.

| Severity | Management |
|---|---|
| Grade 2 | Consider administering dexamethasone 10 mg IV every 6 hours |
| | (or equivalent methylprednisolone) unless subject already |
| | on equivalent dose of steroids for CRS. |
| | Continue dexamethasone use until event is grade ≤1, then |
| | taper over 3 days. |
| | Cohorts D and G (B cell ALL only): administer dexamethasone |
| | 10 mg IV four times a day. |
| Grade 3 | Administer dexamethasone 10 mg IV every 6 hours, unless |
| | subject already on equivalent dose of steroids for CRS. |
| | Continue dexamethasone use until event is grade ≤1, |
| | then taper over 3 days. |
| Grade 4 | Administer methylprednisolone 1000 mg IV per day for |
| | 3 days; if improves manage as above. |

CRS: cytokine release syndrome; ICANS: immune effector cell-associated neurotoxicity syndrome; IV: intravenously.

Headache, which may occur in a setting of fever or after chemotherapy, is a nonspecific symptom. Headache alone may not necessarily be a manifestation of ICANS and further evaluation should be performed. Weakness or balance problem resulting from deconditioning and muscle loss are excluded from definition of ICANS. Similarly, intracranial hemorrhage with or without associated edema may occur due to coagulopathies in these subjects and are also excluded from definition of ICANS. These and other neurotoxicities should be captured in accordance with CTCAE v5.0.

### 7.2.5.1 Human Herpes Virus 6 Encephalitis

Most humans are exposed to HHV-6 during childhood and seroprevalence can approach 100% in adults. HHV-6 is thought to remain clinically latent in most individuals after primary infections and to reactivate to cause disease in persons with severe immunosuppression (Agut et al., 2015; Hanson et al., 2018). Two types of HHV-6 (A and B) have been identified. Although no diseases have clearly been linked to HHV-6A infection, HHV-6B is responsible for the childhood disease exanthem subitem. The virus also exhibits neurotropism and persists in brain tissue in a latent form. HHV-6 encephalitis has been predominantly described in immunocompromised patients following allogeneic HSCT, and has also been described in immunocompromised patients receiving autologous CAR T cell therapies (Bhanushali et al., 2013; Hanson et al., 2018; Hill and Zerr, 2014). Based on data from allogeneic HSCT, immunocompromised patients who are treated with steroids are at higher risk of developing HHV-6 encephalitis.

Diagnosis of HHV-6 encephalitis should be considered in any immunocompromised subject with neurological symptoms (e.g., confusion, memory loss, seizures) following CTX110 infusion. In addition to brain MRI, the following samples are required for diagnostic tests: lumbar puncture for HHV-6 DNA PCR (should be performed within 48 hours of symptoms if clinically feasible) and blood (plasma preferred) for HHV-6 DNA PCR. Diagnosis of HHV-6 encephalitis should be considered in a subject with elevated CSF HHV-6 DNA detected by PCR, elevated blood (plasma preferred) HHV-6 DNA detected by PCR, and acute mental status findings (encephalopathy), or short-term memory loss, or seizures (Hill and Zerr, 2014). Associated brain MRI abnormalities (typically, but not exclusively, non-enhancing, hyperintense lesions in the medial temporal lobes, especially hippocampus and amygdala) may not be seen initially (Ward et al., 2019). Because brain MRI findings may not be present initially, treatment for HHV-6 encephalitis should be considered in the setting of neurological findings and high HHV-6 CSF viral load. CSF protein and cell count often may be unremarkable, although there may be mild protein elevation and mild pleocytosis. Subjects may also experience fever and/or rash (Ward et al., 2019). For any subject suspected to have HHV-6 encephalitis, the CRISPR medical monitor must be contacted.

In subjects diagnosed with HHV-6 encephalitis, treatment with ganciclovir or foscarnet should be initiated. Drug selection should be dictated by the drug's side effects, the subject's comorbidities, and the site's clinical practice. The recommended duration of therapy is 3 weeks or as per site clinical practice (Hill and Zerr, 2014; Ward et al., 2019).

Once treatment is initiated, peripheral blood HHV-6 viral load should be checked weekly by PCR. Decrease in blood viral load should be seen within 1 to 2 weeks after initiation of treatment. If viral load does not decrease following 1 to 2 weeks of treatment, switching to another antiviral agent (ganciclovir or foscarnet) should be considered. Antiviral therapy should be continued for at least 3 weeks and until PCR testing demonstrates clearance of HHV-6 DNA in blood. At the end of the therapy, lumbar puncture should be performed to confirm clearance of HHV-6 DNA in CSF. If possible, immunosuppressive medications (including steroids) should be reduced during treatment for HHV-6 encephalitis; however, this needs to be balanced with the subject's need for steroids, especially if ICANS is also suspected.

For subjects in whom HHV-6 encephalitis is suspected, retrospective assessment of HHV-6 IgG, IgM, and HHV-6 DNA by PCR should be performed from blood samples collected prior to CTX110 infusion, if available.

In subjects with consistently elevated HHV-6 DNA viral load (e.g., >10,000 copies/mL), and especially when viral load does not decrease following initiation of antiviral therapy, attempt should be made to distinguish HHV-6 reactivation from chromosomally integrated HHV-6 (CIHHV-6). If the site has capabilities to do so, CIHHV-6 can be confirmed by evidence of 1 copy of viral DNA/cellular genome, or viral DNA in hair follicles/nails, or by fluorescence in situ hybridization demonstrating HHV-6 integrated into a human chromosome.

In suspected end-organ disease, if biopsy occurs, tissue from the affected organ should be tested for HHV-6 infection by culture, immunochemistry, in situ hybridization, or reverse transcription PCR for mRNA, if the site is able to perform these.

### 7.2.6 B Cell Aplasia

B cell aplasia may occur and can be monitored by following immunoglobulin G blood levels. IV gammaglobulin can be administered for clinically significant hypogammaglobulinemia (systemic infections) according to institutional standard of care.

### 7.2.7 Hemophagocytic Lymphohistiocytosis (HLH)

HLH has been reported after treatment with autologous CD19-directed CAR T cells (Barrett et al., (2014) Curr Opin Pediatr, 26, 43-49; Maude et al., (2014) N Engl J Med, 371, 1507-1517; Maude et al., (2015) Blood, 125, 4017-4023; Porter et al., (2015) Sci Transl Med, 7, 303ra139; Teachey et al., (2013) Blood, 121, 5154-5157. HLH is a clinical syndrome that is a result of an inflammatory response following infusion of CAR T cells in which cytokine production from activated T cells leads to excessive macrophage activation. Signs and symptoms of HLH may include fevers, cytopenias, hepatosplenomegaly, hepatic dysfunction with hyperbilirubinemia, coagulopathy with significantly decreased fibrinogen, and marked elevations in ferritin and C-reactive protein (CRP). Neurologic findings have also been observed (Jordan et al., (2011) Blood, 118, 4041-4052; La Rosée, (2015) Hematology Am Soc Hematol Educ Program, 190-196.

CRS and HLH may possess similar clinical syndromes with overlapping clinical features and pathophysiology. HLH likely occurs at the time of CRS or as CRS is resolving. HLH should be considered if there are unexplained elevated liver function tests or cytopenias with or without other evidence of CRS. Monitoring of CRP and ferritin may assist with diagnosis and define the clinical course.

If HLH is suspected:
Frequently monitor coagulation parameters, including fibrinogen. These tests may be done more frequently than indicated in the schedule of assessments, and frequency should be driven based on laboratory findings.
Fibrinogen should be maintained ≥100 mg/dL to decrease risk of bleeding. Coagulopathy should be corrected with blood products.

Given the overlap with CRS, subjects should also be managed per CRS treatment guidance in Tables 13-15. The IL-1 inhibitor, anakinra or other anti cytokine therapies (such as emapalumab-lzsg) may also be considered following discussion with the medical monitor.

### 7.2.8 Cytopenias

Grade 3 neutropenia and thrombocytopenia, at times lasting more than 28 days post-infusion, have been reported in subjects treated with autologous CD19-directed CAR T cell products (Kymriah USPI, 2017; Yescarta USPI, 2017). Therefore, subjects receiving CTX110 should be monitored for such toxicities and appropriately supported. Consideration should be given to antimicrobial and antifungal prophylaxis for any subject with prolonged neutropenia.

For subjects experiencing grade ≥3 neutropenia, thrombocytopenia, or anemia that has not resolved within 28 days of CTX110 infusion, a complete blood count with differential should be performed weekly until resolution to grade ≤2.

G-CSF may be considered in cases of grade 4 neutropenia 21 days post-CTX110 infusion, when the risk of CRS has passed. G-CSF administration may be considered earlier but must first be discussed with the medical monitor. Antimicrobial and antifungal prophylaxis should be considered for any subject with prolonged neutropenia or on high doses of steroids.

For Cohort C, daratumumab may increase neutropenia and/or thrombocytopenia induced by background therapy. Monitor complete blood cell counts periodically during treatment according to the manufacturer's prescribing information for background therapies. Monitor subjects with neutropenia for signs of infection. Daratumumab dose delay may be required to allow recovery of neutrophils and/or platelets, as per prescribing information.

Consider supportive care with growth factors for neutropenia or transfusions for thrombocytopenia.

### 7.2.9 Graft Versus Host Disease (GvHD)

GvHD is seen in the setting of allogeneic HSCT and is the result of immunocompetent donor T cells (the graft) recognizing the recipient (the host) as foreign. The subsequent immune response activates donor T cells to attack the recipient to eliminate foreign antigen-bearing cells. GvHD is divided into acute, chronic, and overlap syndromes based on both the time from allogeneic HSCT and clinical manifestations. Signs of acute GvHD may include a maculopapular rash; hyperbilirubinemia with jaundice due to damage to the small bile ducts, leading to cholestasis; nausea, vomiting, and anorexia; and watery or bloody diarrhea and cramping abdominal pain (Zeiser and Blazar, (2017) N Engl J Med, 377, 2167-2179.

To support the proposed clinical study, a nonclinical Good Laboratory Practice (GLP)-compliant GvHD and tolerability study was performed in immunocompromised mice at 2 doses that exceed all proposed clinical dose levels by at least 10-fold. Further, due to the specificity of CAR insertion at the TRAC locus, it is highly unlikely for a T cell to be both CAR+ and TCR+. Remaining TCR+ cells are removed during the manufacturing process by immunoaffinity chromatography on an anti-TCR antibody column to achieve <0.15% TCR+ cells in the final product. A dose limit of 7×10⁴ TCR+ cells/kg can be imposed for all dose levels. This limit is lower than the limit of 1×10⁵ TCR+ cells/kg based on published reports on the number of allogeneic cells capable of causing severe GvHD during SCT with haploidentical donors (Bertaina et al., (2014) Blood, 124, 822-826. Through this specific editing, purification, and strict product release criteria, the risk of GvHD following CTX110 should be low, although the true incidence is unknown. Subjects should be monitored closely for signs of acute GvHD following infusion of CTX110. The timing of potential symptoms is unknown. However, given that CAR T cell expansion is antigen-driven and likely occurs only in TCR− cells, it is unlikely that the number of TCR+ cells appreciably increases above the number infused.

Diagnosis and grading of GvHD should be based on published criteria (Harrs et al., (2016) Biol Blood Marrow Transplant, 22, 4-10), as outlined in Table 19.

TABLE 19
Criteria for Grading Acute GvHD

| | Skin | Liver | | Lower GI |
|---|---|---|---|---|
| Stage | (active erythema only) | (bilirubin mg/dL) | Upper GI | (stool output/day) |
| 0 | No active (erythematous) | <2 | No or intermittent | <500 ml/day |
| | GvHD rash | | nausea, vomiting, | or <3 episodes/day |
| | | | or anorexia | |
| 1 | Maculopapular rash <25% BSA | 2-3 | Persistent nausea, | 500-999 ml/day |
| | | | vomiting, or | or 3-4 episodes/day |
| | | | anorexia | |
| 2 | Maculopapular rash 25-50% BSA | 3.1-6 | — | 1000-1500 ml/day |
| | | | | or 5-7 episodes/day |
| 3 | Maculopapular rash >50% BSA | 6.1-15 | — | >1500 ml/day |
| | | | | or >7 episodes/day |
| 4 | Generalized erythroderma (>50% | >15 | — | Severe abdominal pain |
| | BSA) plus bullous formation | | | with or without ileus, |
| | and desquamation >5% BSA | | | or grossly bloody stool |
| | | | | (regardless of stool |
| | | | | volume) |

BSA: body surface area; GI: gastrointestinal; GvHD: graft versus host disease.

Overall GvHD grade can be determined based on most severe target organ involvement.
Grade 0: No stage 1-4 of any organ.
Grade 1: Stage 1-2 skin without liver, upper GI, or lower GI involvement.
Grade 2: Stage 3 rash and/or stage 1 liver and/or stage 1 upper GI and/or stage 1 lower GI.
Grade 3: Stage 2-3 liver and/or stage 2-3 lower GI, with stage 0-3 skin and/or stage 0-1 upper GI.
Grade 4: Stage 4 skin, liver, or lower GI involvement, with stage 0-1 upper GI.

Potential confounding factors that may mimic GvHD such as infections and reactions to medications should be ruled out. Skin and/or GI biopsy should be obtained for confirmation before or soon after treatment has been initiated. In instance of liver involvement, liver biopsy should be attempted if clinically feasible.

Recommendations for management of acute GvHD are outlined in Table 20. To allow for intersubject comparability at the end of the trial, these recommendations shall follow except in specific clinical scenarios in which following them could put the subject at risk.

TABLE 20
Acute GvHD Management

| Grade | Management |
|---|---|
| 1 | Skin: Topical steroids or immunosuppressants; if stage 2: |
| | prednisone 1 mg/kg (or equivalent dose). |
| 2-4 | Initiate prednisone 2 mg/kg daily (or equivalent dose). |
| | IV form of steroid such as methylprednisolone should be |
| | considered if there are concerns with malabsorption. |
| | Steroid taper may begin after improvement is seen after ≥3 |
| | days of steroids. |
| | Taper should be 50% decrease of total daily steroid dose |
| | every 5 days. |
| | GI: In addition to steroids, start anti-diarrheal agents |
| | per standard practice. |

GI: gastrointestinal; IV: intravenous.

Decisions to initiate second-line therapy should be made sooner for subjects with more severe GvHD. For example, secondary therapy may be indicated after 3 days with progressive manifestations of GvHD, after 1 week with persistent grade 3 GvHD, or after 2 weeks with persistent grade 2 GvHD. Second-line systemic therapy may be indicated earlier in subjects who cannot tolerate high-dose glucocorticoid treatment (Martin et al., (2012) Biol Blood Marrow Transplant, 18, 1150-1163). Choice of secondary therapy and when to initiate can be based on conventional practice.

Management of refractory acute GvHD or chronic GvHD can be per institutional guidelines. Anti-infective prophylaxis measures should be instituted per local guidelines when treating subjects with immunosuppressive agents (including steroids).

### 7.2.10 Hypotension and Renal Insufficiency

Hypotension and renal insufficiency have been reported with CAR T cell therapy and should be treated with IV administration of normal saline boluses according to institutional practice guidelines. Dialysis should be considered when appropriate.

### 7.2.11. Special Consideration During COVID-19 Pandemic

Subjects enrolled in this study undergo LD chemotherapy, are immunocompromised, and at increased risk of infections. Hence, the clinical study protocol requires exclusion of subjects in the case of any ongoing active infection during screening, prior to LD chemotherapy, and prior to CTX110 infusion, or delayed infusions (see Section 4.2).

This measure will include subjects with active infection with Severe Acute Respiratory Syndrome Coronavirus-2 (SARS CoV 2), the causal agent of COVID 19 (coronavirus disease 2019). Due to the rapidly changing evidence as well as locoregional differences, local regulations and institutional guidelines shall be followed if the current situation allows a safe conduct of the study for an individual subject at a given time.

### 8. Study Procedures

Both the dose escalation and expansion parts of the study will consist of 3 distinct stages: screening and eligibility confirmation; treatment, consisting of LD chemotherapy and CTX110 infusion (Cohorts D and E), or daratumumab infusion followed by LD chemotherapy and CTX110 infusion (Cohorts C, E and G); and (3) follow-up. During the screening stage, subjects are assessed according to the eligibility criteria outlined above. After enrollment, subjects in Cohorts D, E, and G receive LD chemotherapy followed by infusion of CTX110; subjects in Cohort C receive daratumumab followed by LD chemotherapy, then CTX110 infusion. During follow-up, subjects are assessed for tumor response, disease progression, and survival. Throughout all study stages, subjects will be regularly monitored for safety.

A complete schedule of assessments is provided in Table 21 and Table 22 (all cohorts) and Table 23 (Cohort E only-unique assessments from planned redosing on Day 21 through Month 2 visit). Descriptions of all required study procedures are provided in this section. In addition to protocol-mandated assessments, subjects should be followed per institutional guidelines, and unscheduled assessments should be performed when clinically indicated.

For the purposes of this protocol, there is no Day 0. All visit dates and windows are to be calculated using Day 1 as the date of first CTX110 infusion.

TABLE 21
Schedule of Assessments (Screening to Month 24)

| | Study Stage | | | | | | | | | | | | | | | | | | | |
|---|---|---|---|---|---|---|---|---|---|---|---|---|---|---|---|---|---|---|---|---|
| | | Treatment(Stage 2) | | | Follow-up(Stage 3) | | | | | | | | | | | | | | | |
| | | Day | | | | | | | | | | | | | | | | | | |
| | | D −10 | D −5 | | | | | | | | | | | | | | | | | |
| | Screening | to | to | | | D 3 ± | D 5 ± | D 8 ± | D 10 ± | D 14 ± | D 21 ± | D 28 ± | M 2 ± | M 3 ± | M 6 ± | M 9 ± | M 12 ± | M 15 ± | M 18 ± | M 24 ± |
| | (Stage1) ¹ | D −6 ⁽²⁾ | D −3 | D 1⁽³⁾ | D 2 | 1 d | 2 d | 2 d | 2 d | 2 d | 2 d | 4 d | 7 d | 7 d | 14 d | 14 d | 14 d | 14 d | 14 d | 21 d |
| Informed consent | X | | | | | | | | | | | | | | | | | | | |
| Medical history⁴ | X | | | | | | | | | | | | | | | | | | | |
| Physical exam | X | X | X | X | X | X | X | X | X | X | X | X | X | X | X | X | X | X | X | X |
| Vital signs ⁵ | X | X | X | X | X | X | X | X | X | X | X | X | X | X | X | X | X | X | X | X |
| Height, weight ⁶ | X | X | X | X | | | | X | | X | | X | X | X | X | X | X | X | X | X |
| Pregnancy test ⁷ | X | X | X | | | | | | | | | | | | | | | | | |
| ECOG status | X | | | X | | | | | | | | X | | X | | | | | | |
| Echocardiogram | X | | | | | | | | | | | | | | | | | | | |
| 12-lead ECG ⁸ | X | X | X | X | | | | | | | | X | | | | | | | | |
| Brain MRI | X | | | | | | | | | | | | | | | | | | | |
| Lumbar puncture ⁹ | X | | | | | | | | | | | | | | | | | | | |
| ICE assessment ¹⁰ | X | | | X | X | X | X | X | | | | X | | | | | | | | |
| Patient-reported outcomes ¹¹ | X | | | X | | | | | | | | X | | X | X | X | X | | X | X |

| Concomitant meds ¹² | Continuous | |
|---|---|---|
| Adverse events ¹³ | Continuous | |
| Hospital utilization | | Continuous |

Treatment

| | | | | | | |
|---|---|---|---|---|---|---|
| Daratumumab ¹⁴ | X | | | | X | X |
| LD chemotherapy ¹⁵ | | X* | | | X^{‡} | |
| CTX110 infusion ¹⁶ | | | X* | X**^{‡} | X^{‡} | |

NHL Disease Response/Assessment (Central and Local)

| | | | | | | | | |
|---|---|---|---|---|---|---|---|---|
| PET scan/CT with IV contrast ¹⁷ | X | X | X | X | X | X | X | X |
| BM aspirate/biopsy ¹⁸ | X | X | | | | | | |
| Tumor biopsy ¹⁹ | | X | | | | | | |
| Tumor pathology ²⁰ | X | | | | | | | |

Adult B Cell ALL Disease Response/Assessment

| | | | | | | | | | | |
|---|---|---|---|---|---|---|---|---|---|---|
| BM biopsy and aspirate | X | X | X ²¹ | X | X | X | X | X | X | X |
| (central and local) ^{17 18} | | | | | | | | | | |
| Imaging for | X | X | X | X | X | X | X | X | X | X |
| extramedullary disease | | | | | | | | | | |
| (if applicable) | | | | | | | | | | |
| Peripheral blood | X | | | | | | | | | |
| chimerism (local) ²² | | | | | | | | | | |

Laboratory Assessments (Local)

| | | | | | | | | | | | | | | | | | | | | |
|---|---|---|---|---|---|---|---|---|---|---|---|---|---|---|---|---|---|---|---|---|
| CBC w/differential | X | X | X | X | X | X | X | X | X | X | X | X²³ | X | X | X | X | X | X | X | X |
| Serum chemistry²⁴ | X | X | X | X | X | X | X | X | X | X | X | X | X | X | X | X | X | X | X | X |
| Coagulation parameters | X | X | X | X | X | X | X | X | X | X | | X | | | | | | | | |
| Viral serology ²⁵ | X | | | | | | | | | | | | | | | | | | | |
| Immunoglobulins | X | | | X | | | | | | | | X | X | X | X | X | X | X | X | X |
| Ferritin, CRP | X | | | X | X | X | X | X | X | X | X | X | | | | | | | | |
| TBNK panel ²⁶ | X | X | X | X | | X | X | X | X | X | X | X | X | X | X | X | X | | | |
| B cells (CD19, CD20) | X | | | X | | | | | | | | X | X | X | X | X | X | X | X | X |
| Blood type, Ab screen ²⁷ | | X | | | | | | | | | | | | | | | | | | |

Biomarkers (Blood, Central)

| | | | | | | | | | | | | | | | | | | |
|---|---|---|---|---|---|---|---|---|---|---|---|---|---|---|---|---|---|---|
| CTX110 PK ^{28, 29} | X | | X ³⁰ | X | X | X | X | X | X | X | X | X | X | X | | X | | X |
| | | | pre/ | | | | | | | | | | | | | | | |
| | | | post | | | | | | | | | | | | | | | |
| Cytokines ³¹ | X | | X | X | X | X | X | X | X | X | X | X | | | | | | |
| Anti-CTX110 Ab ^{29,} | X | | | | | | | | | | X | | X | | | X | | X |
| Daratumumab PK ³² | X | X | X | | | | X | | | | X | X | | | | | | |
| DNA | X | | | | | | | | | | | | | | | | | |
| Cell-free DNA | X | | X | | | | | | | | X | X | X | X | X | X | X | X |
| Exploratory biomarkers ³³ | X | X ³⁴ | X | | | X | X | | X | | X | X | X | X | | X | | X |

Ab: antibody; AE: adverse event; B-ALL: B cell acute lymphoblastic leukemia; BM: bone marrow: CBC: complete blood count; CNS: central nervous system; CRISPR: clustered regularly interspaced short palindromic repeats; CRP: C-reactive protein; CT: computed tomography; D or d: day; EC₉₀: 90% effective concentration; ECG: electrocardiogram; ECOG: Eastern Cooperative Oncology Group; HBV: hepatitis B virus; HCV: hepatitis C virus; HIV-1/-2: human immunodeficiency virus type 1 or 2; HSCT: hematopoietic stem cell transplant; ICE: immune effector cell-associated encephalopathy; ICF: informed consent form; IPI: International Prognostic Index; LD: lymphodepleting; LP: lumbar puncture; M: month; MRI: magnetic resonance imaging; PBMC: peripheral blood mononuclear cell; PCR: polymerase chain reaction; PET: positron emission tomography; PK: pharmacokinetic(s); Q: every; TBNK: T-, B-, natural killer (cells).
*NOTE:
For both Part A and Part B, this study will allow for planned redosing with CTX110 for subjects in Cohorts A, E, and F per the redosing criteria disclosed herein. Subjects who are redosed should be followed per the schedule of assessments consistent with the initial dosing. All Stage 1 screening assessmentsmust be repeated, including the additional considerations specified in herein.
**For Cohort E only. See Table 23 for Cohort E assessments from Day 21 through Month 2.
^{‡}For both Part A and Part B, this study will allow for planned redosing with CTX110 for subjects in Cohorts A, E, C and F and redosing after pregression of disease. See descriptions herein for the eligibility for redosing and additional considerations for screening assessments. With the exception of Cohort E, all subjects will be followed according to Table 23 after redosing.
NOTE:
Certain assessments for visits after Day 8 may be performed as in-home or alternate-site visits. Assessments include hospital utilization, changes in health and/or changes in medications, body system assessment, vital signs, weight, PRO questionnaire distribution, and blood sample collections for local andcentral laboratory assessments.
¹ Screening assessments completed within 14 days of informed consent. Subjects allowed 1-time rescreening within 3 months of initial consent.
⁽²⁾ Only for Cohorts C, E and G. All procedures will be performed prior to daratumumab infusion.
⁽³⁾All baseline assessments on Day 1 are to be performed prior to CTX110 infusion unless otherwise specified; refer to Laboratory Manual for details.
⁴Includes complete surgical and cardiac history.
⁵ Includes sitting blood pressure, heart rate, respiratory rate, pulse oximetry, and temperature.
⁶ Height at screening only.
⁷ For female subjects of childbearing potential. Serum pregnancy test at screening. Serum or urine pregnancy test within 72 hours before start of LDchemotherapy (Cohorts A, B, and D) or daratumumab (Cohort C, E, G) or subsequent planned dose of CTX110 (Cohort A, C, D, E, and G).
⁸ Prior to daratumumab infusion (Cohort C, E, and G), prior to LD chemotherapy (Cohorts A, B, and D), and prior to CTX110 infusion (all cohorts).
⁹ Mandatory for Cohort D and G subjects. For Cohorts A, B, C, and E: LP at screening on subjects with high risk for CNS involvement (e.g., high-grade B cell lymphoma with MYC and BCL2 and/or BCL6 rearrangements, subjects with testicular involvement of lymphoma, or subjects with high-risk CNS IPI score). For LPs performed during neurotoxicity, samples should be sent to central laboratory for CTX110 PK and exploratory biomarkers whenever possible.
¹⁰ On Day 1 prior to CTX110 administration. If CNS symptoms persist, ICE assessment should continue to be performed approximately every 2 days untilsymptom resolution to grade 1 or baseline.
¹¹ Patient-reported outcomes surveys should be administered before any visit specific procedures are performed.
¹² All concomitant medications will be collected ≤3 months post-CTX110, after which only select concomitant medications will be collected.
¹³ Collect all AEs from informed consent to 3 months after each CTX110 infusion and collect only SAEs and AESIs from 3 months after last CTX110 infusionthrough Month 24 visit. After Month 24 to Month 60 or after a subject starts a new anticancer therapy after Month 3 study visit, only CTX110-related SAEs and CTX110-related AESIs, and new malignancies will be reported.
¹⁴ Cohort C only: 1 dose of daratumumab 16 mg/kg IV or 1800 mg SC administered ≥1 day prior to starting LD chemotherapy and within 10 days of CTX110 infusion. The first 16 mg/kg IV dose may be split (to 8 mg/kg) over 2 consecutive days. For subjects who achieve SD or better on Day 28, 2 additional doses of daratumumab (16 mg/kg IV or 1800 mg SC) will be administered at the Day 28 (±4 days) and Month 2 (±4 days) visits. Confirmation ofSD state based on PET/CT scan measurement will be required before repeat dosing with daratumumab. Consultation with the medical monitor is required in patients with an occurrence of CRS or with clinical or laboratory signs of systemic inflammation before another dose of daratumumab is given. If a subject experiences severe adverse events related to daratumumab, redosing with daratumumab will not be permitted.
¹⁵ For first CTX110 dose, start LD chemotherapy within 7 days of study enrollment. After completion of LD chemotherapy, ensure washout period of ≥48 hours(but ≤7 days) before CTX110 infusion. Physical exam, weight, and coagulation laboratories performed prior to first dose of LD chemotherapy. Vital signs, CBC, clinical chemistry, and AEs/concomitant medications assessed and recorded daily (i.e., 3 times) during LD chemotherapy.
¹⁶ For first CTX110 dose and other redosings with LD chemotherapy, CTX110 administered 48 hours to 7 days after completion of LD chemotherapy.
¹⁷ Baseline disease assessment (PET scan/CT with IV contrast for subjects with NHL or BM biopsy with imaging for subjects with B-ALL) to be performed within 28 days prior to CTX110 infusion. MRI with contrast allowed if CT is clinically contraindicated, or as required by local regulation. Additional imagingat M 2 allowed.
¹⁸ Additional BM biopsy and aspirate should be performed to confirm CR as part of disease evaluation and to evaluate CTX110 trafficking. BM biopsy and aspirate may also be performed at time of disease relapse. Samples from all BM biopsy and aspirate should be sent for CTX110 PK and exploratory biomarkers. To be performed ±5 days of visit date. If HLH is suspected, BM biopsy and aspirate to be performed as specified herein. In Part B (dose expansion), at Day 28, for NHL subjects BM data will be collected for the first 20 patients. The requirement for BM biopsy and aspirate on Day 28 for subjectsthat have no BM involvement at screening is to be revisited.
¹⁹ Optional: For subjects who have disease amenable to biopsy and who provide separate consent. To be performed ±5 days of visit date.
²⁰ It is preferred that subjects undergo tumor biopsy during screening. However, if a biopsy of relapsed/refractory disease was performed within 3 months prior toenrollment and after the most recent line of therapy, archival tissue may be used. If relapse occurs on study, every attempt should be made to obtain biopsy of relapsed tumor and send to central pathology. Tumor biopsy refers to tissue other than bone marrow.
²¹ Assessments at Months 2 and 3 to confirm CR if not achieved at Month 1.
²² To be performed only in subjects who have received prior allogeneic HSCT.
²³For subjects experiencing grade ≥3 neutropenia, thrombocytopenia, or anemia that has not resolved within 28 days of CTX110 infusion, a complete bloodcount with differential must be performed weekly until resolution to grade ≤2.
²⁴Ferritin and CRP performed at Screening, D 1, D 2, D 3, D 5, D 8, D 10, D 14, D 21, and D 28 only
²⁵ Infectious disease testing (HIV-1, HIV-2, HCV antibody/PCR, HBV surface antigen, HBV surface antibody, HBV core antibody) ≤45 days of enrollment maybe considered for subject eligibility.
²⁶ TBNK panel assessment at screening, before daratumumab infusion (Cohort C), before start of first day of LD chemotherapy (Cohorts A, B, D, and F), beforeCTX110 infusion (all cohorts), then all listed time points. To include 6-color TBNK panel, or equivalent for T, B, and natural killer cells.
²⁷ Blood type and antibody screen per daratumumab prescribing information to prevent interference with blood compatibility testing for subjects in Cohort C aftercohort allocation is confirmed, and before daratumumab infusion.
²⁸ Samples for CTX110 PK should be sent from any LP, BM aspirate, or tissue biopsy performed following CTX110 infusion. In subjects experiencing signs orsymptoms of CRS, neurotoxicity, or suspected HLH, additional blood samples should be drawn at intervals outlined in the laboratory manual.
²⁹ Discontinuation of sample collection may be requested if consecutive tests are negative. Continue sample collection for all listed time points.
³⁰ Two samples collected on Day 1: One pre-CTX110 infusion and one 20 (±5) minutes after the end of CTX110 infusion.
³¹ Additional cytokine samples should be collected daily for the duration of CRS. Day 1 samples to be collected prior to CTX110 infusion. During neurotoxicityand suspected HLH, additional cytokine samples will be collected (see laboratory manual for specific information). Additional cytokine samples should be collected for Cohorts C and G (daratumumab) for 3 to 4 visits in the first 10 days after the second and third dara infusions: TBNK profile, ferritin LDH and PK, physical exam, vitals. This requirement may be revisited after the first 10 subjects are treated in Cohorts C, E and G.
³² Daratumumab-related assessments for Cohort C, E, and G only. Daratumumab PK sample after first daratumumab infusion to be collected immediately prior toinitiation of LD chemotherapy; Day 1 sample collected prior to CTX110 infusion; Day 28 and M 2 samples collected prior to each daratumumab redosing.
³³ Samples for exploratory biomarkers should be sent from any LP or BM aspirate performed following CTX110 infusion. If CRS, neurotoxicity, or HLH occur, samples for assessment of exploratory biomarkers will be collected as instructed in the laboratory manual.
³⁴ Prior to first day of LD chemotherapy only.

TABLE 22
Schedule of Assessments (Months 30-60)

| | M 30 | M 36 | M 42 | M 48 | M 54 | M 60 | | |
|---|---|---|---|---|---|---|---|---|
| | (±21 | (±21 | (±21 | (±21 | (±21 | (±21 | Progressive | Secondary |
| Assessments | days) | days) | days) | days) | days) | days) | Disease | Follow-Up ¹ |
| Vital signs ² | X | X | X | X | X | X | X | X |
| Physical exam | X | X | X | X | X | X | X | X |
| Concomitant medications ³ | X | X | X | X | X | X | X | X |
| Disease assessment ⁴ | X | X | X | X | X | X | X | |
| Tumor biopsy ⁴ | | | | | | | X | |
| CBC with differential ⁵ | X | X | X | X | X | X | X | X |
| Serum chemistry ⁵ | X | X | X | X | X | X | X | X |
| Immunoglobulins ^{5, 6} | X | X | X | X | X | X | X | |
| TBNK profile ^{5, 6} | X | X | X | X | X | X | X | |
| CTX110 persistence (blood, | | X | | X | | X | X | X |
| central) ^{6, 7} | | | | | | | | |
| Cell-free DNA (blood, central) | | | | | | | X | |
| Exploratory biomarkers | | X | | X | | X | X | X |
| (blood, central) | | | | | | | | |
| Anti-CTX110, anti-daratumumab | | X | | X | | X | X | |
| Ab (blood, central) ⁶ | | | | | | | | |
| Patient-reported outcomes | X | X | X | X | X | X | X | |
| Adverse events ⁸ | X | X | X | X | X | X | X | X |

Ab: antibody; AESI: adverse event of special interest; ALL: acute lymphoblastic leukemia; BM: bone marrow; Cas9: CRISPR-associated protein 9; CBC: complete blood count; CRISPR: clustered regularly interspaced short palindromic repeats; CT: computed tomography; NHL: non-Hodgkin lymphoma; PET: positron emission tomography; PK: pharmacokinetic; SAE: serious adverse event; TBNK: T-, B-, natural killer (cells).
NOTE:
Certain assessments for visits after Day 8 may be performed as in-home or alternate-site visits. Assessments include hospital utilization, changes inhealth and/or changes in medications, body system assessment, vital signs, weight, PRO questionnaire distribution, and blood sample collections for local and central laboratory assessments.
¹ Subjects with progressive disease or who undergo stem cell transplant will discontinue the normal schedule of assessments and attend annual study visits. Visits will occur at 12-month intervals. Subjects who partially withdraw consent will undergo these procedures at minimum. One hundred-day transplant-related outcomes will be collected from subjects with B cell ALL who undergo stem cell transplant. These may include survival rate, non-relapse survivalrate and rate of GvHD.
² Includes temperature, blood pressure, pulse rate, and respiratory rate.
³ Only select concomitant medications will be collected.
⁴ Disease assessment will consist of review of physical exam, CBC, clinical chemistry, and lactate dehydrogenase for NHL subjects, and ofphysical exam, CBC with differential, and clinical chemistry for B cell ALL. NHL subjects with suspected malignancy will undergo PET/CT imagingand/or a BM biopsy to confirm relapse. B cell ALL subjects with suspected malignancy will undergo bone marrow biopsy and aspirate. Every attemptshould be made to obtain a biopsy of the relapsed tumor in subjects who progress.
⁵ Assessed at local laboratory. To include 6-color TBNK panel, or equivalent for T, B, and natural killer cells.
⁶ Discontinuation of sample collection may be requested. Continue sample collection for all listed time points.
⁷ Samples for CTX110 PK analysis should be sent to the central laboratory from any lumbar puncture, BM biopsy, or tissue biopsy performed followingCTX110 infusion.
⁸ SAEs and AESIs should be reported until last study visit. Only CTX110-related AESIs, CTX110-related SAEs, and new malignancies will be reported afterMonth 24 to Month 60 or if a subject begins new anticancer therapy after Month 3 study visit.

TABLE 23
Schedule of Unique Assessments for Cohort E: Planned Redosing on Visit Day 21 to Up to Month 2 Visit

| | Study Stage | | | | | | | | | | | | | | | | |
|---|---|---|---|---|---|---|---|---|---|---|---|---|---|---|---|---|---|
| | | | | | | | | | | Unique Assessments for Cohort E | | | | | | | Continue |
| | | Treatment (Stage 2) | | | | | | | | Planned | | | | | | | M3 Follow- |
| | | Initial Dosing on | | Same as Table 15 | | | | | | Redosing on D 21 | | | | | | | up as in |
| | | D 1 after LD Chemo | | Follow-up after initial dose | | | | | | (no LD Chemo) | Follow-up after second dose | | | | | | Table 21 |
| | | Day | | | | | | | | | | | | | | | |
| | Screening | D −5 to | | D 2 ± | D 3 ± | D 5 ± | D 8 ± | D 10 ± | D 14 ± | D 21 ± | D 23 ± | D 25 ± | D 28 ± | D 34 ± | D 41 ± | D 48 ± | M 2 ± |
| | (Stage 1) ¹ | D −3 | D 1 ² | 2 d | 2 d | 2 d | 2 d | 2 d | 2 d | 2 d ² | 2 d | 2 d | 2 d | 2 d | 2 d | 2 d | 7 d |
| Informed consent | X | | | | | | | | | | | | | | | | |
| Medical history ³ | X | | | | | | | | | | | | | | | | |
| Physical exam | X | X | X | X | X | X | X | X | X | X | X | X | X | X | X | X | X |
| Vital signs ⁴ | X | X | X | X | X | X | X | X | X | X | X | X | X | X | X | X | X |
| Height, weight ⁵ | X | X | X | | | | X | | X | | | | X | | | | X |
| Pregnancy test ⁶ | X | X | | | | | | | | X | | | | | | | |
| ECOG status | X | | X | | | | | | | | | | X | | X | | X |
| Echocardiogram | X | | | | | | | | | | | | | | | | |
| 12-lead ECG ⁷ | X | X | X | | | | | | | X | | | X | | | | |
| Brain MRI | X | | | | | | | | | | | | | | | | |
| Lumbar puncture ⁸ | X | | | | | | | | | | | | | | | | |
| ICE assessment ⁹ | X | | X | X | X | X | X | | | X | X | X | X | | | | |
| Patient-reported outcome ¹⁰ | X | | X | | | | | | | X | | | X | | | | |

| Concomitant meds ¹¹ | Continuous | |
|---|---|---|
| Adverse events ¹² | Continuous | |
| Hospital utilization | | Continuous |

Treatment

| | | | |
|---|---|---|---|
| LD chemotherapy ¹³ | X* | | |
| CTX110 infusion ¹⁴ | | X* | X^{‡} |

NHL Disease Response/Assessment (Central and Local)

| | | |
|---|---|---|
| PET scan/CT with IV contrast ¹⁵ | X | X |
| BM aspirate/biopsy ¹⁶ | X | X |
| Tumor biopsy ¹⁷ | | X |
| Tumor pathology ¹⁸ | X | |

Laboratory Assessments (Local)

| | | | | | | | | | | | | | | | | | |
|---|---|---|---|---|---|---|---|---|---|---|---|---|---|---|---|---|---|
| CBC w/differential | X | X | X | X | X | X | X | X | X | X | X | X | X | X | X | X | X ¹⁹ |
| Serum chemistry ²⁰ | X | X | X | X | X | X | X | X | X | X | X | X | X | X | X | X | X |
| Coagulation parameters | X | X | X | X | X | X | X | X | X | X | X | X | X | X | X | X | X |
| Viral serology ²¹ | X | | | | | | | | | | | | | | | | |
| Immunoglobulins | X | | X | | | | | | | X | | | X | X | X | | X |
| TBNK panel ²² | X | X | X | | X | X | X | | X | X | X | X | X | X | X | X | X |
| B cells (CD19, CD20) | X | | X | | | | | | | | | | X | | | | X |

Biomarkers (Blood, Central)

| | | | | | | | | | | | | | | | | | |
|---|---|---|---|---|---|---|---|---|---|---|---|---|---|---|---|---|---|
| CTX110 PK ^{23, 24} | X | | X ²⁵ | X | X | X | X | X | X | X ²⁵ | X | X | X | X | X | X | X |
| | | | pre/ | | | | | | | pre/ | | | | | | | |
| | | | post | | | | | | | post | | | | | | | |
| Cytokines ²⁶ | X | | X | X | X | X | X | X | X | X | X | X | X | X | X | X | X |
| Anti-CTX110 | X | | | | | | | | | X | | | X | | | X | |
| DNA | X | | | | | | | | | | | | | | | | |
| Cell-free DNA | X | | X | | | | | | | | | | X | | | X | X |
| Exploratory biomarkers ²⁷ | X | X ²⁸ | X | | | X | X | | X | X | | | X | | | X | X |

BM: bone marrow: CBC: complete blood count; CNS: central nervous system; CRP: C-reactive protein; CT: computed tomography; D or d: day; ECG: electrocardiogram; ECOG: Eastern Cooperative Oncology Group; HBV: hepatitis B virus; HCV: hepatitis C virus; HIV-1/-2: human immunodeficiency virus type 1 or 2; ICE: immune effector cell-associated encephalopathy; LD: lymphodepleting; LP: lumbar puncture; M: month; MRI: magnetic resonance imaging; PBMC: peripheral blood mononuclear cell; PET: positron emission tomography; PK: pharmacokinetic(s); TBNK: T-, B-, natural killer (cells).
NOTE:
Certain assessments for visits after Day 8 may be performed as in-home or alternate-site visits. Assessments include hospital utilization, changes in health and/or changes in medications, body system assessment, vital signs, weight, PRO questionnaire distribution, and blood sample collections for local and central laboratory assessments.
*NOTE:
This study will allow for redosing with CTX110 for subjects in Cohort E after pregression of disease. These subjects should be followed per Table 20 Schedule of Assessments (Screening to M 24).
¹ Screening assessments completed within 14 days of informed consent. Subjects allowed 1-time rescreening within 3 months of initial consent.
All baseline assessments on Day 1 and all Day 21 assessments are to be performed prior to CTX110 infusion unless otherwise specified; refer to Laboratory Manual for details.
³ Includes complete surgical and cardiac history.
⁴ Includes sitting blood pressure, heart rate, respiratory rate, pulse oximetry, and temperature.
⁵ Height at screening only.
⁶ For female subjects of childbearing potential. Serum pregnancy test at screening. Serum or urine pregnancy test within 72 hours before redosing with CTX110.
⁷ Prior to LD chemotherapy and prior to CTX110 infusion.
⁸ LP at screening on subjects with high risk for CNS involvement (e.g., high-grade B cell lymphoma with MYC and BCL2 and/or BCL6 rearrangements, subjects with testicular involvement of lymphoma, or subjects with high-risk CNS IPI score). For LPs performed during neurotoxicity, samples should be sent to central laboratory for CTX110 PK and exploratory biomarkers whenever possible.
⁹ On Day 1 prior to CTX110 administration. If CNS symptoms persist, ICE assessment should continue to be performed approximately every 2 days until symptom resolution to grade 1 or baseline.
¹⁰ Patient-reported outcome surveys should be administered before any visit specific procedures are performed.
¹¹ All concomitant medications will be collected ≤3 months post-CTX110, after which only select concomitant medications will be collected (Section 6.4.1).
¹² Collect all AEs from informed consent to 3 months after each CTX110 infusion and collect only SAEs and AESIs from 3 months after last CTX110 infusion through Month 24 visit. After Month 24 to Month 60 or after a subject starts a new anticancer therapy after Month 3 study visit, only CTX110-related SAEs and CTX110-related AESIs, and new malignancies are to be reported. See Section 9.7.
¹³ For first CTX110 dose, start LD chemotherapy within 7 days of study enrollment. After completion of LD chemotherapy, ensure washout period of ≥48 hours (but ≤7 days) before CTX110 infusion. Physical exam, weight, and coagulation laboratories performed prior to first dose of LD chemotherapy. Vital signs, CBC, clinical chemistry, and AEs/concomitant medications assessed and recorded daily (i.e., 3 times) during LD chemotherapy.
¹⁴ For first CTX110 dose, CTX110 administered 48 hours to 7 days after completion of LD chemotherapy.
¹⁵ Baseline disease assessment to be performed within 28 days prior to CTX110 infusion. MRI with contrast allowed if CT clinically contraindicated, or as required by local regulation. Additional imaging at M 2 allowed for subjects in Cohort E.
¹⁶ Additional BM biopsy and aspirate should be performed to confirm CR as part of disease evaluation. BM biopsy and aspirate may also be performed at time of disease relapse. Samples from all BM biopsy and aspirate should be sent for CTX110 PK and exploratory biomarkers. To be performed ±5 days of visit date. If HLH is suspected, BM biopsy and aspirate to be performed as specified in Section 7.2.7.
¹⁷ Optional: For subjects who have disease amenable to biopsy and who provide separate consent. To be performed ±5 days of visit date.
¹⁸ It is preferred that subjects undergo tumor biopsy during screening. However, if a biopsy of relapsed/refractory disease was performed within 3 months prior to enrollment and after the most recent line of therapy, archival tissue may be used. If relapse occurs on study, every attempt should be made to obtain biopsy of relapsed tumor and send to central pathology. Tumor biopsy refers to tissue other than bone marrow.
¹⁹ For subjects experiencing grade ≥3 neutropenia, thrombocytopenia, or anemia that has not resolved within 28 days of CTX110 infusion, a complete blood count with differential must be performed weekly until resolution to grade ≤2.
²⁰ Serum chemistry includes ferritin and CRP at all timepoints indicated except Day −5 to Day −3.
²¹ Infectious disease testing (HIV-1, HIV-2, HCV antibody/PCR, HBV surface antigen, HBV surface antibody, HBV core antibody) ≤45 days of enrollment may be considered for subject eligibility.
²² TBNK panel assessment at screening, before start of first day of LD, before CTX110 infusion, then all listed time points. To include 6-color TBNK panel, or equivalent for T, B, and natural killer cells.
²³ Samples for CTX110 PK should be sent from any LP, BM aspirate, or tissue biopsy performed following CTX110 infusion. In subjects experiencing signs or symptoms of CRS, neurotoxicity or suspected HLH, additional blood samples should be drawn at intervals outlined in the laboratory manual.
²⁴ Discontinuation of sample collection may be requested if consecutive tests are negative. Continue sample collection for all listed time points.
²⁵ Two samples collected on Day 1 and Day 21: One pre-CTX110 infusion and one 20 (±5) minutes after the end of CTX110 infusion.
²⁶ Additional cytokine samples should be collected daily for the duration of CRS. Day 1 samples to be collected prior to CTX110 infusion. During neurotoxicity and suspected HLH, additional cytokine samples are collected (see Laboratory Manual for specific info).
²⁷ Samples for exploratory biomarkers should be sent from any LP or BM aspirate performed following CTX110 infusion. If CRS, neurotoxicity or HLH occur, samples for assessment of exploratory biomarkers are collected as instructed in the Laboratory Manual.
²⁸ Prior to first day of LD chemotherapy only.

### 8.1 Subject Screening, Enrollment, and Withdrawal

### 8.1.1 Subject Screening

The screening period begins on the date that the subject informed consent form (ICF) and continues through confirmation of eligibility and enrollment into the study. Once informed consent has been obtained, the subject will be screened to confirm study eligibility as outlined in the schedule of assessments (Table 21). Screening assessments to be completed within 14 days of a subject signing the informed consent.

Subjects will be allowed a one-time rescreening, which may take place within 3 months of the initial consent.

### 8.1.2. Assignment of Subjects to Treatment Cohorts

Cohorts C and E will comprise subjects with NHL, including DLBCL NOS, high grade B cell lymphoma with MYC and BCL2 and/or BCL6 rearrangements, transformed FL, and grade 3b FL. Subjects with B cell ALL are exclusively assigned to Cohort D or G in parallel with other cohorts. CTX110 infusion will begin at DL3 in Cohorts B and C, at DL2 or DL3 for Cohort D and G, and at DL4 for Cohort E. Dosing will be staggered as described herein.

For Part B (NHL cohort expansion), subjects defined as high-risk are capped at 20 subjects. Subjects are defined as high risk prospectively with local results and/or retrospectively with central analysis if any of the following apply (in case of discrepancy, central analysis will take precedent):
A single lesion with largest diameter ≥10 cm and/or SPD ≥500 mm2 (pre LD chemotherapy) as assessed by local and/or central analysis [SPD=sum of the product diameter]; and/or
No history of response to any chemotherapy regimen (PR or better) and/or a DLBCL diagnosis within 6 months of enrollment

### 8.2 Study Assessments

Refer to the schedule of assessments (Table 21 and Table 22 [all cohorts], and Table 23 [Cohort E only]) for the timing of the required procedures.

### 8.2.1. Medical History

Demographic data are collected. Medical history, including a full history of the subject's disease, previous cancer treatments, and response to treatment from date of diagnosis are obtained. Cardiac, neurological, and surgical history are obtained. For trial entry, all subjects must fulfill all inclusion criteria described herein, and have none of the exclusion criteria described herein.

### 8.2.2 Physical Exam

Physical examination, including examination of major body systems, including general appearance, skin, neck, head, eyes, ears, nose, throat, heart, lungs, abdomen, lymph nodes, extremities, and nervous system, are performed at every study visit and the results documented. Changes noted from the exam performed at screening are recorded as an AE.

### 8.2.3. Vital Signs, Including Height and Weight

Vital signs will be recorded at every study visit and include sitting blood pressure, heart rate, respiratory rate, pulse oximetry, temperature, and height. Weight will be obtained according to the schedule in Table 21 (all cohorts) and Table 23 (Cohort E only), and height will only be obtained at screening.

### 8.2.4 ECOG Performance Status

Performance status is assessed at the screening, CTX110 infusion (Day 1), Day 28, and Month 3 visits using the ECOG scale to determine the subject's general well-being and ability to perform activities of daily life. The ECOG performance status scale is provided in Table 24 below.

TABLE 24
Eastern Cooperative Oncology Group Performance Status Scale

| Grade | ECOG Performance Status |
|---|---|
| 0 | Fully active, able to carry on all pre-disease performance |
| | without restriction |
| 1 | Restricted in physically strenuous activity but ambulatory |
| | and able to carry out work of a light or sedentary nature, |
| | e.g., light house work, office work |
| 2 | Ambulatory and capable of all self-care but unable to carry |
| | out any work activities; up and about more than 50% of |
| | waking hours |
| 3 | Capable of only limited self-care; confined to bed or chair |
| | more than 50% of waking hours |
| 4 | Completely disabled; cannot carry on any self-care; totally |
| | confined to bed or chair |
| 5 | Dead |

Developed by the Eastern Cooperative Oncology Group, Robert L. Comis, MD, Group Chair (Oken et al., 1982).

### 8.2.5 Echocardiogram

A transthoracic cardiac echocardiogram (for assessment of left ventricular ejection fraction) will be performed and read by trained medical personnel at screening to confirm eligibility. Additional cardiac assessment is recommended during grade 3 or 4 CRS for all subjects who require >1 fluid bolus for hypotension, who are transferred to the intensive care unit for hemodynamic management, or who require any dose of vasopressor for hypotension (Brudno and Kochenderfer, 2016).

### 8.2.6 Electrocardiogram

Twelve (12)-lead electrocardiograms (ECGs) will be obtained during screening, prior to daratumumab infusion (Cohort C only), prior to LD chemotherapy on the first day of treatment (Cohorts A, B, D, E, and F), and prior to CTX110 administration on Day 1 (all cohorts), and on Day 28. QTc and QRS intervals will be determined from ECGs.

### 8.2.7 NHL Tumor Pathology

Histopathological diagnosis of NHL subtype is based on local laboratory assessment. It is preferred that subjects undergo tumor biopsy during screening. However, if a biopsy of relapsed/refractory disease was performed after completion of last line of therapy and within 3 months prior to enrollment, archival tissue may be used. Bone biopsies and other decalcified tissues are not acceptable due to interference with downstream assays.

Portions of the tissue biopsy will be submitted to a central laboratory for analysis. Requirements for tissue preparation and shipping can be found in the Laboratory Manual. If archival tissue is of insufficient volume or quality to fulfill central laboratory requirements, a biopsy during screening must be performed. Archival tumor tissue samples may be analyzed for markers of aggressive NHL (e.g., MYC, BCL2, BCL6) as well as immune markers in the tumor and surrounding microenvironment (e.g., programmed cell death protein 1, programmed cell death-ligand 1).

### 8.2.8 Brain MRI

To rule out CNS metastasis, a brain MRI will be performed during the screening. Requirements for the acquisition, processing, and transfer of this MRI will be outlined in the Imaging Manual.

### 8.2.9 Lumbar Puncture

A lumbar puncture is performed at screening according to institutional standard procedures in all subjects with adult ALL (Cohort D and G) to rule out presence of malignancy. For Cohorts C and E, lumbar puncture is to be performed in subjects at high risk for CNS involvement. These include subjects with high grade B cell lymphoma with MYC and BCL2 and/or BCL6 rearrangement; subjects with testicular involvement of lymphoma; or subjects with high-risk scores on the CNS IPI, a tool used to estimate risk of CNS relapse/progression in patients with DLBCL treated with R-CHOP (Schmitz et al., 2016). If clinically feasible, for lumbar punctures performed during neurotoxicity, CSF samples should be sent to the central laboratory for exploratory biomarkers and for presence of CTX110 (by PCR). Whenever lumbar puncture is performed in the setting of neurotoxicity evaluation, in addition to the standard panel performed at the site (which should include at least cell count, Gram stain, and Neisseria meningitidis) the following viral panel must be performed:
CSF PCR analysis for HSV-1 and -2, enterovirus, VZV, CMV, and HHV-6

Results of viral panel should be available within 5 business days from draw to support appropriate management of a subject.

### 8.2.10 Immune Effector Cell-Associated Encephalopathy (ICE) Assessment

Neurocognitive assessment will be performed using the ICE assessment. The ICE assessment is a slightly modified version of the CARTOX-10 screening tool, which now includes a test for receptive aphasia. The ICE assessment (Table 17) examines various areas of cognitive function: orientation, naming, following commands, writing, and attention.

The ICE assessment is performed at screening, before administration of CTX110 on Day 1, and on Days 2, 3, 5, 8, and 28. If a subject experiences CNS symptoms, the ICE assessment should continue to be performed approximately every 2 days until resolution of symptoms. To minimize variability, whenever possible the assessment should be performed by the same research staff member who is familiar with or trained in administration of the ICE assessment.

### 8.2.11 PET/CT and Radiologic Disease Response Assessments for NHL

PET/CT (CT must include IV contrast) scans of all sites of disease (including the neck, chest, abdomen, and pelvis) are required. The CT portion of PET/CT should be diagnostic quality, or a standalone CT with IV contrast should be performed. MRI with contrast may be used when CT is clinically contraindicated or as required by local regulation. The baseline PET/CT (with IV contrast) must be performed within 28 days prior to administration of CTX110, and postinfusion scans will be conducted per the schedule of assessments in Table 14 and Table 15 (all cohorts), and Table 16 (Cohort E only). Additional imaging at Month 2 is allowed for subjects in Cohort E. If a subject has symptoms consistent with possible disease progression, an unscheduled PET/CT (with IV contrast) should be performed.

Requirements for the acquisition, processing, and transfer of scans will be outlined in the Imaging Manual. When possible, the imaging modalities, machines, and scanning parameters used to acquire PET/CT should be kept consistent during the study. Tumor burden is quantified at baseline according to Lugano criteria (disclosed herein). Tumor burden assessment are to include the sum of perpendicular diameters (SPD) calculated by aggregating the dimensions of each target (nodal or extra nodal) lesion for a maximum of six target lesions, by multiplying the two longest perpendicular diameters of lesions. Target lesions should be selected from those with the largest size that can be reproducibly measured, representing overall tumor burden across multiple sites and/organs.

### 8.2.11.1. Imaging of Extramedullary Disease in B Cell ALL

Adult B cell ALL subjects with known extramedullary disease at baseline are assessed with imaging within 28 days prior to administration of CTX110, and postinfusion imaging will be conducted per the schedule of assessments in Tables 21-23. A modality appropriate for the anatomical location of disease may be used with the same imaging modality for the duration of participation. Extramedullary disease can be assessed as described in Table 28 below.

### 8.2.12 Bone Marrow Biopsy and Aspirate for NHL

A bone marrow biopsy and aspirate is performed at screening and at Day 28 to evaluate extent of disease. Subjects with history of bone marrow involvement who achieve a CR as determined on PET/CT scan will have a bone marrow biopsy to confirm response assessment. If a subject shows signs of relapse, the biopsy collection should be repeated. A sample of aspirate for presence of CTX110 (detected via PCR) should be sent for central laboratory evaluation at any point when bone marrow analysis is performed. Standard institutional guidelines for the bone marrow biopsy should be followed. Further instructions on processing and shipment are provided in the Laboratory Manual. Excess sample (if available) may be stored for exploratory research.

### 8.2.13 Optional Tumor Biopsy for NHL

To understand more about the trafficking of CTX110 into the tumor tissue and the impact of tumor environment on the function of CTX110, optional tumor biopsies will be obtained from subjects with tumor amenable to biopsy and who provide separate consent for this procedure. The optional tumor biopsy is performed at Day 28. Standard institutional guidelines for the tumor biopsy should be followed.

### 8.2.14 Bone Marrow Biopsy and Aspirate for B Cell ALL

A bone marrow biopsy and aspirate will be performed at screening or within 28 days of CTX110 infusion and assessed locally or centrally to confirm disease pathology and evaluate extent of disease. Remaining bone marrow and aspirate are assessed (locally or centrally) for MRD by multicolor flow cytometry or molecular approaches (e.g., PCR, NGS). Additional bone marrow aspirates may be performed at Months 2 and 3 to confirm complete remission if not achieved at Month 1 (Day 28). If a subject shows signs of relapse, the biopsy and aspirate collection should be repeated.

### 8.2.15 Laboratory Tests

Laboratory samples are collected and analyzed according to the schedule of assessment (Table 21 and Table 22 [all cohorts], and Table 23 [Cohort E only]). Local laboratory tests are summarized in Table 25 below.

TABLE 25
Local Laboratory Tests

| | |
|---|---|
| Hematology | Hematocrit, hemoglobin, red blood cell count, white blood cell count, |
| | neutrophils, lymphocytes, monocytes, basophils, eosinophils, platelet |
| | count, absolute neutrophil count, ABO type,¹ antibody screen^{1,} |
| | peripheral blasts in Cohorts D and G |
| TBNK panel | 6-color TBNK or equivalent commonly staining T cells (CD3, CD4, |
| | CD8), B cells (CD19), and natural killer cells (CD56, CD16) |
| Serum Chemistry | ALT (SGPT), AST (SGOT), bilirubin (total and direct), albumin, |
| | alkaline phosphatase, bicarbonate, blood urea nitrogen, calcium, |
| | chloride, creatinine, eGFR, glucose, lactate dehydrogenase, magnesium, |
| | phosphorus, potassium, sodium, total protein, CRP, ferritin |
| Coagulation | Prothrombin time, activated partial thromboplastin time, international |
| | normalized ratio, fibrinogen |
| Infectious Disease | HIV-1, HIV-2, hepatitis C virus antibody and PCR, hepatitis B surface |
| | antigen, hepatitis B surface antibody, hepatitis B core antibody |
| Immunological | CD19, CD20, IgA, IgG, IgM |
| Serum or Urine | Human chorionic gonadotropin |
| Pregnancy² | |

ALT: alanine aminotransferase; AST: aspartate aminotransferase; CRP: C-reactive protein; CRS: cytokine release syndrome; eGFR: estimated glomerular filtration rate; HHV-6: human herpesvirus 6; HIV-1/-2: human immunodeficiency virus type 1 or 2; HLH: hemophagocytic lymphohistiocytosis; IgA/G/M: immunoglobulin A, G, or M; LD: lymphodepleting; PCR: polymerase chain reaction; SGOT: serum glutamic oxaloacetic transaminase; SGPT: serum glutamic pyruvic transaminase; TNBK: T, B, and natural killer cells.
¹For Cohort C only, if necessary.
²For females of childbearing potential only. Serum pregnancy test required at screening. Serum or urine pregnancy test within 72 hours of start of LD chemotherapy (Cohorts D and E) or daratumumab (Cohort C) or second dose of CTX110 (Cohort E redosing), including the redosing schedule for respective cohorts.

### 8.3. Biomarkers

Blood, bone marrow, tumor, and CSF samples (only in subjects with ICANS) are collected to identify genomic, metabolic, and/or proteomic biomarkers that may be indicative of clinical response, resistance, safety, pharmacodynamic activity, or the mechanism of action of CTX110.

The following labs are drawn for analysis at a central laboratory. Reference the Laboratory Manual for information regarding the blood draw and sample handling for tests sent to the central laboratory for processing. Excess sample (if available) will be stored for exploratory research.

### 8.3.1. CTX110 Pharmacokinetic Analysis

PK analysis of CTX110 cells will be performed on blood samples collected according to the schedule described in Table 21 and Table 22 (all cohorts), and Table 23 (Cohort E only). In subjects experiencing signs or symptoms of CRS, neurotoxicity, and HLH, additional blood samples should be drawn in intervals outlined in the laboratory manual. The time course of the disposition of CTX110 in blood (Tsai et al., 2017) is described using a PCR assay that measures copies of CAR construct per μg DNA. Complementary analyses using flow cytometry to confirm the presence of CAR protein on the cellular surface may also be performed. The trafficking of CTX110 in CSF, bone marrow, or tumor tissues may be evaluated in any of these samples collected as per protocol-specific sampling.

### 8.3.2. Cytokines

Cytokines, including IL-2, IL-6, IL-8, IL-12, IL-15, IL-17a, interferon γ, tumor necrosis factor α, and GM-CSF, will be analyzed in a central laboratory. Correlational analysis performed in multiple prior CAR T cell clinical studies have identified these cytokines, and others, as potential predictive markers for severe CRS and/or neurotoxicity, as summarized in a recent review (Wang and Han, 2018). Blood for cytokines are collected at specified times as described in Table 21 (all cohorts) and Table 23 (Cohort E only). In subjects experiencing signs or symptoms of CRS, neurotoxicity, and HLH, additional samples should be drawn (per the schedule outlined in the laboratory manual).

### 8.3.3 Anti-CTX110 Antibody

The CAR construct is composed of a murine scFv. Blood will be collected throughout the study to assess for potential immunogenicity, per Table 21 and Table 22 (all cohorts), and Table 23 (Cohort E only).

### 8.3.4 Daratumumab Pharmacokinetic Analysis (Cohort C)

PK analysis of daratumumab will be performed on blood samples collected according to the schedule described in Table 21 and Table 22. The trafficking of daratumumab in CSF, bone marrow, or tumor tissues may be evaluated in any of these samples collected as per protocol-specific sampling.

### 8.3.5 Exploratory Research Biomarkers

Exploratory research may be conducted to identify molecular (genomic, metabolic, and/or proteomic) biomarkers and immunophenotypes that may be indicative or predictive of clinical response, resistance, safety, pharmacodynamic activity, and/or the mechanism of action of treatment.

### 9. Safety, Adverse Events, an Study Oversight

Each subject is monitored for clinical and laboratory evidence of AEs on a routine basis throughout the study. AEs in response to a query, observed by site personnel, or reported spontaneously by the subject are recorded. All AEs are followed to a satisfactory conclusion.

### 9.1. Adverse Events

An AE is any untoward medical occurrence in a patient or clinical investigation subject administered a pharmaceutical product and which does not necessarily have a causal relationship with this treatment. An AE can therefore be any unfavorable or unintended sign (including an abnormal laboratory finding, for example), symptom or disease temporally associated with the use of a medicinal (investigational) product whether or not considered related to the medicinal (investigational) product. [(GCP) E6(R2)] In clinical studies, an AE can include an undesirable medical condition occurring at any time, including baseline or washout periods, even if no study treatment has been administered. Additional criteria defining an AE are described below.

The following are considered to be adverse events:
Aggravation of a pre-existing disease or permanent disorder (any clinically significant worsening in the nature, severity, frequency, or duration of a pre-existing condition)
Events resulting from protocol-mandated procedures (e.g. complications from invasive procedures)

The following are not considered to be adverse events:
Medical or surgical procedures including elective or pre-planned such as surgery, endoscopy, tooth extraction, transfusion.
Note: an untoward medical event occurring during the prescheduled elective procedure or routinely scheduled treatment should be recorded as an AE or SAE
Pre-existing diseases or conditions that do not worsen during or after administration of the investigational medicinal product
Hospitalization planned for study treatment infusion or observation
The malignancy under study or signs and symptoms associated with the disease as well as progress or relapse of the underlying malignancy

Only abnormal laboratory results considered to be clinically significant should be reported as AEs (e.g. an abnormal laboratory finding associated with clinical symptoms, of prolonged duration, or that requires additional monitoring and/or medical intervention). Whenever possible, these should be reported as a clinical diagnosis rather than the abnormal parameter itself (i.e. neutropenia versus neutrophil count decreased). Abnormal laboratory results without clinical significant should not be recorded as AEs.

Adverse events can occur before, during, or after treatment, and can be either treatment emergent (i.e., occurring post-CTX110 infusion) or non-treatment emergent. A non-treatment-emergent AE is any new sign or symptom, disease, or other untoward medical event that occurs after written informed consent has been obtained before the subject has received CTX110.

### 9.2. Serious Adverse Event

An AE of any untoward medical consequence must be classified as an SAE if it meets any of the following criteria:
Results in death
Is life-threatening (i.e., an AE that places the subject at immediate risk of death)
Requires in-patient hospitalization or prolongs an existing hospitalization (hospitalizations for scheduled medical or surgical procedures or to conduct scheduled treatments do not meet these criteria)
Results in persistent or significant disability or incapacity
Results in a congenital anomaly or birth defect in the newborn
Other important/significant medical events. Important medical events that may not result in death, be life-threatening, or require hospitalization may be considered serious when, based upon appropriate medical judgment, they may jeopardize the patient or subject and may require medical or surgical intervention to prevent one of the outcomes listed in this definition.

Hospitalization for study treatment infusions, or planned hospitalizations following CTX110 infusion, are not considered SAEs. Furthermore, hospitalizations for observation or prolongation of hospitalization for observation alone should not be reported as an SAE unless they are associated with a medically significant event that meets other SAE criteria.

### 9.3. Adverse Events of Special Interest

Unless specified, all AESI should be reported if occurring after CTX110 infusion and prior to initiation new anticancer therapy. AESIs after CTX110 infusion must be reported, and include:
CTX110 infusion reactions up to Day 28
Opportunistic/invasive infections that occur within the first 28 days and/or beyond if ANC <1000 cells/uL and/or IgG <400 mg/dL with or without IVIG supplementation
Tumor lysis syndrome
Cytokine release syndrome, including cases with overlapping manifestations of HLH
Immune effector cell-associated neurotoxicity syndrome
B cell aplasia persistent 3 months after CTX110 infusion (B cell aplasia in subjects who have received Rituximab will only be reported if persistent at least 6 months after last dose of Rituximab and at least 3 months after last dose of CTX110 infusion
Hypogammaglobulinemia persistent 3 months after CTX110 infusion
Graft versus host disease
Secondary malignancy at any time after CTX110 infusion
Uncontrolled T cell proliferation at any time after CTX110 infusion

Any new hematological or autoimmune disorder Additional information on the required AESI reporting collection period is detailed in Table 25 below.

### 9.4. Adverse Event Severity

AEs are to be graded according to CTCAE version 5.0, with the exception of CRS, neurotoxicity, and GvHD, which will be graded according to the criteria disclosed herein.

When a CTCAE grade or protocol-specified criteria are not available, the toxicity grading in Table 26 can be used.

TABLE 26
Adverse Event Severity

| | |
|---|---|
| Grade 1 | Mild; asymptomatic or mild symptoms; clinical or diagnostic |
| | observations only; intervention not indicated. |
| Grade 2 | Moderate; minimal, local, or noninvasive intervention |
| | indicated; limiting age-appropriate instrumental ADL.¹ |
| Grade 3 | Severe or medically significant but not immediately life- |
| | threatening; hospitalization or prolongation of hospitalization |
| | indicated; disabling; limiting self-care ADL.² |
| Grade 4 | Life-threatening consequences; urgent intervention indicated. |
| Grade 5 | Death related to AE. |

ADL: Activities of Daily Living; AE: adverse event.
¹Instrumental ADL refer to preparing meals, shopping for groceries or clothes, using the telephone, managing money, etc.
²Self-care ADL refer to bathing, dressing and undressing, feeding self, using the toilet, taking medications, and not bedridden.

### 9.5 Adverse Event Causality

The relationship between each AE and CTX110, LD chemotherapy, daratumumab administration, and any protocol-mandated study procedure (all assessed individually) is to be assessed. The assessment of relationship will be made based on the following definitions:
Related: There is a clear causal relationship between the study treatment or procedure and the AE.
Possibly related: There is some evidence to suggest a causal relationship between the study treatment or procedure and the AE, but alternative potential causes also exist.
Not related: There is no evidence to suggest a causal relationship between the study treatment or procedure and the AE.

The temporal association between the timing of the event and administration of the treatment or procedure, a plausible biological mechanism, and other potential causes of the event (e.g., concomitant therapy, underlying disease) is to be considered when making their assessment of causality.

If an SAE is assessed to be not related to any study intervention, an alternative etiology must be provided in the CRF. If the relationship between the SAE and the investigational product is determined to be “possible”, a rationale for the assessment must be provided.

### 9.6 Outcome

The outcome of an AE or SAE classified and reported as follows:
Fatal
Not recovered/not resolved
Recovered/resolved
Recovered/resolved with sequelae
Recovering/resolving
Unknown

TABLE 27
Adverse Event Collection by Study Time Period

| Time Period | AE Reporting Requirements |
|---|---|
| Informed consent to 3 months after | All AEs |
| each CTX110 infusion | |
| 3 months after last CTX110 infusion | SAEs |
| through Month 24 visit | AESIs |
| Month 24 to Month 60 visit or after | CTX110-related SAEs |
| a subject receives a new anticancer | CTX110-related AESIs |
| therapy after Month 3 visit | New malignancies |

AE: adverse event; AESI: adverse event of special interest; SAE: serious adverse event.

If a subject receives a new anticancer therapy within 3 months of the last CTX110 infusion, all SAEs and AESIs should be reported until Month 3. If a subject starts a new anticancer therapy after the Month 3 study visit, only CTX110-related SAEs, CTX110-related AESIs, and new malignancies are to be reported. If a subject does not receive CTX110 therapy after enrollment, the AE reporting period ends 30 days after last study-related procedure (e.g., biopsy, imaging, LD chemotherapy).

### 9.7 Disease Progression

Disease progression and signs and symptoms of disease progression should not be reported as an AE with the following exception:

Atypical or accelerated progression of malignancy under study that in its nature, presentation, or severity differ from the normal course of the disease, with symptoms meeting serious criteria. In this case worsening of underlying condition should be reported as the SAE.

Disease progression with outcome of death within 30 days of study dose regardless of relationship to CTX110 should be recorded as an SAE and reported.

### 9.8. Termination

The treatment may be delayed, suspended, or terminated if one or more of the following events occur:
Life-threatening (grade 4) toxicity attributable to CTX110 that is unmanageable, unexpected, and unrelated to LD chemotherapy
Death related to CTX110 within 30 days of infusion
Grade >2 GvHD in subjects who receive >7×10⁴ TCR⁺ cells/kg prior to the initiation of any new anti cancer therapy including HSCT
After at least 12 subjects are enrolled in cohort expansion and at least 1 of the following occurs:
   >35% grade 3 or 4 neurotoxicity not resolving within 2 weeks to grade ≤2
   >20% grade ≥2 GvHD that is steroid refractory.
   >30% grade 4 CRS
New malignancy (distinct from recurrence/progression of previously treated malignancy)
Lack of efficacy, defined as 2 or fewer objective responses (per central review) after 15 subjects in cohort expansion have 3 months of post-CTX110 assessment.
Any medical condition that would put the subject at risk during continuing study-related treatments or follow up
If a subject is found not to have met eligibility criteria or has a major protocol deviation before the start of LD chemotherapy (Cohorts D, E, and G), or before the start of daratumumab infusion (Cohort C)
If a subject has unresolved infusion reaction due to daratumumab treatment (Cohorts C and G only)

### 10. Statistical Methods

### 10.1 Study Objectives and Hypotheses

The primary objective of Part A is to assess the safety of escalating doses of CTX110 in subjects with relapsed or refractory B cell malignancies to determine the recommended Part B dose.

The primary objective of Part B is to assess the efficacy of CTX110 in subjects with relapsed or refractory B cell malignancies, as measured by objective response rate.

### 10.2. Study Endpoints

### 10.2.1 Primary Endpoints

Dose escalation for all cohorts: The incidence of adverse events, defined as dose-limiting toxicities for each of the cohorts (C, D, and E)

Cohort expansion for subjects with NHL: The objective response rate (CR+PR) per Lugano Response Criteria for Malignant Lymphoma (Cheson et al., 2014), as determined by independent central review

### 10.2.2. Dose Escalation and Cohort Expansion Secondary Efficacy Endpoints

Duration of response/remission will be reported only for subjects who have had objective response events. This is to be assessed using the time between the first objective response and the first disease progression or death due to any cause after the first objective response. Subjects who have not progressed since the first objective response and are still on study at the data cutoff date will be censored at their last assessment date.

Duration of clinical benefit (DOCB) is calculated as the time between the first objective response and the last disease progression or death. Subjects who have not progressed and are still on study at the data cutoff date will be censored at their last assessment date.

Treatment failure free survival (TFFS) is calculated as the time between the first CTX110 infusion and the last disease progression or death due to any cause. Subjects who have not progressed and are still on study at the data cutoff date are censored at their last assessment date.

Overall survival is calculated as the time between date of first dose of CTX110 and death due to any cause. Subjects who are alive at the data cutoff date are censored at their last date known to be alive.

For B cell ALL (Cohort D), ORR (complete remission+CRi) will be assessed.

### 10.2.3. Secondary Safety Endpoints

The frequency and severity of AEs and clinically significant laboratory abnormalities will be summarized and reported according to CTCAE version 5.0, except for CRS (Lee and ASTCT criteria), neurotoxicity (ICANS and CTCAE v5.0), and GvHD (Mount Sinai Acute GVHD International Consortium [MAGIC] criteria).

### 10.2.4. Pharmacokinetics

Pharmacokinetic data will include levels of CTX110 in blood over time as assessed by a PCR assay that measures copies of CAR construct. Analysis of CTX110 in blood may also occur using flow cytometry that detects CAR protein on the cellular surface. Such analysis may be used to confirm the presence of CTX110 in blood and to further characterize other cellular immunophenotypes.

### 10.2.5. Secondary Patient-Reported Outcome Endpoint

Change over time in PROs associated with CTX110 will be evaluated and analyzed as disclosed herein for the PRO surveys administered to subjects in various cohorts.

### 10.2.6. Dose Escalation and Cohort Expansion Exploratory Endpoints

Levels of CTX110 in tissues (trafficking of CTX110 in bone marrow, CSF, and/or tumor tissue may be evaluated in any samples collected per protocol-specific sampling)
Levels of cytokines in blood and other tissues
Incidence of anti-CTX110 antibodies
Levels of B cells and immunoglobulins over time
Impact of anti-cytokine therapy on CTX110 proliferation, CRS, and response
Incidence of autologous or allogeneic HSCT following CTX110 therapy
Incidence and type of subsequent anticancer therapy
Time to complete response/remission, defined as the time between the date of first CTX110 infusion until first documented complete response
First subsequent therapy-free survival, defined as the time between date of first CTX110 infusion and date of first subsequent therapy or death due to any cause
Percentage of subjects with CR achieving MRD remission (Cohort D)
Other genomic, proteomic, metabolic, or pharmacodynamic endpoints

### 10.3 Analysis Sets

The following analysis sets will be evaluated and used for presentation of the data.

### Part A (Dose Escalation)

DLT evaluable set (DES): All subjects who receive CTX110 and complete the DLT evaluation period or discontinue early after experiencing a DLT. For Cohorts A, B, C, D, and F, the DLT evaluation period will begin with first CTX110 infusion and last for 28 days. For Cohort E, the DLT evaluation period will last for 28 days after the second infusion, for a total of approximately 7 weeks (21 days from initial infusion+28 days from second infusion). The DES is used for determination of the recommended Part B dose.

### Part A+Part B (Dose Escalation+Cohort Expansion)

Enrolled set: All subjects enrolled in the study. The enrolled set will be classified according to the assigned dose level of CTX110.
Treated set: All subjects who received any study treatment in the study. The subjects in the treated set will be classified according to the received study treatment.
Modified intent to treat set (mITT): All subjects who received CTX110 infusion. The subjects in mITT will be classified according to the assigned dose level of CTX110. The mITT will be the primary analysis set for clinical activity assessment.
Safety analysis set (SAS): All subjects who received CTX110 infusion. The subjects in SAS will be classified according to the received dose level of CTX110. The SAS will be the primary analysis set for the characterization of CTX110 safety profile.

### 10.4. Interim Analyses

### 10.4.1. Efficacy Interim Analysis

One interim analysis for early efficacy and futility will be performed by independent statistician and reviewed by the DSMB. The interim analysis will occur when 38 (50%) of the planned 77 subjects in the enriched subset of the expanded cohort for NHL have been enrolled in Part B and have 3 months of evaluable tumor response data or have discontinued earlier.

An alpha-spending function according to Lan-Demets (Lan and Demets, 1983) will be used to construct O'Brien-Fleming type of efficacy boundary at the interim analysis. Based on this choice of alpha-spending function, if the interim analysis is performed with 38 subjects (50% of 77), the lower bound of the 2-sided 99.26% exact confidence interval for ORR will need to be greater than 26% to declare statistical significance. As a result, an ORR of at least 19/38=50% will be needed to claim early efficacy at the interim analysis. The demonstration of early efficacy can be used to support regulatory interactions and/or publications. At the primary analysis when 76 subjects in the enriched subset are treated and followed for at least 3 months, 2-sided 95.14% exact confidence interval will be used correspondingly, requiring an ORR of at least 29/76=38% to claim success.

For futility, enrollment of NHL subjects will stop if up to 10 subjects achieve an objective response among these 38 subjects at interim analysis. Based on a non-informative prior on the probability of success, if no more than 10 of the 39 subjects achieve objective response at the interim analysis, the Bayesian predictive probability of having at least 29 responders out of 76 subjects at the final analysis is less than 5%. If there are at least 11 responders (based on central imaging review) at the time the 39th subject has enrolled, screening and enrollment will not be halted, as the minimum criteria for continuing enrollment will have been met. If the true response rate to CTX110 is not different from standard of care, the likelihood of stopping for futility at this analysis is 60.1%.

### 10.5. Planned Method of Analyses

The primary analysis of efficacy occurs after all subjects in the FAS in Part B of the study have had the opportunity to be assessed for response 3 months after CTX110 infusion. A final analysis will occur when all subjects complete or withdraw from the study. Tabulations are produced for appropriate disposition, demographic, baseline, efficacy and safety parameters. By-subject listings will be provided for all data, unless otherwise specified.

### 10.5.1. Efficacy Analysis

The primary endpoint of ORR for all analyses (interim and primary) will be based on independent central review of disease assessments in the FAS. Hierarchical testing will be performed with the null hypothesis tested in the enriched subset of the expanded cohort first, followed by testing in the whole expanded cohort if the null hypothesis is rejected at the first step.

Sensitivity analyses of ORR can be performed. For NHL, Lugano response criteria (Cheson et al, 2014) are to be used and ORR refers to the rate of CR+PR (Tables 8 and 9). For B cell ALL, ORR refers to the rate of complete remission+Cri. See Table 28 below.

Objective response rate is summarized as a proportion with exact 95% confidence intervals. For time-to-event variables such as DOR, DOCB, TFFS, and overall survival, medians with 95% confidence intervals will be calculated using Kaplan-Meier methods.

TABLE 28
B Cell ALL Disease Response

| | | If extra medullary disease is |
|---|---|---|
| Disease Response | Definition | present at baseline |
| Complete remission | ≤5% blasts in the bone marrow; and | Disappearance of measurable |
| (CR) | no evidence of disease; and | and non-measurable nodal |
| | full recovery of peripheral blood | lesions: |
| | counts (platelets ≥100,000/μL and | |
| | ANC ≥1,000/μL) | |
| Complete remission | ≤5% blasts in the bone marrow; and | Nodal masses >1.5 cm in |
| with incomplete blood | no evidence of disease; and | GTD at baseline must have |
| count recovery (CRi) | incomplete recovery of peripheral | regressed to ≤1.5 cm in GTD |
| | blood counts (platelets <100,000/μL | Nodes that were 1.1 to 1.5 |
| | or ANC <1000/μL) | cm in their long axis and more |
| Complete remission | ≤5% blasts in the bone marrow; and | than 1.0 cm in their short axis |
| with partial | No evidence of disease and | before treatment must have |
| hematologic recovery | Partial recovery of peripheral blood | decreased to 1.0 cm in their |
| (CRh) | counts: Platelets ≥50,000/μl, and | short axis after treatment |
| | ANC ≥500/μl | AND |
| | | If testes, spleen, and/or liver |
| | | involvement, they must be |
| | | normal size by imaging or |
| | | physical examination |
| | | AND |
| | | No new lesions |
| Partial Response | Meets Criteria in blood and bone | ≥50% decrease in SPD of up |
| | marrow for CR, CRi or CRh | to 6 of the largest dominant |
| | | masses. Dominant masses |
| | | should be clearly measurable |
| | | in at least 2 perpendicular |
| | | dimensions, and should be |
| | | from different regions of the |
| | | body if possible |
| | | AND |
| | | No increase in size of liver or |
| | | spleen by imaging or physical |
| | | exam |
| | | AND |
| | | If multiple splenic and hepatic |
| | | nodules are present, they must |
| | | regress by ≥50% in SPD. |
| | | There must be a >50% |
| | | decrease in GTD for a single |
| | | nodule |
| | | AND |
| | | No new lesions |
| Progressive disease | ≥25% increase in absolute number of | ≥50% increase from nadir in |
| (PD) | circulating or bone marrow blasts, or | the sum of the products of at |
| Relapsed disease | development of extramedullary | least two lymph nodes, or if a |
| | disease (including CNS disease) | single node is involved at least |
| | Reappearance of blasts in blood or | a 50% increase in the product |
| | bone marrow (>5%) after a CR; or | of the diameters of this one |
| | New development of CNS-3 status or | node; OR |
| | clinical signs of CNS leukemia such | At least a 50% increase in the |
| | as facial nerve palsy, brain/eye | longest diameter of any single |
| | involvement, or hypothalamic | previously identified node |
| | syndrome without another explanation | more than 1 cm in its short |
| | will be considered a CNS relapse. | axis; OR |
| | | ≥50% increase in size of |
| | | splenic, hepatic or any other |
| | | non-nodal lesion OR |
| | | Development of new lesions |

| | |
|---|---|
| Non-response | Not fitting the definition of CR, CRi, CRh or PD and not having relapsed disease |
| Unknown | Assessment is not done, incomplete, or indeterminate |

ALL: acute lymphoblastic leukemia; ANC: absolute neutrophil count; CNS: central nervous system; CNS-3: TotalNucleated Cell Count ≥5 × 10⁶/L with blasts on cytocentrifuge CR: complete remission; CRi: complete remissionwith incomplete blood count recovery; GTD: Greatest Traverse Diameter.
Adapted from National Comprehensive Cancer Network guidelines for treatment of acute lymphoblastic leukemia (NCCN Guidelines v.2.2019, 2019)

### 10.5.2. Safety Analysis

All subjects who receive CTX110, LD chemotherapy, and/or daratumumab are included in the safety analysis set. Clinical AEs will be graded according to CTCAE version 5, except for CR5, which will be graded according to Lee criteria and (Lee et al., 2019), neurotoxicity, which will be graded according to ICANS (Lee et al., 2019) and CTCAE, and GvHD, which will be graded according to MAGIC criteria (Harris et al., 2016). The AEs, SAEs, and AESIs will be summarized and reported according to the intervals in Table 27 where AE collection by study time period is described.

Treatment-emergent adverse events are defined as AEs that start or worsen on or after the initial CTX110 infusion. Vital signs are summarized using descriptive statistics. Frequencies of subjects experiencing at least 1 AE will be reported by body system and preferred term according to MedDRA terminology. Detailed information collected for each AE will include description of the event, duration, whether the AE was serious, intensity, relationship to study drug, action taken, clinical outcome, and whether or not it was a DLT. Emphasis in the analysis is placed on AEs classified as dose-limiting.

### 10.5.3. Pharmacokinetic and Pharmacodynamic Analyses

Incidence of anti-CTX110 antibodies, levels of CTX110 CAR⁺ T cells in blood, and levels of cytokines in serum will be summarized.

### 10.5.4. Biomarker Analyses

Investigation of additional biomarkers may include assessment of blood cells, tumor cells, and other subject-derived tissue. These assessments may evaluate DNA, RNA, proteins, and other biologic molecules derived from those tissues. Such evaluations will inform understanding of factors related to subject's response to CTX110 and the mechanism of action of the investigational product.

### Results

To date, 8 subjects have been enrolled in the Cohort C of this study. Five subjects have received CTX110 at DL3, and 3 subjects have received CTX110 at DL4. In this cohort, CTX110 infusion is preceded by one dose of daratumumab (16 mg/kg, i.v.) and 3 days of LD chemotherapy (30 mg/m²/day fludarabine and 500 mg/m²/day cyclophosphamide). CTX110 infusion occurs 2 to 10 days after completion of LD chemotherapy. One subject achieved CR at Day 28 and has maintained CR for 3+ months (response assessment ongoing). None of the subjects treated in Cohort C has experienced a DLT.

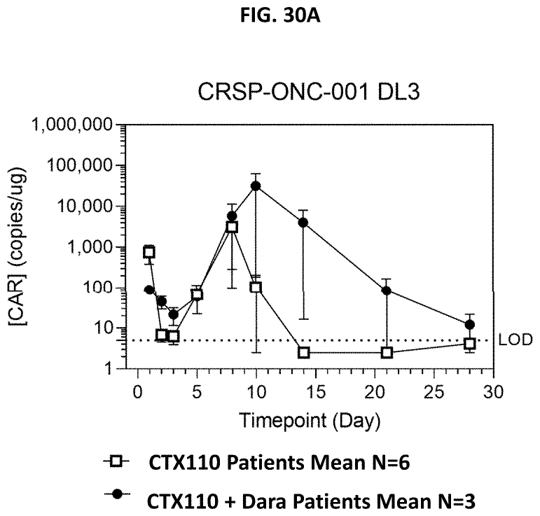

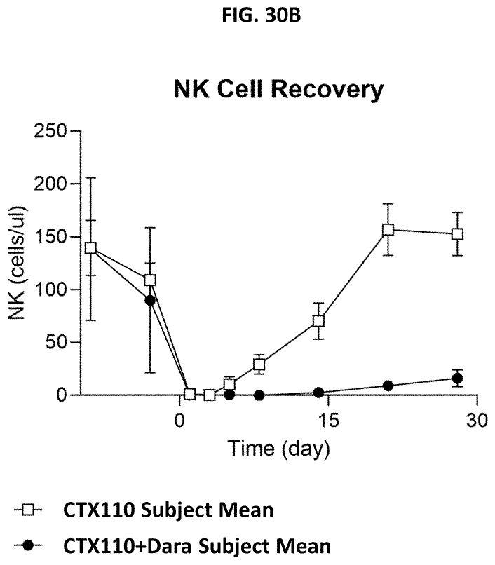

Subjects treated with daratumumab and LD chemotherapy prior to CTX110 administration (Cohort C) showed a significant increase in CAR⁺ T cell expansion and persistence compared to subjects treated with standard LD chemotherapy alone. Maximal expansion occurred at Day 10 and CTX110 was still detectable at Month 2 in at least 1 subject. (FIG. 30A). In addition, subjects treated with daratumumab prior to CTX110 administration showed a dramatically decreased rate of NK cell recovery (FIG. 30B). NK cells were depleted in patients receiving CTX110 and the combination of CTX110 and daratumumab at the time of CTX110 administration (Day 1). However, NK cells levels returned to pretreatment levels by Day 21 in subjects treated without daratumumab (FIG. 30B). By contrast, NK cells remained significantly decreased in subjects treated with daratumumab.

In one subject, genetic sequencing of CTX110 cells recovered from a blood sample 8 days post-infusion showed a preferential expansion of cells with indels in the β2M gene. Such cells would be expected to be MHC Class-I deficient, which could render them vulnerable to clearance by NK cells. Together, these findings support that the inhibition of NK cells with daratumumab lead to the prolonged survival of MHC Class-I deficient CTX110.

SEQUENCE TABLE

| SEQ ID | | |
|---|---|---|
| NO | Description | Sequence |
| 1 | TRAC gene-edit | AAGAGCAACAAATCTGACT |
| 2 | TRAC gene-edit | AAGAGCAACAGTGCTGTGCCTGGAGCAACAAATCTGACT |
| | | AAGAGCAACAAATCTGACT |
| 3 | TRAC gene-edit | AAGAGCAACAGTGCTGGAGCAACAAATCTGACT |
| | | AAGAGCAACAAATCTGACT |
| 4 | TRAC gene-edit | AAGAGCAACAGTGCCTGGAGCAACAAATCTGACT |
| | | AAGAGCAACAAATCTGACT |
| 5 | TRAC gene-edit | AAGAGCAACAGTGCTGACTAAGAGCAACAAATCTGACT |
| 6 | TRAC gene-edit | AAGAGCAACAGTGCTGTGGGCCTGGAGCAACAAATCTGA |
| | | CT AAGAGCAACAAATCTGACT |
| 7 | TRAC gene-edit | AAGAGCAACAGTGCTGGCCTGGAGCAACAAATCTGACT |
| | | AAGAGCAACAAATCTGACT |
| 8 | TRAC gene-edit | AAGAGCAACAGTGCTGTGTGCCTGGAGCAACAAATCTGA |
| | | CT AAGAGCAACAAATCTGACT |
| 9 | B2M gene-edit | CGTGGCCTTAGCTGTGCTCGCGCTACTCTCTCTTTCTGCCT |
| | | GGAGGCTATCCAGCGTGAGTCTCTCCTACCCTCCCGCT |
| 10 | B2M gene-edit | CGTGGCCTTAGCTGTGCTCGCGCTACTCTCTCTTTCGCCTG |
| | | GAGGCTATCCAGCGTGAGTCTCTCCTACCCTCCCGCT |
| 11 | B2M gene-edit | CGTGGCCTTAGCTGTGCTCGCGCTACTCTCTCTTTCTGGA |
| | | GGCTATCCAGCGTGAGTCTCTCCTACCCTCCCGCT |
| 12 | B2M gene-edit | CGTGGCCTTAGCTGTGCTCGCGCTACTCTCTCTTTCTGGAT |
| | | AGCCTGGAGGCTATCCAGCGTGAGTCTCTCCTACCCTCCC |
| | | GCT |
| 13 | B2M gene-edit | CGTGGCCTTAGCTGTGCTCGCGCTATCCAGCGTGAGTCTC |
| | | TCCTACCCTCCCGCT |
| 14 | B2M gene-edit | CGTGGCCTTAGCTGTGCTCGCGCTACTCTCTCTTTCTGTGG |
| | | CCTGGAGGCTATCCAGCGTGAGTCTCTCCTACCCTCCCGC |
| | | T |
| 15 | sgRNA | nnnnnnnnnnnnnnnnnnnnguuuuagagcuagaaauagcaaguuaaaauaaggcu |
| | | aguccguuaucaacuugaaaaaguggcaccgagucggugcuuuu |
| 16 | sgRNA | nnnnnnnnnnnnnnnnnnnnguuuuagagcuagaaauagcaaguuaaaauaaggcu |
| | | aguccguuaucaacuugaaaaaguggcaccgagucggugc |
| 17 | sgRNA | n₍₁₇₋₃₀₎guuuuagagcuagaaauagcaaguuaaaauaaggcuaguccguuauc |
| | | aacuugaaaaaguggcaccgagucggugcu₍₁₋₈₎ |
| 18 | TRAC sgRNA (TA-1) | AGAGCAACAGUGCUGUGGCCguuuuagagcuagaaauagcaaguuaa |
| | unmodified | aauaaggcuaguccguuaucaacuugaaaaaguggcaccgagucggugcUUUU |
| 19 | TRAC sgRNA spacer | AGAGCAACAGUGCUGUGGCC |
| | unmodified | |
| 20 | B2M sgRNA | GCUACUCUCUCUUUCUGGCCguuuuagagcuagaaauagcaaguuaaa |
| | unmodified | auaaggcuaguccguuaucaacuugaaaaaguggcaccgagucggugcUUUU |
| 21 | B2M sgRNA spacer | GCUACUCUCUCUUUCUGGCC |
| | unmodified | |
| 22 | TRAC sgRNA (TA-1) | A*G*A*GCAACAGUGCUGUGGCCguuuuagagcuagaaauagcaagu |
| | modified | uaaaauaaggcuaguccguuaucaacuugaaaaaguggcaccgagucggugcU*U* |
| | *: 2′-O-methyl | U*U |
| | phosphorothioate | |
| | residue | |
| 23 | TRAC sgRNA spacer | A*G*A*GCAACAGUGCUGUGGCC |
| | modified | |
| | *: 2′-O-methyl | |
| | phosphorothioate | |
| | residue | |
| 24 | B2M sgRNA | G*C*U*ACUCUCUCUUUCUGGCCguuuuagagcuagaaauagcaagu |
| | modified | uaaaauaaggcuaguccguuaucaacuugaaaaaguggcaccgagucggugcU*U* |
| | *: 2′-O-methyl | U*U |
| | phosphorothioate | |
| | residue | |
| 25 | B2M sgRNA spacer | G*C*U*ACUCUCUCUUUCUGGCC |
| | modified | |
| | *: 2′-O-methyl | |
| | phosphorothioate | |
| | residue | |
| 26 | TRAC target | AGAGCAACAGTGCTGTGGCC |
| | sequence | |
| 27 | B2M target sequence | GCTACTCTCTCTTTCTGGCC |
| 28 | TRAC target | AGAGCAACAGTGCTGTGGCC(TGG) |
| | sequence with (PAM) | |
| 29 | B2M target sequence | GCTACTCTCTCTTTCTGGCC(TGG) |
| | with (PAM) | |
| 30 | signal peptide | MLLLVTSLLLCELPHPAFLLIP |
| 31 | signal peptide | MALPVTALLLPLALLLHAARP |
| 32 | CD8a transmembrane | IYIWAPLAGTCGVLLLSLVITLY |
| | domain | |
| 33 | 4-1BB nucleotide | AAACGGGGCAGAAAGAAACTCCTGTATATATTCAAACAA |
| | sequence | CCATTTATGAGACCAGTACAAACTACTCAAGAGGAAGAT |
| | | GGCTGTAGCTGCCGATTTCCAGAAGAAGAAGAAGGAGGA |
| | | TGTGAACTG |
| 34 | 4-1BB amino acid | KRGRKKLLYIFKQPFMRPVQTTQEEDGCSCRFPEEEEGGCE |
| | sequence | L |
| 35 | CD28 nucleotide | TCAAAGCGGAGTAGGTTGTTGCATTCCGATTACATGAATA |
| | sequence | TGACTCCTCGCCGGCCTGGGCCGACAAGAAAACATTACC |
| | | AACCCTATGCCCCCCCACGAGACTTCGCTGCGTACAGGTC |
| | | C |
| 36 | CD28 amino acid | SKRSRLLHSDYMNMTPRRPGPTRKHYQPYAPPRDFAAYRS |
| | sequence | |
| 37 | CD3-zeta nucleotide | CGAGTGAAGTTTTCCCGAAGCGCAGACGCTCCGGCATAT |
| | sequence | CAGCAAGGACAGAATCAGCTGTATAACGAACTGAATTTG |
| | | GGACGCCGCGAGGAGTATGACGTGCTTGATAAACGCCGG |
| | | GGGAGAGACCCGGAAATGGGGGGTAAACCCCGAAGAAA |
| | | GAATCCCCAAGAAGGACTCTACAATGAACTCCAGAAGGA |
| | | TAAGATGGCGGAGGCCTACTCAGAAATAGGTATGAAGGG |
| | | CGAACGACGACGGGGAAAAGGTCACGATGGCCTCTACCA |
| | | AGGGTTGAGTACGGCAACCAAAGATACGTACGATGCACT |
| | | GCATATGCAGGCCCTGCCTCCCAGA |
| 38 | CD3-zeta amino acid | RVKFSRSADAPAYQQGQNQLYNELNLGRREEYDVLDKRRG |
| | sequence | RDPEMGGKPRRKNPQEGLYNELQKDKMAEAYSEIGMKGE |
| | | RRRGKGHDGLYQGLSTATKDTYDALHMQALPPR |
| 39 | FMC63-28Z | ATGCTTCTTTTGGTTACGTCTCTGTTGCTTTGCGAACTTCC |
| | (FMC63-CD8[tm]- | TCATCCAGCGTTCTTGCTGATCCCCGATATTCAGATGACT |
| | CD28[co-stimulatory | CAGACCACCAGTAGCTTGTCTGCCTCACTGGGAGACCGA |
| | domain]-CD3z) | GTAACAATCTCCTGCAGGGCAAGTCAAGACATTAGCAAA |
| | | TACCTCAATTGGTACCAGCAGAAGCCCGACGGAACGGTA |
| | | AAACTCCTCATCTATCATACGTCAAGGTTGCATTCCGGAG |
| | | TACCGTCACGATTTTCAGGTTCTGGGAGCGGAACTGACTA |
| | | TTCCTTGACTATTTCAAACCTCGAGCAGGAGGACATTGCG |
| | | ACATATTTTTGTCAACAAGGTAATACCCTCCCTTACACTT |
| | | TCGGAGGAGGAACCAAACTCGAAATTACCGGGTCCACCA |
| | | GTGGCTCTGGGAAGCCTGGCAGTGGAGAAGGTTCCACTA |
| | | AAGGCGAGGTGAAGCTCCAGGAGAGCGGCCCCGGTCTCG |
| | | TTGCCCCCAGTCAAAGCCTCTCTGTAACGTGCACAGTGAG |
| | | TGGTGTATCATTGCCTGATTATGGCGTCTCCTGGATAAGG |
| | | CAGCCCCCGCGAAAGGGTCTTGAATGGCTTGGGGTAATA |
| | | TGGGGCTCAGAGACAACGTATTATAACTCCGCTCTCAAA |
| | | AGTCGCTTGACGATAATAAAAGATAACTCCAAGAGTCAA |
| | | GTTTTCCTTAAAATGAACAGTTTGCAGACTGACGATACCG |
| | | CTATATATTATTGTGCTAAACATTATTACTACGGCGGTAG |
| | | TTACGCGATGGATTATTGGGGGCAGGGGACTTCTGTCAC |
| | | AGTCAGTAGTGCTGCTGCCTTTGTCCCGGTATTTCTCCCA |
| | | GCCAAACCGACCACGACTCCCGCCCCGCGCCCTCCGACA |
| | | CCCGCTCCCACCATCGCCTCTCAACCTCTTAGTCTTCGCC |
| | | CCGAGGCATGCCGACCCGCCGCCGGGGGTGCTGTTCATA |
| | | CGAGGGGCTTGGACTTCGCTTGTGATATTTACATTTGGGC |
| | | TCCGTTGGCGGGTACGTGCGGCGTCCTTTTGTTGTCACTC |
| | | GTTATTACTTTGTATTGTAATCACAGGAATCGCTCAAAGC |
| | | GGAGTAGGTTGTTGCATTCCGATTACATGAATATGACTCC |
| | | TCGCCGGCCTGGGCCGACAAGAAAACATTACCAACCCTA |
| | | TGCCCCCCCACGAGACTTCGCTGCGTACAGGTCCCGAGT |
| | | GAAGTTTTCCCGAAGCGCAGACGCTCCGGCATATCAGCA |
| | | AGGACAGAATCAGCTGTATAACGAACTGAATTTGGGACG |
| | | CCGCGAGGAGTATGACGTGCTTGATAAACGCCGGGGGAG |
| | | AGACCCGGAAATGGGGGGTAAACCCCGAAGAAAGAATC |
| | | CCCAAGAAGGACTCTACAATGAACTCCAGAAGGATAAGA |
| | | TGGCGGAGGCCTACTCAGAAATAGGTATGAAGGGCGAAC |
| | | GACGACGGGGAAAAGGTCACGATGGCCTCTACCAAGGGT |
| | | TGAGTACGGCAACCAAAGATACGTACGATGCACTGCATA |
| | | TGCAGGCCCTGCCTCCCAGA |
| 40 | FMC63-28Z | MLLLVTSLLLCELPHPAFLLIPDIQMTQTTSSLSASLGDRVTI |
| | (FMC63-CD8- | SCRASQDISKYLNWYQQKPDGTVKLLIYHTSRLHSGVPSRF |
| | CD28[co-stimulatory | SGSGSGTDYSLTISNLEQEDIATYFCQQGNTLPYTFGGGTKL |
| | domain]-CD3z) | EITGSTSGSGKPGSGEGSTKGEVKLQESGPGLVAPSQSLSVT |
| | Amino Acid | CTVSGVSLPDYGVSWIRQPPRKGLEWLGVIWGSETTYYNSA |
| | | LKSRLTIIKDNSKSQVFLKMNSLQTDDTAIYYCAKHYYYGG |
| | | SYAMDYWGQGTSVTVSSAAAFVPVFLPAKPTTTPAPRPPTP |
| | | APTIASQPLSLRPEACRPAAGGAVHTRGLDFACDIYIWAPLA |
| | | GTCGVLLLSLVITLYCNHRNRSKRSRLLHSDYMNMTPRRPG |
| | | PTRKHYQPYAPPRDFAAYRSRVKFSRSADAPAYQQGQNQL |
| | | YNELNLGRREEYDVLDKRRGRDPEMGGKPRRKNPQEGLYN |
| | | ELQKDKMAEAYSEIGMKGERRRGKGHDGLYQGLSTATKDT |
| | | YDALHMQALPPR |
| 41 | TRAC-LHA (800 bp) | GAGATGTAAGGAGCTGCTGTGACTTGCTCAAGGCCTTAT |
| | | ATCGAGTAAACGGTAGTGCTGGGGCTTAGACGCAGGTGT |
| | | TCTGATTTATAGTTCAAAACCTCTATCAATGAGAGAGCAA |
| | | TCTCCTGGTAATGTGATAGATTTCCCAACTTAATGCCAAC |
| | | ATACCATAAACCTCCCATTCTGCTAATGCCCAGCCTAAGT |
| | | TGGGGAGACCACTCCAGATTCCAAGATGTACAGTTTGCTT |
| | | TGCTGGGCCTTTTTCCCATGCCTGCCTTTACTCTGCCAGA |
| | | GTTATATTGCTGGGGTTTTGAAGAAGATCCTATTAAATAA |
| | | AAGAATAAGCAGTATTATTAAGTAGCCCTGCATTTCAGGT |
| | | TTCCTTGAGTGGCAGGCCAGGCCTGGCCGTGAACGTTCAC |
| | | TGAAATCATGGCCTCTTGGCCAAGATTGATAGCTTGTGCC |
| | | TGTCCCTGAGTCCCAGTCCATCACGAGCAGCTGGTTTCTA |
| | | AGATGCTATTTCCCGTATAAAGCATGAGACCGTGACTTGC |
| | | CAGCCCCACAGAGCCCCGCCCTTGTCCATCACTGGCATCT |
| | | GGACTCCAGCCTGGGTTGGGGCAAAGAGGGAAATGAGAT |
| | | CATGTCCTAACCCTGATCCTCTTGTCCCACAGATATCCAG |
| | | AACCCTGACCCTGCCGTGTACCAGCTGAGAGACTCTAAA |
| | | TCCAGTGACAAGTCTGTCTGCCTATTCACCGATTTTGATT |
| | | CTCAAACAAATGTGTCACAAAGTAAGGATTCTGATGTGT |
| | | ATATCACAGACAAAACTGTGCTAGACATGAGGTCTATGG |
| | | ACTTCA |
| 42 | TRAC-RHA (800 bp) | TGGAGCAACAAATCTGACTTTGCATGTGCAAACGCCTTCA |
| | | ACAACAGCATTATTCCAGAAGACACCTTCTTCCCCAGCCC |
| | | AGGTAAGGGCAGCTTTGGTGCCTTCGCAGGCTGTTTCCTT |
| | | GCTTCAGGAATGGCCAGGTTCTGCCCAGAGCTCTGGTCA |
| | | ATGATGTCTAAAACTCCTCTGATTGGTGGTCTCGGCCTTA |
| | | TCCATTGCCACCAAAACCCTCTTTTTACTAAGAAACAGTG |
| | | AGCCTTGTTCTGGCAGTCCAGAGAATGACACGGGAAAAA |
| | | AGCAGATGAAGAGAAGGTGGCAGGAGAGGGCACGTGGC |
| | | CCAGCCTCAGTCTCTCCAACTGAGTTCCTGCCTGCCTGCC |
| | | TTTGCTCAGACTGTTTGCCCCTTACTGCTCTTCTAGGCCTC |
| | | ATTCTAAGCCCCTTCTCCAAGTTGCCTCTCCTTATTTCTCC |
| | | CTGTCTGCCAAAAAATCTTTCCCAGCTCACTAAGTCAGTC |
| | | TCACGCAGTCACTCATTAACCCACCAATCACTGATTGTGC |
| | | CGGCACATGAATGCACCAGGTGTTGAAGTGGAGGAATTA |
| | | AAAAGTCAGATGAGGGGTGTGCCCAGAGGAAGCACCATT |
| | | CTAGTTGGGGGAGCCCATCTGTCAGCTGGGAAAAGTCCA |
| | | AATAACTTCAGATTGGAATGTGTTTTAACTCAGGGTTGAG |
| | | AAAACAGCTACCTTCAGGACAAAAGTCAGGGAAGGGCTC |
| | | TCTGAAGAAATGCTACTTGAAGATACCAGCCCTACCAAG |
| | | GGCAGGGAGAGGACCCTATAGAGGCCTGGGACAGGAGC |
| | | TCAATGAGAAAGG |
| 43 | EF1a | GGCTCCGGTGCCCGTCAGTGGGCAGAGCGCACATCGCCC |
| | | ACAGTCCCCGAGAAGTTGGGGGGAGGGGTCGGCAATTGA |
| | | ACCGGTGCCTAGAGAAGGTGGCGCGGGGTAAACTGGGAA |
| | | AGTGATGTCGTGTACTGGCTCCGCCTTTTTCCCGAGGGTG |
| | | GGGGAGAACCGTATATAAGTGCAGTAGTCGCCGTGAACG |
| | | TTCTTTTTCGCAACGGGTTTGCCGCCAGAACACAGGTAAG |
| | | TGCCGTGTGTGGTTCCCGCGGGCCTGGCCTCTTTACGGGT |
| | | TATGGCCCTTGCGTGCCTTGAATTACTTCCACTGGCTGCA |
| | | GTACGTGATTCTTGATCCCGAGCTTCGGGTTGGAAGTGGG |
| | | TGGGAGAGTTCGAGGCCTTGCGCTTAAGGAGCCCCTTCG |
| | | CCTCGTGCTTGAGTTGAGGCCTGGCCTGGGCGCTGGGGCC |
| | | GCCGCGTGCGAATCTGGTGGCACCTTCGCGCCTGTCTCGC |
| | | TGCTTTCGATAAGTCTCTAGCCATTTAAAATTTTTGATGA |
| | | CCTGCTGCGACGCTTTTTTTCTGGCAAGATAGTCTTGTAA |
| | | ATGCGGGCCAAGATCTGCACACTGGTATTTCGGTTTTTGG |
| | | GGCCGCGGGCGGCGACGGGGCCCGTGCGTCCCAGCGCAC |
| | | ATGTTCGGCGAGGCGGGGCCTGCGAGCGCGGCCACCGAG |
| | | AATCGGACGGGGGTAGTCTCAAGCTGGCCGGCCTGCTCT |
| | | GGTGCCTGGCCTCGCGCCGCCGTGTATCGCCCCGCCCTGG |
| | | GCGGCAAGGCTGGCCCGGTCGGCACCAGTTGCGTGAGCG |
| | | GAAAGATGGCCGCTTCCCGGCCCTGCTGCAGGGAGCTCA |
| | | AAATGGAGGACGCGGCGCTCGGGAGAGCGGGCGGGTGA |
| | | GTCACCCACACAAAGGAAAAGGGCCTTTCCGTCCTCAGC |
| | | CGTCGCTTCATGTGACTCCACGGAGTACCGGGCGCCGTCC |
| | | AGGCACCTCGATTAGTTCTCGAGCTTTTGGAGTACGTCGT |
| | | CTTTAGGTTGGGGGGAGGGGTTTTATGCGATGGAGTTTCC |
| | | CCACACTGAGTGGGTGGAGACTGAAGTTAGGCCAGCTTG |
| | | GCACTTGATGTAATTCTCCTTGGAATTTGCCCTTTTTGAGT |
| | | TTGGATCTTGGTTCATTCTCAAGCCTCAGACAGTGGTTCA |
| | | AAGTTTTTTTCTTCCATTTCAGGTGTCGTGA |
| 44 | GM-CSF signal | ATGCTTCTTTTGGTTACGTCTCTGTTGCTTTGCGAACTTCC |
| | peptide | TCATCCAGCGTTCTTGCTGATCCCC |
| 45 | GM-CSF signal | MLLLVTSLLLCELPHPAFLLIP |
| | peptide | |
| 46 | Anti-CD19 scFv | GATATTCAGATGACTCAGACCACCAGTAGCTTGTCTGCCT |
| | | CACTGGGAGACCGAGTAACAATCTCCTGCAGGGCAAGTC |
| | | AAGACATTAGCAAATACCTCAATTGGTACCAGCAGAAGC |
| | | CCGACGGAACGGTAAAACTCCTCATCTATCATACGTCAA |
| | | GGTTGCATTCCGGAGTACCGTCACGATTTTCAGGTTCTGG |
| | | GAGCGGAACTGACTATTCCTTGACTATTTCAAACCTCGAG |
| | | CAGGAGGACATTGCGACATATTTTTGTCAACAAGGTAAT |
| | | ACCCTCCCTTACACTTTCGGAGGAGGAACCAAACTCGAA |
| | | ATTACCGGGTCCACCAGTGGCTCTGGGAAGCCTGGCAGT |
| | | GGAGAAGGTTCCACTAAAGGCGAGGTGAAGCTCCAGGAG |
| | | AGCGGCCCCGGTCTCGTTGCCCCCAGTCAAAGCCTCTCTG |
| | | TAACGTGCACAGTGAGTGGTGTATCATTGCCTGATTATGG |
| | | CGTCTCCTGGATAAGGCAGCCCCCGCGAAAGGGTCTTGA |
| | | ATGGCTTGGGGTAATATGGGGCTCAGAGACAACGTATTA |
| | | TAACTCCGCTCTCAAAAGTCGCTTGACGATAATAAAAGA |
| | | TAACTCCAAGAGTCAAGTTTTCCTTAAAATGAACAGTTTG |
| | | CAGACTGACGATACCGCTATATATTATTGTGCTAAACATT |
| | | ATTACTACGGCGGTAGTTACGCGATGGATTATTGGGGGC |
| | | AGGGGACTTCTGTCACAGTCAGTAGT |
| 47 | CD19 scFv amino | DIQMTQTTSSLSASLGDRVTISCRASQDISKYLNWYQQKPD |
| | acid sequence | GTVKLLIYHTSRLHSGVPSRFSGSGSGTDYSLTISNLEQEDIA |
| | Linker underlined | TYFCQQGNTLPYTFGGGTKLEITGSTSGSGKPGSGEGSTKGE |
| | | VKLQESGPGLVAPSQSLSVTCTVSGVSLPDYGVSWIRQPPR |
| | | KGLEWLGVIWGSETTYYNSALKSRLTIIKDNSKSQVFLKMN |
| | | SLQTDDTAIYYCAKHYYYGGSYAMDYWGQGTSVTVSS |
| 48 | CD8a extracellular | GCTGCTGCCTTTGTCCCGGTATTTCTCCCAGCCAAACCGA |
| | CD8a transmembrane | CCACGACTCCCGCCCCGCGCCCTCCGACACCCGCTCCCAC |
| | 5′ Linker | CATCGCCTCTCAACCTCTTAGTCTTCGCCCCGAGGCATGC |
| | (underlined) | CGACCCGCCGCCGGGGGTGCTGTTCATACGAGGGGCTTG |
| | | GACTTCGCTTGTGATATTTACATTTGGGCTCCGTTGGCGG |
| | | GTACGTGCGGCGTCCTTTTGTTGTCACTCGTTATTACTTTG |
| | | TATTGTAATCACAGGAATCGC |
| 49 | CD8a extracellular | TTTGTCCCGGTATTTCTCCCAGCCAAACCGACCACGACTC |
| | CD8a transmembrane | CCGCCCCGCGCCCTCCGACACCCGCTCCCACCATCGCCTC |
| | (without linker) | TCAACCTCTTAGTCTTCGCCCCGAGGCATGCCGACCCGCC |
| | | GCCGGGGGTGCTGTTCATACGAGGGGCTTGGACTTCGCTT |
| | | GTGATATTTACATTTGGGCTCCGTTGGCGGGTACGTGCGG |
| | | CGTCCTTTTGTTGTCACTCGTTATTACTTTGTATTGTAATC |
| | | ACAGGAATCGC |
| 50 | CD8a extracellular | FVPVFLPAKPTTTPAPRPPTPAPTIASQPLSLRPEACRPAAGG |
| | CD8a transmembrane | AVHTRGLDFACDIYIWAPLAGTCGVLLLSLVITLYCNHRNR |
| 51 | CD19 VH | EVKLQESGPGLVAPSQSLSVTCTVSGVSLPDYGVSWIRQPPR |
| | | KGLEWLGVIWGSETTYYNSALKSRLTIIKDNSKSQVFLKMN |
| | | SLQTDDTAIYYCAKHYYYGGSYAMDYWGQGTSVTVSS |
| 52 | CD19 VL | DIQMTQTTSSLSASLGDRVTISCRASQDISKYLNWYQQKPD |
| | | GTVKLLIYHTSRLHSGVPSRFSGSGSGTDYSLTISNLEQEDIA |
| | | TYFCQQGNTLPYTFGGGTKLEIT |
| 53 | CD19 linker | GSTSGSGKPGSGEGSTKG |
| 54 | LHA to RHA | GAGATGTAAGGAGCTGCTGTGACTTGCTCAAGGCCTTAT |
| | | ATCGAGTAAACGGTAGTGCTGGGGCTTAGACGCAGGTGT |
| | | TCTGATTTATAGTTCAAAACCTCTATCAATGAGAGAGCAA |
| | | TCTCCTGGTAATGTGATAGATTTCCCAACTTAATGCCAAC |
| | | ATACCATAAACCTCCCATTCTGCTAATGCCCAGCCTAAGT |
| | | TGGGGAGACCACTCCAGATTCCAAGATGTACAGTTTGCTT |
| | | TGCTGGGCCTTTTTCCCATGCCTGCCTTTACTCTGCCAGA |
| | | GTTATATTGCTGGGGTTTTGAAGAAGATCCTATTAAATAA |
| | | AAGAATAAGCAGTATTATTAAGTAGCCCTGCATTTCAGGT |
| | | TTCCTTGAGTGGCAGGCCAGGCCTGGCCGTGAACGTTCAC |
| | | TGAAATCATGGCCTCTTGGCCAAGATTGATAGCTTGTGCC |
| | | TGTCCCTGAGTCCCAGTCCATCACGAGCAGCTGGTTTCTA |
| | | AGATGCTATTTCCCGTATAAAGCATGAGACCGTGACTTGC |
| | | CAGCCCCACAGAGCCCCGCCCTTGTCCATCACTGGCATCT |
| | | GGACTCCAGCCTGGGTTGGGGCAAAGAGGGAAATGAGAT |
| | | CATGTCCTAACCCTGATCCTCTTGTCCCACAGATATCCAG |
| | | AACCCTGACCCTGCCGTGTACCAGCTGAGAGACTCTAAA |
| | | TCCAGTGACAAGTCTGTCTGCCTATTCACCGATTTTGATT |
| | | CTCAAACAAATGTGTCACAAAGTAAGGATTCTGATGTGT |
| | | ATATCACAGACAAAACTGTGCTAGACATGAGGTCTATGG |
| | | ACTTCAGGCTCCGGTGCCCGTCAGTGGGCAGAGCGCACA |
| | | TCGCCCACAGTCCCCGAGAAGTTGGGGGGAGGGGTCGGC |
| | | AATTGAACCGGTGCCTAGAGAAGGTGGCGCGGGGTAAAC |
| | | TGGGAAAGTGATGTCGTGTACTGGCTCCGCCTTTTTCCCG |
| | | AGGGTGGGGGAGAACCGTATATAAGTGCAGTAGTCGCCG |
| | | TGAACGTTCTTTTTCGCAACGGGTTTGCCGCCAGAACACA |
| | | GGTAAGTGCCGTGTGTGGTTCCCGCGGGCCTGGCCTCTTT |
| | | ACGGGTTATGGCCCTTGCGTGCCTTGAATTACTTCCACTG |
| | | GCTGCAGTACGTGATTCTTGATCCCGAGCTTCGGGTTGGA |
| | | AGTGGGTGGGAGAGTTCGAGGCCTTGCGCTTAAGGAGCC |
| | | CCTTCGCCTCGTGCTTGAGTTGAGGCCTGGCCTGGGCGCT |
| | | GGGGCCGCCGCGTGCGAATCTGGTGGCACCTTCGCGCCT |
| | | GTCTCGCTGCTTTCGATAAGTCTCTAGCCATTTAAAATTTT |
| | | TGATGACCTGCTGCGACGCTTTTTTTCTGGCAAGATAGTC |
| | | TTGTAAATGCGGGCCAAGATCTGCACACTGGTATTTCGGT |
| | | TTTTGGGGCCGCGGGCGGCGACGGGGCCCGTGCGTCCCA |
| | | GCGCACATGTTCGGCGAGGCGGGGCCTGCGAGCGCGGCC |
| | | ACCGAGAATCGGACGGGGGTAGTCTCAAGCTGGCCGGCC |
| | | TGCTCTGGTGCCTGGCCTCGCGCCGCCGTGTATCGCCCCG |
| | | CCCTGGGCGGCAAGGCTGGCCCGGTCGGCACCAGTTGCG |
| | | TGAGCGGAAAGATGGCCGCTTCCCGGCCCTGCTGCAGGG |
| | | AGCTCAAAATGGAGGACGCGGCGCTCGGGAGAGCGGGC |
| | | GGGTGAGTCACCCACACAAAGGAAAAGGGCCTTTCCGTC |
| | | CTCAGCCGTCGCTTCATGTGACTCCACGGAGTACCGGGC |
| | | GCCGTCCAGGCACCTCGATTAGTTCTCGAGCTTTTGGAGT |
| | | ACGTCGTCTTTAGGTTGGGGGGAGGGGTTTTATGCGATGG |
| | | AGTTTCCCCACACTGAGTGGGTGGAGACTGAAGTTAGGC |
| | | CAGCTTGGCACTTGATGTAATTCTCCTTGGAATTTGCCCT |
| | | TTTTGAGTTTGGATCTTGGTTCATTCTCAAGCCTCAGACA |
| | | GTGGTTCAAAGTTTTTTTCTTCCATTTCAGGTGTCGTGACC |
| | | ACCATGCTTCTTTTGGTTACGTCTCTGTTGCTTTGCGAACT |
| | | TCCTCATCCAGCGTTCTTGCTGATCCCCGATATTCAGATG |
| | | ACTCAGACCACCAGTAGCTTGTCTGCCTCACTGGGAGAC |
| | | CGAGTAACAATCTCCTGCAGGGCAAGTCAAGACATTAGC |
| | | AAATACCTCAATTGGTACCAGCAGAAGCCCGACGGAACG |
| | | GTAAAACTCCTCATCTATCATACGTCAAGGTTGCATTCCG |
| | | GAGTACCGTCACGATTTTCAGGTTCTGGGAGCGGAACTG |
| | | ACTATTCCTTGACTATTTCAAACCTCGAGCAGGAGGACAT |
| | | TGCGACATATTTTTGTCAACAAGGTAATACCCTCCCTTAC |
| | | ACTTTCGGAGGAGGAACCAAACTCGAAATTACCGGGTCC |
| | | ACCAGTGGCTCTGGGAAGCCTGGCAGTGGAGAAGGTTCC |
| | | ACTAAAGGCGAGGTGAAGCTCCAGGAGAGCGGCCCCGGT |
| | | CTCGTTGCCCCCAGTCAAAGCCTCTCTGTAACGTGCACAG |
| | | TGAGTGGTGTATCATTGCCTGATTATGGCGTCTCCTGGAT |
| | | AAGGCAGCCCCCGCGAAAGGGTCTTGAATGGCTTGGGGT |
| | | AATATGGGGCTCAGAGACAACGTATTATAACTCCGCTCTC |
| | | AAAAGTCGCTTGACGATAATAAAAGATAACTCCAAGAGT |
| | | CAAGTTTTCCTTAAAATGAACAGTTTGCAGACTGACGATA |
| | | CCGCTATATATTATTGTGCTAAACATTATTACTACGGCGG |
| | | TAGTTACGCGATGGATTATTGGGGGCAGGGGACTTCTGTC |
| | | ACAGTCAGTAGTGCTGCTGCCTTTGTCCCGGTATTTCTCC |
| | | CAGCCAAACCGACCACGACTCCCGCCCCGCGCCCTCCGA |
| | | CACCCGCTCCCACCATCGCCTCTCAACCTCTTAGTCTTCG |
| | | CCCCGAGGCATGCCGACCCGCCGCCGGGGGTGCTGTTCA |
| | | TACGAGGGGCTTGGACTTCGCTTGTGATATTTACATTTGG |
| | | GCTCCGTTGGCGGGTACGTGCGGCGTCCTTTTGTTGTCAC |
| | | TCGTTATTACTTTGTATTGTAATCACAGGAATCGCTCAAA |
| | | GCGGAGTAGGTTGTTGCATTCCGATTACATGAATATGACT |
| | | CCTCGCCGGCCTGGGCCGACAAGAAAACATTACCAACCC |
| | | TATGCCCCCCCACGAGACTTCGCTGCGTACAGGTCCCGA |
| | | GTGAAGTTTTCCCGAAGCGCAGACGCTCCGGCATATCAG |
| | | CAAGGACAGAATCAGCTGTATAACGAACTGAATTTGGGA |
| | | CGCCGCGAGGAGTATGACGTGCTTGATAAACGCCGGGGG |
| | | AGAGACCCGGAAATGGGGGGTAAACCCCGAAGAAAGAA |
| | | TCCCCAAGAAGGACTCTACAATGAACTCCAGAAGGATAA |
| | | GATGGCGGAGGCCTACTCAGAAATAGGTATGAAGGGCGA |
| | | ACGACGACGGGGAAAAGGTCACGATGGCCTCTACCAAGG |
| | | GTTGAGTACGGCAACCAAAGATACGTACGATGCACTGCA |
| | | TATGCAGGCCCTGCCTCCCAGATAATAATAAAATCGCTAT |
| | | CCATCGAAGATGGATGTGTGTTGGTTTTTTGTGTGTGGAG |
| | | CAACAAATCTGACTTTGCATGTGCAAACGCCTTCAACAAC |
| | | AGCATTATTCCAGAAGACACCTTCTTCCCCAGCCCAGGTA |
| | | AGGGCAGCTTTGGTGCCTTCGCAGGCTGTTTCCTTGCTTC |
| | | AGGAATGGCCAGGTTCTGCCCAGAGCTCTGGTCAATGAT |
| | | GTCTAAAACTCCTCTGATTGGTGGTCTCGGCCTTATCCAT |
| | | TGCCACCAAAACCCTCTTTTTACTAAGAAACAGTGAGCCT |
| | | TGTTCTGGCAGTCCAGAGAATGACACGGGAAAAAAGCAG |
| | | ATGAAGAGAAGGTGGCAGGAGAGGGCACGTGGCCCAGC |
| | | CTCAGTCTCTCCAACTGAGTTCCTGCCTGCCTGCCTTTGCT |
| | | CAGACTGTTTGCCCCTTACTGCTCTTCTAGGCCTCATTCTA |
| | | AGCCCCTTCTCCAAGTTGCCTCTCCTTATTTCTCCCTGTCT |
| | | GCCAAAAAATCTTTCCCAGCTCACTAAGTCAGTCTCACGC |
| | | AGTCACTCATTAACCCACCAATCACTGATTGTGCCGGCAC |
| | | ATGAATGCACCAGGTGTTGAAGTGGAGGAATTAAAAAGT |
| | | CAGATGAGGGGTGTGCCCAGAGGAAGCACCATTCTAGTT |
| | | GGGGGAGCCCATCTGTCAGCTGGGAAAAGTCCAAATAAC |
| | | TTCAGATTGGAATGTGTTTTAACTCAGGGTTGAGAAAACA |
| | | GCTACCTTCAGGACAAAAGTCAGGGAAGGGCTCTCTGAA |
| | | GAAATGCTACTTGAAGATACCAGCCCTACCAAGGGCAGG |
| | | GAGAGGACCCTATAGAGGCCTGGGACAGGAGCTCAATGA |
| | | GAAAGG |
| 55 | spCas9 | MDKKYSIGLDIGTNSVGWAVITDEYKVPSKKFKVLGNTDR |
| | | HSIKKNLIGALLFDSGETAEATRLKRTARRRYTRRKNRICYL |
| | | QEIFSNEMAKVDDSFFHRLEESFLVEEDKKHERHPIFGNIVD |
| | | EVAYHEKYPTIYHLRKKLVDSTDKADLRLIYLALAHMIKFR |
| | | GHFLIEGDLNPDNSDVDKLFIQLVQTYNQLFEENPINASGVD |
| | | AKAILSARLSKSRRLENLIAQLPGEKKNGLFGNLIALSLGLTP |
| | | NFKSNFDLAEDAKLQLSKDTYDDDLDNLLAQIGDQYADLF |
| | | LAAKNLSDAILLSDILRVNTEITKAPLSASMIKRYDEHHQDL |
| | | TLLKALVRQQLPEKYKEIFFDQSKNGYAGYIDGGASQEEFY |
| | | KFIKPILEKMDGTEELLVKLNREDLLRKQRTFDNGSIPHQIHL |
| | | GELHAILRRQEDFYPFLKDNREKIEKILTFRIPYYVGPLARGN |
| | | SRFAWMTRKSEETITPWNFEEVVDKGASAQSFIERMTNFDK |
| | | NLPNEKVLPKHSLLYEYFTVYNELTKVKYVTEGMRKPAFLS |
| | | GEQKKAIVDLLFKTNRKVTVKQLKEDYFKKIECFDSVEISGV |
| | | EDRFNASLGTYHDLLKIIKDKDFLDNEENEDILEDIVLTLTLF |
| | | EDREMIEERLKTYAHLFDDKVMKQLKRRRYTGWGRLSRKL |
| | | INGIRDKQSGKTILDFLKSDGFANRNFMQLIHDDSLTFKEDIQ |
| | | KAQVSGQGDSLHEHIANLAGSPAIKKGILQTVKVVDELVKV |
| | | MGRHKPENIVIEMARENQTTQKGQKNSRERMKRIEEGIKEL |
| | | GSQILKEHPVENTQLQNEKLYLYYLQNGRDMYVDQELDIN |
| | | RLSDYDVDHIVPQSFLKDDSIDNKVLTRSDKNRGKSDNVPS |
| | | EEVVKKMKNYWRQLLNAKLITQRKFDNLTKAERGGLSELD |
| | | KAGFIKRQLVETRQITKHVAQILDSRMNTKYDENDKLIREV |
| | | KVITLKSKLVSDFRKDFQFYKVREINNYHHAHDAYLNAVV |
| | | GTALIKKYPKLESEFVYGDYKVYDVRKMIAKSEQEIGKATA |
| | | KYFFYSNIMNFFKTEITLANGEIRKRPLIETNGETGEIVWDKG |
| | | RDFATVRKVLSMPQVNIVKKTEVQTGGFSKESILPKRNSDK |
| | | LIARKKDWDPKKYGGFDSPTVAYSVLVVAKVEKGKSKKLK |
| | | SVKELLGITIMERSSFEKNPIDFLEAKGYKEVKKDLIIKLPKY |
| | | SLFELENGRKRMLASAGELQKGNELALPSKYVNFLYLASHY |
| | | EKLKGSPEDNEQKQLFVEQHKHYLDEIIEQISEFSKRVILADA |
| | | NLDKVLSAYNKHRDKPIREQAENIIHLFTLTNLGAPAAFKYF |
| | | DTTIDRKRYTSTKEVLDATLIHQSITGLYETRIDLSQLGGD |
| 56 | CD38 | MANCEFSPVSGDKPCCRLSRRAQLCLGVSILVLILVVVLAVVVPR |
| | | WRQQWSGPGTTKRFPETVLARCVKYTEIHPEMRHVDCQSVWDAF |
| | | KGAFISKHPCNITEEDYQPLMKLGTQTVPCNKILLWSRIKDLAHQF |
| | | TQVQRDMFTLEDTLLGYLADDLTWCGEFNTSKINYQSCPDWRKD |
| | | CSNNPVSVFWKTVSRRFAEAACDVVHVMLNGSRSKIFDKNSTFGS |
| | | VEVHNLQPEKVQTLEAWVIHGGREDSRDLCQDPTIKELESIISKRNI |
| | | QFSCKNIYRPDKFLQCVKNPEDSSCTSEI |
| 57 | daratumumab | EVQLLESGGGLVQPGGSLRLSCAVSGFTFNSFAMSWVRQAPGKGL |
| | heavy chain full | EWVSAISGSGGGTYYADSVKGRFTISRDNSKNTLYLQMNSLRAED |
| | sequence | TAVYFCAKDKILWFGEPVFDYWGQGTLVTVSSASTKGPSVFPLAP |
| | | SSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQ |
| | | DWLNGKEYKCKVSNKALPAPIEKTISKAKGQPREPQVYTLPPSREE |
| | | MTKNQVSLTCLVKGFYPSDIAVEWESNGQPENNYKTTPPVLDSDG |
| | | SFFLYSKLTVDKSRWQQGNVFSCSVMHEALHNHYTQKSLSLSPGK |
| 58 | daratumumab | EVQLLESGGGLVQPGGSLRLSCAVSGFTFNSFAMSWVRQAPGKGL |
| | heavy chain variable | EWVSAISGSGGGTYYADSVKGRFTISRDNSKNTLYLQMNSLRAED |
| | region | TAVYFCAKDKILWFGEPVFDYWGQGTLVTVSSAS |
| 59 | daratumumab | EIVLTQSPATLSLSPGERATLSCRASQSVSSYLAWYQQKPQAPRLLI |
| | light chain full | YDASNRATGIPARFSGSGSGTDFTLTISSLEPEDFAVYYCQQRSNW |
| | sequence | PPTFGQGTKVEIKRTVAAPSVFIFPPSDEQLKSGTASVVCLLNNFYP |
| | | REAKVQWKVDNALQSGNSQESVTEQDSKDSTYSLSSTLTLSKAD |
| | | YEKHKVYACEVTHQGLSSPVTKSFNRGEC |
| 60 | daratumumab | EIVLTQSPATLSLSPGERATLSCRASQSVSSYLAWYQQKPGQAPRL |
| | light chain variable | LIYDASNRATGIPARFSGSGSGTDFTLTISSLEPEDFAVYYCQQRSN |
| | region | WPPTFGQGTKVEIK |
| 61 | daratumumab | SFAMS |
| | heavy chain CDR1 | |
| 62 | daratumumab | AISGSGGGTYYADSVKG |
| | heavy chain CDR2 | |
| 63 | daratumumab | DKILWFGEPVFDY |
| | heavy chain CDR3 | |
| 64 | daratumumab | RASQSVSSYLA |
| | light chain CDR1 | |
| 65 | daratumumab | DASNRAT |
| | light chain CDR2 | |
| 66 | daratumumab | QQRSNWPPT |
| | light chain CDR3 | |

*indicates a nucleotide with a 2′-O-methyl phosphorothioate modification.
“n” refers to the spacer sequence at the 5′ end.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for treating a B-cell malignancy in a human patient, the method comprising:
(i) administering to a human patient having a first dose of B-cell malignancy a natural killer (NK) cell inhibitor; and
(ii) administering to the human patient a first dose of a population of genetically engineered T cells after step (ii), wherein the population of genetically engineered T cells comprising T cells that comprise (a) a nucleic acid coding for a chimeric antigen receptor (CAR) that binds CD19;
optionally wherein the genetically engineered T cells are deficient in expression of MHC Class-I.

2. The method of claim 1, the CAR comprises an anti-CD19 single chain variable fragment (scFv) that comprises the same heavy chain complementary determining regions (CDRs) as those in a heavy chain variable region set forth in SEQ ID NO: 51, and the same light chain CDRs as those in a light chain variable region set forth in SEQ ID NO: 52.

3. The method of claim 1, wherein the population of genetically engineered T cells comprise T cells that comprise (b) a disrupted beta 2-microglobulin (β2M) gene.

4. The method of claim 3, wherein the population of genetically engineered T cells comprising T cells that comprise (c) a disrupted T cell receptor alpha constant (TRAC) gene.

4. The method of claim 1, further comprising, between step (i) and step (ii), (iii) subjecting the human patient to a lymphodepletion treatment.

5. The method of claim 1, wherein the first dose of the population of genetically engineered T cells is administered to the human patient at a dose of about 1×10⁷ to about 1×10⁹ CAR⁺ T cells.

6. The method of claim 5, wherein the first dose of the population of genetically engineered T cells is about 3×10⁷, about 1×10⁸, about 3×10⁸, about 4.5×10⁸, about 6×10⁸, or about 9×10⁸ CAR⁺ T cells.

7. The method of claim 1, wherein the population of genetically engineered T cells administered to the human patient per dose contains no more than 7×10⁴ TCR⁺ T cells/kg.

8. The method of claim 1, wherein the NK cell inhibitor comprises an anti-CD38 antibody.

9. The method of claim 8, wherein the anti-CD38 antibody is daratumumab.

10. The method of claim 9, wherein the first dose of daratumumab is about 16 mg/kg via intravenous infusion.

11. The method of claim 10, wherein the first dose of daratumumab is about 16 mg/kg via intravenous infusion, which is administered to the human patient at 8 mg/kg per day for two consecutive days.

12. The method of claim 9, wherein the first dose of daratumumab is about 1800 mg via subcutaneous injection.

13. The method of claim 4, wherein the first dose of NK cell inhibitor is administered to the human patient at least one day prior to the lymphodepletion treatment.

14. The method of claim 4, wherein the first dose of the population of genetically engineered T cells is administered to the human patient within 10 days after the first dose of the NK cell inhibitor.

15. The method of claim 1, further comprising (iv) administering to the human patient at least one subsequent dose of the NK cell inhibitor after step (ii).

16. The method of claim 15, wherein step (iv) comprises administering to the human patient a second dose of the NK inhibitor 24 to 32 days after the first dose of the population of the genetically engineered T cells, and optionally a third dose of the NK inhibitor 56 to 64 days after the first dose of the population of genetically engineered T cells, when the human patient shows stable disease (SD) or better at least 4 weeks after the first dose of the population of genetically engineered T cells.

17. The method of claim 16, wherein the NK inhibitor is daratumumab and wherein the second dose thereof is 16 mg/kg by intravenous infusion or 1800 mg by subcutaneous injection, and/or the third dose thereof is 16 mg/kg by intravenous infusion or 1800 mg by subcutaneous injection.

18. The method of claim claim 4, wherein the lymphodepletion treatment in step (iii) comprises co-administration to the human patient fludarabine at about 30 mg/m² and cyclophosphamide at about 500 mg/m² per day for three days.

19. The method of claim 4, wherein prior to step (iii), the human patient does not show one or more of the following features:
(a) significant worsening of clinical status,
(b) requirement for supplemental oxygen to maintain a saturation level of greater than 91%,
(c) uncontrolled cardiac arrhythmia,
(d) hypotension requiring vasopressor support,
(e) active infection,
(f) grade ≥2 acute neurological toxicity, and
(g) unresolved infusion reaction due to the NK cell inhibitor.

20. The method of claim 4, wherein step (iii) is performed about 2-7 days prior to step (ii).

21. The method of claim 4, wherein after step (iii) and prior to step (ii), the human patient does not show one or more of the following features:
(a) active uncontrolled infection;
(b) worsening of clinical status compared to the clinical status prior to step (iii); and
(c) grade ≥2 acute neurological toxicity.

22. The method of claim 1, further comprising (v) monitoring the human patient for development of acute toxicity after step (ii); and (vi) managing the acute toxicity if the acute toxicity occurs.

23. The method of claim 22, wherein step (v) is performed for at least 28 days after administration of the population of genetically engineered T cells.

24. The method of claim 22, wherein the acute toxicity comprises tumor lysis syndrome (TLS), cytokine release syndrome (CRS), immune effector cellassociated neurotoxicity syndrome (ICANS), B cell aplasia, hemophagocytic lymphohistiocytosis (HLH), cytopenia, graft-versus-host disease (GvHD), hypertension, viral encephalitis, renal insufficiency, or a combination thereof.

25. The method of claim 1, wherein the B cell malignancy is non-Hodgkin lymphoma, which optionally is selected from the group consisting of diffuse large B cell lymphoma (DLBCL), high grade B cell lymphoma with MYC and BCL2 and/or BCL6 rearrangement, transformed follicular lymphoma (FL), and grade 3b FL.

26. The method of claim 25, wherein DLBCL is DLBCL not otherwise specified (NOS).

27. The method of claim 25, wherein the human patient has at least one measurable lesion that is fluorodeoxyglucose positron emission tomography (PET)-positive.

28. The method of claim 25, wherein the B cell malignancy is refractory and/or relapsed.

29. The method of claim 25, wherein the human patient has undergone one or more lines of prior anti-cancer therapies.

30. The method of claim 29, wherein the human patient has undergone two or more lines of prior anti-cancer therapies.

31. The method of claim 29 or claim 30, wherein the prior anti-cancer therapies comprise an anti-CD20 antibody, an anthracycline-containing regimen, or a combination thereof.

32. The method of claim 29, wherein the human patient has refractory or relapsed transformed FL and has undergone at least one line of chemotherapy for disease after transformation to DLBCL.

33. The method of claim 28, wherein the B cell malignancy is refractory, and the human patient has progressive disease on last therapy, or has stable disease following at least two cycles of therapy with duration of stable disease of up to 6 months.

34. The method of claim 25, wherein the human patient has failed prior autologous hematopoietic stem cell transplantation (HSCT) or ineligible for prior autologous HSCT.

35. The method of claim 25, wherein the human patient is subject to an additional anti-cancer therapy after treatment with the population of genetically engineered T cells.

36. The method of claim 25, wherein the human patient has one or more of the following features:
(a) has an Eastern Cooperative Oncology Group (ECOG) performance status 0 or 1;
(b) adequate renal, liver, cardiac, and/or pulmonary function;
(c) free of prior gene therapy or modified cell therapy;
(d) free of prior treatment comprising an anti-CD19 antibody;
(e) free of prior allogeneic HSCT;
(f) free of detectable malignant cells from cerebrospinal fluid;
(g) free of brain metastases;
(h) free of prior central nervous system disorders;
(i) free of unstable angina, arrhythmia, and/or myocardial infarction;
(j) free of uncontrolled infection;
(k) free of immunodeficiency disorders or autoimmune disorders that require immunosuppressive therapy; and
(l) free of infection by human immunodeficiency virus, hepatitis B virus, or hepatitis C virus.

37. The method of claim 25, wherein the human patient is not diagnosed for Burkitt's lymphoma or leukemia.

38. The method of claim 25, further comprising administering to the human patient at least one subsequent dose of the population of genetically engineered T cells.

39. The method of claim 25, wherein the first dose of the population of genetically engineered T cells is at least 3×10⁸ CAR⁺ T cells.

40. The method of claim 38, wherein the human patient receives a second dose of the population of genetically engineered T cells about 4-8 weeks after the first dose of the population of genetically engineered T cells.

41. The method of claim 40, wherein the human patient achieves stable disease (SD), particle response (PR), or complete response (CR) at least 4 weeks after the first dose.

42. The method of claim 38, wherein the human patient receives a subsequent lymphodepletion treatment about 2-7 days prior to each of the subsequent dose of the population of the genetically engineered T cells.

43. The method of claim 38, wherein the human patient experiences significant cytopenias after steps (i)-(iii), and wherein the human patient does not receive subsequent lymphodepletion treatment prior to each of the subsequent dose of the population of the genetically engineered T cells.

44. The method of claim 25, wherein the first dose of the population of genetically engineered T cells is at least 4.5×10⁸ CAR⁺ T cells or at least 6×10⁸ CAR⁺ T cells.

45. The method of claim 38, wherein the human patient receives a second dose of the population of genetically engineered T cells about 7-9 days after the first dose of the population of genetically engineered T cells; and wherein the human patient does not receive a subsequent lymphodepletion treatment prior to the second dose of the population of genetically engineered T cells.

46. The method of claim 45, wherein the human patient receives a third dose of the population of genetically engineered T cells about 4-8 weeks after the first dose of the population of genetically engineered T cells.

47. The method of claim 46, wherein the human patient achieves stable disease (SD), particle response (PR), or complete response (CR) at least 4 weeks after the first dose.

48. The method of claim 46, wherein the human patient receives a subsequent lymphodepletion treatment about 2-7 days prior to the third dose of the population of the genetically engineered T cells.

49. The method of claim 46, wherein the human patient experiences significant cytopenias after steps (i)-(iii), and wherein the human patient does not receive subsequent lymphodepletion treatment prior to the third dose of the population of the genetically engineered T cells.

50. The method of claim 38 wherein the human patient does not receive subsequent doses of the NK inhibitor.

51. The method of claim 38, wherein the subsequent dose(s) is about 3×10⁷, about 1×10⁸, about 3×10⁸, about 4.5×10⁸, about 6×10⁸, or about 9×10⁸ CAR+ T cells.

52. The method of claim 1, wherein the human patient has B-cell acute lymphoblastic leukemia (ALL).

53. The method of claim 52, wherein the human patient has refractory and/or relapsed B cell ALL.

54. The method of claim 52, wherein the human patient
(a) has undergone two or more lines of prior anti-cancer therapies;
(b) has bone marrow relapse after allogeneic hematopoietic stem cell transplantation (HSCT);
(c) is Philadelphia chromosome-positive (Ph+), and is intolerant to or ineligible for tyrosine kinase inhibitor (TKI) therapy, or has progressed after at least 1 line of TKI therapy;
(d) has bone marrow involvement with <50% blasts; and/or
(e) is bone marrow minimal residue disease (MRD) positive with <5% blasts.

55. The method of claim 52, wherein the human patient has one or more of the following features:
(a) has an Eastern Cooperative Oncology Group (ECOG) performance status 0 or 1;
(b) adequate renal, liver, cardiac, and/or pulmonary function;
(c) free of prior gene therapy or modified cell therapy;
(d) free of prior treatment comprising an anti-CD19 antibody;
(e) free of prior allogeneic HSCT;
(f) free of detectable malignant cells from cerebrospinal fluid;
(g) free of brain metastases;
(h) free of prior central nervous system disorders;
(i) free of unstable angina, arrhythmia, and/or myocardial infarction;
(j) free of uncontrolled infection;
(k) free of immunodeficiency disorders or autoimmune disorders that require immunosuppressive therapy;
(l) free of infection by human immunodeficiency virus, hepatitis B virus, or hepatitis C virus; and
(m) free of extramedullary disease.

56. The method of claim 52, further comprising administering to the human patient at least one subsequent dose of the population of genetically engineered T cells.

57. The method of claim 52, wherein the first dose of the population of genetically engineered T cells is at least 3×10⁷ CAR⁺ T cells, 1×10⁸ CAR⁺ T cells or at least 3×10⁸ CAR⁺ T cells.

58. The method of claim 56, wherein the human patient has a decrease in bone marrow blast count of at least 50% about 4 weeks after the first dose of the population of genetically engineered T cells.

59. The method of claim 56, wherein the human patient receives a second dose of the population of genetically engineered T cells about 4 to 8 weeks after the first dose of the population of genetically engineered T cells; optionally wherein the second dose is administered to the subject about 4 weeks after the first dose.

60. The method of claim 59, wherein the human patient is in a morphologic remission and is MRD-remains positive.

61. The method of claim 56, wherein the human patient shows progressive disease (PD) and had prior response.

62. The method of claim 56, wherein the human patient receives a subsequent lymphodepletion treatment about 2-7 days prior to each of the subsequent dose of the population of the genetically engineered T cells.

63. The method of claim 56, wherein the human patient experiences significant cytopenias after steps (i)-(iii), and wherein the human patient does not receive subsequent lymphodepletion treatment prior to each of the subsequent dose of the population of the genetically engineered T cells.

64. The method of claim 56, wherein the subsequent dose(s) is about 3×10⁷, about 1×10⁸, about 3×10⁸, about 4.5×10⁸, about 6×108, or about 9×10⁸ CAR+ T cells.

65. The method of claim 1, wherein the CAR comprises an anti-CD19 single chain variable fragment (scFv) that comprises a heavy chain variable region set forth in SEQ ID NO: 51, and a light chain variable region set forth in SEQ ID NO: 52.

66. The method of claim 65, wherein the anti-CD19 scFv comprises the amino acid sequence of SEQ ID NO: 47.

67. The method of claim 66, wherein the CAR that binds CD19 comprises the amino acid sequence of SEQ ID NO: 40.

68. The method of claim 3, wherein the nucleic acid encoding the CAR is inserted in the disrupted TRAC gene.

69. The method of claim 3, wherein the disrupted TRAC gene comprises a deletion of a fragment comprising the nucleotide sequence of SEQ ID NO: 26.

70. The method of claim 69, wherein the nucleic acid encoding the anti-CD19 CAR is inserted at the site of the deletion in the disrupted TRAC gene.

71. The method of claim 70, wherein the disrupted TRAC gene comprises the nucleotide sequence of SEQ ID NO: 54.

72. The method of claim 3, wherein the disrupted β2M gene in the population of genetically engineered T cells comprises at least one of the nucleotide sequence set forth in SEQ ID NOs: 9-14.

73. The method of claim 1, wherein the population of the genetically engineered T cells is allogeneic to the human patient.

74. The method of claim 1, wherein at least 90% of the T cells in the population of genetically engineered T cells do not express a detectable level of TCR surface protein.

75. The method of claim 1, wherein at least 70% of the T cells in the population of genetically engineered T cells do not express a detectable level of TCR surface protein; wherein at least 50% of the T cells in the population of genetically engineered T cells do not express a detectable level of B2M surface protein; and/or wherein at least 30% of the T cells in the population of genetically engineered T cells express a detectable level of the CAR.

76. The method of claim 75, wherein at least 99.5% of the T cells in the population of genetically engineered T cells do not express a detectable level of TCR surface protein.

77. The method of claim 1, wherein at least 70% of the T cells in the population of genetically engineered T cells do not express a detectable level of B2M surface protein.

78. The method of claim 77, wherein at least 85% of the T cells in the population of the genetically engineered T cells do not express a detectable level of B2M surface protein.

79. The method of claim 1, wherein at least 50% of the T cells in the population of genetically engineered T cells express a detectable level of the CAR.

80. The method of claim 79, wherein at least 70% of the T cells in the population of genetically engineered T cells express a detectable level of the CAR.

81. The method of claim 1, wherein the population of genetically engineered T cells are administered to the human patient via intravenous infusion.

82. The method of claim 1, wherein the population of genetically engineered T cells are suspended in a cryopreservation solution.

83. A method for treating acute lymphoblastic leukemia (ALL) in a human patient, the method comprising:
(i) subjecting a human patient having ALL to a lymphodepletion treatment; and
(ii) administering to the human patient a first dose of a population of genetically engineered T cells after step (i),
wherein the population of genetically engineered T cells comprising T cells comprise (a) a nucleic acid coding for a chimeric antigen receptor (CAR) that binds CD19; and
wherein the first dose of the population of genetically engineered T cells is administered to the human patient at a dose of about 1×10⁷ to about 1×10⁹ CAR+ T cells.

84. The method of claim 83, wherein the CAR comprises an anti-CD19 single chain variable fragment (scFv) that comprises the same heavy chain complementary determining regions (CDRs) as those in a heavy chain variable region set forth in SEQ ID NO: 51, and the same light chain CDRs as those in a light chain variable region set forth in SEQ ID NO: 52.

85. The method of claim 83, wherein the population of genetically engineered T cells comprising T cells that comprise (b) a disrupted T cell receptor alpha constant (TRAC) gene, and/or (c) a disrupted beta 2-microglobulin (β2M) gene.

86. The method of claim 85, wherein the population of genetically engineered T cells comprising T cells that comprise (b) a disrupted T cell receptor alpha constant (TRAC) gene, and (c) a disrupted beta 2-microglobulin (β2M) gene.

87. The method of claim 83, wherein the first dose of the population of genetically engineered T cells is about 3×10⁷, about 1×10⁸, about 3×10⁸, about 4.5×10⁸, about 6×10⁸, or about 9×10⁸ CAR⁺ T cells.

88. The method of claim 83, wherein the first dose of the population of the genetically engineered T cells is at least 1×10⁸ or at least about 3×10⁸ CAR⁺ T cells.

89. The method of claim 83, wherein the population of genetically engineered T cells administered to the human patient per dose contains no more than 7×10⁴ TCR⁺ T cells/kg.

90. The method of claim claim 83, wherein the lymphodepletion treatment in step (i) comprises co-administration to the human patient fludarabine at about 30 mg/m² and cyclophosphamide at about 500 mg/m² per day for three days.

91. The method of claim 83, wherein prior to step (i), the human patient does not show one or more of the following features:
(a) significant worsening of clinical status,
(b) requirement for supplemental oxygen to maintain a saturation level of greater than 91%,
(c) uncontrolled cardiac arrhythmia,
(d) hypotension requiring vasopressor support,
(e) active infection, and
(f) grade ≥2 acute neurological toxicity.

92. The method of claim 83, wherein step (i) is performed about 2-7 days prior to step (ii).

93. The method of claim 83, wherein after step (i) and prior to step (ii), the human patient does not show one or more of the following features:
(a) active uncontrolled infection;
(b) worsening of clinical status compared to the clinical status prior to step (i); and
(c) grade ≥2 acute neurological toxicity.

94. The method of claim 83, further comprising (iii) monitoring the human patient for development of acute toxicity after step (ii); and (iv) managing the acute toxicity if occurs.

95. The method of claim 94, wherein step (iii) is performed for at least 28 days after the first dose of the population of genetically engineered T cells.

96. The method of claim 94, wherein the acute toxicity comprises tumor lysis syndrome (TLS), cytokine release syndrome (CRS), immune effector cell-associated neurotoxicity syndrome (ICANS), B cell aplasia, hemophagocytic lymphohistiocytosis (HLH), cytopenia, graft-versus-host disease (GvHD), hypertension, renal insufficiency, viral encephalitis, or a combination thereof.

97. The method of claim 83, wherein the human patient has B-cell ALL.

98. The method of claim 97, wherein the human patient has refractory and/or relapsed B cell ALL.

99. The method of claim 98, wherein the human patient:
(a) has undergone two or more lines of prior anti-cancer therapies;
(b) has bone marrow relapse after allogeneic hematopoietic stem cell transplantation (HSCT);
(c) is Philadelphia chromosome-positive (Ph+), and is intolerant to or ineligible for tyrosine kinase inhibitor (TKI) therapy, or has progressed after at least 1 line of TKI therapy;
(d) has bone marrow involvement with <50% blasts; and/or
(e) is bone marrow minimal residue disease (MRD) positive with <5% blasts.

100. The method of claim 99, wherein the prior anti-cancer therapies comprise a chemotherapy, an allogeneic stem cell transplantation, or a combination thereof; optionally wherein the chemotherapy comprises vincristine, corticosteroids, an anthracycline-containing regimen, or a combination thereof.

101. The method of claim 83, wherein the human patient is subject to an additional anti-cancer therapy after treatment with the population of genetically engineered T cells.

102. The method of claim 83, wherein the human patient has one or more of the following features:
(a) has an Eastern Cooperative Oncology Group (ECOG) performance status 0 or 1;
(b) adequate renal, liver, cardiac, and/or pulmonary function;
(c) free of prior gene therapy or modified cell therapy;
(d) free of prior treatment comprising an anti-CD19 antibody;
(e) free of prior allogeneic HSCT;
(f) free of detectable malignant cells from cerebrospinal fluid;
(g) free of brain metastases;
(h) free of prior central nervous system disorders;
(i) free of unstable angina, arrhythmia, and/or myocardial infarction;
(j) free of uncontrolled infection;
(k) free of immunodeficiency disorders or autoimmune disorders that require immunosuppressive therapy;
(l) free of infection by human immunodeficiency virus, hepatitis B virus, or hepatitis C virus;
(m) free of Burkitt's lymphoma or leukemia; and
(n) free of isolated extramedullary disease.

103. The method of claim 83, further comprising (iii) administering to the human patient at least one subsequent dose of the population of genetically engineered T cells.

104. The method of claim 103, wherein the human patient has a decrease in bone marrow blast count of at least 50% about 4 weeks after the first dose of the population of genetically engineered T cells.

105. The method of claim 103, wherein the human patient receives a second dose of the population of genetically engineered T cells about 4 to 8 weeks after the first dose of the population of genetically engineered T cells.

106. The method of claim 105, wherein the human patient is in a morphologic remission and is MRD-remains positive.

107. The method of claim 103, wherein the human patient shows progressive disease (PD) and had prior response.

108. The method of claim 103, wherein the human patient receives a subsequent lymphodepletion treatment about 2-7 days prior to each of the subsequent dose of the population of the genetically engineered T cells.

109. The method of claim 103, wherein the human patient experiences significant cytopenias after steps (i)-(iii), and wherein the human patient does not receive subsequent lymphodepletion treatment prior to each of the subsequent dose of the population of the genetically engineered T cells.

110. The method of claim 103, wherein the subsequent dose(s) is about 3×10⁷, about 1×10⁸, about 3×10⁸, about 4.5×10⁸, about 6×10⁸, or about 9×10⁸ CAR⁺ T cells.

111. The method of claim 83, wherein the CAR comprises an anti-CD19 single chain variable fragment (scFv) that comprises a heavy chain variable region set forth in SEQ ID NO: 51, and a light chain variable region set forth in SEQ ID NO: 52.

112. The method of claim 111, wherein the anti-CD19 scFv comprises the amino acid sequence of SEQ ID NO: 47.

113. The method of claim 112, wherein the CAR that binds CD19 comprises the amino acid sequence of SEQ ID NO: 40.

114. The method of claim 86, wherein the disrupted TRAC gene comprises a deletion of a fragment comprising the nucleotide sequence of SEQ ID NO: 26.

115. The method of claim 114, wherein the nucleic acid encoding the anti-CD19 CAR is inserted at the site of the deletion in the disrupted TRAC gene.

116. The method of claim 115, wherein the disrupted TRAC gene comprises the nucleotide sequence of SEQ ID NO: 54.

117. The method of claim 86, wherein the disrupted 62M gene in the population of genetically engineered T cells comprises at least one of the nucleotide sequence set forth in SEQ ID NOs: 9-14.

118. The method of claim 83, wherein the population of genetically engineered T cells is allogeneic to the human patient.

119. The method of claim 83, wherein at least 90% of the T cells in the population of genetically engineered T cells do not express a detectable level of TCR surface protein.

120. The method of claim 83, wherein at least 70% of the T cells in the population of genetically engineered T cells do not express a detectable level of TCR surface protein, wherein at least 50% of the T cells in the population of genetically engineered T cells do not express a detectable level of B2M surface protein; and/or wherein at least 30% of the T cells in the population of genetically engineered T cells express a detectable level of the CAR.

121. The method of claim 120, wherein at least 99.5% of the T cells in the population of genetically engineered T cells do not express a detectable level of TCR surface protein.

122. The method of claim 83, wherein at least 70% of the T cells in the population of genetically engineered T cells do not express a detectable level of B2M surface protein.

123. The method of claim 122, wherein at least 85% of the T cells in the population of the genetically engineered T cells do not express a detectable level of B2M surface protein.

124. The method of claim 83, wherein at least 50% of the T cells in the population of genetically engineered T cells express a detectable level of the CAR.

125. The method of claim 124, wherein at least 70% of the T cells in the population of genetically engineered T cells express a detectable level of the CAR.

126. The method of claim 83, wherein the population of genetically engineered T cells are administered to the human patient via intravenous infusion.

127. The method of claim 83, wherein the population of genetically engineered T cells are suspended in a cryopreservation solution.
